(19) United States
(12) Patent Application Publication
(10) Pub. No.: US 2021/0300943 A1
(43) Pub. Date: Sep. 30, 2021

(54) OGA INHIBITOR COMPOUNDS

(71) Applicant: Janssen Pharmaceutica NV, Beerse (BE)

(72) Inventors: José Manuel Bartolomé-Nebreda, Toledo (ES); Andrés Avellno Trabanco-Suárez, Olias del Rey (ES); Carlos Manuel Martinez-Viturro, Olias del Rey (ES); Francisca Deigado-Jiménez, Madrid (ES); Susana Coride-Ceide, Toledo (ES); Juan Antonio Vega Ramiro, Olias del Rey (ES)

(21) Appl. No.: 17/253,432

(22) PCT Filed: Jun. 20, 2019
(86) PCT No.: PCT/EP2019/066384

§ 371 Date: Dec. 17, 2020

(30) Foreign Application Priority Data
Jun. 20, 2018 (EP) 183824467.3

(51) Int. Cl. C07D 498/04; C07D 491/056; C07D 405/14; C07D 491/048

(52) U.S. Cl. CPC C07D 498/04; C07D 491/048; C07D 405/14; C07D 491/056

(57) ABSTRACT

The present invention relates to O-GlcNAc hydrolase (OGA) inhibitors. The invention is also directed to pharmaceutical compositions comprising such compounds, to processes for preparing such compounds and compositions, and to the use of such compounds and compositions for the prevention and treatment of disorders in which inhibition of OGA is beneficial, such as tauopathies, in particular Alzheimer's disease or progressive supranuclear palsy; and neurodegenerative diseases accompanied by a tau pathology, in particular amyotrophic lateral sclerosis or frontotemporal lobe dementia caused by C9ORF72 mutations.

### FIELD OF THE INVENTION

The present invention relates to O-GlcNAc hydrolase (OGA) inhibitors, having the structure shown in Formula (I)

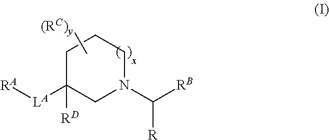

wherein the radicals are as defined in the specification. The invention is also directed to pharmaceutical compositions comprising such compounds, to processes for preparing such compounds and compositions, and to the use of such compounds and compositions for the prevention and treatment of disorders in which inhibition of OGA is beneficial, such as tauopathies, in particular Alzheimer's disease or progressive supranuclear palsy; and neurodegenerative diseases accompanied by a tau pathology, in particular amyotrophic lateral sclerosis or frontotemporal lobe dementia caused by C9ORF72 mutations.

### BACKGROUND OF THE INVENTION

O-GlcNAcylation is a reversible modification of proteins where N-acetyl-D-glucosamine residues are transferred to the hydroxyl groups of serine- and threonine residues yield O-GlcNAcylated proteins. More than 1000 of such target proteins have been identified both in the cytosol and nucleus of eukaryotes. The modification is thought to regulate a huge spectrum of cellular processes including transcription, cytoskeletal processes, cell cycle, proteasomal degradation, and receptor signalling.

O-GlcNAc transferase (OGT) and O-GlcNAc hydrolase (OGA) are the only two proteins described that add (OGT) or remove (OGA) O-GlcNAc from target proteins. OGA was initially purified in 1994 from spleen preparation and 1998 identified as antigen expressed by meningiomas and termed MGEA5, consists of 916 amino (102915 Dalton) as a monomer in the cytosolic compartment of cells. It is to be distinguished from ER- and Golgi-related glycosylation processes that are important for trafficking and secretion of proteins and different to OGA have an acidic pH optimum, whereas OGA display highest activity at neutral pH.

The OGA catalytic domain with its double aspartate catalytic center resides in the N-terminal part of the enzyme which is flanked by two flexible domains. The C-terminal part consists of a putative HAT (histone acetyl transferase domain) preceded by a stalk domain. It has yet still to be proven that the HAT-domain is catalytically active.

O-GlcNAcylated proteins as well as OGT and OGA themselves are particularly abundant in the brain and neurons suggesting this modification plays an important role in the central nervous system. Indeed, studies confirmed that G-GlcN Acylation represents a key regulatory mechanism contributing to neuronal communication, memory formation and neurodegenerative disease. Moreover, it has been shown that OGT is essential for embryogenesis in several animal models and ogt null mice are embryonic lethal. OGA is also indispensable for mammalian development. Two independent studies have shown that OGA homozygous null mice do not survive beyond 24-48 hours after birth. Oga deletion has led to defects in glycogen mobilization in pups and it caused genomic instability linked cell cycle arrest in MEFs derived from homozygous knockout embryos. The heterozygous animals survived to adulthood however they exhibited alterations in both transcription and metabolism.

It is known that perturbations in O-GlcNAc cycling impact chronic metabolic diseases such as diabetes, as well as cancer. Oga heterozygosity suppressed intestinal tumorigenesis in an Apc−/+ mouse cancer model and the Oga gene (MGEA5) is a documented human diabetes susceptibility locus.

In addition, O-GlcNAc-modifications have been identified on several proteins that are involved in the development and progression of neurodegenerative diseases and a correlation between variations of O-GlcNAc levels on the formation of neurofibrillary tangle (NFT) protein by Tau in Alzheimer's disease has been suggested. In addition, O-GlcNAcylation of alpha-synuclein in Parkinson's disease has been described.

In the central nervous system six splice variants of tau have been described. Tau is encoded on chromosome 17 and consists in its longest splice variant expressed in the central nervous system of 441 amino acids. These isoforms differ by two N-terminal inserts (exon 2 and 3) and exon 10 which lie within the microtubule binding domain. Exon 10 is of considerable interest in tauopathies as it harbours multiple mutations that render tau prone to aggregation as described below. Tau protein binds to and stabilizes the neuronal microtubule cytoskeleton which is important for regulation of the intracellular transport of organelles along the axonal compartments. Thus, tau plays an important role in the formation of axons and maintenance of their integrity. In addition, a role in the physiology of dendritic spines has been suggested as well.

Tau aggregation is either one of the underlying causes for a variety of so called tauopathies like PSP (progressive supranuclear palsy), Down's syndrome (DS), FTLD (frontotemporal lobe dementia), FTDP-17 (frontotemporal dementia with Parkinsonism-17), Pick's disease (PD), CBD (corticobasal degeneration), agryophilic grain disease (AGD), and AD (Alzheimer's disease). In addition, tau pathology accompanies additional neurodegenerative diseases like amyotrophic lateral sclerosis (ALS) or FTLD cause by C9ORF72 mutations. In these diseases, tau is post-translationally modified by excessive phosphorylation which is thought to detach tau from microtubules and makes it prone to aggregation. O-GlcNAcylation of tau regulates the extent of phosphorylation as serine or threonine residues carrying O-GlcNAc-residues are not amenable to phosphorylation. This effectively renders tau less prone to detaching from microtubules and reduces aggregation into neurotoxic tangles which ultimately lead to neurotoxicity and neuronal cell death. This mechanism may also reduce the cell-to-cell spreading of tau-aggregates released by neurons via along interconnected circuits in the brain which has recently been discussed to accelerate pathology in tau-related dementias. Indeed, hyperphosphorylated tau isolated from brains of AD-patients showed significantly reduced O-GlcNAcylation levels.

An OGA inhibitor administered to JNPL3 tau transgenic mice successfully reduced NFT formation and neuronal loss without apparent adverse effects. This observation has been confirmed in another rodent model of tauopathy where the expression of mutant tau found in FTD can be induced (tg4510). Dosing of a small molecule inhibitor of OGA was efficacious in reducing the formation of tau-aggregation and attenuated the cortical atrophy and ventricle enlargement.

Moreover, the O-GlcNAcylation of the amyloid precursor protein (APP) favours processing via the non-amyloidogenic route to produce soluble APP fragment and avoid cleavage that results in the AD associated amyloid-beta (Aβ) formation.

Maintaining O-GlcNAcylation of tau by inhibition of OGA represents a potential approach to decrease tau-phosphorylation and tau-aggregation in neurodegenerative diseases mentioned above thereby attenuating or stopping the progression of neurodegenerative tauopathy-diseases.

WO2012/117219 (Summit Corp. plc., published 7 Sep. 2012) describes N-[[5-(hydroxymethyl)pyrrohdin-2-yl]methyl]alkylamide and N-alkyl-2-[5-(hydroxymethyl)pyrrolidin-2-yl]acetamide derivatives as OGA inhibitors. WO2014/159234 (Merck Patent GMBH, published 2 Oct. 2014) discloses mainly 4-phenyl or benzyl-piperidine and piperazine compounds substituted at the 1-position with an acetamido-thiazolylmethyl or acetamidoxazolylmethyl substituent and the compound N-[5-[(3-phenyl-1-piperidyl)methyl]thiazol-2-yl]acetamide; WO2016/0300443 (Asceneuron S. A., published 3 Mar. 2016), WO2017/144633 and WO2017/0114639 (Asceneuron S. A., published 31 Aug. 2017) disclose 1,4-disubstituted piperidines or piperazines as OGA inhibitors; WO2017/144637 (Asceneuron S. A, published 31 Aug. 2017) discloses more particular 4-substituted 1-[1-(1,3-benzodioxol-5-yl)ethyl]-piperazine; 1-[1-(2,3-dihydrobenzofuran-5-yl)ethyl]-; 1-[1-(2,3-dihydrobenzofuran-6-yl)ethyl]-; and 1-[1-(2,3-dihydro-1,4-benzodioxin-6-yl)ethyl]-piperazine derivatives as OGA inhibitors; WO2017/106254 (Merck Sharp & Dohme Corp.) describes substituted N-[5-[(4-methylene-1-piperidyl)methyl]thiazol-2-yl]acetamide compounds as OGA inhibitors.

There is still a need for OGA inhibitor compounds with an advantageous balance of properties, for example with improved potency, good bioavailability, pharmacokinetics, and brain penetration, and/or better toxicity profile. It is accordingly an object of the present invention to provide compounds that overcome at least some of these problems.

### SUMMARY OF THE INVENTION

The present invention is directed to compounds of Formula (I)

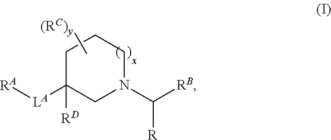

and the tautomers and the stereoisomeric forms thereof, wherein

R^{A} is a heteroaryl radical selected from the group consisting of pyridin-2-yl, pyridin-3-yl, pyridin-4-yl, pyridazin-3-yl, pyrimidin-4-yl, pyrimidin-5-yl, and pyrazin-2-yl; or is an aryl radical selected from phenyl; each of which may be optionally substituted with 1, 2 or 3 substituents each independently selected from the group consisting of halo; cyano; C₁₋₄alkyl optionally substituted with 1, 2, or 3 independently selected halo substituents; —C(O)NR^{a}R^{aa}; NR^{a}R^{aa}; and C₁₋₄alkyloxy optionally substituted with 1, 2, or 3 independently selected halo substituents; wherein

R^{a} and R^{aa} are each independently selected from the group consisting of hydrogen and C₁₋₄alkyl optionally substituted with 1, 2, or 3 independently selected halo substituents;

L^{A} is selected from the group consisting of a covalent bond, —CH₂—, —O—, —OCH₂—, —CH₂O—, —NH—, —N(CH₃)—, —NHCH₂— and —CH₂NH—;

x represents 1;

R is H or CH₃; and

R^{B} is a bicyclic radical of formula (b-1), (b-2) or (b-3)

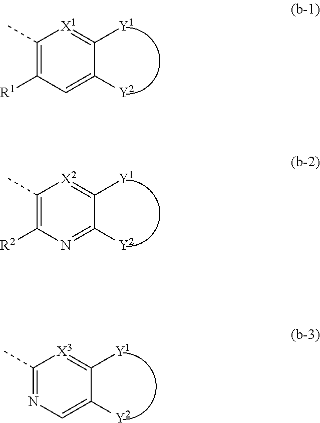

wherein

R¹ and R² are each selected from the group consisting of hydrogen, fluoro and methyl;

X¹, X² and X³ each represent CH, CF or N;

—Y¹—Y²— forms a bivalent radical selected from the group consisting of

—O(CH₂)ₘO— (c-1);

—O(CH₂)ₙ— (c-2);

—(CH₂)ₙO— (c-3);

—O(CH₂)ₚNR³— (c-4);

—NR³(CH₂)_{P}O— (c-5);

—O(CH₂)(CO)NR³— (c-6);

—NR³(CO)(CH₂)O— (c-7);

—(CH₂)ₙNR³(CO)— (c-8);

—(CO)NR³(CH₂)ₙ— (c-9); and

—N═CH(CO)NR³— (c-10);

wherein

m is 1 or 2;

n and p each independently represent 2 or 3;

each R³ is independently H or C₁₋₄alkyl;

R^{C} is selected from the group consisting of fluoro, methyl, hydroxy, methoxy, trifluoromethyl, and difluoromethyl;

R^{D} is selected from the group consisting of hydrogen, fluoro, methyl, hydroxy, methoxy, trifluoromethyl, and difluoromethyl; and

y represents 0, 1 or 2;

with the provisos that
a) R^{C} is not hydroxy or methoxy when present at the carbon atom adjacent to the nitrogen atom of the piperidinediyl or pyrrolidinediyl ring;
b) R^{C} or R^{D} cannot be selected simultaneously from hydroxy or methoxy when R^{C} is present at the carbon atom adjacent to C—R^{D};
c) R^{D} is not hydroxy or methoxy when L^{A} is —O—, —OCH₂—, —CH₂O—, —NH—, —N(CH₃)—, —NH(CH₂)— or —(CH₂)NH—;

and the pharmaceutically acceptable salts and the solvates thereof.

Illustrative of the invention is a pharmaceutical composition comprising a pharmaceutically acceptable carrier and any of the compounds described above. An illustration of the invention is a pharmaceutical composition made by mixing any of the compounds described above and a pharmaceutically acceptable carrier. Illustrating the invention is a process for making a pharmaceutical composition comprising mixing any of the compounds described above and a pharmaceutically acceptable carrier.

Exemplifying the invention are methods of preventing or treating a disorder mediated by the inhibition of O-GlcNAc hydrolase (OGA), comprising administering to a subject in need thereof a therapeutically effective amount of any of the compounds or pharmaceutical compositions described above.

Further exemplifying the invention are methods of inhibiting OGA, comprising administering to a subject in need thereof a prophylactically or a therapeutically effective amount of any of the compounds or pharmaceutical compositions described above.

An example of the invention is a method of preventing or treating a disorder selected from a tauopathy, in particular a tauopathy selected from the group consisting of Alzheimer's disease, progressive supranuclear palsy, Down's syndrome, frontotemporal lobe dementia, frontotemporal dementia with Parkinsonism-17, Pick's disease, corticobasal degeneration, and agryophilic grain disease; or a neurodegenerative disease accompanied by a tau pathology, in particular a neurodegenerative disease selected from amyotrophic lateral sclerosis or frontotemporal lobe dementia caused by C9ORF72 mutations, comprising administering to a subject in need thereof, a prophylactically or a therapeutically effective amount of any of the compounds or pharmaceutical compositions described above.

Another example of the invention is any of the compounds described above for use in preventing or treating a tauopathy, in particular a tauopathy selected from the group consisting of Alzheimer's disease, progressive supranuclear palsy, Down's syndrome, frontotemporal lobe dementia, frontotemporal dementia with Parkinsonism-17, Pick's disease, corticobasal degeneration, and agryophilic grain disease; or a neurodegenerative disease accompanied by a tau pathology, in particular a neurodegenerative disease selected from amyotrophic lateral sclerosis or frontotemporal lobe dementia caused by C9ORF72 mutations, in a subject in need thereof.

### DETAILED DESCRIPTION OF THE INVENTION

The present invention is directed to compounds of Formula (I), as defined herein before, and pharmaceutically acceptable addition salts and solvates thereof. The compounds of Formula (I) are inhibitors of O-GlcNAc hydrolase (OGA) and may be useful in the prevention or treatment of tauopathies, in particular a tauopathy selected from the group consisting of Alzheimer's disease, progressive supranuclear palsy, Down's syndrome, frontotemporal lobe dementia, frontotemporal dementia with Parkinsonism-17, Pick's disease, corticobasal degeneration, and agryophilic grain disease; or may be useful in the prevention or treatment of neurodegenerative diseases accompanied by a tau pathology, in particular a neurodegenerative disease selected from amyotrophic lateral sclerosis or frontotemporal lobe dementia caused by C9ORF72 mutations.

In an embodiment, the invention is directed to compounds of Formula (I) as referred to herein, and the tautomers and the stereoisomeric forms thereof, wherein R^{A} is a heteroaryl radical selected from the group consisting of pyridin-2-yl, pyridin-3-yl, pyridin-4-yl, pyridazin-3-yl, pyrimidin-4-yl, pyrimidin-5-yl, and pyrazin-2-yl, each of which may be optionally substituted with 1, 2 or 3 substituents each independently selected from the group consisting of halo; cyano; C₁₋₄alkyl optionally substituted with 1, 2, or 3 independently selected halo substituents; —C(O)NR^{a}R^{aa}; NR^{a}R^{aa}; and C₁₋₄alkyloxy optionally substituted with 1, 2, or 3 independently selected halo substituents; wherein R^{a} and R^{aa} are each independently selected from the group consisting of hydrogen and C₁₋₄alkyl optionally substituted with 1, 2, or 3 independently selected halo substituents;

L^{A} is selected from the group consisting of a covalent bond, —CH₂—, —O—, —OCH₂—, —CH₂O—, —NH—, —N(CH₃)—, —NHCH₂— and —CH₂NH—;

x represents 1;

R is H or CH₃; and

R^{B} is a bicyclic radical of formula (b-1), (b-2) or (b-3)

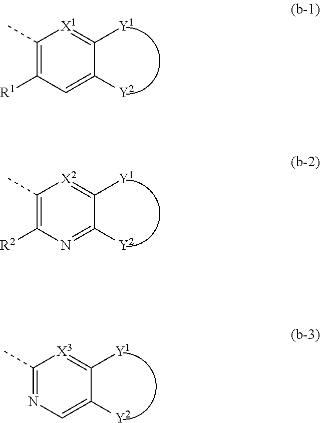

wherein

R¹ and R² are each selected from the group consisting of hydrogen, fluoro and methyl;

X¹, X² and X³ each represent CH, CF or N;

—Y¹—Y²— forms a bivalent radical selected from the group consisting of

—O(CH₂)ₘO— (c-1);

—O(CH₂)ₙ— (c-2);

—(CH₂)ₙO— (c-3);

—O(CH₂)ₚNR³— (c-4);

—NR³(CH₂)_{P}O— (c-5);

—O(CH₂)(CO)NR³— (c-6);

—NR³(CO)(CH₂)O— (c-7);

—(CH₂)ₙNR³(CO)— (c-8);

—(CO)NR³(CH₂)ₙ— (c-9); and

—N═CH(CO)NR³— (c-10);

wherein

m is 1 or 2;

n and p each independently represent 2 or 3;

each R³ is independently H or C₁₋₄alkyl;

R^{C} is selected from the group consisting of fluoro, methyl, hydroxy, methoxy, trifluoromethyl, and difluoromethyl;

R^{D} is selected from the group consisting of hydrogen, fluoro, methyl, hydroxy, methoxy, trifluoromethyl, and difluoromethyl; and

y represents 0, 1 or 2;

with the provisos that

a) R^{C} is not hydroxy or methoxy when present at the carbon atom adjacent to the nitrogen atom of the piperidinediyl or pyrrolidinediyl ring;

b) R^{C} or R^{D} cannot be selected simultaneously from hydroxy or methoxy when R^{C} is present at the carbon atom adjacent to C—R^{D};

c) R^{D} is not hydroxy or methoxy when L^{A} is —O—, —OCH₂—, —CH₂O—, —NH—, —N(CH₃)—, —NH(CH₂)— or —(CH₂)NH—;

and the pharmaceutically acceptable salts and the solvates thereof.

In a particular embodiment, the invention is directed to compounds of Formula (I) as referred to herein, and the tautomers and the stereoisomeric forms thereof, wherein

R^{A} is a heteroaryl radical selected from the group consisting of pyridin-2-yl, pyridin-3-yl, pyridin-4-yl, pyridazin-3-yl, pyrimidin-4-yl, pyrimidin-5-yl, and pyrazin-2-yl; or is an aryl radical selected from phenyl; each of which may be optionally substituted with 1, 2 or 3 substituents each independently selected from the group consisting of halo; C₁₋₄alkyl optionally substituted with 1, 2, or 3 independently selected halo substituents; and C₁₋₄alkyloxy optionally substituted with 1, 2, or 3 independently selected halo substituents.

In a particular embodiment, the invention is directed to compounds of Formula (I) as referred to herein, and the tautomers and the stereoisomeric forms thereof, wherein

R^{A} is a heteroaryl radical selected from the group consisting of pyridin-2-yl, pyridin-4-yl, pyrimidin-4-yl, and pyrazin-2-yl; or is an aryl radical selected from phenyl; each of which may be optionally substituted with 1, 2 or 3 substituents each independently selected from the group consisting of halo; C₁₋₄alkyl optionally substituted with 1, 2, or 3 independently selected halo substituents; and C₁₋₄alkyloxy optionally substituted with 1, 2, or 3 independently selected halo substituents.

In a further embodiment, the invention is directed to compounds of Formula (I) as referred to herein, and the tautomers and the stereoisomeric forms thereof, wherein

R^{A} is a heteroaryl radical selected from the group consisting of pyridin-2-yl, pyridin-4-yl, pyrimidin-4-yl, and pyrazin-2-yl; each of which may be optionally substituted with 1, 2 or 3 substituents each independently selected from the group consisting of halo; C₁₋₄alkyl optionally substituted with 1, 2, or 3 independently selected halo substituents; and C₁₋₄alkyloxy optionally substituted with 1, 2, or 3 independently selected halo substituents; or is an aryl radical selected from phenyl and optionally substituted with 1, 2 or 3, independently selected halo substituents, in particular 1 halo substituent.

In a particular embodiment, the invention is directed to compounds of Formula (I) as referred to herein, and the tautomers and the stereoisomeric forms thereof, wherein

R^{A} is a heteroaryl radical selected from the group consisting of pyridin-4-yl, pyrimidin-4-yl, and pyrazin-2-yl, each of which may be optionally substituted with 1, 2 or 3 substituents each independently selected from the group consisting of halo; C₁₋₄alkyl optionally substituted with 1, 2, or 3 independently selected halo substituents; and C₁₋₄alkyloxy optionally substituted with 1, 2, or 3 independently selected halo substituents;

and the pharmaceutically acceptable salts and the solvates thereof.

In a further embodiment, the invention is directed to compounds of Formula (I) as referred to herein, and the tautomers and the stereoisomeric forms thereof, wherein

R^{A} is a heteroaryl radical selected from the group consisting of pyridin-4-yl, pyrimidin-4-yl, and pyrazin-2-yl, each of which may be optionally substituted with 1, 2 or 3 substituents each independently selected from the group consisting of C₁₋₄alkyl optionally substituted with 1, 2, or 3 independently selected halo substituents; and C₁₋₄alkyloxy optionally substituted with 1, 2, or 3 independently selected halo substituents;

and the pharmaceutically acceptable salts and the solvates thereof.

In an additional embodiment, the invention is directed to compounds of Formula (I) as referred to herein, and the tautomers and the stereoisomeric forms thereof, wherein

R^{A} is pyridin-4-yl or pyrimidin-4-yl, each of which may be optionally substituted with 1, 2 or 3 substituents each independently selected from the group consisting of C₁₋₄alkyl optionally substituted with 1, 2, or 3 independently selected halo substituents; and C₁₋₄alkyloxy optionally substituted with 1, 2, or 3 independently selected halo substituents.

In an additional embodiment, the invention is directed to compounds of Formula (I) as referred to herein, and the tautomers and the stereoisomeric forms thereof, wherein

R^{A} is phenyl optionally substituted with 1, 2 or 3, independently selected halo substituents, in particular 1 halo substituent.

In a further embodiment, the invention is directed to compounds of Formula (I), as referred to herein, and the tautomers and the stereoisomeric forms thereof, wherein L^{A} is selected from the group consisting of a covalent bond, —CH₂—, —O—, —OCH₂—, —CH₂O—, and —NHCH₂—.

In another embodiment, the invention is directed to compounds of Formula (I), as referred to herein, and the tautomers and the stereoisomeric forms thereof, wherein L^{A} is selected from the group consisting of covalent bond, —OCH₂—, and —NHCH₂—.

In a further embodiment, the invention is directed to compounds of Formula (I), as referred to herein, and the tautomers and the stereoisomeric forms thereof, wherein L^{A} is selected from the group consisting of —CH₂—, —O—, —OCH₂—, —CH₂O—, —NH—, —N(CH₃)—, —NHCH₂— and —CH₂NH—.

In another embodiment, the invention is directed to compounds of Formula (I), as referred to herein, and the tautomers and the stereoisomeric forms thereof, wherein L^{A} is selected from the group consisting of —CH₂—, —O—, —OCH₂—, —CH₂O—, and —NHCH₂—.

In another embodiment, the invention is directed to compounds of Formula (I), as referred to herein, and the tautomers and the stereoisomeric forms thereof, wherein L^{A} is selected from the group consisting of —OCH₂—, —CH₂O—, and —NHCH₂—.

In yet another embodiment, the invention is directed to compounds of Formula (I), as referred to herein, and the tautomers and the stereoisomeric forms thereof, wherein R^{B} is a bicyclic radical of formula (b-1) or (b-2).

In a further embodiment, the invention is directed to compounds of Formula (I), as referred to herein, and the tautomers and the stereoisomeric forms thereof, wherein R^{B} is a bicyclic radical of formula (b-1) or (b-2), wherein R¹ is selected from the group consisting of hydrogen, fluoro and methyl; R² is hydrogen or fluoro; X¹ is N or CH; and X² is CH.

In a further embodiment, the invention is directed to compounds of Formula (I), as referred to herein, and the tautomers and the stereoisomeric forms thereof, wherein R^{B} is a bicyclic radical of formula (b-1) or (b-2), wherein R¹ is selected from the group consisting of hydrogen, fluoro and methyl; R² is hydrogen; X¹ is N or CH; and X² is CH.

In another embodiment, the invention is directed to compounds of Formula (I), as referred to herein, and the tautomers and the stereoisomeric forms thereof, wherein R^{B} is a bicyclic radical of formula (b-1) or (b-2), wherein R¹ is selected from the group consisting of hydrogen, fluoro and methyl; R² is hydrogen or fluoro; X¹ is N or CH; X² is CH; and —Y¹—Y²— forms a bivalent radical selected from the group consisting of (c-1), (c-2), (c-4) and (c-6).

In another embodiment, the invention is directed to compounds of Formula (I), as referred to herein, and the tautomers and the stereoisomeric forms thereof, wherein R^{B} is a bicyclic radical of formula (b-1) or (b-2), wherein R¹ is selected from the group consisting of hydrogen, fluoro and methyl; R² is hydrogen; X¹ is N or CH; X² is CH; and —Y¹—Y²— forms a bivalent radical selected from the group consisting of (c-1), (c-2), (c-4) and (c-6).

In a further embodiment, the invention is directed to compounds of Formula (I), and the tautomers and the stereoisomeric forms thereof, wherein R^{B} is a bicyclic radical of formula (b-1) or (b-2), wherein R¹ is selected from the group consisting of hydrogen, fluoro and methyl; R² is hydrogen or fluoro; X¹ is N or CH; X² is CH; and —Y¹—Y²— forms a bivalent radical selected from the group consisting of (c-1), (c-2), (c-4) and (c-6), wherein m is 2; n is 2 or 3; and p is 2.

In a further embodiment, the invention is directed to compounds of Formula (I), and the tautomers and the stereoisomeric forms thereof, wherein R^{B} is a bicyclic radical of formula (b-1) or (b-2), wherein R¹ is selected from the group consisting of hydrogen, fluoro and methyl; R² is hydrogen; X¹ is N or CH; X² is CH; and —Y¹—Y²— forms a bivalent radical selected from the group consisting of (c-1), (c-2), (c-4) and (c-6), wherein m is 2; n is 2 or 3; and p is 2.

In a further embodiment, the invention is directed to compounds of Formula (I), as referred to herein, and the tautomers and the stereoisomeric forms thereof, wherein R^{D} is selected from the group consisting of hydrogen, fluoro, and methyl.

In a further embodiment, the invention is directed to compounds of Formula (I), as referred to herein, and the tautomers and the stereoisomeric forms thereof, wherein R^{D} is hydrogen or methyl.

In a further embodiment, the invention is directed to compounds of Formula (I), as referred to herein, and the tautomers and the stereoisomeric forms thereof, wherein y represents 0 or 1.

In a further embodiment, the invention is directed to compounds of Formula (I), as referred to herein, and the tautomers and the stereoisomeric forms thereof, wherein y represents 0.

In a further embodiment, the invention is directed to compounds of Formula (I), as referred to herein, and the tautomers and the stereoisomeric forms thereof, wherein y represents 1.

In another embodiment, the invention is directed to compounds of Formula (I), as referred to herein, and the tautomers and the stereoisomeric forms thereof, wherein R^{A} is a heteroaryl radical selected from the group consisting of pyridin-4-yl, pyrimidin-4-yl, and pyrazin-2-yl, each of which may be optionally substituted with 1, 2 or 3 substituents each independently selected from the group consisting of halo; C₁₋₄alkyl optionally substituted with 1, 2, or 3 independently selected halo substituents; and C₁₋₄alkyloxy optionally substituted with 1, 2, or 3 independently selected halo substituents; L^{A} is selected from the group consisting of a covalent bond, —CH₂—, —O—, —OCH₂—, —CH₂O—, and —NHCH₂—;

x represents 1;

R is H or CH₃; and

R^{B} is a bicyclic radical of formula (b-1) or (b-2), wherein

R¹ and R² are each selected from the group consisting of hydrogen, fluoro and methyl;

X¹, X² and X³ each represent CH, CF or N;

—Y¹—Y²— forms a bivalent radical selected from the group consisting of (c-1), (c-2), (c-4) and (c-6); wherein

m is 1 or 2;

n and p each independently represent 2 or 3;

each R³ is independently H or C₁₋₄alkyl;

R^{C} is fluoro or methyl;

R^{D} is selected from the group consisting of hydrogen, fluoro, and methyl; and

y represents 0 or 1;

and the pharmaceutically acceptable salts and the solvates thereof.

In a further embodiment, the invention is directed to compounds of Formula (I), and the tautomers and the stereoisomeric forms thereof, wherein R^{B} is selected from the group consisting of

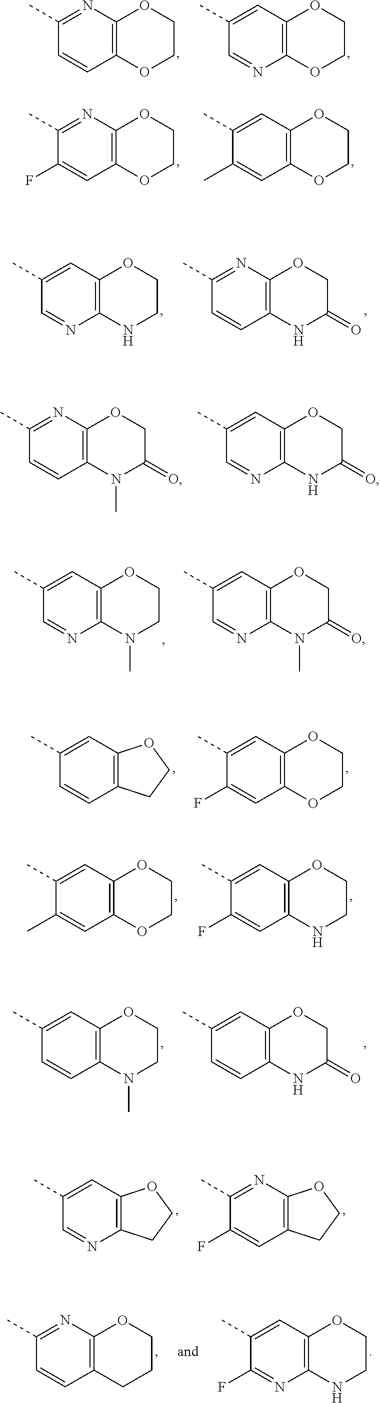

and the pharmaceutically acceptable salts and the solvates thereof.

In a further embodiment, the invention is directed to compounds of Formula (I), and the tautomers and the stereoisomeric forms thereof, wherein R^{B} is selected from the group consisting of

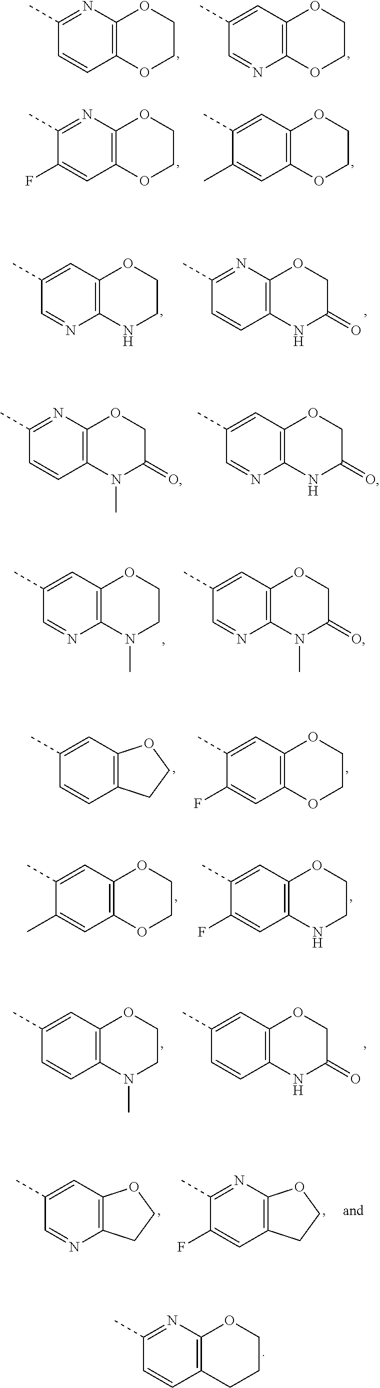

In a further embodiment, the invention is directed to compounds of Formula (I), and the tautomers and the stereoisomeric forms thereof, wherein R^{B} is a bicyclic radical selected from any one of (b-1a) to (b-2b)

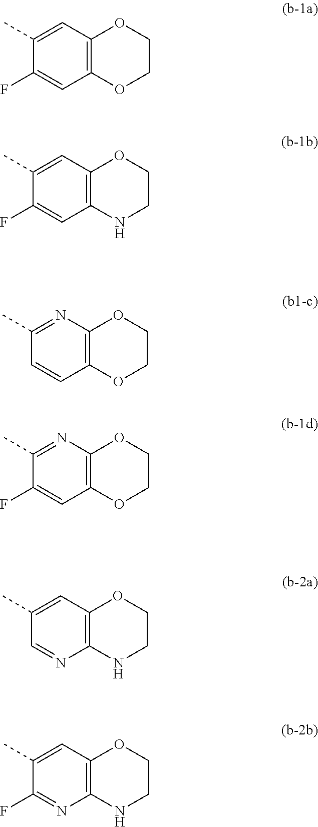

In a further embodiment, the invention is directed to compounds of Formula (I), as referred to herein, and the tautomers and the stereoisomeric forms thereof, wherein

R^{A} is pyridin-4-yl, optionally substituted with 1 or 2 substituents each independently selected from the group consisting of C₁₋₄alkyl and C₁₋₄alkyloxy;

L^{A} is selected from the group consisting of a covalent bond, —OCH₂— and —NHCH₂—;

x represents 1;

R is CH₃; and

R^{B} is a bicyclic radical of formula (b-1) or (b-2), wherein

R¹ and R² are each selected from the group consisting of hydrogen, fluoro and methyl;

X¹, X² and X³ each represent CH, CF or N;

—Y¹—Y²— forms a bivalent radical of formula (b1-c) or (b-1d)

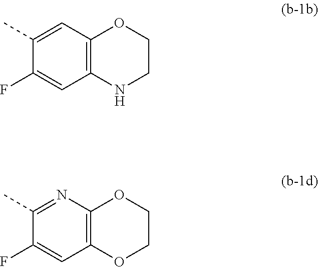

R^{D} is hydrogen; and

y is 0;

and the pharmaceutically acceptable salts and the solvates thereof.

### Definitions

“Halo” shall denote fluoro, chloro and bromo; “C₁₋₄alkyl” shall denote a straight or branched saturated alkyl group having 1, 2, 3 or 4 carbon atoms, respectively e.g. methyl, ethyl, 1-propyl, 2-propyl, butyl, 1-methyl-propyl, 2-methyl-1-propyl, 1,1-dimethylethyl, and the like; “C₁₋₄alkyloxy” shall denote an ether radical wherein C₁₋₄alkyl is as defined before. When reference is made to L^{A}, the definition is to be read from left to right, with the left part of the linker bound to R^{A} and the right part of the linker bound to the pyrrolidinediyl or piperidinediyl ring. Thus, when L^{A} is, for example, —O—CH₂—, then R^{A}-L^{A}- is R^{A}—O—CH₂—. When R^{C} is present more than once, where possible, it may be bound at the same carbon atom of the pyrrolidinediyl or piperidinediyl ring, and each instance may be different.

In general, whenever the term “substituted” is used in the present invention, it is meant, unless otherwise indicated or is clear from the context, to indicate that one or more hydrogens, in particular 1 to 3 hydrogens, preferably 1 or 2 hydrogens, more preferably 1 hydrogen, on the atom or radical indicated in the expression using “substituted” are replaced with a selection of substituents from the indicated group, provided that the normal valency is not exceeded, and that the substitution results in a chemically stable compound, i.e. a compound that is sufficiently robust to survive isolation to a useful degree of purity from a reaction mixture, and formulation into a therapeutic agent.

The term “subject” as used herein, refers to an animal, preferably a mammal, most preferably a human, who is or has been the object of treatment, observation or experiment. As used herein, the term “subject” therefore encompasses patients, as well as asymptomatic or presymptomatic individuals at risk of developing a disease or condition as defined herein.

The term “therapeutically effective amount” as used herein, means that amount of active compound or pharmaceutical agent that elicits the biological or medicinal response in a tissue system, animal or human that is being sought by a researcher, veterinarian, medical doctor or other clinician, which includes alleviation of the symptoms of the disease or disorder being treated. The term “prophylactically effective amount” as used herein, means that amount of active compound or pharmaceutical agent that substantially reduces the potential for onset of the disease or disorder being prevented.

As used herein, the term “composition” is intended to encompass a product comprising the specified ingredients in the specified amounts, as well as any product which results, directly or indirectly, from combinations of the specified ingredients in the specified amounts.

Hereinbefore and hereinafter, the term “compound of Formula (I)” is meant to include the addition salts, the solvates and the stereoisomers thereof.

The terms “stereoisomers” or “stereochemically isomeric forms” hereinbefore or hereinafter are used interchangeably.

The invention includes all stereoisomers of the compound of Formula (I) either as a pure stereoisomer or as a mixture of two or more stereoisomers.

Enantiomers are stereoisomers that are non-superimposable mirror images of each other. A 1:1 mixture of a pair of enantiomers is a racemate or racemic mixture. Diastereomers (or diastereoisomers) are stereoisomers that are not enantiomers, i.e. they are not related as mirror images. If a compound contains a double bond, the substituents may be in the E or the Z configuration. If a compound contains a disubstituted cycloalkyl group, the substituents may be in the cis or trans configuration. Therefore, the invention includes enantiomers, diastereomers, racemates, E isomers, Z isomers, cis isomers, trans isomers and mixtures thereof.

The absolute configuration is specified according to the Cahn-Ingold-Prelog system. The configuration at an asymmetric atom is specified by either R or S. Resolved compounds whose absolute configuration is not known can be designated by (+) or (−) depending on the direction in which they rotate plane polarized light.

When a specific stereoisomer is identified, this means that said stereoisomer is substantially free, i.e. associated with less than 50%, preferably less than 20%, more preferably less than 10%, even more preferably less than 5%, in particular less than 2% and most preferably less than 1%, of the other isomers. Thus, when a compound of formula (I) is for instance specified as (R), this means that the compound is substantially free of the (S) isomer; when a compound of formula (I) is for instance specified as E, this means that the compound is substantially free of the Z isomer; when a compound of formula (I) is for instance specified as cis, this means that the compound is substantially free of the trans isomer.

For use in medicine, the addition salts of the compounds of this invention refer to non-toxic “pharmaceutically acceptable addition salts”. Other salts may, however, be useful in the preparation of compounds according to this invention or of their pharmaceutically acceptable addition salts. Suitable pharmaceutically acceptable addition salts of the compounds include acid addition salts which may, for example, be formed by mixing a solution of the compound with a solution of a pharmaceutically acceptable acid such as hydrochloric acid, sulfuric acid, fumaric acid, maleic acid, succinic acid, acetic acid, benzoic acid, citric acid, tartaric acid, carbonic acid or phosphoric acid. Furthermore, where the compounds of the invention carry an acidic moiety, suitable pharmaceutically acceptable addition salts thereof may include alkali metal salts, e.g., sodium or potassium salts; alkaline earth metal salts, e.g., calcium or magnesium salts; and salts formed with suitable organic ligands, e.g., quaternary ammonium salts.

Representative acids which may be used in the preparation of pharmaceutically acceptable addition salts include, but are not limited to, the following: acetic acid, 2,2-dichloroactic acid, acylated amino acids, adipic acid, alginic acid, ascorbic acid, L-aspartic acid, benzenesulfonic acid, benzoic acid, 4-acetamidobenzoic acid, (+)-camphoric acid, camphorsulfonic acid, capric acid, caproic acid, caprylic acid, cinnamic acid, citric acid, cyclamic acid, ethane-1,2-disulfonic acid, ethanesulfonic acid, 2-hydroxy-ethanesulfonic acid, formic acid, fumaric acid, galactaric acid, gentisic acid, glucoheptonic acid, D-gluconic acid, D-glucoronic acid, L-glutamic acid, beta-oxo-glutaric acid, glycolic acid, hippuric acid, hydrobromic acid, hydrochloric acid, (+)-L-lactic acid, (±)-DL-lactic acid, lactobionic acid, maleic acid, (−)-L-malic acid, malonic acid, (±)-DL-mandelic acid, methanesulfonic acid, naphthalene-2-sulfonic acid, naphthalene-1,5-disulfonic acid, l-hydroxy-2-naphthoic acid, nicotinic acid, nitric acid, oleic acid, orotic acid, oxalic acid, palmitic acid, pamoic acid, phosphoric acid, L-pyroglutamic acid, salicylic acid, 4-amino-salicylic acid, sebacic acid, stearic acid, succinic acid, sulfuric acid, tannic acid, (+)-L-tartaric acid, thiocyanic acid, p-toluenesulfonic acid, trifluoromethylsulfonic acid, and undecylenic acid. Representative bases which may be used in the preparation of pharmaceutically acceptable addition salts include, but are not limited to, the following: ammonia, L-arginine, benethamine, benzathine, calcium hydroxide, choline, dimethylethanol-amine, diethanolamine, diethylamine, 2-(diethylamino)-ethanol, ethanolamine, ethylene-diamine, V-methyl-glucamine. hydrabamine, 1H-imidazole. L-lysine, magnesium hydroxide, 4-(2-hydroxyethyl)-morpholine, piperazine, potassium hydroxide, 1-(2-hydroxyethyl)-pyrrolidine, secondary amine, sodium hydroxide, triethanolamine, tromethamine and zinc hydroxide.

The names of compounds were generated according to the nomenclature rules agreed upon by the Chemical Abstracts Service (CAS) or according to the nomenclature rules agreed upon by the International Union of Pure and Applied Chemistry (IUPAC).

Preparation of the Final Compounds

The compounds according to the invention can generally be prepared by a succession of steps, each of which is known to the skilled person. In particular, the compounds can be prepared according to the following synthesis methods.

The compounds of Formula (I) may be synthesized in the form of racemic mixtures of enantiomers which can be separated from one another following art-known resolution procedures. The racemic compounds of Formula (I) may be converted into the corresponding diastereomeric salt forms by reaction with a suitable chiral acid. Said diastereomeric salt forms are subsequently separated, for example, by selective or fractional crystallization and the enantiomers are liberated therefrom by alkali. An alternative manner of separating the enantiomeric forms of the compounds of Formula (I) involves liquid chromatography using a chiral stationary phase. Said pure stereochemically isomeric forms may also be derived from the corresponding pure stereochemically isomeric forms of the appropriate starting materials, provided that the reaction occurs stereospecifically.

### Experimental Procedure 1

The final compounds of Formula (I-a) can be prepared by reacting an intermediate compound of Formula (II) with a compound of Formula (XV) according to reaction scheme (1). The reaction is performed in a suitable reaction-inert solvent, such as, for example, dichloromethane, a metal hydride, such as, for example sodium triacetoxyborohydride, sodium cyanoborohydride or sodium borohydride and may require the presence of a suitable base, such as, for example, triethylamine, and/or a Lewis acid, such as, for example titanium tetraisopropoxide or titanium tetrachloride, under thermal conditions, such as, 0° C. or room temperature, or 140° C., for example for 1 hour or 24 hours. In reaction scheme (1) all variables are defined as in Formula (I).

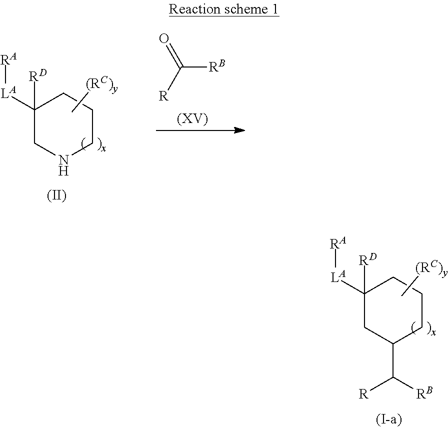

### Experimental Procedure 2

Additionally final compounds of Formula (I-a) can be prepared by reacting an intermediate compound of Formula (II) with a compound of Formula (XVI) according to reaction scheme (2). The reaction is performed in a suitable reaction-inert solvent, such as, for example, acetonitrile, a suitable base, such as, for example, triethylamine or diisopropylethylamine, under thermal conditions, such as, 0° C. or room temperature, or 75° C., for example for 1 hour or 24 hours. In reaction scheme (2) all variables are defined as in Formula (I), and wherein halo is chloro, bromo or iodo.

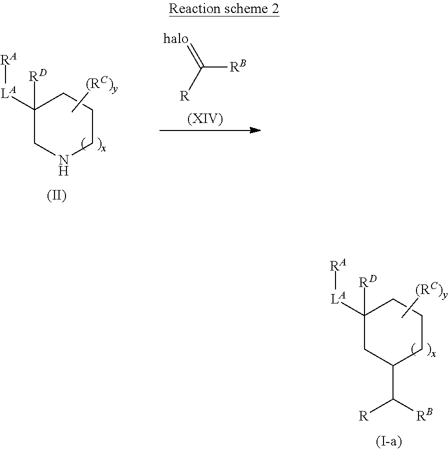

### Experimental Procedure 3

Additionally, final compounds of Formula (I), wherein R is CH₃, herein referred to as (I-b), can be prepared by reacting an intermediate compound of Formula (II) with a compound of Formula (XVII) followed by reaction of the formed imine derivative with and intermediate compound of Formula (XVIII) according to reaction scheme (3). The reaction is performed in a suitable reaction-inert solvent, such as, for example, anhydrous dichloromethane, a Lewis acid, such as, for example titanium tetraisopropoxide or titanium tetrachloride, under thermal conditions, such as, 0° C. or room temperature, for example for 1 hour or 24 hours. In reaction scheme (3) all variables are defined as in Formula (I), and wherein halo is chloro, bromo or iodo

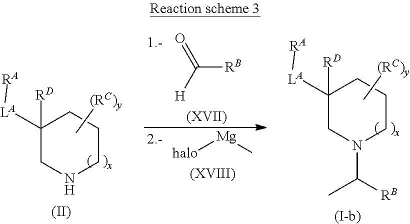

### Experimental Procedure 4

Additionally final compounds of Formula (I) wherein L^{A} is —NH—CH₂—, herein referred to as (I-c), can be prepared by reacting an intermediate compound of Formula (III) with a compound of Formula (V) according to reaction scheme (4). The reaction is performed in the presence of a palladium catalyst, such as, for example tris(dibenzylideneacetone)dipalladium(0), a ligand, such as, for example 2-dicyclohexylphosphino-2′-(N,N-dimethylamino)biphenyl, a base, such as, for example sodium tert-butoxide, a suitable reaction-inert solvent, such as, for example, anhydrous 1,4-dioxane, under thermal conditions, such as, 100° C., for example for 4 hour or 24 hours. In reaction scheme (4) all variables are defined as in Formula (I), and wherein halo is chloro, bromo or iodo

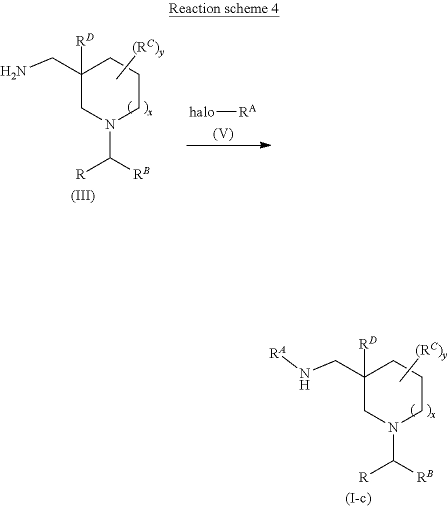

### Experimental Procedure 5

Intermediate compounds of Formula (II) can be prepared cleaving a protecting group in an intermediate compound of Formula (IV) according to reaction scheme (5). In reaction scheme (5) all variables are defined as in Formula (I), and PG is a suitable protecting group of the nitrogen function such as, for example, tert-butoxycarbonyl (Boc), ethoxy carbonyl, benzyl, benzyloxycarbonyl (Cbz). Suitable methods for removing such protecting groups are widely known to the person skilled in the art and comprise but are not limited to: Boc deprotection: treatment with a protic acid, such as, for example, trifluoroacetic acid, in a reaction inert solvent, such as, for example, dichloromethane; ethoxy carbonyl deprotection: treatment with a strong base, such as, for example, sodium hydroxide, in a reaction inert solvent such as for example wet tetrahydrofuran; benzyl deprotection: catalytic hydrogenation in the presence of a suitable catalyst, such as, for example, palladium on carbon, in a reaction inert solvent, such as, for example, ethanol; benzyloxycarbonyl deprotection: catalytic hydrogenation in the presence of a suitable catalyst, such as, for example, palladium on carbon, in a reaction inert solvent, such as, for example, ethanol.

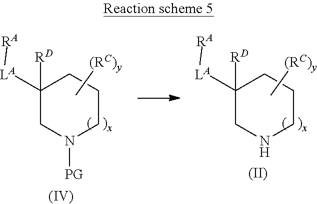

### Experimental Procedure 6

Intermediate compounds of Formula (IV-a) can be prepared by “Negishi coupling” reaction of a halo compound of Formula (V) with an organozinc compound of Formula (VI) according to reaction scheme (6). The reaction is performed in a suitable reaction-inert solvent, such as, for example, tetrahydrofuran, and a suitable catalyst, such as, for example, Pd(OAc)₂, a suitable ligand for the transition metal, such as, for example, 2-dicyclohexylphosphino-2′,6′-diisopropoxybiphenyl [CAS: 787618-22-8], under thermal conditions, such as, for example, room temperature, for example for 1 hour. In reaction scheme (6) all variables are defined as in Formula (I), L^{A} is a bond or CH₂ and halo is preferably bromo or iodo. PG is defined as in Formula (IV).

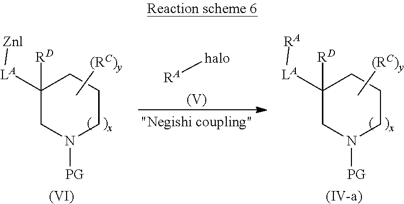

### Experimental Procedure 7

Intermediate compounds of Formula (VI) can be prepared by reaction of a halo compound of Formula (VII) with zinc according to reaction scheme (7). The reaction is performed in a suitable reaction-inert solvent, such as, for example, tetrahydrofuran, and a suitable salt, such as, for example, lithium chloride, under thermal conditions, such as, for example, 40° C., for example in a continuous-flow reactor. In reaction scheme (7) all variables are defined as in Formula (I), L^{A} is a bond or CH₂ and halo is preferably iodo. PG is defined as in Formula (IV).

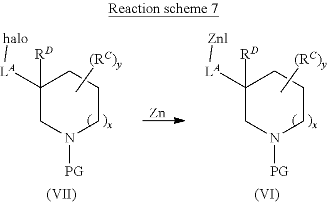

### Experimental Procedure 8

Intermediate compounds of Formula (IV) wherein R^{D} is H, herein referred to as (IV-b), can be prepared by hydrogenation reaction of an alkene compound of Formula (VIII) according to reaction scheme (8). The reaction is performed in a suitable reaction-inert solvent, such as, for example, methanol, and a suitable catalyst, such as, for example, palladium on carbon, and hydrogen, under thermal conditions, such as, for example, room temperature, for example for 3 hours. In reaction scheme (8) all variables are defined as in Formula (I) and PG is defined as in Formula (IV).

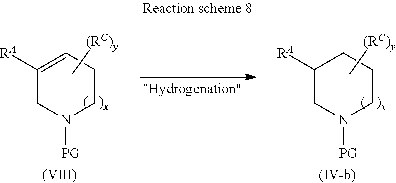

### Experimental Procedure 9

Intermediate compounds of Formula (VIII) can be prepared by “Suzuki coupling” reaction of an alkene compound of Formula (IX) and a halo derivative of Formula (V) according to reaction scheme (9). The reaction is performed in a suitable reaction-inert solvent, such as, for example, 1,4-dioxane, and a suitable catalyst, such as, for example, tetrakis(triphenylphosphine)palladium(0), a suitable base, such as, for example, NaHCO₃ (aq. sat. soltn.), under thermal conditions, such as, for example, 130° C., for example for 30 min under microwave irradiation. In reaction scheme (9) all variables are defined as in Formula (I), halo is preferably bromo or iodo, L^{A} is a bond, and PG is defined as in Formula (IV), L^{A} is a bond and R^{D} is H.

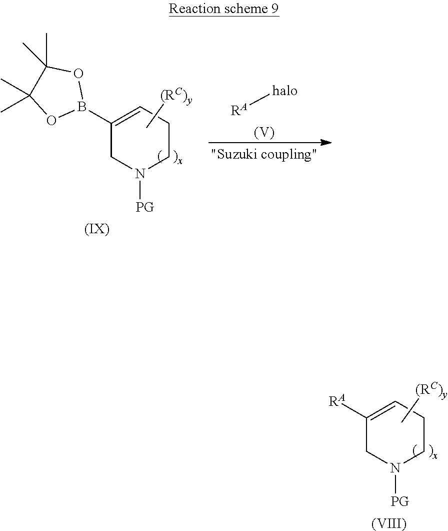

### Experimental Procedure 10

Intermediate compounds of Formula (IV-c) can be prepared by reaction of a hydroxy compound of Formula (X) and a halo derivative of Formula (V) according to reaction scheme (10). The reaction is performed in a suitable reaction-inert solvent, such as, for example, dimethylformamide or dimethylsulfoxide, and a suitable base, such as, sodium hydride or potassium tert-butoxide, under thermal conditions, such as, for example, 50° C., for example for 48 hour. In reaction scheme (10) all variables are defined as in Formula (I), L^{A} is a bond or CH₂ and halo is preferably chloro, bromo or fluoro. PG is defined as in Formula (IV).

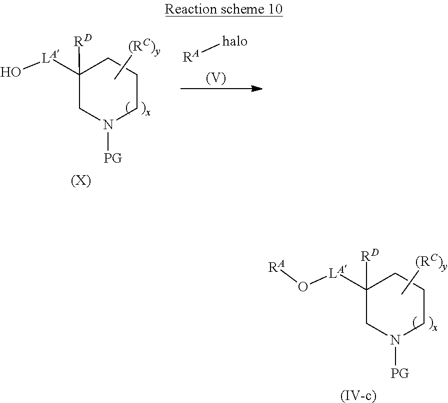

### Experimental Procedure 11

Alternatively intermediate compounds of Formula (IV-c) can be prepared by “Mitsunobu reaction” of a hydroxy compound of Formula (X) and a hydroxy derivative of Formula (XI) according to reaction scheme (11). The reaction is performed in a suitable reaction-inert solvent, such as, for example, toluene, a phosphine, such as, triphenylphosphine, a suitable coupling agent, such as, for example DIAD (CAS: 2446-83-5), under thermal conditions, such as, for example, 70° C., for example for 17 hour. In reaction scheme (11) all variables are defined as in Formula (I), L^{A} is a bond or CH₂ and halo is preferably chloro, bromo or fluoro. PG is defined as in Formula (IV).

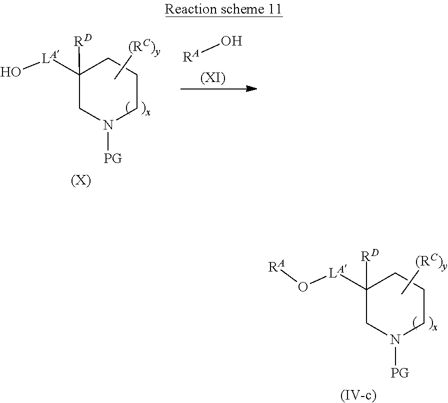

### Experimental Procedure 12

Intermediate compounds of Formula (III) can be prepared cleaving the protecting group in an intermediate compound of Formula (XI) according to reaction scheme (12). The reaction is performed in the presence of hydrazine hydrate in a suitable reaction-inert solvent, such as, for example, ethanol, under thermal conditions, such as, for example, 80° C., for example for 2 hour. In reaction scheme (12) all variables are defined as in Formula (I).

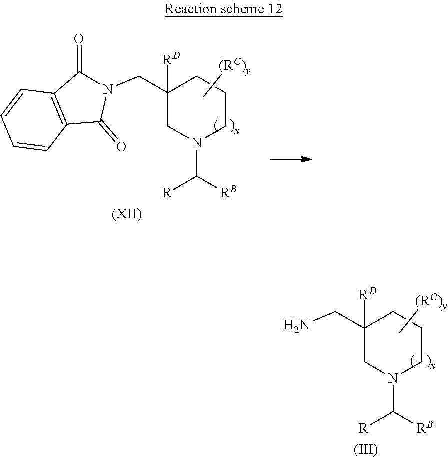

### Experimental Procedure 13

Intermediate compounds of Formula (XII) can be prepared by reacting an intermediate compound of Formula (XIII) with phthalimide according to reaction scheme (13). The reaction is performed in the presence of a phosphine, such as, for example triphenylphosphine, a suitable coupling agent, such as, for example diisopropyl azodicarboxylate in a suitable reaction-inert solvent, such as, for example, dry tetrahydrofuran, under thermal conditions, such as, for example, room temperature, for example for 24 hour. In reaction scheme (13) all variables are defined as in Formula (I).

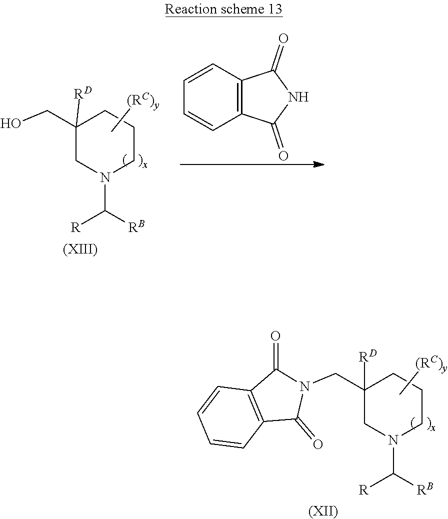

### Experimental Procedure 14

Intermediate compounds of Formula (XIII) can be prepared by deprotecting the alcohol group in an intermediate compound of Formula (XIV) according to reaction scheme (14). The reaction is performed in the presence of a fluoride source, such as, for example tetrabutylammonium fluoride, in a suitable reaction-inert solvent, such as, for example, dry tetrahydrofuran, under thermal conditions, such as, for example, room temperature, for example for 16 hour. In reaction scheme (13) all variables are defined as in Formula (I) and PG¹ is selected from the group consisting of trimethylsilyl, tert-butyldimethylsilyl, triisopropylsilyl or tert-butyldiphenylsilyl.

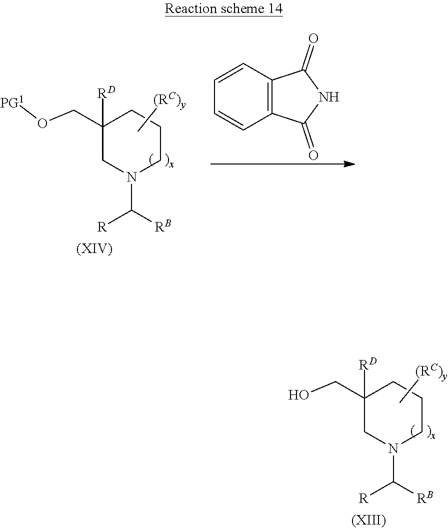

Intermediates of Formulae (V), (VII), (IX), (XV), (XVI), (XVII) and (XVIII) are commercially available or can be prepared by know procedures to those skilled in the art.

Pharmacology

The compounds of the present invention and the pharmaceutically acceptable compositions thereof inhibit O-GlcNAc hydrolase (OGA) and therefore may be useful in the treatment or prevention of diseases involving tau pathology, also known as tauopathies, and diseases with tau inclusions. Such diseases include, but are not limited to Alzheimer's disease, amyotrophic lateral sclerosis and parkinsonism-dementia complex, argyrophilic grain disease, chronic traumatic encephalopathy, corticobasal degeneration, diffuse neurofibrillary tangles with calcification, Down's syndrome, Familial British dementia, Familial Danish dementia, Frontotemporal dementia and parkinsonism linked to chromosome 17 (caused by MAPT mutations), Frontotemporal lobar degeneration (some cases caused by C9ORF72 mutations), Gerstmann-Straussler-Scheinker disease, Guadeloupean parkinsonism, myotonic dystrophy, neurodegeneration with brain iron accumulation, Niemann-Pick disease, type C, non-Guamanian motor neuron disease with neurofibrillary tangles, Pick's disease, postencephalitic parkinsonism, prion protein cerebral amyloid angiopathy, progressive subcortical gliosis, progressive supranuclear palsy, SLC9A6-related mental retardation, subacute sclerosing panencephalitis, tangle-only dementia, and white matter tauopathy with globular glial inclusions.

As used herein, the term “treatment” is intended to refer to all processes, wherein there may be a slowing, interrupting, arresting or stopping of the progression of a disease or an alleviation of symptoms, but does not necessarily indicate a total elimination of all symptoms. As used herein, the term “prevention” is intended to refer to all processes, wherein there may be a slowing, interrupting, arresting or stopping of the onset of a disease.

The invention also relates to a compound according to the general Formula (I), a stereoisomeric form thereof or a pharmaceutically acceptable acid or base addition salt thereof, for use in the treatment or prevention of diseases or conditions selected from the group consisting of Alzheimer's disease, amyotrophic lateral sclerosis and parkinsonism-dementia complex, argyrophilic grain disease, chronic traumatic encephalopathy, corticobasal degeneration, diffuse neurofibrillary tangles with calcification, Down's syndrome, Familial British dementia, Familial Danish dementia, Frontotemporal dementia and parkinsonism linked to chromosome 17 (caused by MAPT mutations), Frontotemporal lobar degeneration (some cases caused by C9ORF72 mutations), Gerstmann-Straussler-Scheinker disease, Guadeloupean parkinsonism, myotonic dystrophy, neurodegeneration with brain iron accumulation, Niemann-Pick disease, type C, non-Guamanian motor neuron disease with neurofibrillary tangles, Pick's disease, postencephalitic parkinsonism, prion protein cerebral amyloid angiopathy, progressive subcortical gliosis, progressive supranuclear palsy, SLC9A6-related mental retardation, subacute sclerosing panencephalitis, tangle-only dementia, and white matter tauopathy with globular glial inclusions.

The invention also relates to a compound according to the general Formula (I), a stereoisomeric form thereof or a pharmaceutically acceptable acid or base addition salt thereof, for use in the treatment, prevention, amelioration, control or reduction of the risk of diseases or conditions selected from the group consisting of Alzheimer's disease, amyotrophic lateral sclerosis and parkinsonism-dementia complex, argyrophilic grain disease, chronic traumatic encephalopathy, corticobasal degeneration, diffuse neurofibrillary tangles with calcification, Down's syndrome, Familial British dementia, Familial Danish dementia, Frontotemporal dementia and parkinsonism linked to chromosome 17 (caused by MAPT mutations), Frontotemporal lobar degeneration (some cases caused by C9ORF72 mutations), Gerstmann-Straussler-Scheinker disease, Guadeloupean parkinsonism, myotonic dystrophy, neurodegeneration with brain iron accumulation, Niemann-Pick disease, type C, non-Guamanian motor neuron disease with neurofibrillary tangles, Pick's disease, postencephalitic parkinsonism, prion protein cerebral amyloid angiopathy, progressive subcortical gliosis, progressive supranuclear palsy, SLC9A6-related mental retardation, subacute sclerosing panencephalitis, tangle-only dementia, and white matter tauopathy with globular glial inclusions.

In particular, the diseases or conditions may in particular be selected from a tauopathy, more in particular a tauopathy selected from the group consisting of Alzheimer's disease, progressive supranuclear palsy, Down's syndrome, frontotemporal lobe dementia, frontotemporal dementia with Parkinsonism-17, Pick's disease, corticobasal degeneration, and agryophilic grain disease; or the diseases or conditions may in particular be neurodegenerative diseases accompanied by a tau pathology, more in particular a neurodegenerative disease selected from amyotrophic lateral sclerosis or frontotemporal lobe dementia caused by C9ORF72 mutations.

Preclinical States in Alzheimer's and Tauopathy Diseases:

In recent years the United States (US) National Institute for Aging and the International Working Group have proposed guidelines to better define the preclinical (asymptomatic) stages of AD (Dubois B, et al. Lancet Neurol. 2014; 13:614-629; Sperling, R A, et al. Alzheimers Dement. 2011; 7:280-292). Hypothetical models postulate that Aβ accumulation and tau-aggregation begins many years before the onset of overt clinical impairment. The key risk factors for elevated amyloid accumulation, tau-aggregation and development of AD are age (ie, 65 years or older), APOE genotype, and family history. Approximately one third of clinically normal older individuals over 75 years of age demonstrate evidence of Aβ or tau accumulation on PET amyloid and tau imaging studies, the latter being less advanced currently. In addition, reduced Abeta-levels in CSF measurements are observed, whereas levels of non-modified as well as phosphorylated tau are elevated in CSF. Similar findings are seen in large autopsy studies and it has been shown that tau aggregates are detected in the brain as early as 20 years of age and younger. Amyloid-positive (Aβ+) clinically normal individuals consistently demonstrate evidence of an “AD-like endophenotype” on other biomarkers, including disrupted functional network activity in both functional magnetic resonance imaging (MRI) and resting state connectivity, fluorodeoxyglucose ¹⁸F (FDG) hypometabolism, cortical thinning, and accelerated rates of atrophy. Accumulating longitudinal data also strongly suggests that Aβ+ clinically normal individuals are at increased risk for cognitive decline and progression to mild cognitive impairment (MCI) and AD dementia. The Alzheimer's scientific community is of the consensus that these Aβ+ clinically normal individuals represent an early stage in the continuum of AD pathology. Thus, it has been argued that intervention with a therapeutic agent that decreases Aβ production or the aggregation of tau is likely to be more effective if started at a disease stage before widespread neurodegeneration has occurred. A number of pharmaceutical companies are currently testing BACE inhibition in prodromal AD.

Thanks to evolving biomarker research, it is now possible to identify Alzheimer's disease at a preclinical stage before the occurrence of the first symptoms. All the different issues relating to preclinical Alzheimer's disease such as, definitions and lexicon, the limits, the natural history, the markers of progression and the ethical consequences of detecting the disease at the asymptomatic stage, are reviewed in Alzheimer's & Dementia 12 (2016) 292-323.

Two categories of individuals may be recognized in preclinical Alzheimer's disease or tauopathies. Cognitively normal individuals with amyloid beta or tau aggregation evident on PET scans, or changes in CSF Abeta, tau and phospho-tau are defined as being in an “asymptomatic at risk state for Alzheimer's disease (AR-AD)” or in a “asymptomatic state of tauopathy”. Individuals with a fully penetrant dominant autosomal mutation for familial Alzheimer's disease are said to have “presymptomatic Alzheimer's disease”. Dominant autosomal mutations within the tau-protein have been described for multiple forms of tauopathies as well.

Thus, in an embodiment, the invention also relates to a compound according to the general Formula (I), a stereoisomeric form thereof or a pharmaceutically acceptable acid or base addition salt thereof, for use in control or reduction of the risk of preclinical Alzheimer's disease, prodromal Alzheimer's disease, or tau-related neurodegeneration as observed in different forms of tauopathies.

As already mentioned hereinabove, the term “treatment” does not necessarily indicate a total elimination of all symptoms, but may also refer to symptomatic treatment in any of the disorders mentioned above. In view of the utility of the compound of Formula (I), there is provided a method of treating subjects such as warm-blooded animals, including humans, suffering from or a method of preventing subjects such as warm-blooded animals, including humans, suffering from any one of the diseases mentioned hereinbefore.

Said methods comprise the administration, i.e. the systemic or topical administration, preferably oral administration, of a prophylactically or a therapeutically effective amount of a compound of Formula (I), a stereoisomeric form thereof, a pharmaceutically acceptable addition salt or solvate thereof, to a subject such as a warm-blooded animal, including a human.

Therefore, the invention also relates to a method for the prevention and/or treatment of any of the diseases mentioned hereinbefore comprising administering a prophylactically or a therapeutically effective amount of a compound according to the invention to a subject in need thereof.

The invention also relates to a method for modulating O-GlcNAc hydrolase (OGA) activity, comprising administering to a subject in need thereof, a prophylactically or a therapeutically effective amount of a compound according to the invention and as defined in the claims or a pharmaceutical composition according to the invention and as defined in the claims.

A method of treatment may also include administering the active ingredient on a regimen of between one and four intakes per day. In these methods of treatment the compounds according to the invention are preferably formulated prior to administration. As described herein below, suitable pharmaceutical formulations are prepared by known procedures using well known and readily available ingredients.

The compounds of the present invention, that can be suitable to treat or prevent any of the disorders mentioned above or the symptoms thereof, may be administered alone or in combination with one or more additional therapeutic agents. Combination therapy includes administration of a single pharmaceutical dosage formulation which contains a compound of Formula (I) and one or more additional therapeutic agents, as well as administration of the compound of Formula (I) and each additional therapeutic agent in its own separate pharmaceutical dosage formulation. For example, a compound of Formula (I) and a therapeutic agent may be administered to the patient together in a single oral dosage composition such as a tablet or capsule, or each agent may be administered in separate oral dosage formulations.

A skilled person will be familiar with alternative nomenclatures, nosologies, and classification systems for the diseases or conditions referred to herein. For example, the fifth edition of the Diagnostic & Statistical Manual of Mental Disorders (DSM-5™) of the American Psychiatric Association utilizes terms such as neurocognitive disorders (NCDs) (both major and mild), in particular, neurocognitive disorders due to Alzheimer's disease. Such terms may be used as an alternative nomenclature for some of the diseases or conditions referred to herein by the skilled person.

Pharmaceutical Compositions

The present invention also provides compositions for preventing or treating diseases in which inhibition of O-GlcNAc hydrolase (OGA) is beneficial, such as Alzheimer's disease, progressive supranuclear palsy, Down's syndrome, frontotemporal lobe dementia, frontotemporal dementia with Parkinsonism-17, Pick's disease, corticobasal degeneration, agryophilic grain disease, amyotrophic lateral sclerosis or frontotemporal lobe dementia caused by C9ORF72 mutations, said compositions comprising a therapeutically effective amount of a compound according to formula (I) and a pharmaceutically acceptable carrier or diluent.

While it is possible for the active ingredient to be administered alone, it is preferable to present it as a pharmaceutical composition. Accordingly, the present invention further provides a pharmaceutical composition comprising a compound according to the present invention, together with a pharmaceutically acceptable carrier or diluent. The carrier or diluent must be “acceptable” in the sense of being compatible with the other ingredients of the composition and not deleterious to the recipients thereof.

The pharmaceutical compositions of this invention may be prepared by any methods well known in the art of pharmacy. A therapeutically effective amount of the particular compound, in base form or addition salt form, as the active ingredient is combined in intimate admixture with a pharmaceutically acceptable carrier, which may take a wide variety of forms depending on the form of preparation desired for administration. These pharmaceutical compositions are desirably in unitary dosage form suitable, preferably, for systemic administration such as oral, percutaneous or parenteral administration; or topical administration such as via inhalation, a nose spray, eye drops or via a cream, gel, shampoo or the like. For example, in preparing the compositions in oral dosage form, any of the usual pharmaceutical media may be employed, such as, for example, water, glycols, oils, alcohols and the like in the case of oral liquid preparations such as suspensions, syrups, elixirs and solutions; or solid carriers such as starches, sugars, kaolin, lubricants, binders, disintegrating agents and the like in the case of powders, pills, capsules and tablets. Because of their ease in administration, tablets and capsules represent the most advantageous oral dosage unit form, in which case solid pharmaceutical carriers are obviously employed. For parenteral compositions, the carrier will usually comprise sterile water, at least in large part, though other ingredients, for example, to aid solubility, may be included. Injectable solutions, for example, may be prepared in which the carrier comprises saline solution, glucose solution or a mixture of saline and glucose solution. Injectable suspensions may also be prepared in which case appropriate liquid carriers, suspending agents and the like may be employed. In the compositions suitable for percutaneous administration, the carrier optionally comprises a penetration enhancing agent and/or a suitable wettable agent, optionally combined with suitable additives of any nature in minor proportions, which additives do not cause any significant deleterious effects on the skin. Said additives may facilitate the administration to the skin and/or may be helpful for preparing the desired compositions. These compositions may be administered in various ways, e.g., as a transdermal patch, as a spot-on or as an ointment.

It is especially advantageous to formulate the aforementioned pharmaceutical compositions in dosage unit form for ease of administration and uniformity of dosage. Dosage unit form as used in the specification and claims herein refers to physically discrete units suitable as unitary dosages, each unit containing a predetermined quantity of active ingredient calculated to produce the desired therapeutic effect in association with the required pharmaceutical carrier. Examples of such dosage unit forms are tablets (including scored or coated tablets), capsules, pills, powder packets, wafers, injectable solutions or suspensions, teaspoonfuls, tablespoonfuls and the like, and segregated multiples thereof.

The exact dosage and frequency of administration depends on the particular compound of Formula (I) used, the particular condition being treated, the severity of the condition being treated, the age, weight, sex, extent of disorder and general physical condition of the particular patient as well as other medication the individual may be taking, as is well known to those skilled in the art. Furthermore, it is evident that said effective daily amount may be lowered or increased depending on the response of the treated subject and/or depending on the evaluation of the physician prescribing the compounds of the instant invention.

Depending on the mode of administration, the pharmaceutical composition will comprise from 0.05 to 99% by weight, preferably from 0.1 to 70% by weight, more preferably from 0.1 to 50% by weight of the active ingredient, and, from 1 to 99.95% by weight, preferably from 30 to 99.9% by weight, more preferably from 50 to 99.9% by weight of a pharmaceutically acceptable carrier, all percentages being based on the total weight of the composition.

The present compounds can be used for systemic administration such as oral, percutaneous or parenteral administration; or topical administration such as via inhalation, a nose spray, eye drops or via a cream, gel, shampoo or the like. The compounds are preferably orally administered. The exact dosage and frequency of administration depends on the particular compound according to Formula (I) used, the particular condition being treated, the severity of the condition being treated, the age, weight, sex, extent of disorder and general physical condition of the particular patient as well as other medication the individual may be taking, as is well known to those skilled in the art. Furthermore, it is evident that said effective daily amount may be lowered or increased depending on the response of the treated subject and/or depending on the evaluation of the physician prescribing the compounds of the instant invention.

The amount of a compound of Formula (I) that can be combined with a carrier material to produce a single dosage form will vary depending upon the disease treated, the mammalian species, and the particular mode of administration. However, as a general guide, suitable unit doses for the compounds of the present invention can, for example, preferably contain between 0.1 mg to about 1000 mg of the active compound. A preferred unit dose is between 1 mg to about 500 mg. A more preferred unit dose is between 1 mg to about 300 mg. Even more preferred unit dose is between 1 mg to about 100 mg. Such unit doses can be administered more than once a day, for example, 2, 3, 4, 5 or 6 times a day, but preferably 1 or 2 times per day, so that the total dosage for a 70 kg adult is in the range of 0.001 to about 15 mg per kg weight of subject per administration. A preferred dosage is 0.01 to about 1.5 mg per kg weight of subject per administration, and such therapy can extend for a number of weeks or months, and in some cases, years. It will be understood, however, that the specific dose level for any particular patient will depend on a variety of factors including the activity of the specific compound employed; the age, body weight, general health, sex and diet of the individual being treated; the time and route of administration; the rate of excretion; other drugs that have previously been administered; and the severity of the particular disease undergoing therapy, as is well understood by those of skill in the area.

A typical dosage can be one 1 mg to about 100 mg tablet or 1 mg to about 300 mg taken once a day, or, multiple times per day, or one time-release capsule or tablet taken once a day and containing a proportionally higher content of active ingredient. The time-release effect can be obtained by capsule materials that dissolve at different pH values, by capsules that release slowly by osmotic pressure, or by any other known means of controlled release.

It can be necessary to use dosages outside these ranges in some cases as will be apparent to those skilled in the art. Further, it is noted that the clinician or treating physician will know how and when to start, interrupt, adjust, or terminate therapy in conjunction with individual patient response.

The invention also provides a kit comprising a compound according to the invention, prescribing information also known as “leaflet”, a blister package or bottle, and a container. Furthermore, the invention provides a kit comprising a pharmaceutical composition according to the invention, prescribing information also known as “leaflet”, a blister package or bottle, and a container. The prescribing information preferably includes advice or instructions to a patient regarding the administration of the compound or the pharmaceutical composition according to the invention. In particular, the prescribing information includes advice or instruction to a patient regarding the administration of said compound or pharmaceutical composition according to the invention, on how the compound or the pharmaceutical composition according to the invention is to be used, for the prevention and/or treatment of a tauopathy in a subject in need thereof. Thus, in an embodiment, the invention provides a kit of parts comprising a compound of Formula (I) or a stereoisomeric for thereof, or a pharmaceutically acceptable salt or a solvate thereof, or a pharmaceutical composition comprising said compound, and instructions for preventing or treating a tauopathy. The kit referred to herein can be, in particular, a pharmaceutical package suitable for commercial sale.

For the compositions, methods and kits provided above, one of skill in the art will understand that preferred compounds for use in each are those compounds that are noted as preferred above. Still further preferred compounds for the compositions, methods and kits are those compounds provided in the non-limiting Examples below.

### EXPERIMENTAL PART

Hereinafter, the term “m.p.” means melting point, “min” means minutes, “ACN” means acetonitrile, “aq.” means aqueous, “Boc” means tert-butyloxycarbonyl, “DavePhos” means 2-dicyclohexylphosphino-2′-(N,N-dimethylamino)biphenyl, “DIAD” means diisopropyl azodicarboxylate, “DMAP” means 4-(dimethylamino)pyridine, “DMF” means dimethylformamide, “r.t.” or “RT” means room temperature, “rac” or “RS” means racemic, “sat.” means saturated, “SFC” means supercritical fluid chromatography, “SFC-MS” means supercritical fluid chromatography/mass spectrometry, “LC-MS” means liquid chromatography/mass spectrometry, “HPLC” means high-performance liquid chromatography, “i-PrOH” means isopropyl alcohol, “RP” means reversed phase, “Rt” means retention time (in minutes), “[M+H]⁺” means the protonated mass of the free base of the compound, “wt” means weight, “THF” means tetrahydrofuran, “Et₂O” means diethylether, “EtOAc” means ethyl acetate, “Et₃N” means triethylamine, “DCM” means dichloromethane, “MeOH” means methanol, “sat” means saturated, “soltn” means solution, “sol.” means solution, “EtOH” means ethanol, “TFA” means trifluoroacetic acid, “2-MeTHF” means 2-methyl-tetrahydrofuran, “NMP” means N-methylpyrrolidone, “AIBN” means 2,2′-azobis(2-methylpropionitrile, “m-CPBA” means 3-chloroperbenzoic acid, “Pd(OAc)₂” or “(OAc)₂Pd” means palladium(II) acetate, “Pd₂(dba)₃” means tris(dibenzybdeneacetone)dipalladium(0), “Pd(PPh₃)₄” means tetrakis(triphenylphosphine)palladium(0), “PdCl₂(dppf)” means [1,1′-bis(diphenylphosphino)ferrocene]dichloropalladium(II), “PdCl₂(PPh₃)₂” means bis(triphenylphosphine)palladium(II) dichloride, “RuPhos” means 2-dicyclohexylphosphino-2′,6′-diisopropoxybiphenyl, “t-BuXPhos” means 2-di-tert-butylphosphino-2′,4′,6′-triisopropylbiphenyl and “TMSCl” means trimethylsilyl chloride.

Whenever the notation “RS” is indicated herein, it denotes that the compound is a racemic mixture at the indicated centre, unless otherwise indicated. The stereochemical configuration for centres in some compounds has been designated “R” or “S” when the mixture(s) was separated; for some compounds, the stereochemical configuration at indicated centres has been designated as “*R” or “*S” when the absolute stereochemistry is undetermined although the compound itself has been isolated as a single stereoisomer and is enantiomerically/diastereomerically pure. The enantiomeric excess of compounds reported herein was determined by analysis of the racemic mixture by supercritical fluid chromatography (SFC) followed by SFC comparison of the separated enantiomer(s).

Flow chemistry reactions were performed in a Vapourtec R2+R4 unit using standard reactors provided by the vendor.

Microwave assisted reactions were performed in a single-mode reactor: Initiator™ Sixty EXP microwave reactor (Biotage AB), or in a multimode reactor: MicroSYNTH Labstation (Milestone, Inc.).

Thin layer chromatography (TLC) was carried out on silica gel 60 F254 plates (Merck) using reagent grade solvents. Open column chromatography was performed on silica gel, particle size 60 Å, mesh=230-400 (Merck) using standard techniques. Automated flash column chromatography was performed using ready-to-connect cartridges, on irregular silica gel, particle size 15-40 μm (normal phase disposable flash columns) on different flash systems: either a SPOT or LAFLASH systems from Armen Instrument, or PuriFlash®430evo systems from Interchim, or 971-FP systems from Agilent, or Isolera 1SV systems from Biotage.

Preparation of the Intermediates

Preparation of Intermediate 1

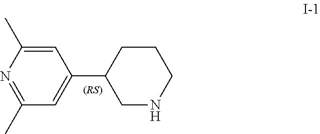

Amberlyst®15 hydrogen form (CAS: 39389-20-3, 11.1 g, loading 4.7 meq/g) was added to a stirred solution of intermediate 30 (2.84 g, 9.78 mmol) in MeOH (50 mL) at rt. The mixture was shaken in a solid phase reactor at rt for 16 h. The resin was filtered and washed with MeOH (this fraction was discarded) and then with a 7N solution of NH₃ in MeOH. The filtrate was concentrated in vacuo to yield intermediate 1 (1.2 g, 64%) as an orange oil.

Preparation of Intermediate 2

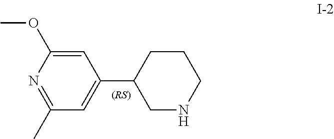

Intermediate 2 was prepared following an analogous procedure to the one described for the synthesis of intermediate 1 using intermediate 31 as starting material. Intermediate 2 was purified by reverse phase HPLC (stationary phase: C18 XBridge 30×100 mm 5 μm, mobile phase: gradient from 80% NH₄HCO₃ 0.25% solution in water, 20% CH₃CN to 60% NH₄HCO₃ 0.25% solution in water, 40% CH₃CN).

Preparation of Intermediate 3

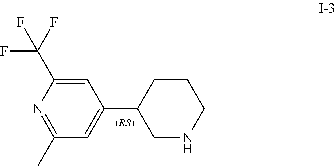 [SMILES: Cc1cc(C2CCCNC2)cc(C(F)(F)F)n1]

Intermediate 3 was prepared following an analogous procedure to the one described for the synthesis of intermediate 1 using intermediate 32 as starting material. Intermediate 3 was purified by reverse phase HPLC (stationary phase: C18 XBridge 30×100 mm 5 μm, mobile phase: gradient from 80% NH₄HCO₃ 0.25% solution in water, 20% CH₃CN to 60% NH₄HCO₃ 0.25% solution in water, 40% CH₃CN).

Preparation of Intermediate 4

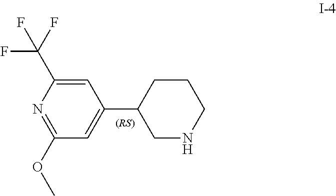 [SMILES: COc1cc(C2CCCNC2)cc(C(F)(F)F)n1]

Intermediate 4 was prepared following an analogous procedure to the one described for the synthesis of intermediate 1 using intermediate 33 as starting material. Intermediate 4 was purified by reverse phase HPLC (stationary phase: C18 XBridge 30×100 mm 5 μm, mobile phase: gradient from 80% NH₄HCO₃ 0.25% solution in water, 20% CH₃CN to 60% NH₄HCO₃ 0.25% solution in water, 40% CH₃CN).

Preparation of Intermediate 5

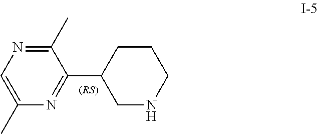

To a solution of intermediate 34 (130 mg, 0.69 mmol) in EtOH (11 mL), Pd/C 10% (73.1 mg, 0.69 mmol) was added under a N₂ atmosphere. The mixture was stirred under hydrogen atmosphere at rt for 18 h. The reaction mixture was filtered over a pad of dicalite and the pad was then rinsed with ethanol. The combined filtrates were concentrated in vacuo to yield intermediate 5 (130 mg, 99%).

Preparation of Intermediate 6

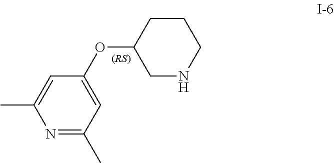

Intermediate 6 was prepared following an analogous procedure to the one described for the synthesis of intermediate 1 using intermediate 35 as starting material.

Preparation of Intermediate 7

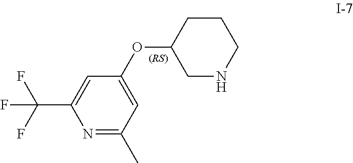 [SMILES: Cc1cc(OC2CCCNC2)cc(C(F)(F)F)n1]

Intermediate 7 was prepared following an analogous procedure to the one described for the synthesis of intermediate 1 using intermediate 36 as starting material.

Preparation of Intermediate 8

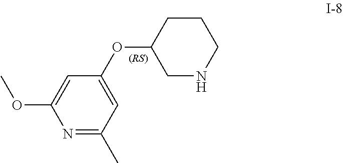 [SMILES: COc1cc(OC2CCCNC2)cc(C)n1]

Intermediate 8 was prepared following an analogous procedure to the one described for the synthesis of intermediate 1 using intermediate 37 as starting material.

Preparation of Intermediate 9

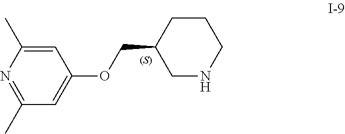

Intermediate 9 was prepared following an analogous procedure to the one described for the synthesis of intermediate 1 using intermediate 38 as starting material.

Preparation of Intermediate 10

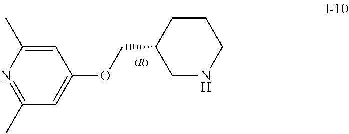

Intermediate 10 was prepared following an analogous procedure to the one described for the synthesis of intermediate 1 using intermediate 39 as starting material.

Preparation of Intermediate 11

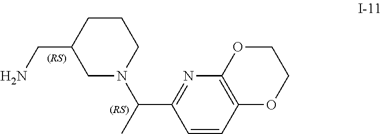 [SMILES: CC(c1ccc2c(n1)OCCO2)N1CCCC(CN)C1]

Hydrazine hydrate (0.135 mL, 2.38 mmol) was added to a solution of intermediate 54 (243 mg, 0.6 mmol) in EtOH (10 mL) at rt and the mixture was stirred at 80° C. for 2 h. The solvents were evaporated in vacuo and the residue thus obtained was triturated with DIPE. The solid was filtered off and the filtrate was concentrated in vacuo to give a residue that was purified by flash column chromatography (silica; NH₃ 7N in MeOH in DCM 0/100 to 10/90). The desired fractions were collected and concentrated in vacuo to yield intermediate 11 (106 mg, 63%) as a pale yellow sticky solid.

Preparation of Intermediate 12

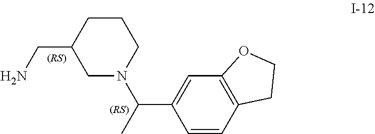 [SMILES: CC(c1ccc2c(c1)OCC2)N1CCCC(CN)C1]

Intermediate 12 was prepared following an analogous procedure to the one described for the synthesis of intermediate 11 using intermediate 55 as starting material.

Preparation of Intermediate 13

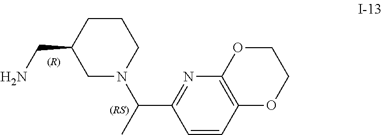

Intermediate 13 was prepared following an analogous procedure to the one described for the synthesis of intermediate 11 using intermediate 56 as starting material.

Preparation of Intermediate 14

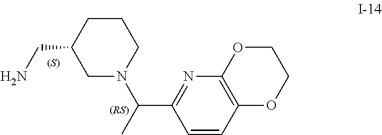

Intermediate 14 was prepared following an analogous procedure to the one described for the synthesis of intermediate 11 using intermediate 57 as starting material.

Preparation of Intermediate 15

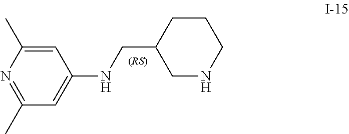 [SMILES: Cc1cc(NCC2CCCNC2)cc(C)n1]

HCl (25.6 mL, 102 mmol) was added dropwise to a stirred solution of intermediate 58 (2.23 g, 6.84 mmol) in MeOH (15.8 mL) at 0° C. The mixture was stirred at rt for 16 h. The solvent was evaporated in vacuo and the residue thus obtained was purified by reverse phase chromatography: 95% [25 mM NH₄HCO₃]—5% [acetonitrile:MeOH (1:1)] to 63% [25 mM NH₄HCO₃]—37% [acetonitrile:MeOH (1:1)]. The desired fractions were collected and concentrated in vacuo at 60° C. Acetonitrile (10 mL×3 times) was added and the solvent was evaporated in vacuo to yield intermediate 15 (1.3 g, 87%) as a yellow foam.

Preparation of Intermediate 16

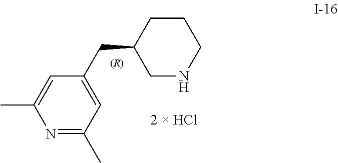

A 2-MeTHF (182.6 mL) solution of intermediate 40 (18.26 g, 59.98 mmol) was charged to a 400 mL reactor equipped with overhead stirrer under nitrogen. The resulting clear orange solution was cooled down to 0° C. and HCl (149.9 mL, 599.8 mmol, 4M solution in 1,4-dioxane) was added dropwise, maintaining the internal temperature below 5° C. Reaction mixture was stirred for 30 min at this temperature and warmed to 20° C. afterwards. A solid (bis HCl salt) crystallized with time. After 1 h at 20° C., the slurry was warmed to 50° C. and stirred for an extra 2 h. After that time, contents were cooled down to 0° C. and slurry filtered off. The wet cake was washed with 2-MeTHF (50 mL) and dried under vacuum at 50° C. overnight to yield intermediate 16 (16.18 g, 97%, 2×HCl salt) as a white solid.

Preparation of Intermediate 17

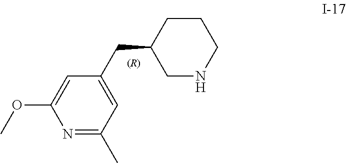

Intermediate 17 was prepared following an analogous procedure to the one described for the synthesis of intermediate 1 using intermediate 41 as starting material.

Preparation of Intermediate 18

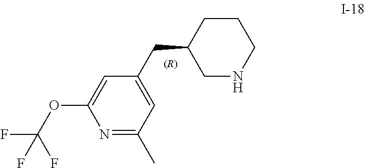

Intermediate 18 was prepared following an analogous procedure to the one described for the synthesis of intermediate 1 using intermediate 42 as starting material.

Preparation of Intermediate 19

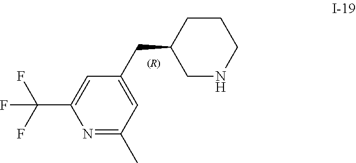

Intermediate 19 was prepared following an analogous procedure to the one described for the synthesis of intermediate 1 using intermediate 43 as starting material.

Preparation of Intermediate 30

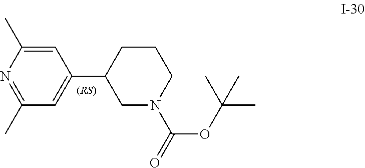 [SMILES: Cc1cc(C2CCCN(C(=O)OC(C)(C)C)C2)cc(C)n1]

A solution of intermediate 63 (3.8 g, 13.18 mmol) in EtOH (250 mL) was hydrogenated in a H-cube® (Pd/C 10%, 2 cycles, rt, full H₂, 1 mL/min). The solvent was evaporated in vacuo to yield intermediate 30 (2.7 g, 71%) as a colorless oil that was used in the next step without further purification.

Preparation of Intermediate 31

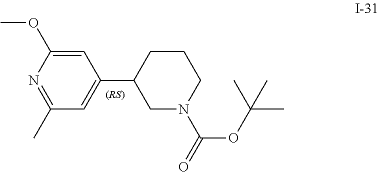 [SMILES: COc1cc(C2CCCN(C(=O)OC(C)(C)C)C2)cc(C)n1]

Intermediate 31 was prepared following an analogous procedure to the one described for the synthesis of intermediate 30 using intermediate 64 as starting material.

Preparation of Intermediate 32

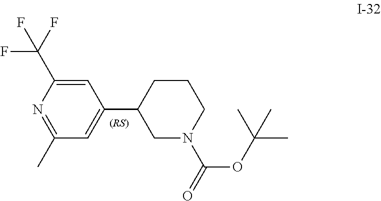 [SMILES: Cc1cc(C2CCCN(C(=O)OC(C)(C)C)C2)cc(C(F)(F)F)n1]

Intermediate 32 was prepared following an analogous procedure to the one described for the synthesis of intermediate 30 using intermediate 65 as starting material.

Preparation of Intermediate 33

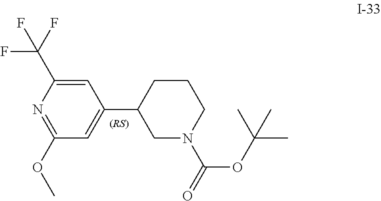 [SMILES: COc1cc(C2CCCN(C(=O)OC(C)(C)C)C2)cc(C(F)(F)F)n1]

Intermediate 33 was prepared following an analogous procedure to the one described for the synthesis of intermediate 30 using intermediate 66 as starting material.

Preparation of Intermediate 34

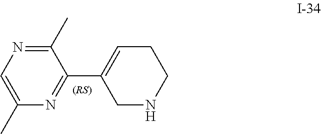

Intermediate 34 was prepared following an analogous procedure to the one described for the synthesis of intermediate 1 using intermediate 67 as starting material.

Preparation of Intermediate 35

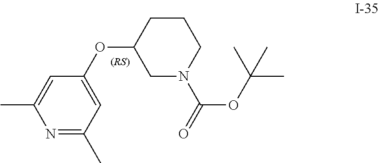 [SMILES: Cc1cc(OC2CCCN(C(=O)OC(C)(C)C)C2)cc(C)n1]

Sodium hydride (0.30 g, 7.45 mmol) was added to a stirred solution of l-Boc-3-hydroxypiperidine (CAS: 85275-45-2; 1.5 g, 7.45 mmol) in DMF (6 mL) at 0° C. and the mixture was stirred for 30 min. Then the mixture was allowed to warm to rt and a solution of 2,6-dimethyl-4-chloropyridine (CAS: 3512-75-2; 0.95 mL, 7.45 mmol) in DMF (1 mL) was added drop wise. The mixture was stirred at rt for 16 h and then at 60° C. for 6 h. After cooling to rt, water was added and the mixture was extracted with EtOAc. The organic layer was dried over Na₂SO₄, filtered and concentrated in vacuo. The residue was purified by flash chromatography (silica gel, EtOAc in heptane: 0/100 to 30/70). The desired fractions were collected and concentrated in vacuo to yield intermediate 35 (0.42 g, 18%) as a colorless oil.

Preparation of Intermediate 36

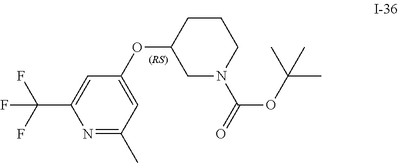 [SMILES: Cc1cc(OC2CCCN(C(=O)OC(C)(C)C)C2)cc(C(F)(F)F)n1]

Trimethylboroxine (CAS: 823-96-1; 2.74 mL, 19.85 mmol), Pd(OAc)₂ (CAS: 3375-31-3; 0.124 g, 0.55 mmol) and tricyclohexylphosphoniumtetrafluoroborate (CAS: 58656-04-5; 0.406 g, 1.10 mmol) were added to a stirred suspension of K₂CO₃ (2.03 g, 14.71 mmol) and intermediate 68 (2.8 g, 7.35 mmol) in deoxygenated 1,4-dioxane (21.4 mL). The mixture was stirred at 100° C. for 4 h under N₂ atmosphere. After cooling to rt, the mixture was washed with water and extracted with DCM. The organic layer was separated, dried (MgSO₄), filtered and the solvents were evaporated in vacuo. The crude product was purified by flash column chromatography (silica; EtOAc in heptane: 0/100 to 30/70). The desired fractions were collected and concentrated in vacuo to yield intermediate 36 (2.63 g, 99%) as a dark-brown oil.

Preparation of Intermediate 37

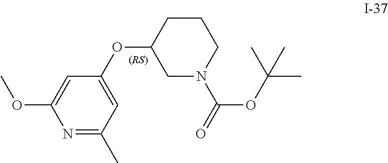 [SMILES: COc1cc(OC2CCCN(C(=O)OC(C)(C)C)C2)cc(C)n1]

Intermediate 37 was prepared following an analogous procedure to the one described for the synthesis of intermediate 35 using 1-Boc-3-hydroxypiperidine (CAS: 85275-45-2) and 4-bromo-2-methoxy-6-methylpyridine (CAS: 1083169-00-9) as starting materials.

Preparation of Intermediate 38

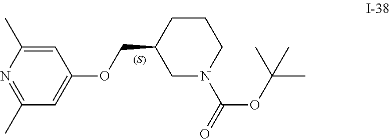

Intermediate 38 was prepared following an analogous procedure to the one described for the synthesis of intermediate 35 using (S)-1-N-Boc-3-hydroxymethyl-piperidine (CAS: 140695-84-7) and 4-chloro-2,6-dimethylpyridine (CAS: 3512-75-2) as starting materials.

Preparation of Intermediate 39

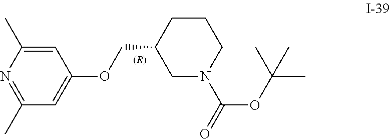

Intermediate 39 was prepared following an analogous procedure to the one described for the synthesis of intermediate 35 using (R)-1-N-Boc-3-hydroxymethyl-piperidine (CAS: 140695-85-8) and 4-chloro-2,6-dimethylpyridine (CAS: 3512-75-2) as starting materials.

Preparation of Intermediate 40

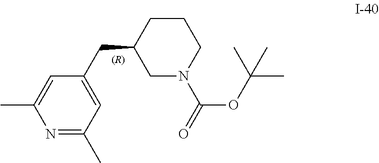

To a 400 mL reactor equipped with overhead stirrer and temperature probe, 4-bromo-2,6-dimethylpyridine (21 g, 113 mmol) was charged under N₂ atmosphere at rt. A THF solution of intermediate 69 (366 mL, 124.44 mmol, 0.34 M solution in THF) was then added followed by N,N,N′,N′-tetramethylethylenediamine (18.66 mL, 124.4 mmol) and contents were degassed by N₂ sparging (5 min).

Bis(triphenylphosphine)palladium(II) dichloride (CAS: 13965-03-2; 1.588 g, 2.263 mmol) was then added and contents degassed again by N₂ sparging for another 5 min. After this, the reaction mixture was warmed to 50° C. and stirred at this temperature for 1 h. The reaction mixture was then cooled down to 20° C. and quenched with a 1:1 mixture of 32% aq. NH₃ and sat. NH₄Cl (200 mL). Water (100 mL) was added followed by EtOAc (200 mL). The resulting biphasic solution was filtered through a pad of Celite® to remove the palladium black residue. Phases were then separated and aqueous back-extracted with EtOAc (200 mL). Combined organic extracts were dried over MgSO₄, solids filtered and solvents distilled under reduced pressure to dryness. Crude material was purified by normal phase column chromatography (silica, EtOAc in heptane 0/100 to 50/50). Desired fractions were collected and concentrated under reduced pressure to yield intermediate 40 (34.44 g, 89% yield) as an orange oil.

Preparation of Intermediate 41

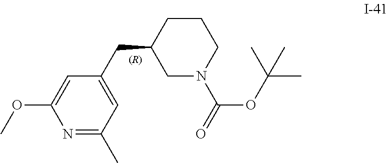

Intermediate 41 was prepared following an analogous procedure to the one described for the synthesis of intermediate 40 using intermediate 69 and 4-bromo-2-methoxy-6-methylpyridine (CAS: 1083169-00-9) as starting materials.

Preparation of Intermediate 42

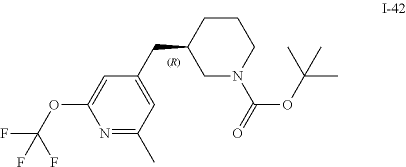

Potassium carbonate (390 mg, 2.82 mmol) was added to a stirred solution of intermediate 70 (1.21 g, 1.41 mmol) in 1,4-dioxane (4.11 mL) and it was deoxigenated with a N₂ flow for 5 min. Then, trimethylboroxine (0.53 mL, 3.81 mmol), Pd(OAc)₂ (23.7 mg, 0.11 mmol) and tricyclohexylphosphoniumtetrafluoroborate (CAS: 58656-04-5; 77.9 mg, 0.211 mmol) were added. The mixture was stirred at 100° C. for 2 h under N₂ atmosphere. After cooling, the water was added and the mixture was extracted with DCM. The organic layer was separated, dried (MgSO₄), filtered and the solvents evaporated in vacuo. The crude product was purified by flash column chromatography (silica; EtOAc in heptane: 0/100 to 30/70). The desired fractions were collected and concentrated in vacuo to yield intermediate 42 (219 mg, 69%) as a dark brown oil, used in the next step without further purification.

Preparation of Intermediate 43

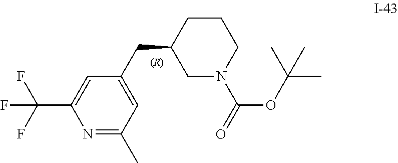

Intermediate 43 was prepared following an analogous procedure to the one described for the synthesis of intermediate 42 using intermediate 71 as starting material.

Preparation of Intermediate 54

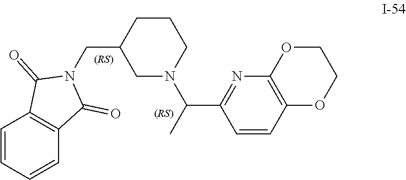 [SMILES: CC(c1ccc2c(n1)OCCO2)N1CCCC(CN2C(=O)c3ccccc3C2=O)C1]

A mixture of intermediate 75 (160 mg, 0.57 mmol), phthalimide (93 mg, 0.63 mmol) and triphenylphosphine (226 mg, 0.86 mmol) in dry THF (20 mL) was stirred under nitrogen at rt. Then, DIAD (CAS: 2446-83-5; 0.17 mL, 0.86 mmol) was added and the mixture was stirred at rt overnight. The solvents were evaporated in vacuo. The crude product was purified by flash column chromatography (silica; MeOH in DCM 0/100 to 5/95). The desired fractions were collected and concentrated in vacuo to intermediate 54 (243 mg, quantitative) as a yellow sticky solid.

Preparation of Intermediate 55

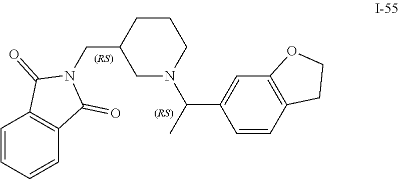 [SMILES: CC(c1ccc2c(c1)OCC2)N1CCCC(CN2C(=O)c3ccccc3C2=O)C1]

Intermediate 55 was prepared following an analogous procedure to the one described for the synthesis of intermediate 54 using intermediate 76 as starting material.

Preparation of Intermediate 56

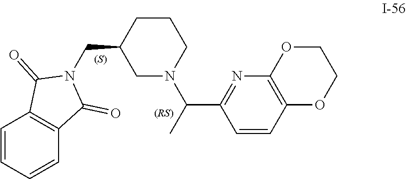

Intermediate 56 was prepared following an analogous procedure to the one described for the synthesis of intermediate 35 using intermediate 77 as starting material.

Preparation of Intermediate 57

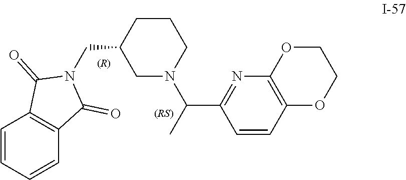

Intermediate 56 was prepared following an analogous procedure to the one described for the synthesis of intermediate 54 using intermediate 78 as starting material.

Preparation of Intermediate 58

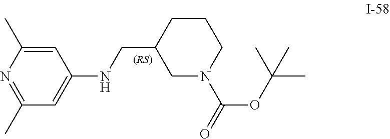

To a mixture of 2′-(dicyclohexylphosphino)-N,N-dimethyl-[1,1′-biphenyl]-2-amine (38.6 mg, 0.1 mmol), tris(dibenzylideneacetone)dipalladium (0) (CAS: 51364-51-3: 64 mg, 0.07 mmol) and sodium tert-butoxide (202 mg, 2.1 mmol) in 1,4-dioxane (3.96 mL), 4-chloro-2,6-dimethylpyridine (0.178 mL, 1.4 mmol) and 3-(aminomethyl)-1-Boc-piperidine (360 mg, 1.68 mmol) were added and the reaction mixture was heated at 100° C. in a sealed tube for 18 h. The reaction mixture was filtered over a pad of dicalite and rinsed with DCM. The filtrate was concentrated and the residue purified by flash column chromatography (silica:ammonia in methanol in DCM, 0/100 to 5/95). The desired fractions were collected and concentrated in vacuo to yield intermediate 58 (445 mg, 99%) as an oil.

Preparation of Intermediate 59

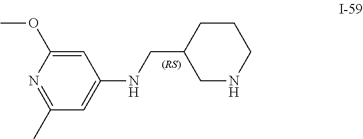

Intermediate 59 was prepared following an analogous procedure to the one described for the synthesis of intermediate 15 using intermediate 60 as starting material.

Preparation of Intermediate 60

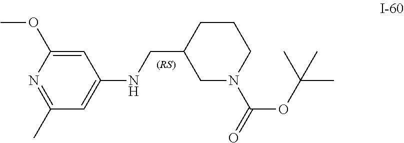 [SMILES: COc1cc(NCC2CCCN(C(=O)OC(C)(C)C)C2)cc(C)n1]

Intermediate 60 was prepared following an analogous procedure to the one described for the synthesis of intermediate 58 using 3-(aminomethyl)-1-Boc-piperidine and 4-bromo-2-methoxy-6-methylpyridine (CAS: 1083169-00-9) as starting materials.

Preparation of Intermediate 61

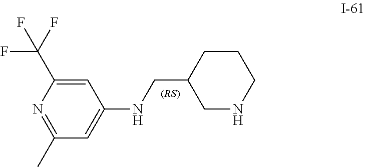 [SMILES: Cc1cc(NCC2CCCNC2)cc(C(F)(F)F)n1]

Intermediate 61 was prepared following an analogous procedure to the one described for the synthesis of intermediate 15 using intermediate 62 as starting material.

Preparation of Intermediate 62

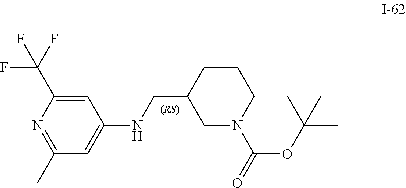 [SMILES: Cc1cc(NCC2CCCN(C(=O)OC(C)(C)C)C2)cc(C(F)(F)F)n1]

Trimethylboroxine (0.49 mL, 3.53 mmol) was added to a stirred suspension of intermediate 79 (1.16 g, 2.94 mmol), K₃PO₄ (1.25 g, 5.9 mmol), 2-dicyclohexylphosphino-2′,4′,6′-triisopropylbiphenyl (CAS: 564483-18-7; 140 mg, 0.29 mmol) and tris(dibenzylideneacetone)dipalladium (0) (CAS: 51364-51-3: 134 mg, 0.15 mmol) in 1,4-dioxane (25 mL) under N₂ atmosphere. The mixture was stirred at 95° C. overnight. Water and EtOAc were added. The organic layer was separated, dried (MgSO₄) and filtered and the solvents evaporated in vacuo. The crude was purified by flash column chromatography (silica; EtOAc in heptane 0/100 to 50/50). The desired fractions were collected and concentrated in vacuo to yield intermediate 62 (1.1 g, 95%) a pale yellow sticky solid.

Preparation of Intermediate 63

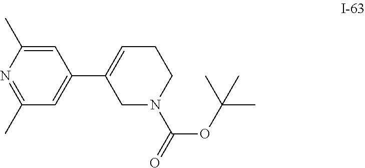 [SMILES: Cc1cc(C2=CCCN(C(=O)OC(C)(C)C)C2)cc(C)n1]

1,4-Dioxane (25.5 mL) followed by Na₂CO₃ (25.5 mL, aqueous saturated solution) were added to a stirred mixture of 4-chloro-2,6-dimethylpyridine (1.9 mL, 15 mmol), tert-butyl 5-(4,4,5,5-tetramethyl-1,3,2-dioxaborolan-2-yl)-3,6-dihydropyridine-1(2H)-carboxylate (CAS: 885693-20-9; 5.1 g, 16.5 mmol) and Pd(PPh₃)₄ (1.04 g, 0.89 mmol) in a sealed tube and under N₂ atmosphere. The mixture was stirred at 130° C. for 30 min under microwave irradiation. The mixture was treated with water and extracted with DCM. The organic layer was separated, dried (Na₂SO₄), filtered and the solvents evaporated in vacuo. The crude product was purified by flash column chromatography (silica, EtOAc in heptane 0/100 to 100/0). The desired fractions were collected and concentrated in vacuo to yield intermediate 63 (3.6 g, 83%) as a colorless oil.

Preparation of Intermediate 64

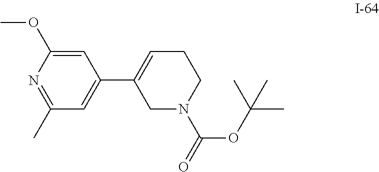 [SMILES: COc1cc(C2=CCCN(C(=O)OC(C)(C)C)C2)cc(C)n1]

Intermediate 64 was prepared following an analogous procedure to the one described for the synthesis of intermediate 63 using tert-butyl 5-(4,4,5,5-tetramethyl-1,3,2-dioxaborolan-2-yl)-3,6-dihydropyridine-1(2H)-carboxylate (CAS: 885693-20-9) and 4-bromo-2-methoxy-6-methylpyridine (CAS: 1083169-00-9) as starting materials.

Preparation of Intermediate 65

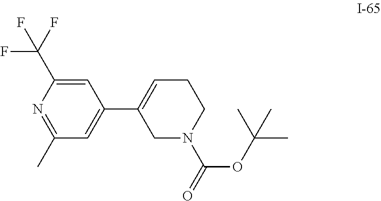 [SMILES: Cc1cc(C2=CCCN(C(=O)OC(C)(C)C)C2)cc(C(F)(F)F)n1]

Pd(OAc)₂ (34 mg, 0.15 mmol) and tricyclohexylphosphonium tetrafluoroborate (CAS: 58656-04-5; 111.6 mg, 0.30 mmol) were added to a stirred solution of intermediate 80 (4.5 g, 12.4 mmol), trimethylboroxine (0.763 mL, 5.46 mmol) and K₂CO₃ (0.84 g, 6.06 mmol) in 1,4-dioxane (8.83 mL) and then the mixture was deoxygenated with a N₂ flow for 5 min. The mixture was stirred at 100° C. for 2 h under N₂ atmosphere. After cooling, the mixture was washed with water and extracted with DCM. The organic layer was separated, dried (MgSO₄), filtered and the solvents evaporated in vacuo. The crude product was purified by flash column chromatography (silica; EtOAc in heptane: 0/100 to 15/85). The desired fractions were collected and concentrated in vacuo to yield intermediate 65 (4.25 g, 89%) as pale yellow oil.

Preparation of Intermediate 66

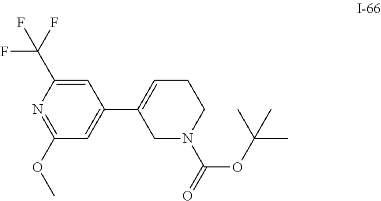 [SMILES: COc1cc(C2=CCCN(C(=O)OC(C)(C)C)C2)cc(C(F)(F)F)n1]

To a mixture of 2-chloro-4-iodo-6-trifluoromethylpyridine (CAS: 205444-22-0; 3 g, 9.76 mmol), tert-butyl 5-(4,4,5,5-tetramethyl-1,3,2-dioxaborolan-2-yl)-3,6-dihydropyridine-1(2H)-carboxylate (CAS: 885693-20-9; 3.62 g, 11.71 mmol) and K₃PO₄ (6.21 g, 29.3 mmol) in MeOH (60 mL), trans-bis(dicyclohexylamine)palladium(II) acetate (CAS: 628339-96-8; 114.2 mg, 0.2 mmol) was added and the reaction mixture was stirred at rt for 18 h. The reaction was filtered through Celite®, washed the Celite® pad rinsed with EtOAc and the combined filtrates were evaporated in vacuo. The crude product was purified by flash column chromatography (silica; ethyl acetate in heptane: 0/100 to 20/80). The desired fractions were collected and concentrated in vacuo to yield a colorless oil (3.4 g, contains intermediate 66 and the 2-chloro-6-trifluoromethylpyridyl coupling product). This oil was taken up in dry MeOH (50 mL) and sodium methoxide (2.14 mL, 9.37 mmol, 25% solution on MeOH) was added. The mixture was stirred at rt for 16 h. Then water was added and the desired product extracted with DCM. The organic layer was separated, dried (Na₂SO₄), filtered and the solvent evaporated in vacuo. The crude product was purified by flash column chromatography (silica; DCM in heptane: 20/80 to 100/0).

The desired fractions were collected and concentrated in vacuo to yield intermediate 66 (3.1 g) as a colorless oil.

Preparation of Intermediate 67

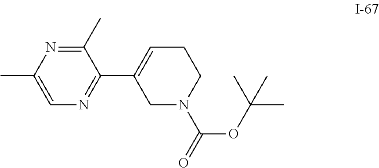 [SMILES: Cc1cnc(C2=CCCN(C(=O)OC(C)(C)C)C2)c(C)n1]

Intermediate 67 was prepared following an analogous procedure to the one described for the synthesis of intermediate 63 using tert-butyl 5-(4,4,5,5-tetramethyl-1,3,2-dioxaborolan-2-yl)-3,6-dihydropyridine-1(2H)-carboxylate (CAS: 885693-20-9) and 2-chloro-3,5-dimethylpyrazine (CAS: 38557-72-1) as starting materials.

Preparation of Intermediate 68

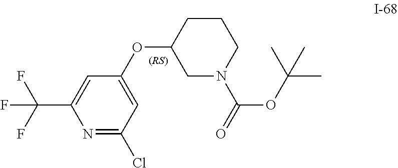 [SMILES: CC(C)(C)OC(=O)N1CCCC(Oc2cc(Cl)nc(C(F)(F)F)c2)C1]

Intermediate 68 was prepared following an analogous procedure to the one described for the synthesis of intermediate 35 using 1-Boc-3-hydroxypiperidine (CAS: 85275-45-2) and 2-chloro-4-iodo-6-(trifluoromethyl)pyridine (CAS: 205444-22-0) as starting materials.

Preparation of Intermediate 69

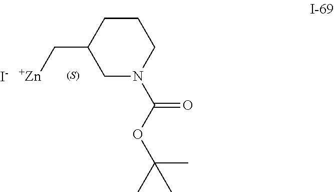

A solution of 3S-iodomethylpiperidine-1-carboxylic acid tert-butyl ester (CAS: 384829-99-6; 47.9 g, 147.3 mmol) in THF (292.8 mL) was pumped through a column containing activated zinc (14.45 g, 221 mmol) at 40° C. under N₂ at a flow rate of 1.5 mL/min. The resulting solution was collected over molecular sieves under N₂ atmosphere to yield intermediate 69 as a clear light brown solution. This solution was titrated twice against iodine in THF (0.34 M) and used as such in the next step.

Preparation of Intermediate 70

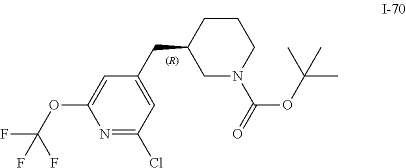

Intermediate 69 (1.1 equiv; 0.26 mL, 0.32 M in THF) was added to 2-chloro-4-iodo-6-(trifluoromethoxy)pyridine (CAS: 1221171-96-5; 681 mg, 2.1 mmol) and bis(tri-tert-butylphosphine)palladium(0) (53 mg, 0.1 mmol) under N₂ atmosphere. The mixture was stirred at rt for 16 h. Then more intermediate 69 (1.1 equiv; 0.26 mL, 0.32 M in THF) and bis(tri-tert-butylphosphine)palladium(0) (53 mg, 0.1 mmol) were added under N₂ atmosphere and the mixture was stirred at 65° C. for 3 h. After cooling to rt the mixture was treated with a mixture of aq. sat. NH₄Cl and NH₄OH (1:1) and extracted with EtOAc. The organic layer was separated, dried (Na₂SO₄), filtered and the solvents evaporated in vacuo. The crude product was purified by flash column chromatography (silica; EtOAc in heptane: 0/100 to 20/80). The desired fractions were collected and concentrated in vacuo to yield intermediate 70 (332.4 mg, 77% pure) as pale yellow oils, used in the next step without further purification

For the above reaction Zn was activated as follows: A solution of TMSCl (2.2 mL) and 1-bromo-2-chloroethane (0.5 mL) in THF (10 mL) was passed through the column containing Zn at a flow of 1 mL/min.

Preparation of Intermediate 71

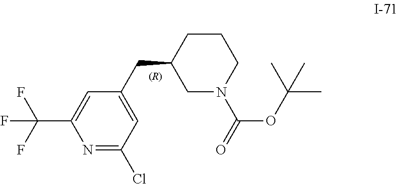

Intermediate 71 was prepared following an analogous procedure to the one described for the synthesis of intermediate 70 using intermediate 69 and 2-chloro-4-iodo-6-(trifluoromethyl)pyridine (CAS: 205444-22-0) as starting materials.

Preparation of Intermediate 75

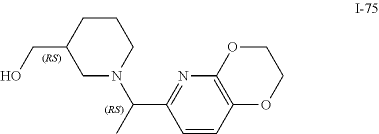 [SMILES: CC(c1ccc2c(n1)OCCO2)N1CCCC(CO)C1]

Tetrabutylammonium fluoride hydrate (CAS: 22206-57-1; 1.18 g, 4.24 mmol) was added to a stirred solution of intermediate 82 in THF (13 mL) at rt. The mixture was stirred at rt for 8 h. The solvents evaporated in vacuo. The crude product was purified by flash column chromatography (silica; MeOH in DCM 0/100 to 10/90). The desired fractions were collected and concentrated in vacuo to yield intermediate 75 (509 mg, 86%) as a pale yellow sticky solid.

Preparation of Intermediate 76

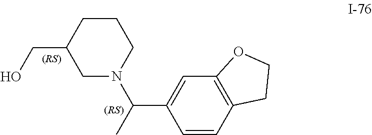 [SMILES: CC(c1ccc2c(c1)OCC2)N1CCCC(CO)C1]

Intermediate 76 was prepared following an analogous procedure to the one described for the synthesis of intermediate 75 using intermediate 83 as starting material.

Preparation of Intermediate 77

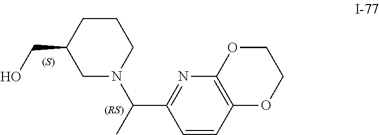

Intermediate 77 was prepared following an analogous procedure to the one described for the synthesis of intermediate 75 using intermediate 84 as starting material.

Preparation of Intermediate 78

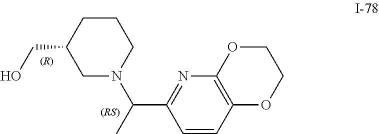

Intermediate 78 was prepared following an analogous procedure to the one described for the synthesis of intermediate 75 using intermediate 85 as starting material.

Preparation of Intermediate 79

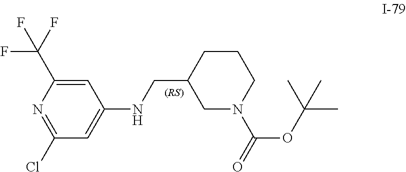 [SMILES: CC(C)(C)OC(=O)N1CCCC(CNc2cc(Cl)nc(C(F)(F)F)c2)C1]

Intermediate 79 was prepared following an analogous procedure to the one described for the synthesis of intermediate 58 using 3-(aminomethyl)-1-Boc-piperidine and 2-chloro-4-iodo-6-(trifluoromethyl)pyridine (CAS: 205444-22-0) as starting materials.

Preparation of Intermediate 80

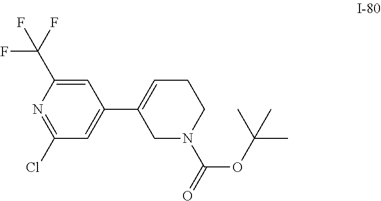 [SMILES: CC(C)(C)OC(=O)N1CCC=C(c2cc(Cl)nc(C(F)(F)F)c2)C1]

Intermediate 80 was prepared following an analogous procedure to the one described for the synthesis of intermediate 66 using tert-butyl 5-(4,4,5,5-tetramethyl-1,3,2-dioxaborolan-2-yl)-3,6-dihydropyridine-1(2H)-carboxylate (CAS: 885693-20-9) and 2-chloro-4-iodo-6-trifluoromethylpyridine (CAS: 205444-22-0) as starting materials.

Preparation of Intermediate 82

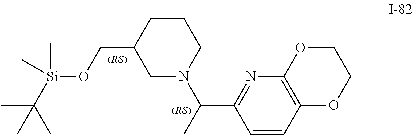 [SMILES: CC(c1ccc2c(n1)OCCO2)N1CCCC(CO[Si](C)(C)C(C)(C)C)C1]

2,3-Dihydro-[1,4]dioxino[2,3-b]pyridine-6-carboxaldehyde (CAS: 615568-24-6; 232 mg, 1.4 mmol) and titanium(IV) isopropoxide (1.03 mL, 3.51 mmol) were added to a solution of tert-butyldimethyl[(3-piperidinyl)methoxy]silane (CAS: 876147-50-1, 269 mg, 1.17 mmol) in anhydrous THF (3 mL) at rt and the reaction mixture was stirred at rt for 18 h. The volatiles were evaporated vacuo. Then, anhydrous THF (3 mL) was added and the reaction was cooled to 0° C. and methyl magnesium bromide (4.18 mL, 5.85 mmol, 1.4 M in THF) was added dropwise and the reaction mixture was stirred at 0° C. for 15 mins and at rt for 15 h. NH₄Cl (aq sat soltn) was added and the mixture was extracted with DCM (10 mL×3 times). The combined organic extracts were dried over MgSO₄ and concentrated in vacuo. The crude product was purified by flash column chromatography (silica; EtOAc in heptane 0/100 to 40/60). The desired fractions were collected and concentrated in vacuo to yield intermediate 82 (346 mg, 75%) as a colorless oil.

Preparation of Intermediate 83

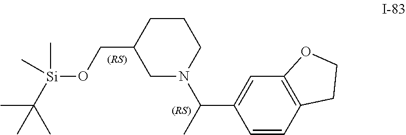 [SMILES: CC(c1ccc2c(c1)OCC2)N1CCCC(CO[Si](C)(C)C(C)(C)C)C1]

Intermediate 83 was prepared following an analogous procedure to the one described for the synthesis of intermediate 82 using of tert-butyldimethyl[(3-piperidinyl)methoxy]silane (CAS: 876147-50-1) and 2,3-dihydrobenzofuran-6-carboxaldehyde (CAS: 55745-96-5) as starting materials.

Preparation of Intermediate 84

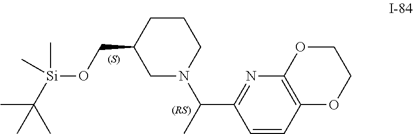

Intermediate 84 was prepared following an analogous procedure to the one described for the synthesis of intermediate 82 using of tert-butyldimethyl[(3-(S)-piperidinyl)methoxy]silane and 2,3-dihydro-[1,4]dioxino[2,3-b]pyridine-6-carboxaldehyde as starting materials.

Preparation of Intermediate 85

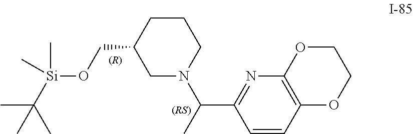

Intermediate 84 was prepared following an analogous procedure to the one described for the synthesis of intermediate 82 using of tert-butyldimethyl[3-(R)-piperidinyl)methoxy]silane and 2,3-dihydro-[1,4]dioxino[2,3-b]pyridine-6-carboxaldehyde as starting materials.

Preparation of Intermediate 86

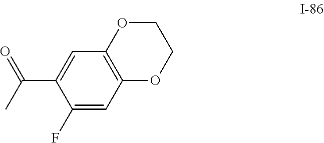

To a mixture of 1-(2,3-dihydro-1,4-benzodioxin-6-yl)ethenone (2.1 g, 11.78 mmol) in 1-butyl-3-methylimidazolium tetrafluoroborate (CAS 174501-65-6; 7 mL), N-fluoro-N′-(chloromethyl)triethylenediaminebis(tetrafluoroborate) (CAS140681-55-6; 10.43 g, 29.5 mmol) was added. The reaction mixture was heated at 70° C. for 16 h. Then it was cooled to rt, treated with water and extracted with EtOAc (2×15 mL). The combined organic layer was evaporated in vacuo to afford an oil which was purified by flash column chromatography (SiO₂, EtOAc in Heptane, 0/100 to 10/90). The desired fractions were concentrated to yield intermediate 86 (1.1 g, 48%) as white solid.

Preparation of Intermediate 87

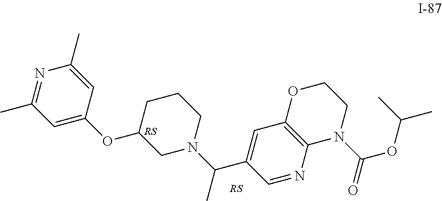 [SMILES: Cc1cc(OC2CCCN(C(C)c3cnc4c(c3)OCCN4C(=O)OC(C)C)C2)cc(C)n1]

Titanium(IV) isopropoxide (0.22 mL, 0.73 mmol) and tert-butyl 7-formyl-2H-pyrido[3,2-b][1,4]oxazine-4(3H)-carboxylate (CAS: 1287312-62-2; 153.7 mg, 0.58 mmol) were added to a stirred solution of intermediate 6 (100 mg, 0.48 mmol) in anhydrous DCM (2 mL). The reaction mixture was stirred at rt for 20 h. Then the reaction was cooled to 0° C. and methyl magnesium bromide (1.73 mL, 2.43 mmol, 1.4 M in THF) was added dropwise and the reaction mixture was stirred at 0° C. for 5 min. and at rt for 2 h. Then NH₄Cl (aq sat soltn) was added and the product extracted with DCM. The organic layer was separated, dried (MgSO₄), filtered and the solvents evaporated in vacuo to yield intermediate 87 (100 mg, 45%, mixture of diastereoisomers) as a yellow oil.

Preparation of Intermediate 88

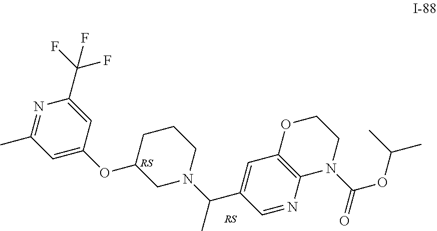 [SMILES: Cc1cc(OC2CCCN(C(C)c3cnc4c(c3)OCCN4C(=O)OC(C)C)C2)cc(C(F)(F)F)n1]

Intermediate 88 was prepared following an analogous procedure to the one described for the synthesis of intermediate 87 using intermediate 7 and tert-butyl 7-formyl-2H-pyrido[3,2-b][1,4]oxazine-4(3H)-carboxylate (CAS: 1287312-62-2) as starting materials.

Preparation of Intermediate 89 and Final Compound 125

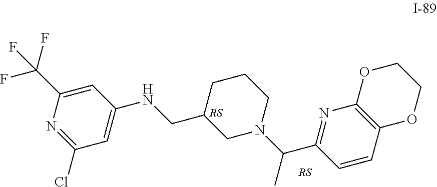 [SMILES: CC(c1ccc2c(n1)OCCO2)N1CCCC(CNc2cc(Cl)nc(C(F)(F)F)c2)C1]

Intermediate 89 was prepared following an analogous procedure to the one described for the synthesis of product 21 using intermediate 11 (188 mg, 0.63 mmol) and 2-chloro-4-iodo-6-trifluoromethylpyridine (205444-22-0) as starting materials.

Preparation of Intermediate 90 and Final Compound 126

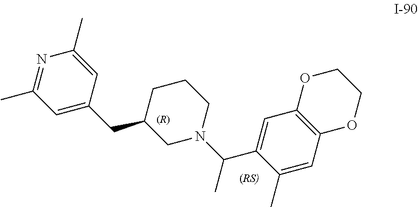

Intermediate 16 (100 mg, 0.49 mmol) was added to a stirred solution of 5-methyl-2,3-dihydrobenzo[b][1,4]dioxine-6-carboxylic acid (CAS: 924871-41-0; 95 mg, 0.49 mmol), N-[(dimethylamino)-1H-1,2,3-triazolo-[4,5-b]pyridin-1-ylmethylene]-N-methylmethanaminium hexafluorophosphate N-oxide (CAS: 148893-10-1; 307 mg, 0.81 mmol) and diisopropylethylamine (0.337 mL, 1.96 mmol) in DMF (2.56 mL) and the mixture was stirred at rt under N₂ atmosphere for 16 h. Then the mixture was diluted with a saturated aqueous solution of NaHCO₃ and extracted with EtOAc. The organic layer was separated, washed with brine, dried (MgSO₄), filtered and the solvents evaporated in vacuo. The crude product was purified by flash column chromatography (silica gel, EtOAc in heptane 0/100 to 100/0). The desired fractions were collected and evaporated in vacuo and the residue was purified by flash column chromatography (MeOH in DCM from 0/100 to 10/90). The desired fractions were collected and the solvents evaporated in vacuo to yield intermediate 90 (87 mg, 47%) as a colorless oil.

Preparation of Intermediate 91

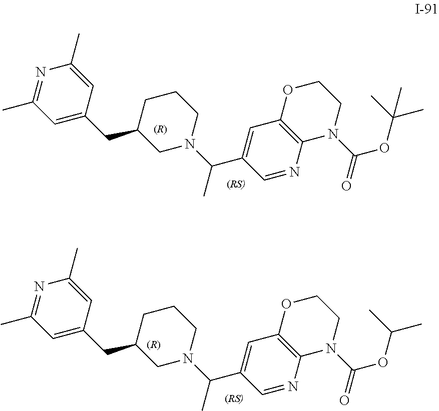

Intermediate 91 (mixture of carbamates) was prepared following an analogous procedure to the one described for the synthesis of product 1 using intermediate 16 and tert-butyl 7-formyl-2H-pyrido[3,2-b][1,4]oxazine-4(3H)-carboxylate (CAS: 1287312-62-2) as starting materials.

Preparation of Intermediate 92 and Final Compound 127

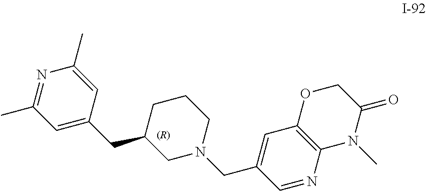

Intermediate 92 was prepared following an analogous procedure to the one described for the synthesis of product 43 using intermediate 16 (2×HCl salt) and intermediate 93 as starting materials.

Preparation of Intermediate 93

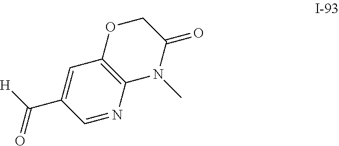

Sodium periodate (2.91 g, 13.6 mmol) followed by osmium tetroxide (0.472 mL, 0.035 mmol, 2.5% in t-BuOH) and 2,6-dimethylpyridine (0.71 mL, 6.11 mmol) were added to a stirred solution of intermediate 94 (508 mg, 2.67 mmol) in 1,4-dioxane (25 mL) and water (7.5 mL) in a sealed tube and under N₂ atmosphere. The mixture was stirred at rt for 16 h. The mixture was treated with water, filtered and washed with EtOAc. The filtrate was extracted with additional EtOAc. The organic layer was separated, dried (MgSO₄), filtered and the solvents evaporated in vacuo. The crude product was purified by flash column chromatography (SiO₂, EtOAc in DCM 0/100 to 100/0). The desired fractions were collected and concentrated in vacuo to yield intermediate 93 (230 mg, 45%) as a white solid.

Preparation of Intermediate 94

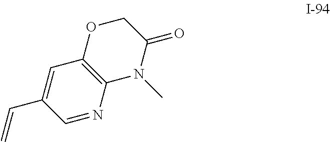

Potassium carbonate (7.5 mL, 10% aq soltn) followed by 4,4,5,5-tetramethyl-2-vinyl-1,3,2-dioxaborolane (CAS: 75927-49-0; 0.65 mL, 3.83 mmol) and Pd(PPh₃)₄ (365 mg, 0.31 mmol) were added to a stirred solution of 7-bromo-4-methyl-2,3-dihydro-4H-pyrido[3,2-b][1,4]oxazin-3-one (CAS: 122450-97-9) in 1,4-dioxane (7.5 mL) in a sealed tube and under N₂ atmosphere. The mixture was stirred at 150° C. for 15 min under microwave irradiation. The mixture was treated with water and extracted with DCM. The organic layer was separated, dried (MgSO₄), filtered and the solvents evaporated in vacuo. The crude product was purified by flash column chromatography (SiO₂, EtOAc in heptane 0/100 to 100/0). The desired fractions were collected and concentrated in vacuo to yield intermediate 94 (516 mg, 87%) as a white solid.

Preparation of Intermediate 95

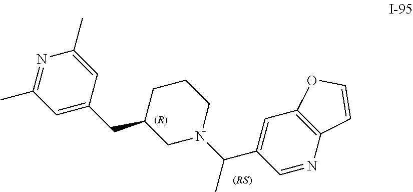

Intermediate 95 was prepared following an analogous procedure to the one described for the synthesis of product 2 using intermediate 16 and 1-{furo[3,2-b]pyridin-6-yl}ethan-1-one (CAS: 1203499-00-6) as starting materials.

Preparation of Intermediate 97

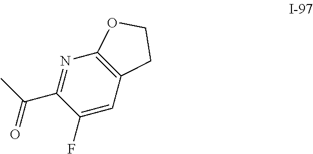

To a mixture of intermediate 98 (340 mg, 2.071 mmol) in dry THF (20 mL), methyl magnesium bromide (2.071 mL, 2.9 mmol, 1.4 M in THF) was added at 0° C. After completion of the addition, the reaction was stirred for 16 h at rt. The mixture was quenched with 1M aq HCl and stirred for 30 min, then the crude was basified with NH₄OH until pH 8. The solution was extracted with EtOAc (2×5 mL) The combined organic extracts were dried (Na₂SO₄), filtered and evaporated to dryness to give a residue that was purified by flash column chromatography (SiO₂, EtOAc in heptane 0/100 to 20/80). The desired fractions were collected and concentrated to yield intermediate 97 (150 mg, 40%) as a colorless oil.

Preparation of Intermediate 98

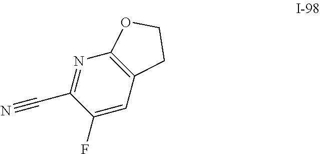 [SMILES: N#Cc1nc2c(cc1F)CCO2]

To a mixture of intermediate 99 (400 mg, 2.57 mmol) in acetonitrile (7 mL), trimethylsilyl cyanide (CAS: 7677-24-9; 1.29 mL, 10.3 mmol) and triethylamine (0.9 mL, 6.47 mmol) were added. The mixture was stirred at 90° C. for 24 h. The mixture was cooled and treated with water and extracted with EtOAc (2×10 mL). The combined organic extracts were dried over MgSO₄ and the solvent was evaporated in vacuo to give a residue that was purified by flash column chromatography (SiO₂, EtOAc in heptane 0/100 to 30/60). The desired fractions were collected and concentrated in vacuo to intermediate 98 (320 mg, 76%) as an oil.

Preparation of Intermediate 99

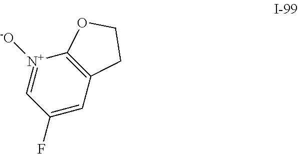

To a mixture of 5-fluoro-2,3-dihydrofuro[2,3-b]pyridine (CAS: 1356542-41-0; 500 mg, 3.6 mmol) in DCM (15 mL), meta-chloroperbenzoic acid (806 mg, 4.7 mmol) was added at rt. The mixture was stirred at 25° C. for 36 h. The solvent was removed in vacuo, and the residue thus obtained was purified by silica gel column chromatography (silica; EtOAc in heptane 0/100 to 30/70 then DCM in MeOH 0/100 to 6/94). The desired fractions were collected and concentrated in vacuo to afford intermediate 99 (400 mg, 72%) as white solid.

Preparation of Intermediate 100

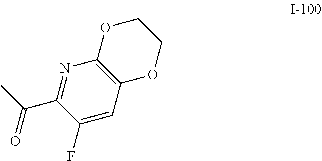

To a mixture of intermediate 101 (1.6 g, 5.7 mmol) in toluene (15 mL), bis(triphenylphosphine)palladium(II) dichloride (400 mg, 0.57 mmol) and tributyl(1-ethoxyvinyl)tin (CAS: 97674-02-7; 2.5 mL, 7.4 mmol) were added. The mixture was heated at 92° C. for 16 h, then the crude was cooled and treated with aqueous 2N HCl (5 mL) and the mixture was stirred for 2 h. The crude was neutralised with an aqueous saturated solution of NaHCO₃ and extracted with EtOAc and the combined organic layers were evaporated in vacuo. The crude was purified by flash column chromatography (SiO₂, MeOH in DCM 0/100 to 5/95). The desired fractions were collected and concentrated in vacuo to yield intermediate 100 (850 mg, 76%) as orange solid.

Preparation of Intermediate 101

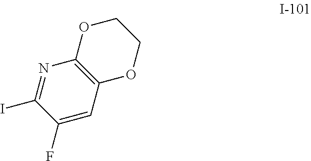 [SMILES: Fc1cc2c(nc1I)OCCO2]

To a mixture of intermediate 102 (5 g, 12.2 mmol) in t-BuOH (6.91 mL), potassium tert-butoxide (206 mg, 1.83 mmol) was added at rt. The mixture was heated at 90° C. for 3 h. After cooling, the solvent was removed in vacuo and the residue was diluted with water. The aqueous solution was extracted with EtOAc (3×12 mL). The combined organic layers were washed with brine (2×10 mL), separated and dried over anhydrous Na₂SO₄ and concentrated. The crude was purified by flash column chromatography (SiO₂, MeOH in DCM 0/100 to 5/95). The desired fractions were collected and concentrated in vacuo to yield intermediate 101 (1.6 g, 47%) as white solid.

Preparation of Intermediate 102

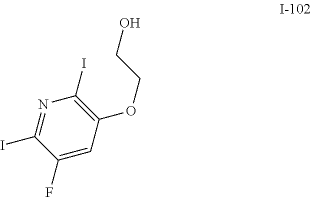 [SMILES: OCCOc1cc(F)c(I)nc1I]

To a mixture of intermediate 103 (8 g, 15.3 mmol) in THF (120 mL), tetrabutylammonium fluoride (15.3 mL, 15. mmol, 1M solution in THF) was added the mixture was stirred for 3 h at rt. Water was added and the crude was extracted with EtOAc. The organic phase was dried (Na₂SO₄) and evaporated in vacuo to afford an oil which was purified by column chromatography (SiO₂, MeOH in DCM, 0/100 to 5/95). The desired fractions were concentrated to yield intermediate 102 (5.8 g, 92%) as oil.

Preparation of Intermediate 103

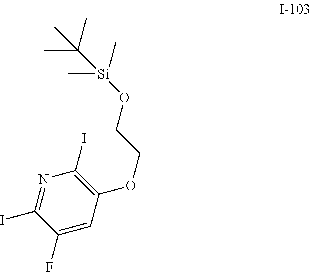 [SMILES: CC(C)(C)[Si](C)(C)OCCOc1cc(F)c(I)nc1I]

A mixture of intermediate 104 (6.1 g, 16.7 mmol), (2-bromoethoxy)dimethyl-tert-butylsilane (4.4 gm 18.4 mmol), and potassium tert-butoxide (5.08 g, 36.78 mmol) in DMF (15 mL) was heated at 90° C. for 5 h. The crude was cooled and treated with water and extracted with EtOAc (2×20 mL). The combined organic extracts were evaporated in vacuo to afford a residue that was purified by column chromatography (SiO₂, EtOAc in heptane, 0/100 to 20/80). The desired fractions were concentrated in vacuo to yield intermediate 103 (8.1 g, 93%) as an oil.

Preparation of Intermediate 104

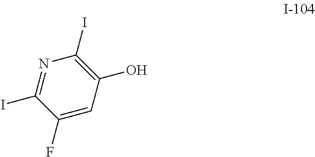

To a solution of 3-fluoro-5-hydroxypyridine (2 g, 17.7 mmol) in Na₂CO₃ (30 mL, aq. sat. soltn.) and water (10 mL), 12 (9.2 g, 36.25 mmol) was added and the mixture was stirred for 16 h at rt. The reaction mixture was quenched with an aqueous saturated solution of Na₂S₂O₃ and the solution pH was adjusted to pH=5 by addition of aqueous HCl. The reaction mixture was extracted with EtOAc (3×70 mL) and the combined organic layer was dried over MgSO₄, filtered and evaporated in vacuo to yield intermediate 104 (6.02 g, 93%) as a yellow solid.

Preparation of Intermediate 105

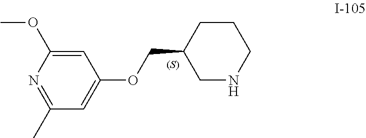

Intermediate 105 was prepared following an analogous procedure to the one described for the synthesis of intermediate 1 using intermediate 106 as starting material.

Preparation of Intermediate 106

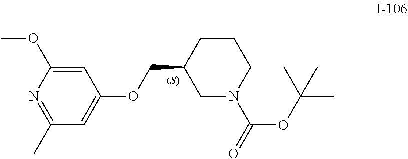

Intermediate 106 was prepared following an analogous procedure to the one described for the synthesis of intermediate 35 using (S)-1-Boc-3-(hydroxymethyl)piperidine (CAS: 140695-84-7) and 4-bromo-2-methoxy-6-methylpyridine (CAS: 1083169-00-9) as starting materials.

Preparation of Intermediate 107

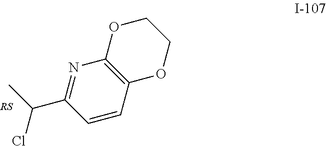

Thionyl chloride (6.51 mL, 89 mmol) was added to a solution of intermediate 108 (4.04 g, 22.3 mmol) in DCM (150 mL) at 0° C. The mixture was stirred at rt for 12 h. Water (80 mL) was added and the mixture was extracted with DCM (80 mL×3). The combined organic layers were dried (Na₂SO₄), filtered and evaporated in vacuo to yield crude intermediate 107 (3.53 g, 79%) as a brown oil that solidified upon standing.

Preparation of Intermediate 108

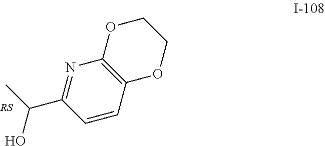

Sodium borohydride (3.54 g, 94 mmol) was added to a solution of 1-(2,3-dihydro-[1,4]dioxino[2,3-b]pyridin-6-yl)ethenone (CAS: 1254044-25-1; 4.5 g, 23.4 mmol) in EtOH (109 mL) at 0° C. The mixture was stirred at rt for 10 min. Water was added and the mixture was extracted with DCM (80 mL×3). The organic layers were combined, dried (Na₂SO₄), filtered and concentrated in vacuo to yield intermediate 108 (4.04 g, 95%) as a pale yellow oil.

Preparation of Intermediate 109

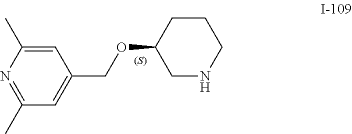

Intermediate 109 was prepared following an analogous procedure to the one described for the synthesis of intermediate 1 using intermediate 110 as starting material.

Preparation of Intermediate 110

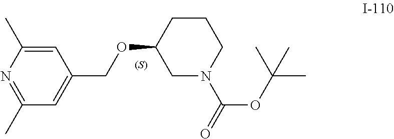

Intermediate 110 was prepared following an analogous procedure to the one described for the synthesis of intermediate 35 using (S)-1-Boc-3-hydroxypiperidine (CAS: 140695-84-7) and 4-chloromethyl-2,6-dimethylpyridine (CAS: 1083169-00-9) as starting materials.

Preparation of Intermediate 111

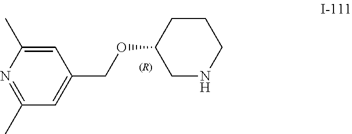

Intermediate 111 was prepared following an analogous procedure to the one described for the synthesis of intermediate 1 using intermediate 112 as starting material.

Preparation of Intermediate 112

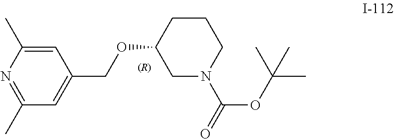

Intermediate 112 was prepared following an analogous procedure to the one described for the synthesis of intermediate 35 using (R)-1-Boc-3-hydroxypiperidine (CAS: 140695-84-7) and 4-chloromethyl-2,6-dimethylpyridine (CAS: 1083169-00-9) as starting materials.

Preparation of Intermediate 113

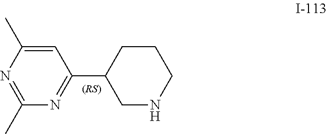

Intermediate 113 was prepared following an analogous procedure to the one described for the synthesis of intermediate 1 using intermediate 114 as starting material.

Preparation of Intermediate 114

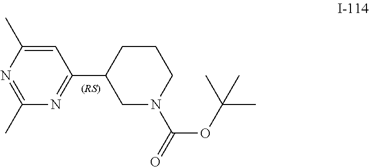 [SMILES: Cc1cc(C2CCCN(C(=O)OC(C)(C)C)C2)nc(C)n1]

Intermediate 114 was prepared following an analogous procedure to the one described for the synthesis of intermediate 30 using intermediate 115 as starting material.

Preparation of Intermediate 115

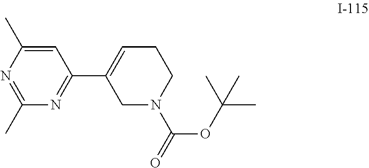 [SMILES: Cc1cc(C2=CCCN(C(=O)OC(C)(C)C)C2)nc(C)n1]

Intermediate 115 was prepared following an analogous procedure to the one described for the synthesis of intermediate 63 using tert-butyl 5-(4,4,5,5-tetramethyl-1,3,2-dioxaborolan-2-yl)-3,6-dihydropyridine-1(2H)-carboxylate (CAS: 885693-20-9) and 4-chloro-2,6-dimethylpiridine as starting materials.

Preparation of Intermediate 116

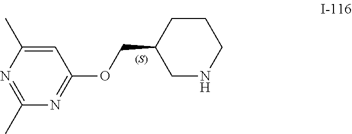

Intermediate 116 was prepared following an analogous procedure to the one described for the synthesis of intermediate 1 using intermediate 117 as starting material.

Preparation of Intermediate 117

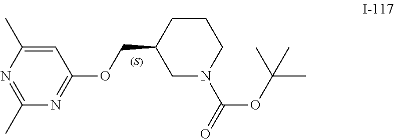

Intermediate 117 was prepared following an analogous procedure to the one described for the synthesis of intermediate 35 using (S)-1-Boc-3-(hydroxymethyl)piperidine (CAS: 140695-84-7) and 4-chloro-2,6-dimethylpiridine as starting materials.

Preparation of Intermediate 120

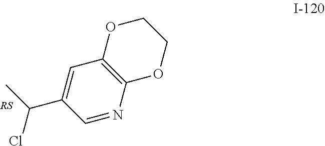

Intermediate 120 was prepared following an analogous procedure to the one described for the synthesis of intermediate 107 using intermediate 121 as starting material.

Preparation of Intermediate 121

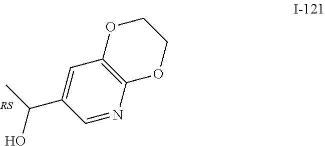

Intermediate 121 was prepared following an analogous procedure to the one described for the synthesis of intermediate 108 using 1-(2,3-dihydro-[1,4]dioxino[2,3-b]pyridin-7-yl)-ethanone (CAS: 1254044-15-9) as starting material.

Preparation of Intermediate 123

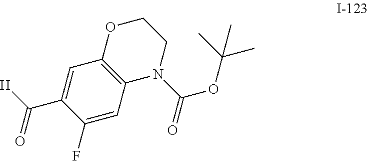 [SMILES: CC(C)(C)OC(=O)N1CCOc2cc(C=O)c(F)cc21]

Intermediate 123 was prepared following an analogous procedure to the one described for the synthesis of intermediate 93 using intermediate 124 as starting material.

Preparation of Intermediate 124

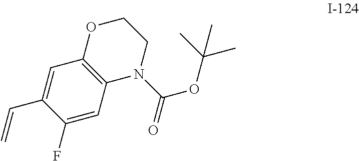 [SMILES: C=Cc1cc2c(cc1F)N(C(=O)OC(C)(C)C)CCO2]

Intermediate 124 was prepared following an analogous procedure to the one described for the synthesis of intermediate 94 using intermediate 125 as starting material.

Preparation of Intermediate 125

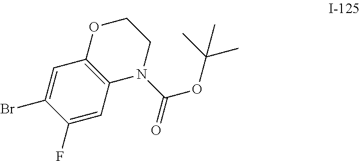 [SMILES: CC(C)(C)OC(=O)N1CCOc2cc(Br)c(F)cc21]

Borane dimethyl sulphide complex (1.65 mL, 17.4 mmol) was added dropwise to a stirred suspension of 7-bromo-6-fluoro-2H-benzo[b][1,4]oxazin-3(4H)-one (CAS: 1260829-35-3; 2.1 g, 8.53 mmol) in THF (44 mL) in a round-bottom flask under a condenser and under N₂ atmosphere. The mixture was stirred at reflux temperature for 2 h. The mixture was cooled at 0° C. and MeOH (12 mL) was added dropwise. The mixture was stirred at rt for 1 h. The solvent was evaporated in vacuo. The crude taken up in THF (44 mL) and cooled at 0° C. Boc-anhydride (CAS: 24424-99-5; 2.65 mL, 12.4 mmol) was added in one portion followed by lithium bis(trimethylsilyl)amide (12.1 mL, 12.1 mmol, 1M solution in THF) dropwise and the mixture was stirred at 0° C. for 1 h and at rt for 60 h. The mixture was treated with aq sat NH₄Cl and extracted with EtOAc. The organic layer was separated, dried (MgSO₄), filtered and the solvents evaporated in vacuo. The crude product was purified by flash column chromatography (SiO₂, EtOAc in heptane 0/100 to 70/30). The desired fractions were collected and concentrated in vacuo to yield intermediate 125 (2.8 g, 99%) as a yellow oil

Preparation of Intermediate 129

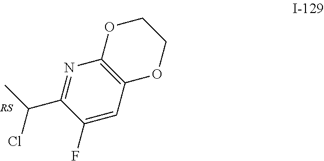

Intermediate 129 was prepared following an analogous procedure to the one described for the synthesis of intermediate 107 using intermediate 130 as starting material.

Preparation of Intermediate 130

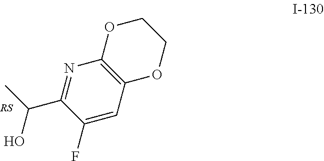

Intermediate 130 was prepared following an analogous procedure to the one described for the synthesis of intermediate 108 using intermediate 100 as starting material.

Preparation of Intermediates 46 and 47

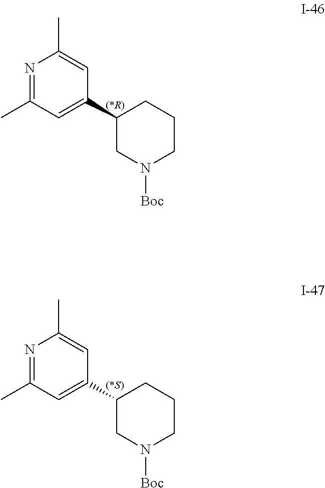 [SMILES: Cc1cc([C@H]2CCCN(C(=O)OC(C)(C)C)C2)cc(C)n1 ; Cc1cc([C@@H]2CCCN(C(=O)OC(C)(C)C)C2)cc(C)n1]

Pd/C (10%, 1.18 g, 1.11 mmol) was added to a stirred solution of intermediate 63 (3.20 g, 11.1 mmol) in EtOH (64.1 mL). The reaction mixture was hydrogenated (atmospheric pressure) at room temperature for 16 h. The mixture was filtered through a pad of Celite® and washed with MeOH. The filtrate was concentrated in vacuo. The residue was purified by flash column chromatography (silica, EtOAc in heptane, gradient from 0/100 to 80/20). The desired fractions were collected and concentrated in vacuo. The residue was purified via chiral SFC (stationary phase: CHIRALPAK IC 5 μm 250*30 mm, mobile phase: 65% CO₂, 35% i-PrOH (0.3% i-PrNH₂)) to afford intermediate 46 (1.30 g, 40%) and intermediate 47 (1.44 g, 44%).

Preparation of Intermediate 131

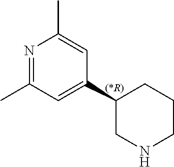

A solution of intermediate 46 (1.30 g, 4.48 mmol) in MeOH (34.4 mL) was added to a closed reactor containing Amberlyst®15 hydrogen form (CAS: 39389-20-3). The mixture was shaken in a solid phase reactor at room temperature for 16 h. The resin was washed with MeOH (the fraction was discarded) and NH₃ (7N in MeOH) (34 mL) was added. The mixture was shaken in a solid phase reactor for 2 h. The resin was filtered off and washed with NH₃ (7N in MeOH) (3×34 mL; 30 min shaken). The filtrates were concentrated in vacuo to yield intermediate 131 (820 mg, 96%) as a brown oil.

Preparation of Intermediate 132

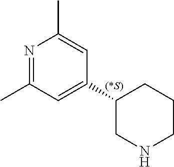

Intermediate 132 was prepared following an analogous procedure to the one described for the synthesis of intermediate 131 using intermediate 47 as starting material.

Preparation of Intermediate 133

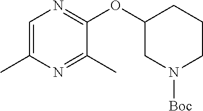 [SMILES: Cc1cnc(OC2CCCN(C(=O)OC(C)(C)C)C2)c(C)n1]

NaH (60% dispersion in mineral oil, 219 mg, 5.47 mmol) was added to a solution of N-Boc-3-hydroxypiperidine (CAS: 85275-45-2; 1.00 g, 4.97 mmol) in THF (15 mL) at 0° C. under N₂ atmosphere. The mixture was stirred at 0° C. for 30 min and 2-chloro-3,5-dimethylpyrazine (CAS: 38557-72-1; 628 μL, 5.22 mmol) was added dropwise. The reaction mixture was stirred at 60° C. for 20 h. The reaction was quenched with NH₄Cl (sat.) and extracted with EtOAc. The organic layer was dried (MgSO₄), filtered and evaporated in vacuo. The crude product was purified by flash column chromatography (silica, EtOAc in heptane, gradient from 0/100 to 40/60). The desired fractions were collected and concentrated in vacuo to afford intermediate 133 (920 mg, 60%) as a colorless oil.

Preparation of Intermediate 134

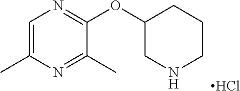

HCl (4M in 1,4-dioxane, 5 mL, 20 mmol) was added to a solution of intermediate 133 (0.95 g, 3.09 mmol) in 1,4-dioxane (5 mL). The reaction mixture was stirred at room temperature for 16 h and the solvent was evaporated in vacuo. The residue was treated with DCM and NaHCO₃ (sat.). The product was extracted with DCM and EtOH (9/1).

The organic layer was dried (MgSO₄), filtered and the solvents were evaporated in vacuo to give intermediate 134 (400 mg, 62%).

Preparation of Intermediate 135

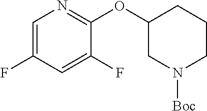 [SMILES: CC(C)(C)OC(=O)N1CCCC(Oc2ncc(F)cc2F)C1]

Intermediate 135 was prepared following an analogous procedure to the one described for the synthesis of intermediate 133 using N-Boc-3-hydroxypiperidine (CAS: 85275-45-2) and 2,3,5-trifluoropyridine (CAS: 76469-41-5) as starting materials.

Preparation of Intermediate 136

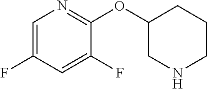 [SMILES: Fc1cnc(OC2CCCNC2)c(F)c1]

Intermediate 136 was prepared following an analogous procedure to the one described for the synthesis of intermediate 134 using intermediate 135 as starting material.

Preparation of Intermediate 137

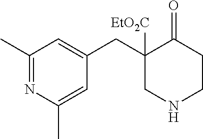 [SMILES: CCOC(=O)C1(Cc2cc(C)nc(C)c2)CNCCC1=O]

K₂CO₃ (2.36 g, 17.1 mmol) was added to a solution of 1-tert-butyl 3-ethyl 4-oxopiperidine-1,3-dicarboxylate (CAS: 98977-34-5; 1.55 g, 5.70 mmol) in acetone (30 mL) and the reaction mixture was stirred for 20 h at 50° C. The mixture was filtered through a pad of Celite®. The filtrate was diluted with EtOAc and water. The aqueous phase was extracted with EtOAc. The organic layer was dried (MgSO₄), filtered and the solvents were evaporated in vacuo. The crude product was purified by flash column chromatography (silica, EtOAc in heptane, gradient from 30/70 to 100/0). The desired fractions were collected and concentrated in vacuo to afford intermediate 137 (1.4 g, 63%).

Preparation of Intermediate 138

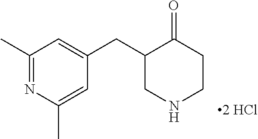 [SMILES: Cc1cc(CC2CNCCC2=O)cc(C)n1]

HCl (6M, 20 mL, 120 mmol) was added to a solution of intermediate 137 (1.30 g, 3.33 mmol) in 1,4-dioxane (6 mL). The reaction mixture was stirred at 120° C. for 4 days.

The reaction mixture was cooled down and the solvent was evaporated in vacuo and co-evaporated with toluene to give intermediate 138.2HCl (1.4 g, quant., 71% purity).

Preparation of Intermediate 139

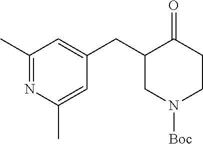 [SMILES: Cc1cc(CC2CN(C(=O)OC(C)(C)C)CCC2=O)cc(C)n1]

A solution of di-tert-butyl-dicarbonate (CAS: 24424-99-5; 2.25 g, 10.3 mmol) in THF (10 mL) was added to a solution of intermediate 138.2HCl (1.50 g, 6.87 mmol, 71% purity) in THF (30 mL) and H₂O (10 mL). Na₂CO₃ (2.19 g, 20.6 mmol) was added and the reaction mixture was stirred for 1 h at room temperature. The reaction mixture was diluted with EtOAc and water. The aqueous phase was extracted with EtOAc. The organic layer was dried (MgSO₄), filtered and the solvents were evaporated in vacuo.

The crude product was purified by flash column chromatography (silica, MeOH in DCM, gradient from 0/100 to 03/97). The desired fractions were collected and evaporated in vacuo to afford intermediate 139 (0.9 g, 41%) as a colorless oil.

Preparation of Intermediate 140

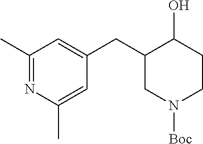 [SMILES: Cc1cc(CC2CN(C(=O)OC(C)(C)C)CCC2O)cc(C)n1]

NaBH₄ (28.5 mg, 0.75 mmol) was added to a solution of intermediate 139 (200 mg, 0.63 mmol) in EtOH (5 mL) at 0° C. The reaction mixture was stirred at room temperature for 2 h, quenched with NH₄Cl (sat.) and extracted with EtOAc. The combined organic layers were dried (MgSO₄), filtered and concentrated in vacuo to afford intermediate 140 (200 mg, 99%) as an oil which crystallized upon standing.

Preparation of Intermediate 141

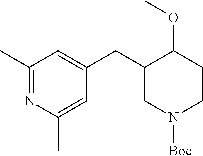 [SMILES: COC1CCN(C(=O)OC(C)(C)C)CC1Cc1cc(C)nc(C)c1]

NaH (60% dispersion in mineral oil, 37.4 mg, 0.94 mmol) was added to a stirred solution of intermediate 140 (200 mg, 0.62 mmol) in DMF (3 mL) at 0° C. The mixture was stirred for 5 min and iodomethane (77.7 μL, 1.25 mmol) was added. The reaction mixture was warmed to room temperature and stirred for 1 h. The mixture was diluted with NH₄Cl (10%) and extracted with EtOAc. The organic layer was dried (MgSO₄), filtered and the solvents were evaporated in vacuo to afford intermediate 141 (205 mg, 98%) as an oil.

Preparation of Intermediate 142

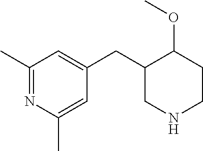 [SMILES: COC1CCNCC1Cc1cc(C)nc(C)c1]

A solution of intermediate 141 (205 mg, 0.61 mmol) in MeOH (19.1 mL) was added to a closed reactor containing Amberlyst®15 hydrogen form (CAS: 39389-20-3; 652 mg, 3.07 mmol). The reaction mixture was shaken in a solid phase reactor at room temperature for 16 h. The resin was washed with MeOH (the fraction was discarded) and with NH₃ (7N in MeOH). The filtrates were concentrated in vacuo to afford intermediate 142 (140 mg, 97%) as a pale brown oil.

Preparation of Intermediate 143

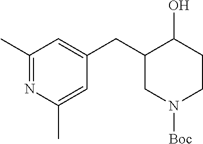 [SMILES: Cc1cc(CC2CN(C(=O)OC(C)(C)C)CCC2O)cc(C)n1]

NaBH₄ (28.5 mg, 0.75 mmol) was added to a solution of intermediate 139 (200 mg, 0.63 mmol) in EtOH (5 mL) at 0° C. The reaction mixture was stirred at room temperature for 16 h and quenched with NH₄Cl (sat. solution). The mixture was extracted with EtOAc. The combined organic layers were dried (MgSO₄), filtered and concentrated in vacuo to afford intermediate 143 (200 mg, 99%) as an oil which solidified upon standing.

Preparation of Intermediate 144

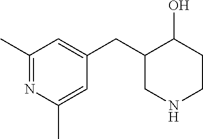 [SMILES: Cc1cc(CC2CNCCC2O)cc(C)n1]

A solution of intermediate 143 (200 mg, 0.62 mmol) in MeOH (20 mL) was added to a closed reactor containing Amberlyst®15 hydrogen form (CAS: 39389-20-3; 664 mg, 3.12 mmol). The reaction mixture was shaken in a solid phase reactor at room temperature for 16 h. The resin was washed with MeOH (the fraction was discarded) and with NH₃ (7N in MeOH). The filtrates were concentrated in vacuo to give intermediate 144 (135 mg, 98%) as a pale brown oil.

Preparation of Intermediate 145

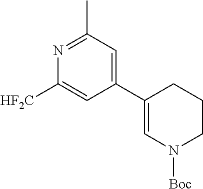

1,4-Dioxane (7.05 mL), 4-bromo-2-(difluoromethyl)-6-methylpyridine (CAS: 1226800-12-9; 500 mg, 2.25 mmol) and Na₂CO₃ (sat. solution, 10 mL) were successively added to a stirred mixture of 5-(4,4,5,5-tetramethyl-[1,3,2]dioxaborolane-2-yl)-3,6-dihydro-2H-pyridine-1-carboxylic acid tert-butyl ester (CAS: 885693-20-9; 696 mg, 2.25 mmol) and Pd(PPh₃)₄ (156 mg, 0.14 mmol) in a sealed tube and under N₂ atmosphere. The reaction mixture was stirred at 130° C. for 30 min under microwave irradiation. The mixture was treated with water and extracted with DCM. The organic layer was dried (Na₂SO₄), filtered and the solvents were evaporated in vacuo. The crude product was purified by flash column chromatography (silica, EtOAc in DCM, gradient from 0/100 to 50/50). The desired fractions were collected and concentrated in vacuo to afford intermediate 145 (685 mg, 94%).

Preparation of Intermediate 146

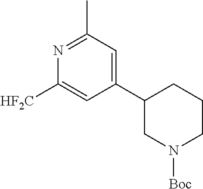

To a solution of intermediate 145 (649 mg. 2.00 mmol) in EtOH (12.6 mL) was added Pd/C (10%, 213 mg, 0.20 mmol) under N₂ atmosphere. The reaction mixture was hydrogenated (atmospheric pressure) at room temperature for 18 h. The reaction mixture was filtered through a pad of Celite® and the filtrate was concentrated in vacuo to give intermediate 146 (605 mg, 93%) as a colorless oil which solidified upon standing.

Preparation of Intermediate 147

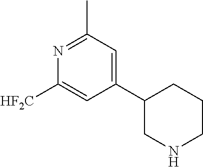

HCl (4M in 1,4-dioxane, 12.4 mL, 49.6 mmol) was added to intermediate 146 (600 mg, 1.84 mmol) and the reaction mixture was stirred at room temperature for 3 h. The reaction was concentrated to dryness. The residue was purified by ion exchange chromatography (isolute SCX2 cartridge) eluting with MeOH, then with NH₃ (7M in MeOH). The desired fraction was collected and concentrated in vacuo to yield intermediate 147 (395 mg, 95%) as a colorless oil.

Preparation of Intermediate 148

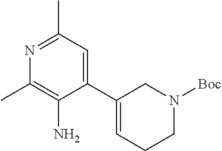 [SMILES: Cc1cc(C2=CCCN(C(=O)OC(C)(C)C)C2)c(N)c(C)n1]

Intermediate 148 was prepared following an analogous procedure to the one described for the synthesis of intermediate 145 using 4-chloro-2,6-dimethylpyridin-3-amine (CAS: 37652-11-2) and 5-(4,4,5,5-tetramethyl-[1,3,2]dioxaborolane-2-yl)-3,6-dihydro-2H-pyridine-1-carboxylic acid tert-butyl ester (CAS: 885693-20-9) as starting materials.

Preparation of Intermediate 149

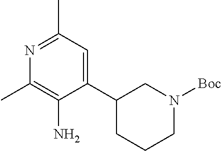 [SMILES: Cc1cc(C2CCCN(C(=O)OC(C)(C)C)C2)c(N)c(C)n1]

Intermediate 149 was prepared following an analogous procedure to the one described for the synthesis of intermediate 146 using intermediate 148 as starting material.

Preparation of Intermediate 150

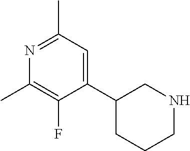

A mixture of intermediate 149 (600 mg, 1.96 mmol) and nitrosyl tetrafluoroborate (CAS: 688 mg, 5.89 mmol) in DCM (6 mL) was stirred at room temperature for 18 h.

The solvent was removed in vacuo and the residue was purified by ion exchange chromatography (isolute SCX-2 cartridge) eluting with MeOH, then with NH₃ (7N in MeOH) (3 times). The desired fractions were collected and the solvent was evaporated in vacuo. The product was purified by RP HPLC (stationary phase: C18 XBridge 30×100 mm 5 μm), mobile phase: NH₄HCO₃ (0.25% solution in water)/CH₃CN, gradient from 95/5 to 70/30) to give intermediate 150 (180 mg, 28%, 64% purity) as a colorless oil.

Preparation of Intermediate 151

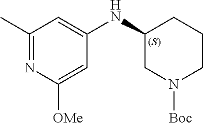

NaOt-Bu (238 mg, 42.48 mmol) was added to a stirred suspension of Pd₂dba₃ (45.3 mg, 49.5 μmol) and t-BuXPhos (63.1 mg, 0.15 mmol) in 1,4-dioxane (15 mL) in a sealed tube and under N₂ atmosphere. The reaction mixture was stirred at 95° C. for 5 min. A solution of 4-bromo-2-methoxy-6-methylpyridine (CAS: 1083169-00-9; 200 mg, 0.99 mmol) and (S)-(+)-3-amino-1-Boc-piperidine (CAS: 625471-18-3; 258 mg, 1.29 mmol) in 1,4-dioxane (5 mL) was added to the reaction mixture under N₂ at 95° C. The reaction mixture was stirred at 100° C. for 30 min. The reaction mixture was diluted with NaHCO₃ (sat. solution) and extracted with EtOAc. The organic layer was dried (MgSO₄), filtered and the solvents were evaporated in vacuo. The crude product was purified by flash column chromatography (silica, EtOAc in heptane, gradient from 5/95 to 100/0). The desired fractions were collected and concentrated in vacuo to yield intermediate 151 (310 mg. 94%) as a light yellow oil.

Preparation of Intermediate 152

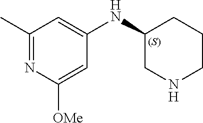

HCl (1.4M in 1,4-dioxane, 1.21 mL, 4.82 mmol) was added dropwise to intermediate 151 (310 mg, 0.96 mmol) at 0° C. The reaction mixture was stirred at room temperature for 16 h and the solvent was evaporated in vacuo. The crude product was purified by flash column chromatography (silica, NH₃ (7N in MeOH) in DCM, gradient from 0/100 to 10/90). The desired fractions were collected and concentrated in vacuo to yield intermediate 152 (170 mg, 80%) as a colorless oil.

Preparation of Intermediate 153

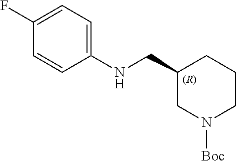

Sodium cyanoborohydride (CAS: 25895-60-7; 110 mg, 1.41 mmol) was added to a stirred mixture of 4-fluoroaniline (CAS: 371-40-4; 0.14 mL, 1.41 mmol), tert-butyl (3S)-3-formylpiperidine-1-carboxylate (CAS: 1008562-87-5; 200 mg. 0.94 mmol) and acetic acid (0.12 mL, 2.06 mmol) in MeOH (15 mL) at room temperature. The reaction mixture was stirred at 40° C. for 16 h. The solvent was evaporated in vacuo. NaHCO₃ (sat. solution) and EtOAc were added. The aqueous layer was extracted with EtOAc (twice). The combined organic extracts were dried (MgSO₄), filtered and the solvents were evaporated in vacuo. The crude product was purified by flash column chromatography (silica, EtOAc in heptane, gradient from 0/100 to 20/80). The desired fractions were collected and concentrated in vacuo to afford intermediate 153 (265 mg, 92%) as a light yellow oil.

Preparation of Intermediate 154

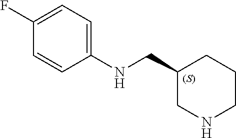

Intermediate 153 (265 mg, 0.86 mmol) was solved in HCl (4M in 1,4-dioxane, 1.07 mL, 4.30 mmol). The reaction mixture was stirred at room temperature for 2 h and the solvent was evaporated in vacuo. The residue was dissolved in MeOH (1.5 mL) and Amberlyst® A26 hydroxide (CAS: 39339-85-0; 1.14 g, 3.44 mmol) was added. The mixture was stirred at room temperature until pH was 7. The resin was removed by filtration and the solvents were evaporated in vacuo. The desired fractions were collected and concentrated in vacuo to afford intermediate 154 (172 mg, 96%) as a light yellow oil.

Preparation of Intermediate 155

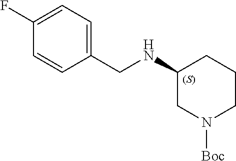

Intermediate 155 was prepared following an analogous procedure to the one described for the synthesis of intermediate 153 using (S)-(+)-3-amino-1-Boc-piperidine (CAS: 625471-18-3) and 4-fluorobenzaldehyde (CAS: 459-57-4; 0.43 mL, 4.03 mmol) as starting materials.

Preparation of Intermediate 156

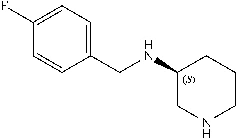

Intermediate 156 was prepared following an analogous procedure to the one described for the synthesis of intermediate 154 using intermediate 155 as starting material.

Preparation of Intermediate 157

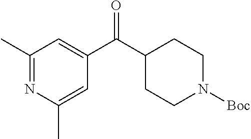 [SMILES: Cc1cc(C(=O)C2CCN(C(=O)OC(C)(C)C)CC2)cc(C)n1]

n-Butyl lithium (2.5M in hexane, 3.67 mL, 9.46 mmol) was added to a mixture of 4-bromo-2,6-dimethylpyridine (CAS: 5093-70-9; 1.55 g, 8.33 mmol) in THF (25 mL) at −78° C. under N₂ atmosphere. The reaction mixture was stirred at −78° C. for 30 min and a solution of tert-butyl 4-(methoxy(methyl)carbamoyl)piperidine-1-carboxylate (CAS: 139290-70-3; 2.50 g, 9.16 mmol) in THF (5 mL) was added at −78° C. The reaction mixture was stirred at −78° C. for 1 h. NH₄Cl (sat. solution) was added at −78° C. and the mixture was extracted with EtOAc (2×10 mL). The organic layer was dried (Na₂SO₄), filtered and concentrated in vacuo. The crude mixture was purified by flash column chromatography (SiO₂, EtOAc in heptane, gradient from 0/100 to 20/80). The desired fractions were collected and concentrated in vacuo to yield intermediate 157 (1.44 g, 54%) as a yellow oil that solidified upon standing.

Preparation of Intermediate 158

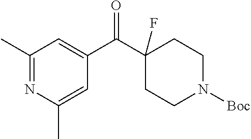 [SMILES: Cc1cc(C(=O)C2(F)CCN(C(=O)OC(C)(C)C)CC2)cc(C)n1]

Lithium bis(trimethylsilyl)amide solution (1M, 4.98 mL, 4.98 mmol) was added to a mixture of intermediate 157 (1.44 g, 4.52 mmol) in THF (111 mL), at −78° C. The mixture was stirred at −10° C. for 1 h and the mixture was cooled to −78° C. A solution of N-fluorobenzenesulfonimide (CAS: 133745-75-2; 1.57 g, 4.98 mmol) in THF (12.3 mL) was added and the reaction mixture was stirred at −78° C. for 1 h, then −50° C. for 2 h. NH₄Cl (sat. solution) was added and the mixture was extracted with EtOAc. The organic layer was dried (Na₂SO₄), filtered and evaporated in vacuo. The crude mixture was purified by flash column chromatography (SiO₂, MeOH in DCM, gradient from 0/100 to 7/93, then EtOAc in heptane, gradient from 0/100 to 100/0). The desired fractions were collected and concentrated in vacuo to afford intermediate 158 (963.7 mg, 41%, 65% purity) as a yellow oil that solidified upon standing.

Preparation of Intermediate 159

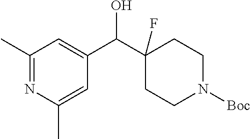 [SMILES: Cc1cc(C(O)C2(F)CCN(C(=O)OC(C)(C)C)CC2)cc(C)n1]

NaBH₄ (0.13 g, 3.44 mmol) was added to a mixture of intermediate 158 (964 mg, 2.87 mmol, 65% purity) in MeOH (19.3 mL) at 0° C. The reaction mixture was stirred at room temperature for 2 h, quenched with NaOH (1M) (2 mL) and extracted with EtOAc (2×30 mL). The combined organic layers were dried (Na₂SO₄), filtered and concentrated in vacuo to yield intermediate 159 (1.07 g, 81%, 73% purity) as a light yellow oil.

Preparation of Intermediate 160

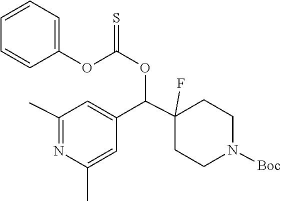 [SMILES: Cc1cc(C(OC(=S)Oc2ccccc2)C2(F)CCN(C(=O)OC(C)(C)C)CC2)cc(C)n1]

O-phenyl chlorothionoformate (CAS: 1005-56-7; 1.43 g, 8.27 mmol) was added to a mixture of intermediate 159 (1.40 g, 4.14 mmol, 73% purity) and DMAP (75.8 mg, 0.62 mmol) in DCM (33.6 mL). Et₃N (1.44 mL, 10.3 mmol) was added and the reaction mixture was stirred for 72 h at room temperature. NH₄Cl (sat. solution) was added and the mixture was extracted with EtOAc. The organic layer was washed with brine, dried (Na₂SO₄), filtered and concentrated in vacuo. The crude mixture was purified by flash column chromatography (SiO₂, EtOAc in DCM, gradient from 0/100 to 100/0, then MeOH in DCM, gradient from 0/100 to 15/85). The desired fractions were collected and concentrated in vacuo to afford intermediate 160 (623 mg, 32%) as a light yellow foam.

Preparation of Intermediate 161

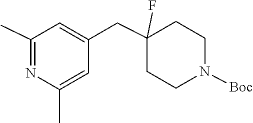 [SMILES: Cc1cc(CC2(F)CCN(C(=O)OC(C)(C)C)CC2)cc(C)n1]

Tributyltin hydride (CAS: 688-73-3; 1.07 mL, 3.98 mmol) was added to a mixture of intermediate 160 (630 mg, 1.33 mmol) and AIBN (CAS: 78-67-1; 21.8 mg, 0.13 mmol) in toluene (19 mL). the reaction mixture was stirred at 110° C. for 2 h. The reaction mixture was cooled down and the solvent was evaporated in vacuo. The crude mixture was purified by flash column chromatography (SiO₂, DCM in heptane, gradient from 0/100 to 100/0; then MeOH in DCM, gradient from 0/100 to 15/85). The desired fractions were collected and concentrated in vacuo to yield intermediate 160 (457.6 mg, 88%, 82% purity) as a light yellow oil.

Preparation of Intermediate 162

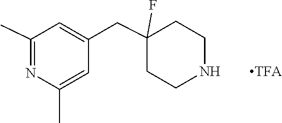 [SMILES: Cc1cc(CC2(F)CCNCC2)cc(C)n1]

TFA (0.92 mL, 12.0 mmol) was added to a mixture of intermediate 161 (458 mg, 1.42 mmol, 82% purity) in DCM (2.29 mL). The reaction mixture was stirred at room temperature for 3 h and the solvent was evaporated in vacuo to afford intermediate 162.TFA (250 mg, 42%, 81% purity) as a light yellow oil. 150 mg of intermediate 162.TFA were neutralized with NaHCO₃ (sat. solution) and extracted with DCM (2×10 mL) and with MeOH and DCM (2/8). The organic layer was dried (Na₂SO₄), filtered and concentrated in vacuo to afford intermediate 162 (100 mg, 32%) as an orange oil.

Preparation of Intermediate 163

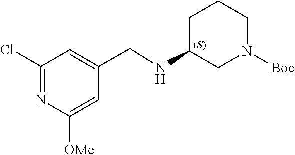

(S)-(+)-3-Amino-1-Boc-piperidine (CAS: 625471-18-3; 117 mg, 0.58 mmol) and 2-methoxy-6-methylpyridine-4-carbaldehyde (CAS: 951795-43-0; 100 mg, 0.58 mmol) were dissolved in CH₃CN (3 mL). The reaction mixture was stirred at room temperature for 30 min, and sodium triacetoxyborohydride (371 mg, 1.75 mmol) was added. The resulting mixture was stirred at room temperature for 16 h. The mixture was diluted with NaHCO₃ (sat., aq.) and DCM. The aqueous layer was extracted with DCM (twice). The combined organic layers were dried (MgSO₄), filtered and the solvents were evaporated in vacuo. The crude mixture was purified by flash column chromatography (SiO₂, EtOAc in heptane, gradient from 0/100 to 50/50) to afford intermediate 163 (161 mg, 77%).

Preparation of Intermediate 164

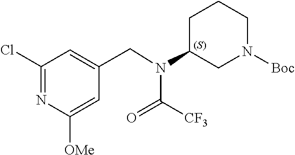

Trifluoroacetic anhydride (0.5 mL, 3.23 mmol) was added dropwise to a stirred mixture of intermediate 163 (1.00 g, 2.81 mmol) and DIPEA (0.64 mL, 3.65 mmol) in DCM (13 mL) under N₂ atmosphere at room temperature. The reaction mixture was stirred for 16 h. The reaction was quenched with HCl (1M) and extracted with DCM. The organic layer was washed with NaHCO₃ (sat., aq.) and brine, dried (MgSO₄), filtered and the solvents were evaporated in vacuo. The crude mixture was purified by flash column chromatography (SiO₂, EtOAc in heptane, gradient from 0/100 to 30/70) to afford intermediate 164 (1.1 g, 87%).

Preparation of Intermediate 165

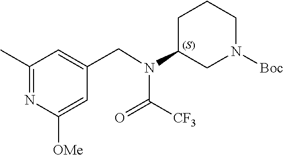

Intermediate 164 (900 mg, 1.99 mmol) and methylboronic acid (CAS: 13061-96-6; 304 mg, 4.98 mmol) were added to a stirred solution of Na₂CO₃ (633 mg, 5.98 mmol), 1,4-dioxane (4.98 mL) and H₂O (1.25 mL) under N₂ atmosphere. PdCl₂(dppf).DCM (81.3 mg, 99.6 μmol) was added and the reaction mixture was stirred at 105° C. for 16 h. Additional amount of methylboronic acid (1.25 eq), PdCl₂(dppf).DCM (0.025 eq) and Na₂CO₃ (1.5 eq) were added under N₂ atmosphere. The reaction mixture was stirred at 105° C. for 16 h. The mixture was diluted with NaHCO₃ and extracted with EtOAc. The organic layer was dried (MgSO₄), filtered and the solvents were evaporated in vacuo. The crude mixture was purified by flash column chromatography (SiO₂, EtOAc in heptane, gradient from 0/100 to 20/80) to afford intermediate 165 (690 mg, 80%).

Preparation of Intermediate 166

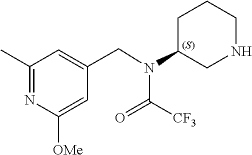

HCl (4M in 1,4-dioxane, 2.00 mL, 8.00 mmol) was added dropwise to intermediate 165 (690 mg, 1.60 mmol) at 0° C. The reaction mixture was stirred at room temperature for 16 h and solvent was evaporated in vacuo. The crude mixture was purified by flash column chromatography (SiO₂, MeOH/NH₃ in DCM, gradient from 0/100 to 10/90). The desired fractions were collected and concentrated in vacuo to afford intermediate 166 (317 mg, 59%).

Preparation of Intermediate 167

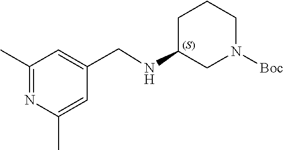

Intermediate 167 was prepared following an analogous procedure to the one described for the synthesis of intermediate 163 using (S)-(+)-3-Amino-1-Boc-piperidine (CAS: 625471-18-3) and 2,6-dimethyl-4-pyridine carboxaldehyde (CAS: 18206-06-9) as starting materials.

Preparation of Intermediate 168

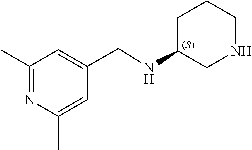

HCl (4M in 1,4-dioxane, 2.26 mL, 9.03 mmol) was added dropwise to intermediate 167 (577 mg, 1.81 mmol) at 0° C. The reaction mixture was stirred at room temperature for 16 h and the solvent was removed in vacuo. The crude mixture was purified by flash column chromatography (SiO₂, MeOH/NH₃ in DCM, gradient from 0/100 to 10/90) to afford intermediate 168 (320 mg, 80%).

Preparation of Intermediate 169

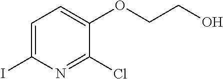 [SMILES: OCCOc1ccc(I)nc1Cl]

[1,3]-Dioxolan-2-one (CAS: 96-49-1; 1.03 g, 117 mmol) and K₂CO₃ (16.2 g, 117 mmol) were added portionwise to a stirred solution of 2-chloro-3-hydroxy-6-iodo-pyridine (CAS: 185220-68-2; 20.0 g, 78.3 mmol) in DMF (300 mL) under a N₂ atmosphere at 20° C. The reaction mixture was warmed to 100° C. and stirred for 1 h. Then the reaction mixture was warmed to 150° C. and stirred for 1 h. The reaction mixture was cooled to room temperature and additional quantity of [1,3]-dioxolan-2-one (2.76 g) and K₂CO₃ (5.41 g) were added. The reaction mixture was warmed to 150° C. and the reaction mixture was stirred for 1 h. Water was added and the product was extracted with EtOAc. The organic layer was dried (MgSO₄), filtered and concentrated in vacuo. The crude product was purified by column chromatography (silica, EtOAc in heptane, gradient from 0/100 to 100/0), The residue was purified a second time under the same conditions. The desired fractions were concentrated in vacuo to afford intermediate 169 (10 g, 43%) as a white solid.

Preparation of Intermediate 170

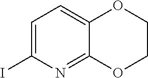 [SMILES: Ic1ccc2c(n1)OCCO2]

KOH (6.26 mL, 25.0 mmol) and 18-crown-6 (530 mg, 2.00 mmol) were added portionwise to a stirred solution of intermediate 169 (5.00 g, 16.7 mmol) in toluene (200 mL) under N₂ atmosphere at 20° C. The reaction mixture was stirred at 110° C. for 5 h, diluted with water and the product was extracted with EtOAc. The organic layer was dried (MgSO₄), filtered and concentrated in vacuo. The crude product was purified by flash column chromatography (silica, EtOAc in heptane, gradient from 0/100 to 100/0). The desired fractions were collected and concentrated in vacuo to yield intermediate 170 (3.0 g, 68%) as a white solid.

Preparation of Intermediate 171

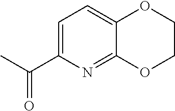

Tributyl(1-ethoxyvinyl)tin (CAS: 97674-02-7; 7.8 mL, 23.1 mmol) and Pd(PPh₃)₂C12 (133 mg, 0.19 mmol) were added to a stirred solution of intermediate 170 (5.00 g, 19.0 mmol) in toluene (111 mL). The reaction mixture was stirred at 120° C. for 12 h. HCl (2M in H₂O, 95 mL, 9.5 mmol) was added at 0° C. and the mixture was stirred at room temperature for 12 h. NaHCO₃ (sat., aq.) was added and the organic layer was extracted with DCM. The combined organic layers were dried (Na₂SO₄), filtered and concentrated in vacuo. The crude mixture was purified by flash column chromatography (silica, EtOAc in heptane, gradient from 0/100 to 100/0). The fractions were collected and concentrated in vacuo to afford intermediate 171 (1.5 g, 44%) as a brown solid.

Preparation of Intermediate 172

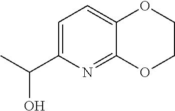

NaBH₄ (1.27 g, 33.5 mmol) was added to a solution of intermediate 171 (1.50 g, 8.37 mmol) in EtOH (39.1 mL) at 0° C. The reaction mixture was stirred at room temperature for 10 min. Water was added and the mixture was extracted with DCM.

The combined organic layers were dried (Na₂SO₄), filtered and concentrated in vacuo to afford intermediate 172 (1.50 g, 99%) as a colorless oil which was used in the next step without further purification.

Preparation of Intermediate 173

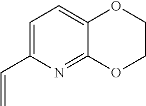

A mixture of intermediate 170 (15.0 g, 57.0 mmol), tributyl(vinyl)tin (CAS: 7486-35-3; 29.9 g, 94.3 mmol) and Pd(PPh₃)₂C2 (400 mg, 0.57 mmol) in toluene (300 mL) was stirred for 16 h at 120° C. The reaction mixture was quenched with CsF (aq., 200 mL) and extracted with EtOAc (3×600 mL). The combined organic extracts were washed with brine, dried (Na₂SO₄), filtered and evaporated in vacuo. The crude product was purified by flash column chromatography (silica, petroleum ether/EtOAc, gradient from 1/0 to 10/4) to afford intermediate 173 (7.5 g, 81%) as a yellow oil.

Preparation of Intermediate 174

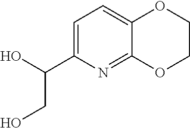 [SMILES: OCC(O)c1ccc2c(n1)OCCO2]

A mixture of intermediate 173 (7.5 g, 45.9 mmol), 4-methylmorpholine N-oxide (CAS: 7529-22-8; 9.69 g, 82.7 mmol) and potassium osmate (VI) dihydrate (CAS: 10022-66-9; 169 mg, 0.46 mmol) in THF (100 mL), CH₃CN (50 mL) and H₂O (25 mL) was stirred at room temperature overnight. The reaction mixture was quenched Na₂S₂O₃ (aq.) (100 mL) and extracted with EtOAc (3×100 mL). The combined organic extracts were washed with brine, dried (Na₂SO₄), filtered and evaporated in vacuo. The crude product was used in the next step without any purification.

Preparation of Intermediate 175

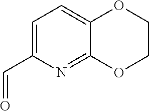 [SMILES: O=Cc1ccc2c(n1)OCCO2]

To a mixture of intermediate 174 (7.00 g, 35.5 mmol) in CH₃CN (30 mL) and H₂O (30 mL) was added sodium periodate (CAS: 7790-28-5; 15.2 g, 71.0 mmol). The reaction mixture was stirred at room temperature overnight, quenched with water (50 mL) and extracted with EtOAc (3×150 mL). The combined organic extracts were washed with brine, dried (Na₂SO₄), filtered and evaporated in vacuo. The crude product was combined with another fraction (20.3 mmol) and purified by flash column chromatography (silica, petroleum ether/EtOAc, gradient from 1/0 to 1/1) to afford intermediate 175 (7.84 g, 85%) as white a solid.

Preparation of Intermediate 176

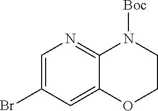 [SMILES: CC(C)(C)OC(=O)N1CCOc2cc(Br)cnc21]

Lithium bis(trimethylsilyl)amide (1M in THF, 1.1 equiv.) was added dropwise over 10 min to a stirred mixture of 7-bromo-3,4-dihydro-2H-pyrido[3,2-b][1,4]oxazine (CAS: 34950-82-8; 3.00 g, 14.0 mmol) and di-tert-butyl dicarbonate (CAS: 24424-99-5; 1.1 equiv.) in THF (67.8 mL) at 0° C. and under N₂ atmosphere. The reaction mixture was stirred at 0° C. for 2 h and additional quantity of boc-anhydride (0.52 equiv.) in THF (10 mL) was added at 0° C. The reaction mixture was stirred at 0° C. for 1 h, treated with NH₄Cl (sat.) and extracted with EtOAc. The organic layer was dried (Na₂SO₄), filtered and the solvents were evaporated in vacuo. The crude product was purified by flash column chromatography (SiO₂, EtOAc in DCM, gradient from 0/100 to 2/98). The desired fractions were collected and concentrated in vacuo to afford intermediate 176 (3.66 g, 83%) as a beige solid.

Preparation of Intermediate 177

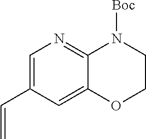 [SMILES: C=Cc1cnc2c(c1)OCCN2C(=O)OC(C)(C)C]

Pd(PPh₃)₄ (0.67 g, 0.58 mmol) followed by vinylboronic acid pinacol ester (CAS: 75927-49-0; 2.46 mL, 14.5 mmol) were added to a deoxygenated solution of intermediate 176 (3.66 g, 11.6 mmol) in a saturated aqueous solution of K₂CO₃ (29 mL) and 1,4-dioxane (57.9 mL) under N₂ atmosphere. The reaction mixture was stirred at 80° C. for 18 h. The mixture was treated with water and extracted with EtOAc. The organic layer was dried (Na₂SO₄), filtered and the solvents were evaporated in vacuo. The crude product was purified by flash column chromatography (SiO₂, EtOAc in DCM, gradient from 0/100 to 5/95). The desired fractions were collected and concentrated in vacuo to afford intermediate 177 (2.69 g, 88%) as a brown solid.

Preparation of Intermediate 178

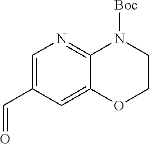 [SMILES: CC(C)(C)OC(=O)N1CCOc2cc(C=O)cnc21]

Sodium periodate (CAS: 7790-28-5; 4.9 g, 22.9 mmol) followed by osmium tetroxide (2.5% in t-BuOH, 1.89 mL, 0.14 mmol) were added to a stirred mixture of intermediate 177 (2.69 g, 10.2 mmol) in 1,4-dioxane (79.3 mL) and H₂O (31.7 mL) under N₂ atmosphere. The reaction mixture was stirred at room temperature for 4.5 h, treated with Na₂S₂O₃ (sat. solution) and extracted with EtOAc. The organic layer was dried (Na₂SO₄), filtered and the solvents were evaporated in vacuo. The crude product was purified by flash column chromatography (SiO₂, EtOAc in heptane, gradient from 0/100 to 40/60). The desired fractions were collected and concentrated in vacuo to afford intermediate 178 (1.93 g, 71%) as a white solid.

Preparation of Intermediate 179

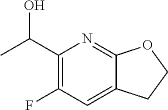

NaBH₄ (55.5 mg, 1.47 mmol) was added to a solution of intermediate 97 (133 mg, 0.73 mmol) in EtOH (3 mL) at 0° C. The reaction mixture was stirred at room temperature for 30 min and the reaction was quenched with NH₄Cl (sat., aq.). The mixture was extracted with DCM. The combined organic layers were dried (MgSO₄), filtered and concentrated in vacuo to afford intermediate 179 (130 mg, 97%) as an oil.

Preparation of Intermediate 180

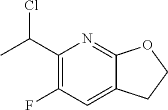

Thionyl chloride (0.8 mL, 11.0 mmol) was added to a solution of intermediate 179 (500 mg, 2.73 mmol) in DCM (12 mL) at 0° C. The reaction mixture was stirred at room temperature for 2 h, diluted with water and extracted with DCM. The combined organic layers were dried (MgSO₄), filtered and evaporated in vacuo to yield intermediate 180 (520 mg) which was used without any purification in the next step.

Preparation of Intermediate 181

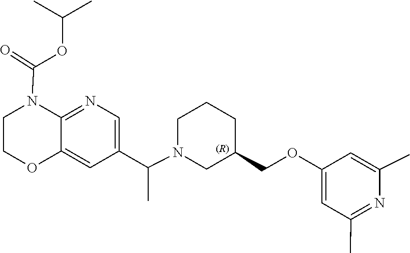

Intermediate 178 (126 mg, 0.48 mmol) and Ti(Oi-Pr)₄ (CAS: 546-68-9; 0.20 mL, 0.68 mmol) were added to a solution of intermediate 10 (100 mg, 0.45 mmol) in DCM (1.47 mL) and the reaction mixture was stirred at room temperature for 16 h. The reaction mixture was cooled to 0° C. and methylmagnesium bromide (1.4M solution, 1.62 mL, 2.27 mmol) was added dropwise. The reaction mixture was stirred at room temperature for 2 h. NH₄Cl (sat. solution) was added and the mixture was extracted with DCM. The organic layer was dried (MgSO₄), filtered and the solvents were evaporated in vacuo. The crude product was purified by flash column chromatography (silica, NH₃ (7M in MeOH) in DCM, gradient from 0/100 to 10/90) The desired fractions were collected and concentrated in vacuo to yield intermediate 181 (148.9 mg, 70%) as a yellow oil.

Preparation of Intermediate 182

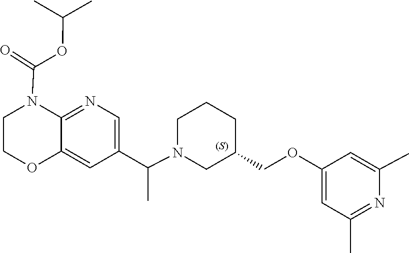

Intermediate 182 was prepared following an analogous procedure to the one described for the synthesis of intermediate 181 using intermediate 9 and intermediate 178 as starting materials.

The crude product was purified by flash column chromatography (silica, NH₃ (7M in MeOH) in DCM, gradient from 0/100 to 10/90) The desired fractions were collected and concentrated in vacuo to afford intermediate 182 (154 mg, 73%) as a yellow oil.

Preparation of Intermediate 183

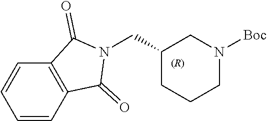

A mixture of (3S)-3-(hydroxymethyl)piperidine, N-Boc-protected (CAS: 140695-84-7; 2.5 g, 11.6 mmol), phthalimide (CAS: 85-41-6; 1.88 g, 12.8 mmol) and triphenylphosphine (4.57 g, 17.4 mmol) in anhydrous THF (138 mL) was stirred under N₂ atmosphere. DIAD (CAS: 2446-83-5; 3.45 mL, 17.4 mmol) was added and the reaction mixture was stirred at room temperature overnight. The mixture was diluted with water and extracted with EtOAc. The organic layer was dried (MgSO₄), filtered and the solvents were evaporated in vacuo. The crude product was purified by flash column chromatography (silica, EtOAc in heptane, gradient from 0/100 to 30/70). The desired fractions were collected and concentrated in vacuo to afford intermediate 183 (3.94 g, 99%) as a yellow oil.

Preparation of Intermediate 184

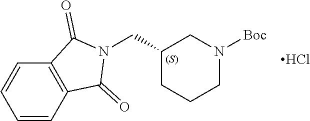

HCl (4M in 1,4-dioxane, 6.7 mL, 26.8 mmol) was added to a stirred solution of intermediate 183 (834 mg, 2.42 mmol) in 1,4-dioxane (12 mL). The reaction mixture was stirred at room temperature for 4 h. Then the solvent was evaporated in vacuo to give intermediate 184.HCl (807.3 mg) as a white solid, that was used in next step without further purification.

Preparation of Intermediate 185

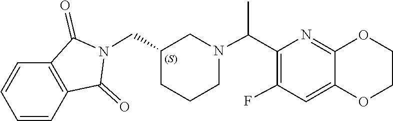

K₂CO₃ (1.18 g, 8.57 mmol) was added to a stirred solution of intermediate 184.HCl (802 mg, 2.86 mmol) and intermediate 130 (559 mg, 2.57 mmol in anhydrous CH₃CN (22.3 mL). The reaction mixture was stirred at 70° C. for 2 days. The reaction mixture was diluted with EtOAc and filtered through Celite®. The solvents were evaporated in vacuo. The crude product was purified by flash column chromatography (silica; MeOH in DCM, gradient from 0/100 to 1/99). The desired fractions were collected, and the solvents were evaporated in vacuo to yield intermediate 185 (638 mg, 52%) as a white solid.

Preparation of Intermediate 186

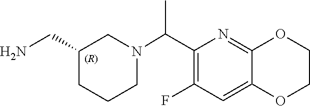

Hydrazine monohydrate (0.29 mL, 6.00 mmol) was added to a stirred solution of intermediate 185 (638 mg, 1.50 mmol) in EtOH (12 mL). The reaction mixture was stirred at 80° C. for 1 h. The precipitate was triturated with DIPE, filtered and the filtrate was dried (MgSO₄), filtered and concentrated in vacuo. The crude product was purified by flash column chromatography (silica; NH₃ (7M in MeOH)/DCM, gradient from 0/100 to 4/96). The desired fractions were collected and concentrated in vacuo to afford intermediate 186 (378 mg, 85%) as a colorless oil.

Preparation of Intermediate 187

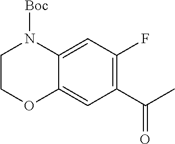 [SMILES: CC(=O)c1cc2c(cc1F)N(C(=O)OC(C)(C)C)CCO2]

Tributyl(1-ethoxyvinyl)tin (CAS: 97674-02-7; 5.59 mL, 16.6 mmol) and Pd(PPh₃)₂C12 (1.06 g, 1.51 mmol) were added to a stirred solution of intermediate 125 (5.00 g, 15.1 mmol) in 1,4-dioxane (100 mL) in a sealed tube and under N₂ atmosphere. The reaction mixture was stirred at 80° C. overnight. Then HCl (1M in H₂O, 7.53 mL) was added and the mixture was stirred at room temperature for 20 min. The mixture was treated with NaHCO₃ (sat. solution) and ice water and extracted with DCM. The organic layer was dried (MgSO₄), filtered and the solvents were evaporated in vacuo. The crude product was purified by flash column chromatography (silica, EtOAc in heptane, gradient from 0/100 to 50/50) (twice). The desired fractions were collected and concentrated in vacuo. The residue was dissolved in DCM and triturated with heptane to afford intermediate 187 (1.98 g, 45%) as a cream solid.

Preparation of Intermediate 188

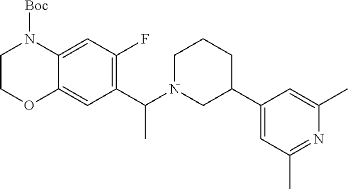 [SMILES: Cc1cc(C2CCCN(C(C)c3cc4c(cc3F)N(C(=O)OC(C)(C)C)CCO4)C2)cc(C)n1]

Intermediate 187 (195 mg, 0.66 mmol) and Ti(Oi-Pr)₄ (CAS: 546-68-9; 0.23 mL, 079 mmol) were added to a solution of intermediate 1 (100 mg, 0.53 mmol) in DCM (1.86 mL). The reaction mixture was stirred at room temperature for 16 h, cooled to 0° C. and sodium cyanoborohydride (CAS: 25895-60-7; 76.6 mg, 1.22 mmol) was added dropwise. The reaction mixture was stirred at room temperature for 2 h. NH₄Cl (sat. solution) was added and the mixture was extracted with DCM. The organic layer was dried (MgSO₄), filtered and the solvents were evaporated in vacuo. The crude product was purified by flash column chromatography (silica, NH₃ (7N in MeOH) in DCM, gradient from 0/100 to 10/90). The desired fractions were collected and concentrated in vacuo to yield intermediate 188 (77.9 mg, 32%) as a yellow oil.

Preparation of Intermediate 189

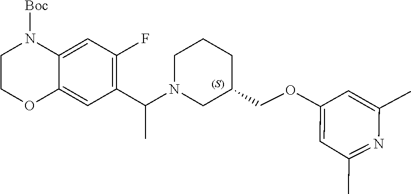

Intermediate 189 was prepared following an analogous procedure to the one described for the synthesis of intermediate 188 using intermediate 187 and intermediate 9 as starting materials.

The crude product was purified by flash column chromatography (silica, MeOH in DCM, gradient from 0/100 to 10/90). The desired fractions were collected and concentrated in vacuo to yield intermediate 189 (79 mg, 35%) as a yellow oil.

Preparation of Intermediate 190

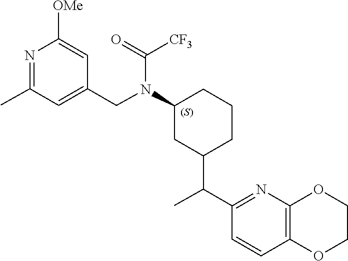

Intermediate 107 (130 mg, 0.65 mmol) was added to a mixture of intermediate 166 (180 mg, 0.54 mmol) and K₂CO₃ (150 mg, 1.09 mmol) in CH₃CN (5 mL). The reaction mixture was stirred at 75° C. for 48 h. The solvent was removed and the crude product purified by flash column chromatography (silica, MeOH in DCM, gradient from 0/100 to 4/96). The desired fractions were collected and concentrated in vacuo to afford intermediate 190 (171 mg, 63%) as a colorless oil.

Preparation of Intermediate 191

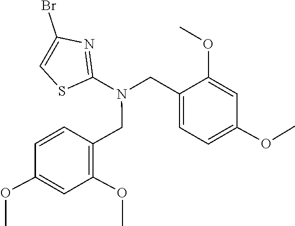

2,4-Dibromo-thiazole ([CAS 4175-77-3], 50 g, 205.83 mmol), N-[(2,4-dimethoxyphenyl)methyl]-2,4-dimethoxy-benzenemethanamine ([CAS 20781-23-1], 65.33 g, 205, 83 mmol) and Na₂CO₃ (65.51 g, 618 mmol) in CH₃CN (500 mL) was heated for 36 hours. The mixture was concentrated and dissolved in EtOAc (1000 mL).

The mixture was washed with water (50 mL) and brine, dried over MgSO₄, and concentrated to give crude product, which was purified by column chromatography on silica gel (petroleum ether/EtOAc, from 100/0 to 70/30) to give intermediate 191 (70 g, 70%) as a yellow solid.

Preparation of Intermediate 192

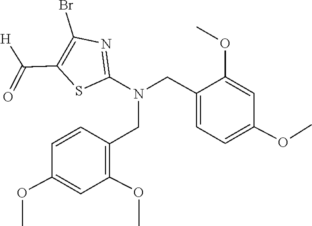 [SMILES: COc1ccc(CN(Cc2ccc(OC)cc2OC)c2nc(Br)c(C=O)s2)c(OC)c1]

To a solution of intermediate 191 (15 g, 31.29 mmol) in anhydrous THF (20 mL) was added dropwise LDA (34.42 mL, 34.42 mmol) at a rate so the temperature did not exceed −70° C. The resulting solution was stirred at −78° C. for 30 min. Then DMF (2.52 g, 34.42 mmol) was added dropwise as a solution in THF (20 mL) and the mixture was allowed to warm up to room temperature. The reaction was quenched with saturated NH₄Cl (30 mL). The mixture was extracted with EtOAc (2×50 mL). The combined organic layers were washed with brine, dried over MgSO₄, and concentrated. The crude was purified by flash chromatography on silica gel (petroleum ether/EtOAc, from 100/0 to 80/20) to yield intermediate 192 (8 g, 45%) as a light yellow solid.

Preparation of Intermediate 193

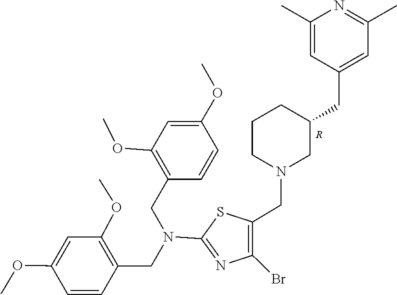

Intermediate 192 (2006.23 mg, 3.95 mmol) was added to intermediate (3R)-34 from WO2018/109202 (729 mg, 3.57 mmol) at RT. After 30 min, sodium triacetoxyborohydride (1512.43 mg, 7.14 mmol) was added to the mixture at RT and the RM was stirred for 48 h at RT. The crude was quenched with NH₃/H₂O and extracted with EtOAc. The organic layer was separated, dried (Na₂SO₄), filtered and the solvent was evaporated in vacuo. The residue was purified by automated flash chromatography (silica, 10% MeOH in DCM 0/100 to 5/95). Desired fractions were collected, concentrated under vacuo to yield intermediate 193 (1.1 g, 44%) as a sticky solid.

Preparation of Intermediate 194

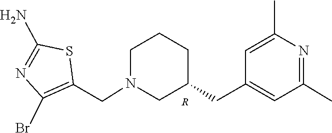

A mixture of intermediate 193 (1050 mg, 1.51 mmol) in TFA (26.25 mL) was stirred at RT under a nitrogen atmosphere for 1.5 h. The solvent was evaporated and the mixture was taken in water, basified with K₂CO₃ and extracted with DCM. The organic layer was dried over MgSO₄ and concentrated. The residue was purified on a column with silica gel, eluent DCM/MeOH (100/0 to 90/10). The pure fractions were evaporated, yielding intermediate 194 (521 mg, 87%) as a white solid.

Preparation of Intermediate 195

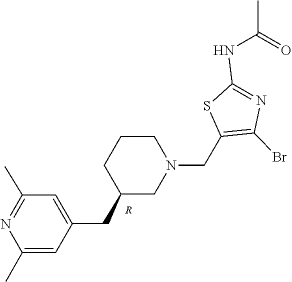

Acetic anhydride (7.75 mg, 0.076 mmol) was added dropwise to a solution of intermediate 194 (20 mg, 0.051 mmol) in 1,4-dioxane (15 mL) while stirring. After the addition was complete, the reaction was heated at 60° C. for 2 h, then at 110° C. for 4 h. The RM was evaporated, taken up in water/0.5 g NaHCO₃/DCM. The organic layer was separated, dried over MgSO₄ and concentrated. The residue was purified on a column with silica gel, eluent: DCM/MeOH (100/0 to 95/5). The pure fractions were concentrated, yielding intermediate 195 (135 mg, 41%) as a pale yellow foam.

Preparation of [³H]-Ligand for Occupancy Study

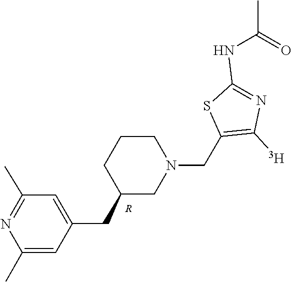

Compound 28 from WO2018/109202 was labelled with [³H] as follows: Intermediate 195 (4.10 mg, 9.38 μmol) and Palladium supported on Carbon (10%, 14.4 mg) were suspended in DMF (0.2 mL) and DIPEA (12 μL, 70.6 μmol) was added. The suspension was degassed three times and stirred under an atmosphere of Tritium gas (4.2 Ci, 525 mbar initial pressure) for 2 h 47 min at RT (end pressure was 311 mbar, no more consumption of gas was observed). The solvent was removed in vacuo, and labile tritium was exchanged by adding MeOH (0.3 mL), stirring the solution, and removing the solvent again under vacuo. This process was repeated twice. Finally, the well dried solid was extracted with EtOH (5 mL) and the suspension was filtered through a 0.2 μm nylon membrane (Macherey-Nagel Polyamide syringe filter CHROMAFIL®Xtra PA-20/25), obtaining a clear solution.

The radiochemical purity (RCP) of the crude material was determined to be 56% using the following HPLC system: Waters Atlantis T3, 5 μm, 4.6×250 mm; solvents A: water+0.05% TFA, B: acetonitrile+0.05% TFA; 0 min 0% B; 10 min 30% B; 10.2-14.5 min 95% B; 15 min 0% B; 254 nm; 1.0 mL/min; 30° C.

The crude was purified by HPLC: Waters Atlantis T3, 5 μm, 10×250 mm; solvents A: water+0.1% TFA; B: acetonitrile+0.1% TFA; 0 min 0% B, 15 min 45% B; 4.7 mL/min; 25° C. The target compound eluted at 9.5 min, and isolated from the HPLC solvent mixture by solid phase extraction. Therefore, the HPLC solution was neutralized with an aqueous solution of NaHCO₃ and the volume of the fractions were partially reduced at the rotary evaporator. Then the product was extracted with a Phenomenex StrataX cartridge (33 μm Polymeric Reversed Phase, 100 mg, 3 mL; 8B-S100-EB) which was eluted with EtOH (5 mL). The extracted product showed an RCP of >99% and the specific activity (SA) was determined to be 10.7 Ci/mmol (396 GBq/mmol, determined by MS). Two batches 250 μCi (9.25 MBq) in 0.25 mL EtOH (1 mCi/mL) and 38.8 mCi in 5 mL EtOH of [³H]-ligand were isolated.

Preparation of Final Compounds

E1. Preparation of Product 1

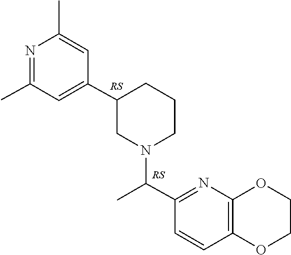 [SMILES: Cc1cc(C2CCCN(C(C)c3ccc4c(n3)OCCO4)C2)cc(C)n1]

2,3-Dihydro-[1,4]dioxino[2,3-b]pyridine-6-carboxaldehyde (60 mg, 0.315 mmol) and titanium(IV) isopropoxide (0.312 mL, 0.444 mmol) were added to a stirred solution of intermediate 1 (60 mg, 0.315 mmol) in DCM (1.1 mL) at rt and under N₂ atmosphere. The mixture was stirred at rt for 16 h. Then it was cooled at 0° C. and methyl magnesium bromide (1.3 mL, 1.83 mmol, 1.4 M in THF/toluene) was added dropwise. The mixture was stirred at this temperature for 15 min and at rt for 16 h. The mixture was treated with sat NH₄Cl, diluted with DCM and the mixture filtered through a pad of diatomaceus earth. The organic layer was separated, dried (MgSO₄), filtered and the solvents evaporated in vacuo. The crude product was purified by flash column chromatography (silica, MeOH in EtOAc in 0/100 to 10/90). The desired fractions were collected and concentrated in vacuo to yield product 1 (110 mg, 99%, mixture of diastereisomers) as a pale yellow oil.

E2. Preparation of Product 2

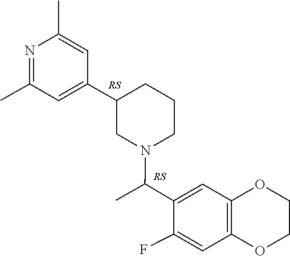 [SMILES: Cc1cc(C2CCCN(C(C)c3cc4c(cc3F)OCCO4)C2)cc(C)n1]

Titanium(IV) isopropoxide (0.13 mL, 0.441 mmol) and sodium cyanoborohydride (33.3 mg, 0.53 mmol) were added sequentially to a mixture of intermediate 1 (100 mg, 0.441 mmol), intermediate 86 (86.5 mg, 0.441 mmol) and triethylamine (0.184 mL, 1.323 mmol) in 1,2 dichloroethane (1.79 mL) at rt. The mixture was stirred at 80° C. for 16 h in a sealed tube. The mixture was treated with water and diluted with DCM and filtered through Celite®. The organic layer separated. dried (Na₂SO₄), filtered and the solvent evaporated in vacuo. The crude product was purified by flash column chromatography (silica, MeOH in EtOAc, 0/100 to 10/90). The desired fractions were collected and evaporated in vacuo to yield a residue that was further purified by RP HPLC (stationary phase: C18 XBridge 30×100 mm 5 μm, mobile phase: gradient from 90% NH₄HCO₃ 0.25% solution in water, 10% CH₃CN to 0% NH₄HCO₃ 0.25% solution in water, 100% CH₃CN). The desired fractions were collected and evaporated in vacuo to yield product 2 (35 mg, 21%, mixture of diastereoisomers) as a white solid.

E3. Preparation of Products 3, 4, 5 and 6

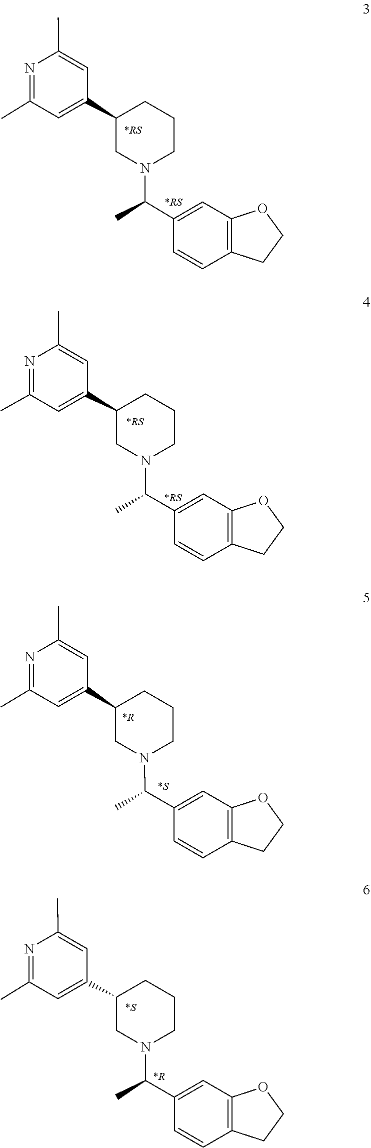 [SMILES: Cc1cc([C@H]2CCCN([C@H](C)c3ccc4c(c3)OCC4)C2)cc(C)n1 ; Cc1cc([C@H]2CCCN([C@@H](C)c3ccc4c(c3)OCC4)C2)cc(C)n1 ; Cc1cc([C@H]2CCCN([C@@H](C)c3ccc4c(c3)OCC4)C2)cc(C)n1 ; Cc1cc([C@@H]2CCCN([C@H](C)c3ccc4c(c3)OCC4)C2)cc(C)n1]

Titanium(IV) isopropoxide (0.15 mL, 0.51 mmol) was added to a stirred solution of intermediate 1 (65 mg, 0.34 mmol) and 2,3-dihydrobenzofuran-6-carboxaldehyde (55.7 mg, 0.38 mmol) in anhydrous DCM (1.18 mL) at rt and under N₂ atmosphere. The mixture was stirred at rt for 16 h. Then the mixture was cooled at 0° C. and methyl magnesium bromide (1.22 mL, 1.71 mmol, 1.4 M in THF/toluene) was added dropwise. The mixture was stirred at this temperature for 15 min and then at rt for 2 h. The mixture was treated with sat. NH₄Cl and extracted with DCM. The phases were filtered through Celite® and then the organic layer was separated, dried (Na₂SO₄), filtered and the solvents evaporated in vacuo. The crude product was purified by flash column chromatography (silica; 7N solution of ammonia in methanol in DCM 0/100 to 2/98). The desired fractions were collected and the solvents evaporated in vacuo to yield a mixture of racemic disatereoisomers (97 mg, 84%). This mixture was then purified by RP HPLC (stationary phase: C18 XBridge 30×100 mm 5 μm, mobile phase: gradient from 60% NH₄HCO₃ 0.25% solution in water, 40% CH₃CN to 43% NH₄HCO₃ 0.25% solution in water, 57% CH₃CN). The desired fractions were collected and the solvents partially concentrated in vacuo. The aqueous phases were extracted with EtOAc, separated, dried (Na₂SO₄), filtered and the solvents evaporated in vacuo to yield product 3 (45 mg, 39%) as pale yellow oil and impure product 3 (40 mg). Impure product 3 was purified by RP HPLC (stationary phase: C18 XBridge 30×100 mm 5 μm, mobile phase: gradient from 54% NH₄HCO₃ 0.25% solution in water, 46% CH₃CN to 36% NH₄HCO₃ 0.25% solution in water, 64% CH₃CN). The desired fractions were collected and the solvents partially concentrated in vacuo. The aqueous phase was extracted with EtOAc, separated, dried (Na₂SO₄), filtered and the solvent evaporated in vacuo to yield product 4 (38.1 mg, 34%) as colorless oil.

Product 4 was subjected to purification via chiral SFC (Stationary phase: CHIRALPAK AD-H 5 μm 250*30 mm, Mobile phase: 85% CO₂, 15% iPrOH (0.3% iPrNH₂)) yielding two fractions that were dissolved in DCM and washed with NaHCO₃ to yield product 5 (11 mg) and product 6 (12 mg) all as pale yellow oils.

E4. Preparation of Product 7

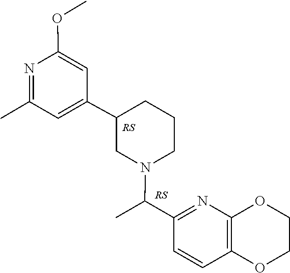 [SMILES: COc1cc(C2CCCN(C(C)c3ccc4c(n3)OCCO4)C2)cc(C)n1]

Product 7 was prepared following an analogous procedure to the one described for the synthesis of product 1 using intermediate 2 (100 mg, 0.48 mmol) and 2,3-dihydro-[1,4]dioxino[2,3-b]pyridine-6-carboxaldehyde as starting materials. Product 7 was purified by RP HPLC (stationary phase: C18 XBridge 30×100 mm 5 μm, mobile phase: gradient from 60% NH₄HCO₃ 0.25% solution in water, 40% CH₃CN to 43% NH₄HCO₃ 0.25% solution in water, 57% CH₃CN) and was isolated (62 mg, 34%, mixture of diastereoisomers) as a colorless oil.

E5. Preparation of Product 8

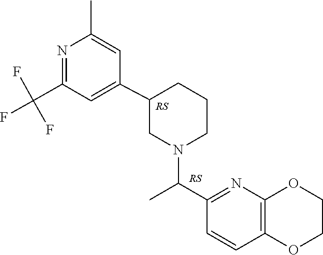 [SMILES: Cc1cc(C2CCCN(C(C)c3ccc4c(n3)OCCO4)C2)cc(C(F)(F)F)n1]

Product 8 was prepared following an analogous procedure to the one described for the synthesis of product 1 using intermediate 3 (80 mg, 0.32 mmol) and 2,3-dihydro-[1,4]dioxino[2,3-b]pyridine-6-carboxaldehyde as starting materials. Product 8 was purified by RP HPLC (stationary phase: C18 XBridge 30×100 mm 5 μm, mobile phase: gradient from 60% NH₄HCO₃ 0.25% solution in water, 40% CH₃CN to 43% NH₄HCO₃ 0.25% solution in water, 57% CH₃CN) and was isolated (112 mg, 84%, mixture of diastereoisomers) as a colorless oil.

E6. Preparation of Product 9

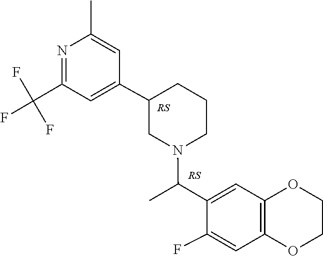 [SMILES: Cc1cc(C2CCCN(C(C)c3cc4c(cc3F)OCCO4)C2)cc(C(F)(F)F)n1]

Product 9 was prepared following an analogous procedure to the one described for the synthesis of product 2 using intermediate 3 (57 mg, 0.23 mmol) and intermediate 86 as starting materials. Product 9 was purified by RP HPLC (stationary phase: C18 XBridge 30×100 mm 5 μm, mobile phase: gradient from 47% NH₄HCO₃ 0.25% solution in water, 53% CH₃CN to 30% NH₄HCO₃ 0.25% solution in water, 70% CH₃CN) and was isolated (38 mg, 38%, mixture of diastereoisomers) as a white solid.

E7. Preparation of Product 10

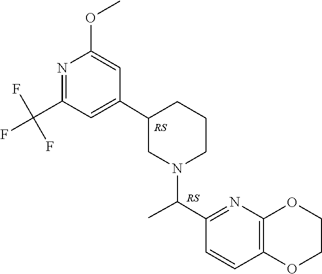 [SMILES: COc1cc(C2CCCN(C(C)c3ccc4c(n3)OCCO4)C2)cc(C(F)(F)F)n1]

Product 10 was prepared following an analogous procedure to the one described for the synthesis of product 1 using intermediate 4 (100 mg, 0.38 mmol) and 2,3-dihydro-[1,4]dioxino[2,3-b]pyridine-6-carboxaldehyde as starting materials. Product 10 was purified by RP HPLC (stationary phase: C18 XBridge 30×100 mm 5 μm, mobile phase: gradient from 60% NH₄HCO₃ 0.25% solution in water, 40% CH₃CN to 43% NH₄HCO₃ 0.25% solution in water, 57% CH₃CN) and was isolated (67 mg, 41%, mixture of diastereoisomers) as a colorless oil.

E8. Preparation of Product 11

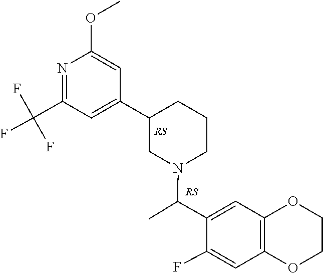 [SMILES: COc1cc(C2CCCN(C(C)c3cc4c(cc3F)OCCO4)C2)cc(C(F)(F)F)n1]

Product 11 was prepared following an analogous procedure to the one described for the synthesis of product 2 using intermediate 4 (100 mg, 0.38 mmol) and intermediate 86 as starting materials. Product 11 was purified by RP HPLC (stationary phase: C18 XBridge 30×100 mm 5 μm, mobile phase: gradient from 47% NH₄HCO₃ 0.25% solution in water, 53% CH₃CN to 30% NH₄HCO₃ 0.25% solution in water, 70% CH₃CN) and was isolated (38 mg, 22%, mixture of diastereoisomers) as a colorless oil.

E9. Preparation of Product 12

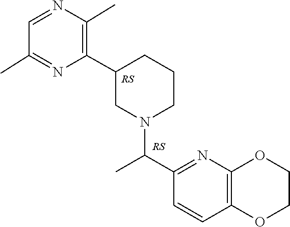 [SMILES: Cc1cnc(C)c(C2CCCN(C(C)c3ccc4c(n3)OCCO4)C2)n1]

Product 12 was prepared following an analogous procedure to the one described for the synthesis of product 1 using intermediate 5 (65 mg, 0.34 mmol) and 2,3-dihydro-[1,4]dioxino[2,3-b]pyridine-6-carboxaldehyde as starting materials. Product 12 was purified by RP HPLC (stationary phase: C18 XBridge 30×100 mm 5 μm, mobile phase: gradient from 54% NH₄HCO₃ 0.25% solution in water, 46% CH₃CN to 36% NH₄HCO₃ 0.25% solution in water, 64% CH₃CN) and was isolated (10 mg, 8%, mixture of diastereoisomers) as a colorless oil.

E10. Preparation of Product 13

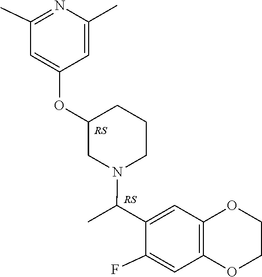 [SMILES: Cc1cc(OC2CCCN(C(C)c3cc4c(cc3F)OCCO4)C2)cc(C)n1]

Product 13 was prepared following an analogous procedure to the one described for the synthesis of product 2 using intermediate 6 (100 mg, 0.48 mmol) and intermediate 86 as starting materials. Product 13 was purified by RP HPLC (stationary phase: C18 XBridge 30×100 mm 5 μm, mobile phase: gradient from 54% NH₄HCO₃ 0.25% solution in water, 46% CH₃CN to 36% NH₄HCO₃ 0.25% solution in water, 64% CH₃CN) and was isolated (5 mg, 3%, mixture of diastereoisomers) as a colorless oil.

E11. Preparation of Product 14

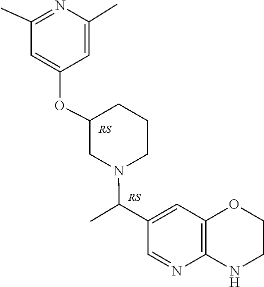 [SMILES: Cc1cc(OC2CCCN(C(C)c3cnc4c(c3)OCCN4)C2)cc(C)n1]

A solution of LiOH (11.8 mg, 0.49 mmol) in water (0.76 mL) was added to a stirred solution of intermediate 87 (64 mg, 0.14 mmol) in 1,4-dioxane (0.76 mL) in a sealed tube. The mixture was stirred at 80° C. for 16 h. The mixture was diluted with water and extracted with DCM. The organic layer was separated, dried (MgSO₄), filtered and the solvents evaporated in vacuo. The crude product was purified by flash column chromatography (silica, 7M solution of ammonia in MeOH in DCM 0/100 to 30/70). The residue was purified by RP HPLC (Stationary phase: C18 XBridge 30×100 mm 5 μm, mobile phase: gradient from 75% NH₄HCO₃ 0.25% solution in water, 25% CH₃CN to 57% NH₄HCO₃ 0.25% solution in water, 43% CH₃CN). The residue was diluted with an aq sat solution of NaHCO₃ and extracted with DCM. The organic layer was separated, dried (MgSO₄), filtered and the solvents evaporated in vacuo yielding product 14 (9 mg, 17%, mixture of diastereoisomers) as a white solid.

E12. Preparation of Product 15

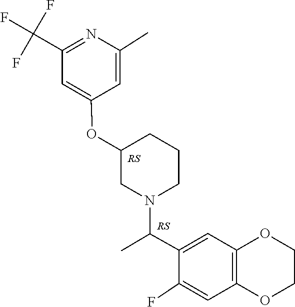 [SMILES: Cc1cc(OC2CCCN(C(C)c3cc4c(cc3F)OCCO4)C2)cc(C(F)(F)F)n1]

Product 15 was prepared following an analogous procedure to the one described for the synthesis of product 2 using intermediate 7 (100 mg, 0.38 mmol) and intermediate 86 as starting materials. Product 15 was purified by RP HPLC (stationary phase: C18 XBridge 30×100 mm 5 μm, mobile phase: gradient from 54% NH₄HCO₃ 0.25% solution in water, 46% CH₃CN to 36% NH₄HCO₃ 0.25% solution in water, 64% CH₃CN) and was isolated (22.8 mg, 13%, mixture of diastereoisomers) as a colorless oil.

E13. Preparation of Products 16 and 17

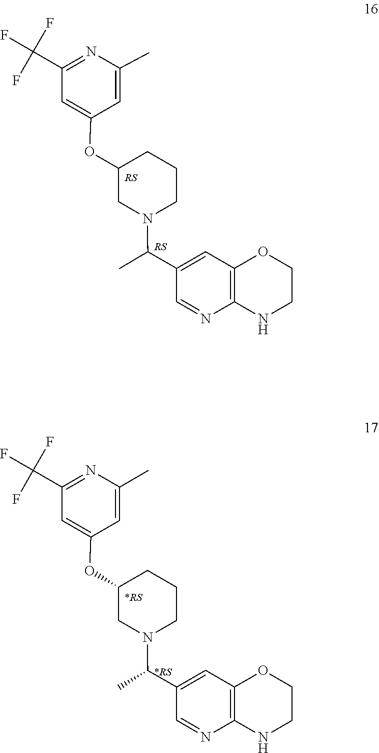 [SMILES: Cc1cc(OC2CCCN(C(C)c3cnc4c(c3)OCCN4)C2)cc(C(F)(F)F)n1 ; Cc1cc(O[C@@H]2CCCN([C@@H](C)c3cnc4c(c3)OCCN4)C2)cc(C(F)(F)F)n1]

Product 16 and product 17 were prepared following an analogous procedure to the one described for the synthesis of product 14 using intermediate 88 (140.9 mg, 0.10 mmol, 37% pure as starting material. Product 16 was purified twice by RP HPLC (stationary phase: C18 XBridge 30×100 mm 5 μm, mobile phase: gradient from 60% NH₄HCO₃ 0.25% solution in water, 40% CH₃CN to 43% NH₄HCO₃ 0.25% solution in water, 57% CH₃CN) and was isolated (25.6 mg, 59%, mixture of diastereoisomers) as a colorless oil. In addition, and from the same purification, product 17 (13 mg, 30%, single diastereoisomer, racemic) was isolated as a colorless oil.

E14. Preparation of Product 18

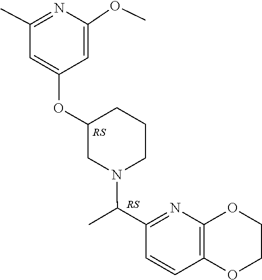 [SMILES: COc1cc(OC2CCCN(C(C)c3ccc4c(n3)OCCO4)C2)cc(C)n1]

Product 18 was prepared following an analogous procedure to the one described for the synthesis of product 1 using intermediate 8 (100 mg, 0.45 mmol) and 2,3-dihydro-[1,4]dioxino[2,3-b]pyridine-6-carboxaldehyde as starting materials. Product 18 was purified by RP HPLC (stationary phase: C18 XBridge 30×100 mm 5 μm, mobile phase: gradient from 90% NH₄HCO₃ 0.25% solution in water, 10% CH₃CN to 65% NH₄HCO₃ 0.25% solution in water, 35% CH₃CN) and was isolated (9.4 mg, 5%, mixture of diastereoisomers) as a colorless oil.

E15. Preparation of Product 19

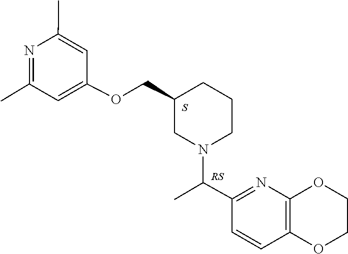

Product 19 was prepared following an analogous procedure to the one described for the synthesis of product 1 using intermediate 9 (200 mg, 0.91 mmol) and 2,3-dihydro-[1,4]dioxino[2,3-b]pyridine-6-carboxaldehyde as starting materials. Product 19 was isolated (209 mg, 60%, mixture of diastereoisomers) as a colorless oil.

E16. Preparation of Products 20, 108 and 109

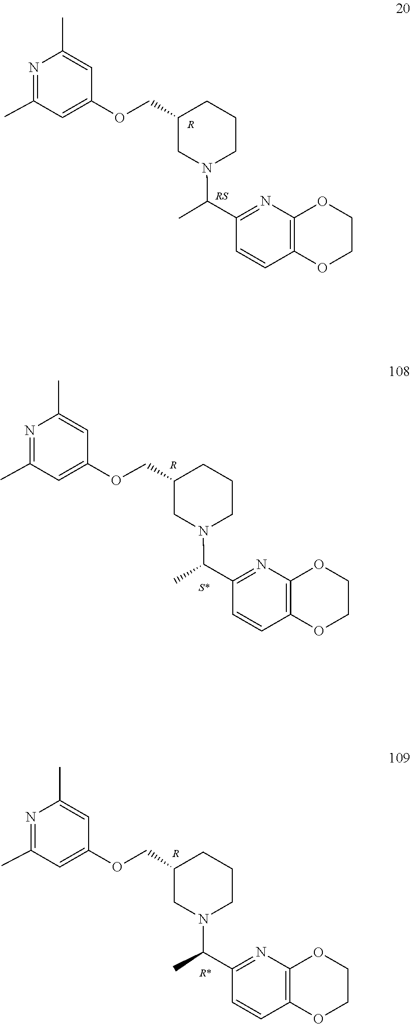

Product 20 was prepared following an analogous procedure to the one described for the synthesis of product 1 using intermediate 10 (200 mg, 0.91 mmol) and 2,3-dihydro-[1,4]dioxino[2,3-b]pyridine-6-carboxaldehyde as starting materials. Product 20 was isolated (263.3 mg, 76%, mixture of diastereoisomers) as a colorless oil.

Product 20 (250 mg) was purified via chiral SFC (stationary phase: CHIRACEL OJ-H 5 μm 250*30 mm, mobile phase: 70% CO₂, 30% MeOH (0.3% iPrNH₂)) yielding product 108 (116 mg, 33%) and product 109 (107 mg, 31%).

E17. Preparation of Product 21

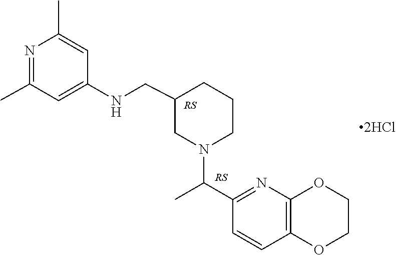 [SMILES: Cc1cc(NCC2CCCN(C(C)c3ccc4c(n3)OCCO4)C2)cc(C)n1]

N₂ was bubbled through a solution of 4-bromo-2,6-dimethylpyridine (66.4 mg, 0.36 mmol) in 1,4-dioxane (6 mL). Then sodium tert-butoxyde (68.6 mg, 0.71 mmol), 2-dicyclohexylphosphino-2′-(N,N-dimethylamino)biphenyl (14 mg, 0.036 mmol) and tris(dibenzylideneacetone)dipalladium(0) (16.3 mg, 0.018 mmol) were added at rt while N₂ was bubbled. Then intermediate 11 (106 mg, 0.37 mmol) was added. Then the vial was capped and the mixture was stirred at 100° C. overnight. The mixture was cooled to rt, diluted with EtOAc and 0.5 mL of NH₄Cl sat., filtered over a Celite® pad and the solvents evaporated in vacuo. The crude product was purified by flash column chromatography (silica; MeOH in DCM 0/100 to 10/90). The desired fractions were collected and concentrated in vacuo to yield a crude product that was further purified by reverse phase from 95% [65 mM NH₄OAc+ACN (90:10)]—5% [ACN] to 63% [65 mM NH₄OAc+ACN (90:10)]—37% [ACN]. The desired fractions were collected and concentrated in vacuo. To remove remaining NH₄Ac the product containing fraction was purified again by reverse phase from 81% [H₂O (25 mM NH₄HCO₃)-19%[ACN] to 45% [H₂O (25 mM NH₄HCO₃)]—55% [ACN)]. The desired fractions were collected and concentrated to give product 21 (50 mg, 36%) as a colorless sticky solid. This material was taken up in DCM and treated with 2 eq of HCl 4N in 1,4-dioxane. The solvents evaporated in vacuo and the product was triturated with diethyl ether to yield product 21 (2×HCl salt, 48 mg, 29%) as a white solid.

E18. Preparation of Product 22

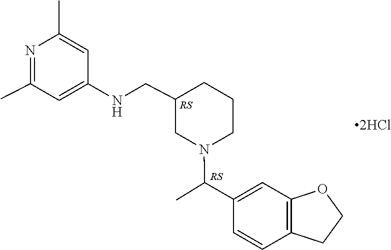 [SMILES: Cc1cc(NCC2CCCN(C(C)c3ccc4c(c3)OCC4)C2)cc(C)n1]

Product 22 was prepared following an analogous procedure to the one described for the synthesis of product 21 using intermediate 12 (152 mg, 0.57 mmol) and 4-bromo-2,6-dimethylpyridine as starting materials. Product 22 was isolated (2×HCl salt, 126 mg, 52%, mixture of diastereoisomers) as white solid.

E19. Preparation of Product 23

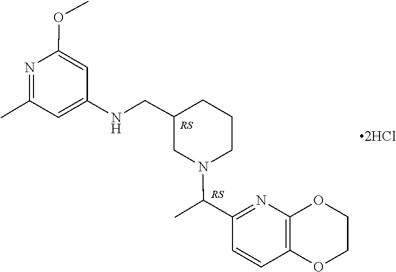 [SMILES: COc1cc(NCC2CCCN(C(C)c3ccc4c(n3)OCCO4)C2)cc(C)n1 ; "·2HCl"]

Product 23 was prepared following an analogous procedure to the one described for the synthesis of product 21 using intermediate 11 (132 mg, 0.47 mmol) and 4-bromo-2-methoxy-6-methylpyridine (CAS: 1083169-00-9) as starting materials. Product 23 was isolated (2×HCl salt, 30 mg, 14%, mixture of diastereoisomers) as white solid.

E20. Preparation of Products 24, 25 and 26

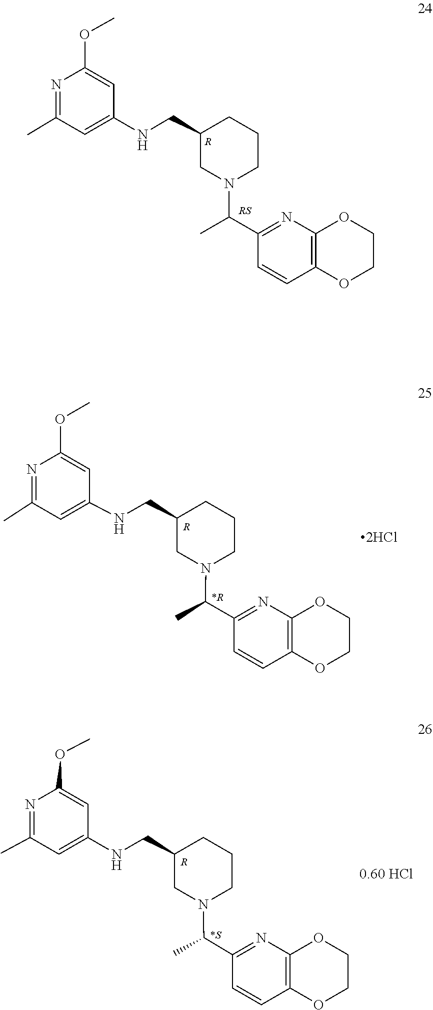

Product 24 was prepared following an analogous procedure to the one described for the synthesis of product 21 using intermediate 13 (680 mg, 2.4 mmol) and 4-bromo-2-methoxy-6-methylpyridine (CAS: 1083169-00-9) as starting materials. Product 24 was isolated (613 mg, 65%, mixture of diastereoisomers) as a sticky solid. Product 24 (604 mg, 1.47 mmol) was purified via chiral SFC (stationary phase: CHIRALPAK IC 5 μm 250*30 mm, mobile phase: 50% CO₂, 50% iPrOH (0.3% iPrNH₂)) yielding product 25 (267 mg, 45%) and product 26 (200 mg, 34%) both as sticky solids. Product 25 and product 26 were taken up in diethyl ether and treated with 4 eq of HCl 4N in diethyl ether. The solvents evaporated in vacuo and the products were triturated with diethyl ether to yield product 25 (HCl salt, 157 mg, 24%) and product 26 (0.6×HCl salt, 129 mg, 21%) both as white solids.

E21. Preparation of Products 27, 28 and 29

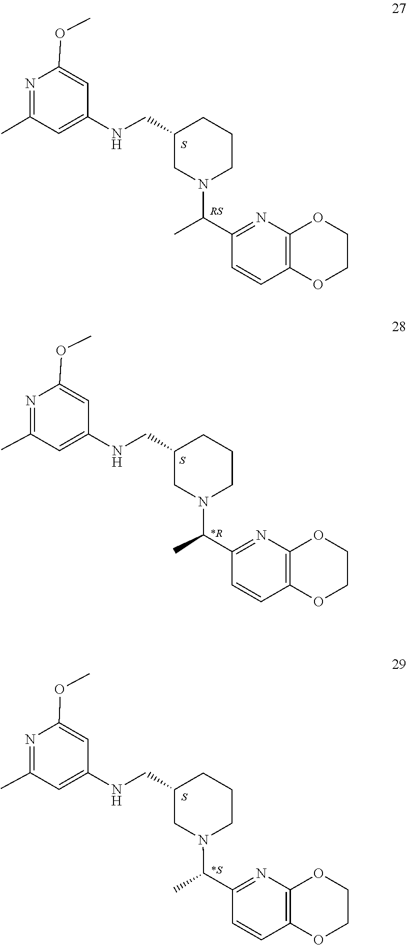

Product 27 was prepared following an analogous procedure to the one described for the synthesis of product 21 using intermediate 14 (627 mg, 2.26 mmol) and 4-bromo-2-methoxy-6-methylpyridine (CAS: 1083169-00-9) as starting materials. Product 27 was isolated (613 mg, 71%, mixture of diastereoisomers) as orange sticky solid. Product 27 (592 mg, 1.47 mmol) was purified via chiral SFC (stationary phase: CHIRALPAK IC 5 μm 250*30 mm, mobile phase: 50% CO₂, 50% iPrOH (0.6% iPrNH₂)) yielding product 28 (267 mg, 45%) and product 29 (200 mg, 34%) both as sticky solids.

E22. Preparation of Product 30

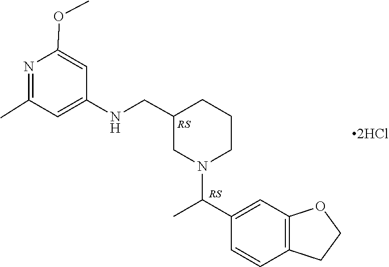 [SMILES: COc1cc(NCC2CCCN(C(C)c3ccc4c(c3)OCC4)C2)cc(C)n1]

Product 30 was prepared following an analogous procedure to the one described for the synthesis of product 21 using intermediate 12 (152 mg, 0.57 mmol) and 4-bromo-2-methoxy-6-methylpyridine (CAS: 1083169-00-9) as starting materials. Product 30 was isolated (2×HCl salt, 105 mg, 41%, mixture of diastereoisomers) as white solid.

E23. Preparation of Products 31, 32, 33, 34 and 35

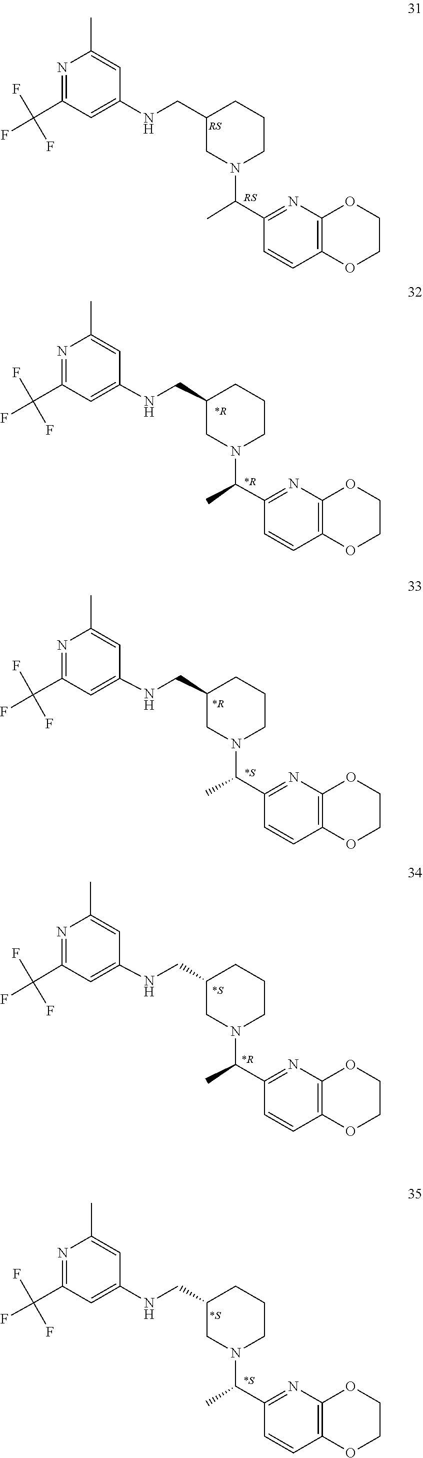 [SMILES: Cc1cc(NCC2CCCN(C(C)c3ccc4c(n3)OCCO4)C2)cc(C(F)(F)F)n1 ; Cc1cc(NC[C@H]2CCCN([C@H](C)c3ccc4c(n3)OCCO4)C2)cc(C(F)(F)F)n1 ; Cc1cc(NC[C@H]2CCCN([C@@H](C)c3ccc4c(n3)OCCO4)C2)cc(C(F)(F)F)n1 ; Cc1cc(NC[C@@H]2CCCN([C@H](C)c3ccc4c(n3)OCCO4)C2)cc(C(F)(F)F)n1 ; Cc1cc(NC[C@@H]2CCCN([C@@H](C)c3ccc4c(n3)OCCO4)C2)cc(C(F)(F)F)n1]

Intermediate 89 (118 mg, 0.26 mmol) and trimethylboroxine (0.043 mL, 0.31 mmol) were added to a stirred suspension of K₃PO₄ (82 mg, 0.39 mmol), 2-dicyclohexylphosphino-2′,4′,6′-triisopropylbiphenyl (12.3 mg, 0.026 mmol) and tris(dibenzylideneacetone)dipalladium(0) (11.8 mg, 0.013 mmol) in 1,4-dioxane (5 mL) under nitrogen. The mixture was stirred at 100° C. 2-Dicyclohexylphosphino-2′,4′,6′-triisopropylbiphenyl (6.15 mg, 0.013 mmol) and tris(dibenzylideneacetone)dipalladium(0) (5.9 mg, 0.0065 mmol) were added to the mixture at rt and under nitrogen atmosphere. Water and EtOAc were added. The organic layer was separated, dried (MgSO₄) and filtered and the solvents evaporated in vacuo. The crude was purified by flash column chromatography (silica; EtOAc in heptane 0/100 to 50/50). The desired fractions were collected and concentrated in vacuo to yield impure product 31 (90 mg) as yellow solid.

Impure product 31 (90 mg) was purified by reverse phase chromatography 70% [25 mM NH₄HCO₃]—30% [ACN:MeOH 1:1] to 27% [25 mM NH₄HCO₃]—73% [ACN:MeOH 1:1]. The desired fractions were collected and concentrated at 60° C. ACN (10 mL×3 times) was added and concentrated at 60° C. to yield pure product 31 (83 mg, 73%) as a colorless oil.

Product 31 (83 mg) was purified via chiral SFC (Stationary phase: Lux-Cellulose-4 5 μm 250*21.2 mm, Mobile phase: 75% CO₂, 25% iPrOH (0.3% iPrNH₂)) yielding product 32 (14 mg, 17%), product 33 (16 mg, 19%) and 26 mg of a mixture of product 34 and product 35. This mixture (26 mg) was purified via chiral SFC (Stationary phase: CHIRACEL OJ-H 5 μm 250*20 mm, Mobile phase: 90% CO₂, 10% MeOH (0.3% iPrNH₂)) yielding product 34 (13 mg, 16%) and product 35 (13 mg, 16%).

E24. Preparation of Product 36

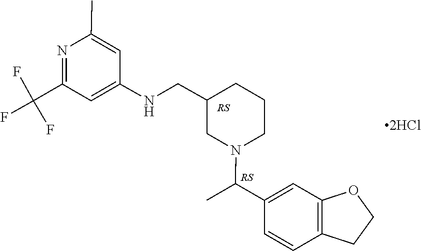 [SMILES: Cc1cc(NCC2CCCN(C(C)c3ccc4c(c3)OCC4)C2)cc(C(F)(F)F)n1]

Product 36 was prepared following an analogous procedure to the one described for the synthesis of product 2 using intermediate 61 (84.2 mg, 0.31 mmol) and 1-(2,3-dihydro-benzofuran-6-yl)-ethanone (CAS: 374706-07-7) as starting materials. Product 36 was purified by reverse phase from 72% [65 mM NH₄OAc+ACN (90:10)]—28% [MeCN:MeOH (1:1)] to 36% [65 mM NH₄OAc+ACN (90:10)]—64% [MeCN:MeOH (1:1)]. The desired fractions were collected and evaporated in vacuo with MeCN/water (1:1). Then the residue was diluted with water and extracted with DCM to product 36 (23 mg) as a colorless sticky solid. The product was taken up in DCM and treated with 2 eq. of HCl 4N in dioxane. The solvents were evaporated in vacuo and the product was triturated with diethyl ether, filtered and dried to yield product 36 (2×HCl salt, 16.9 mg, 11%) as a white solid.

E25. Preparation of Product 37

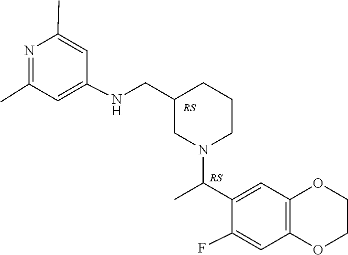 [SMILES: Cc1cc(NCC2CCCN(C(C)c3cc4c(cc3F)OCCO4)C2)cc(C)n1]

Product 37 was prepared following an analogous procedure to the one described for the synthesis of product 1 using intermediate 15 (112 mg, 0.51 mmol) and intermediate 86 as starting materials. Product 37 was purified by reverse phase from 81% [25 mM NH₄HCO₃]—19% [MeCN:MeOH (1:1)] to 45% [25 mM NH₄HCO₃]—55% [MeCN:MeOH (1:1)]. Product 37 was isolated (9 mg, 4%, mixture of diastereoisomers) as white foam after trituration with diethyl ether.

E26. Preparation of Product 38

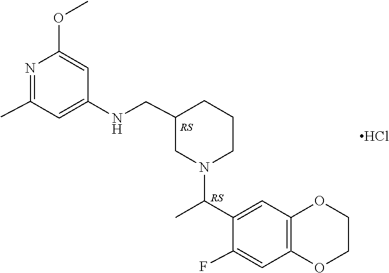 [SMILES: COc1cc(NCC2CCCN(C(C)c3cc4c(cc3F)OCCO4)C2)cc(C)n1 ; Cl]

Product 38 was prepared following an analogous procedure to the one described for the synthesis of product 1 using intermediate 59 (112 mg, 0.51 mmol) and intermediate 86 as starting materials. Product 38 was purified by reverse phase from 59% [25 mM NH₄HCO₃]—41% [MeCN:MeOH (1:1)] to 17% [25 mM NH₄HCO₃]—83% [MeCN:MeOH (1:1)]. Product 38 was isolated (73 mg, 34%, mixture of diastereoisomers) as colorless oil. The product 38 was then taken up in DCM and treated with 2 eq. of HCl 4N in dioxane. The solvents were evaporated in vacuo and the product was triturated with diethyl ether, filtered and dried to yield product 38 (1×HCl salt, 69 mg, 30%) as a white solid.

E27. Preparation of Product 39

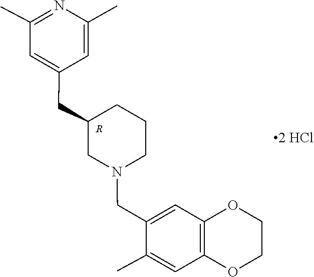

Lithium aluminium hydride (0.31 mL, 0.31 mm, 1M solution in THF) was added dropwise to a stirred solution of intermediate 90 (78 mg, 0.2 mmol) in THF (2 mL) at 0° C. in a sealed tube and under N₂ atmosphere. The mixture was stirred at 0° C. for 5 min and at rt for 2 h. The mixture was cooled at 0° C. and treated with EtOAc and Na₂SO₄.10H₂O. The mixture was stirred at rt for 30 min and it was filtered through a Celite® pad and washed with EtOAc. The filtrate was concentrated and the crude was purified by RP HPLC (Stationary phase: C18 XBridge 30×100 mm 5 μm, mobile phase: gradient from 60% 10 mM NH₄CO₃H pH 9 solution in water, 40% CH₃CN to 43% 10 mM NH₄CO₃H pH 9 solution in water, 57% CH₃CN). The desired fractions were collected and the solvents evaporated in vacuo. The residue was dissolved in diethyl ether and converted into its HCl salt by treatment with 4N HCl in 1,4-dioxane. The solid formed was filtered and dried to yield product 39 (2×HCl salt, 17.7 mg, 20%) as a white foam.

E28. Preparation of Products 40, 41 and 42

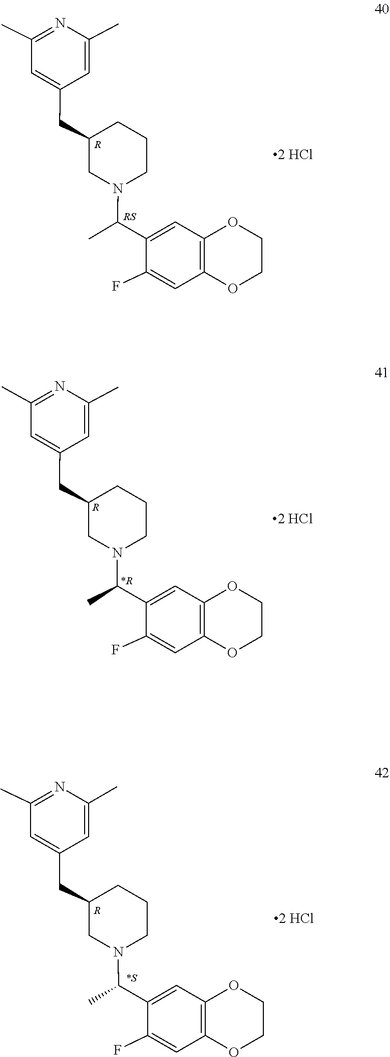

Product 40 was prepared following an analogous procedure to the one described for the synthesis of product 2 using intermediate 16 (124.4 mg, 0.45 mmol, 2×HCl salt) and intermediate 86 as starting materials. Product 40 was purified by RP HPLC (stationary phase: C18 XBridge 30×100 mm 5 μm, mobile phase: gradient from 54% 0.1% NH₄CO₃H/NH₄OH pH 9 solution in water, 46% CH₃CN to 64% 0.1% NH₄CO₃H/NH₄OH pH 9 solution in water, 36% CH₃CN), yielding product 40 (65 mg, 41%, mixture of diastereoisomers) as an oil.

Product 40 was purified via chiral SFC (Stationary phase: Chiralcel OD-H 5 μm 250×21.2 mm, Mobile phase: 80% CO₂, 20% iPrOH (0.3% iPrNH₂)) yielding product 41 (29 mg, 18%) and product 42 (24 mg, 15%) both as oils.

Product 41 (29 mg) was then taken up in MeOH (1 mL) and treated with HCl (5 mL, 6N in MeOH) for 2 h. The solvents were evaporated in vacuo and the product was triturated with diisopropyl ether, filtered and dried to yield product 41 (2×HCl salt, 32 mg) as a cream color solid.

Product 42 (24 mg) was then taken up in MeOH (1 mL) and treated with HCl (0.55 mL, 6N in MeOH) for 2 h. The solvents were evaporated in vacuo and the product was triturated with diisopropyl ether, filtered and dried to yield product 42 (2×HCl salt, 32 mg) as a cream color solid.

E29. Preparation of Product 43

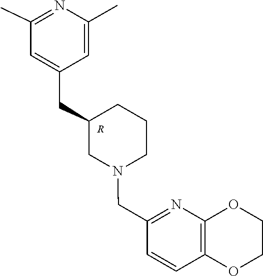

Triethylamine (0.18 mL, 1.33 mmol) followed by 2,3-dihydro-[1,4]dioxino[2,3-b]pyridine-6-carboxaldehyde (59 mg, 0.36 mmol) were added to a stirred suspension of intermediate 16 (90 mg, 0.32 mmol, 2×HCl salt) in DCM (1.7 mL) in a sealed tube and under N₂ atmosphere. The mixture was stirred at rt for 30 min and then sodium triacetoxyborohydride (59 mg, 0.36 mmol) was added. The mixture was stirred at rt for 16 h. The mixture was treated with sat NaHCO₃ and extracted with DCM. The organic layer was separated, dried (MgSO₄), filtered and the solvents evaporated in vacuo. The crude product was purified by flash column chromatography (SiO₂, 7N solution of NH₃ in MeOH in DCM 0/100 to 5/95). The desired fractions were collected and concentrated in vacuo to yield product 43 (96 mg, 84%) as a pale yellow oil.

E30. Preparation of Products 44, 45 and 46

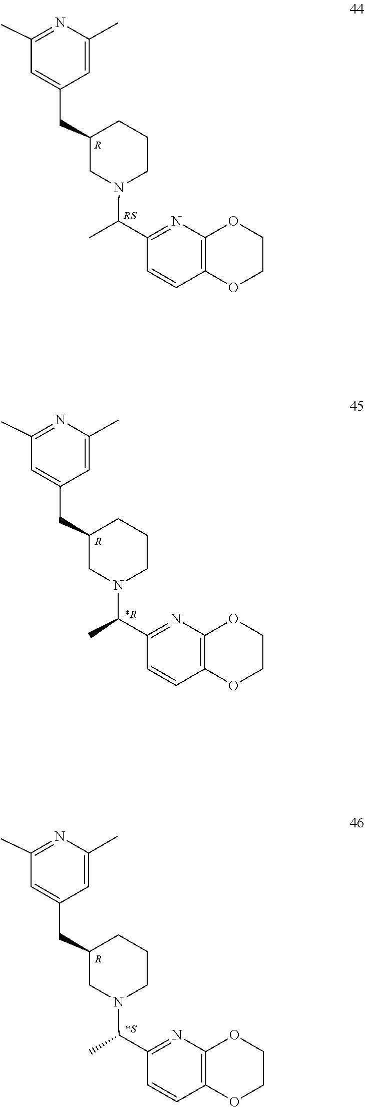

Product 44 was prepared following an analogous procedure to the one described for the synthesis of product 2 using intermediate 16 (95 mg, 0.34 mmol, 2×HCl salt) and 1-(2,3-dihydro-[1,4]dioxino[2,3-b]pyridin-6-yl)ethenone (CAS: 1254044-25-1) as starting materials. Product 44 was purified by RP HPLC (stationary phase: C18 XBridge 30×100 mm 5 μm, mobile phase: gradient from 75% NH₄HCO₃ 0.25% solution in water, 25% CH₃CN to 57% NH₄HCO₃ 0.25% solution in water, 43% CH₃CN) yielding product 44 (62 mg, 49%, mixture of diastereoisomers) as a colorless oil.

Product 44 (50 mg) was purified via chiral SFC (stationary phase: chiralpak IC 5 μm 250*21.2 mm, mobile phase: 60% CO₂, 40% iPrOH (0.3% iPrNH₂)) yielding product 45 (24 mg, 19%) and product 46 (23 mg, 18%) both as yellow oils.

E31. Preparation of Product 47

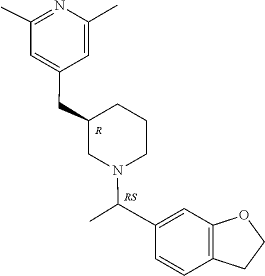

Product 47 was prepared following an analogous procedure to the one described for the synthesis of product 1 using intermediate 16 (100 mg, 0.49 mmol, 2×HCl salt) and intermediate 2,3-dihydro-1-benzofuran-6-carbaldehyde as starting materials. Product 47 was purified by RP HPLC (stationary phase: C18 XBridge 30×100 mm 5 □m, mobile phase: gradient from 90% NH₄HCO₃ 0.25% solution in water, 10% CH₃CN to 65% NH₄HCO₃ 0.25% solution in water, 35% CH₃CN) yielding product 47 (62 mg, 49%, mixture of diastereoisomers) as a creamy sticky solid.

E32. Preparation of Product 48

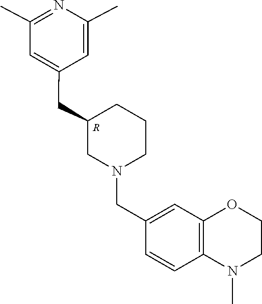

Product 48 was prepared following an analogous procedure to the one described for the synthesis of product 43 using intermediate 16 (85 mg, 0.42 mmol) and 4-methyl-3,4-dihydro-2H-1,4-benzoxazine-7-carbaldehyde (CAS: 141103-93-7) as starting materials. Product 48 was purified by RP HPLC (stationary phase: C18 XBridge 30×100 mm 5 μm, mobile phase: gradient from 81% 10 mM NH₄CO₃H pH 9 solution in water, 19% CH₃CN to 64% 10 mM NH₄CO₃H pH 9 solution in water, 36% CH₃CN) yielding product 48 (33 mg, 22%) as a colorless oil.

E33. Preparation of Product 49

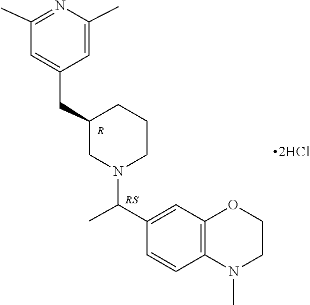

Product 49 was prepared following an analogous procedure to the one described for the synthesis of product 1 using intermediate 16 (100 mg, 0.36 mmol, 2×HCl) and 4-methyl-3,4-dihydro-2H-1,4-benzoxazine-7-carbaldehyde (CAS: 141103-93-7) as starting materials. Product 49 was purified by RP HPLC (stationary phase: C18 XBridge 30×100 mm 5 μm, mobile phase: gradient from 60% NH₄HCO₃ 0.25% solution in water, 40% CH₃CN to 43% NH₄HCO₃ 0.25% solution in water, 57% CH₃CN), yielding product 49 which was then taken up in MeOH and treated with HCl (6N solution in i-PrOH). The solvents were evaporated in vacuo to yield product 49 (60 mg, 37%, 2×HCl salt, mixture of diastereoisomers) as a white solid.

E34. Preparation of Products 50, 51 and 52

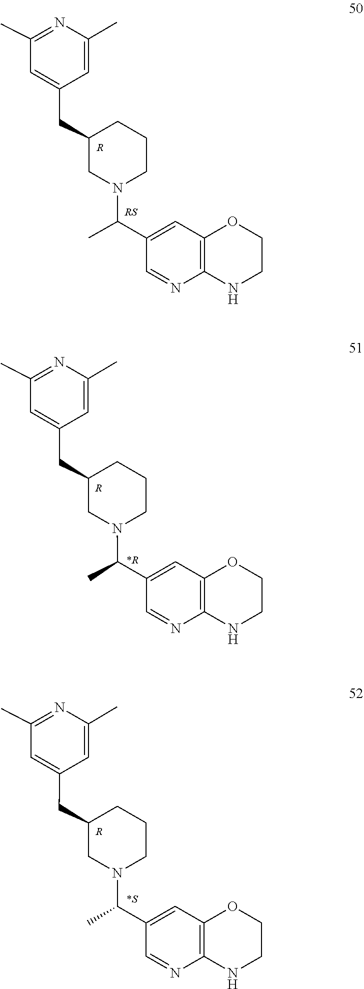

Product 50 was prepared following an analogous procedure to the one described for the synthesis of product 14 using intermediate 91 (168 mg, 0.37 mmol) as starting material. Product 50 (81 mg, 59%, mixture of diastereoisomers) was isolated as a white foam. Product 50 (70 mg) was subjected to purification via chiral SFC (stationary phase: CHIRACEL OJ-H 5 μm 250*30 mm, mobile phase: 90% CO₂, 10% EtOH (0.3% iPrNH₂)) yielding product 51 (19 mg, 14%) and product 52 (22 mg, 16%) both as pale yellow foams.

E35. Preparation of Product 53

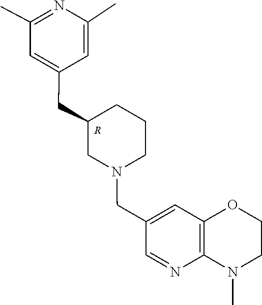

Borane dimethylsulfide complex (0.05 mL, 0.53 mmol) was added dropwise to a stirred solution of intermediate 92 (74 mg, 0.19 mmol) in THF (1 ML) in a sealed tube and under N₂ atmosphere. The mixture was stirred at 60° C. for 2 h. The mixture was cooled at 0° C. and MeOH (5 mL) was added. The mixture was stirred at rt for 1 h. The solvent was evaporated in vacuo. The crude was dissolved with MeOH (10 mL) in a sealed tube and under N₂ atmosphere and the mixture was stirred at 70° C. for 8 h. The solvent was evaporated in vacuo. The crude product was purified by RP HPLC (Stationary phase: C18 XBridge 30×100 mm 5 μm, mobile phase: gradient from 75% NH₄HCO₃ 0.25% solution in water, 25% CH₃CN to 57% NH₄HCO₃ 0.25% solution in water, 43% CH₃CN). The desired fractions were collected and extracted with EtOAc. The organic layer was separated, dried (MgSO₄), filtered and the solvents evaporated in vacuo to yield product 53 (31 mg, 71%) as a pale yellow oil.

E36. Preparation of Product 54

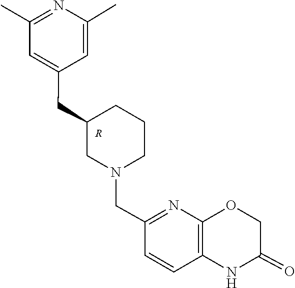

Product 54 was prepared following an analogous procedure to the one described for the synthesis of product 43 using intermediate 16 (164 mg, 0.59 mmol, 2×HCl salt) and 2-oxo-1H-pyrido[2,3-b][1,4]oxazine-6-carbaldehyde (CAS: 1417554-43-8) as starting materials. Product 54 was purified by RP HPLC (stationary phase: Sunfire™ Prep C18 OBD 30×100 mm 5 μm, Mobile phase: gradient from 60% 0.1% HCO₂H solution in H₂O, 40% CH₃CN to 43% 0.1% HCO₂H solution in H₂O, 57% CH₃CN), yielding 21 mg of a residue that was taken up in DCM and washed with NaHCO₃ (aq. sat. sltn.). The organic layer was separated, dried (Na₂SO₄), filtered and evaporated in vacuo to give product 54 (19.1 mg, 9%) as a colorless sticky oil.

E37. Preparation of Product 55

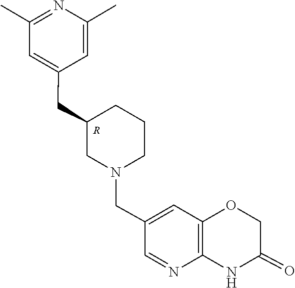

Product 55 was prepared following an analogous procedure to the one described for the synthesis of product 43 using intermediate 16 (261 mg, 0.94 mmol, 2×HCl salt) and 3-oxo-4H-pyrido[3,2-b][1,4]oxazine-7-carbaldehyde as starting materials. Product 55 was purified by RP HPLC (stationary phase: C18 XBridge 30×100 mm 5 □m, mobile phase: gradient from 75% NH₄HCO₃ 0.25% solution in water, 25% CH₃CN to 57% NH₄HCO₃ 0.25% solution in water, 43% CH₃CN), to give a residue which was washed with aqueous saturated NaHCO₃ solution and DCM. The organic layer was separated, dried (Na₂SO₄), filtered and evaporated in vacuo to give impure product 55 (57.8 mg, 80&% pure) as a white solid Impure product 55 (57.8 mg, 80&% pure) was purified by RP HPLC (stationary phase: Sunfire™ Prep C18 OBD 30×100 mm 5 μm, mobile phase: gradient from 54% 0.1% HCO₂H solution in H₂O, 46% CH₃CN to 36% 0.1% HCO₂H solution in H₂O, 64% CH₃CN), yielding product 55 (23 mg, 6.6%) as a white solid.

E38. Preparation of Product 56

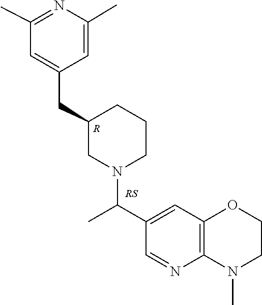

Product 56 was prepared following an analogous procedure to the one described for the synthesis of product 53 using product 62 (90 mg, 0.23 mmol) as starting material. Product 56 was purified by RP HPLC (stationary phase: C18 XBridge 30×100 mm 5 μm, mobile phase: gradient from 75% NH₄HCO₃ 0.25% solution in water, 25% CH₃CN to 57% NH₄HCO₃ 0.25% solution in water, 43% CH₃CN). The desired fractions were collected and extracted with EtOAc. The organic layer was separated, dried (MgSO₄), filtered and the solvents evaporated in vacuo to yield product 56 (54 mg, 62%, mixture of diastereoisomers) as a pale yellow oil.

E39. Preparation of Product 57

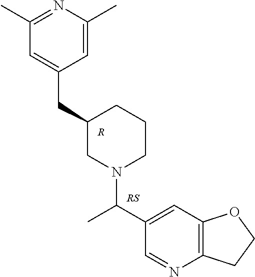

A solution of intermediate 95 (40 mg, 0.11 mmol) in EtOH (2.5 mL) was hydrogenated in a H-cube® reactor (1 mL/min, 35 mm Pd/C 10% cartridge, full H₂ mode, rt, 25° C., 1 cycle, 50° C., 2 cycles). The solvent was evaporated in vacuo and the crude product was purified by RP HPLC (stationary phase: C18 XBridge 30×100 mm 5 μm, mobile phase: gradient from 75% NH₄HCO₃ 0.25% solution in water, 25% CH₃CN to 57% NH₄HCO₃ 0.25% solution in water, 43% CH₃CN). The desired fractions were collected and extracted with EtOAc. The organic layer was separated, dried (MgSO₄), filtered and the solvents evaporated in vacuo to yield product 57 (7.3 mg, 18%, mixture of diastereoisomers) as a colorless oil.

E40. Preparation of Product 58

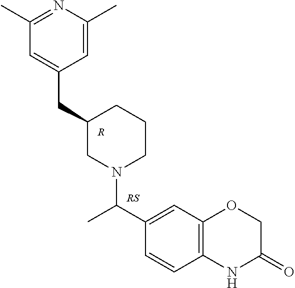

Product 58 was prepared following an analogous procedure to the one described for the synthesis of product 1 using intermediate 16 (99 mg, 0.48 mmol) and 3-oxo-3,4-dihydro-2H-benz[1,4]oxazine-7-carboxaldehyde as starting materials. Product 58 was isolated as a pale yellow oil (55 mg, 30%, mixture of diastereoisomers) that solidified upon standing.

E41. Preparation of Products 59, 60 and 61

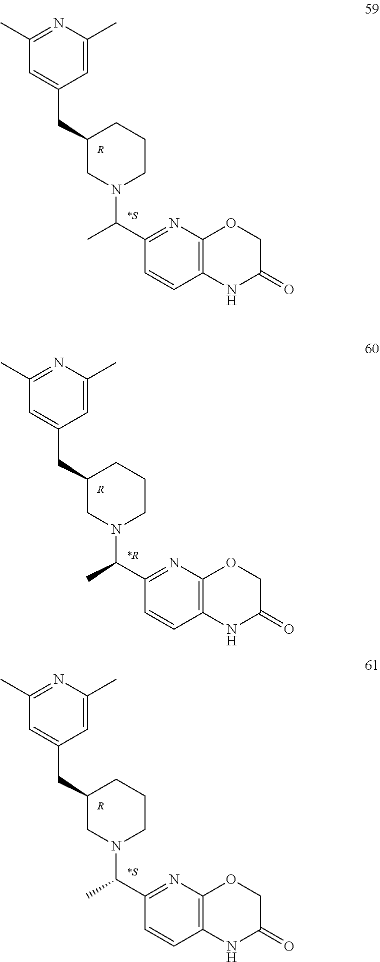

Product 59 was prepared following an analogous procedure to the one described for the synthesis of product 1 using intermediate 16 (91 mg, 0.44 mmol) and 2-oxo-1H-pyrido[2,3-b][1,4]oxazine-6-carbaldehyde (CAS: 1417554-43-8) as starting materials. Product 59 (95 mg, 56%, mixture of diastereoisomers) was isolated as a pale yellow foam.

Product 59 (80 mg) was purified via chiral SFC (Stationary phase: CHIRACEL OJ-H 5 μm 250*30 mm, Mobile phase: 90% CO₂, 10% EtOH (0.3% iPrNH₂)) yielding product 60 (30 mg, 18%) and product 61 (24 mg, 14%) both as white foams.

E42. Preparation of Product 62

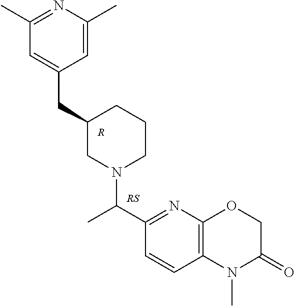

Product 62 was prepared following an analogous procedure to the one described for the synthesis of product 1 using intermediate 16 (116 mg, 0.57 mmol) and 3-oxo-4H-pyrido[3,2-b][1,4]oxazine-7-carbaldehyde as starting materials. Product 62 (103 mg, 45%, mixture of diastereoisomers) was isolated as a yellow oil.

E43. Preparation of Products 63, 64 and 65

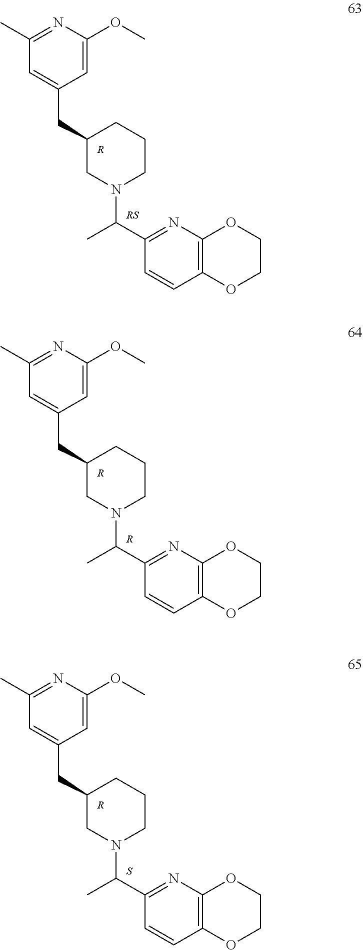

Product 63 was prepared following an analogous procedure to the one described for the synthesis of product 1 using intermediate 17 (100 mg, 0.45 mmol) and 2,3-Dihydro[1,4]dioxino[2,3-b]pyridine-6-carbaldehyde (CAS: 615568-24-6) as starting materials. Crude product 63 was purified by RP HPLC (stationary phase: C18 XBridge 30×100 mm 5 μm, mobile phase: gradient from 60% NH₄HCO₃ 0.25% solution in water, 40% CH₃CN to 43% NH₄HCO₃ 0.25% solution in water, 57% CH₃CN), yielding product 63 (102 mg, 59%, mixture of diastereoisomers), product 64 (9.9 mg, 6%, single racemic diastereoisomer) and product 65 (36 mg, 21%, single racemic diastereoisomer), all as colorless oils.

E44. Preparation of Product 66

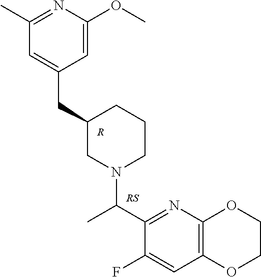

Product 66 was prepared following an analogous procedure to the one described for the synthesis of product 2 using intermediate 17 (100 mg, 0.47 mmol) and intermediate 86 as starting materials. Product 66 was purified by RP HPLC (stationary phase: C18 XBridge 30×100 mm 5 μm, mobile phase: gradient from 47% NH₄HCO₃ 0.25% solution in water, 53% CH₃CN to 30% NH₄HCO₃ 0.25% solution in water, 70% CH₃CN). The desired fractions were collected and concentrated in vacuo. The residue thus obtained was dissolved in EtOAc and washed with an aq sat sol of NaHCO₃. The organic phases were separated, dried (Na₂SO₄), filtered and concentrated in vacuo to yield product 66 (61 mg, 33%, mixture of diastereoisomers) as a colorless oil.

E45. Preparation of Product 67

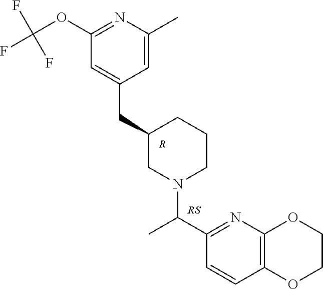

Product 67 was prepared following an analogous procedure to the one described for the synthesis of product 1 using intermediate 18 (100 mg, 0.46 mmol) and 2,3-dihydro[1,4]dioxino[2,3-b]pyridine-6-carbaldehyde (CAS: 615568-24-6) as starting materials. Product 67 was purified by RP HPLC (stationary phase: C18 XBridge 30×100 mm 5 μm, mobile phase: gradient from 54% NH₄HCO₃ 0.25% solution in water, 46% CH₃CN to 36% NH₄HCO₃ 0.25% solution in water, 64% CH₃CN). The desired fractions were collected and concentrated in vacuo. The residue thus obtained was dissolved in EtOAc and washed with an aq sat sol of NaHCO₃. The organic phases were separated, dried (Na₂SO₄), filtered and concentrated in vacuo to yield product 67 (45 mg, 28%, mixture of diastereoisomers) as a colorless oil.

E46. Preparation of Product 68

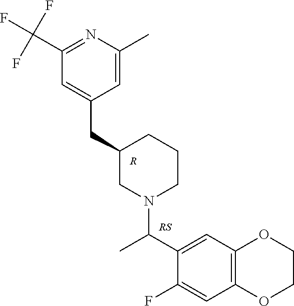

Product 68 was prepared following an analogous procedure to the one described for the synthesis of product 2 using intermediate 19 (100 mg, 0.39 mmol) and intermediate 86 as starting materials. Product 68 was purified by RP HPLC (stationary phase: C18 XBridge 30×100 mm 5 μm, mobile phase: gradient from 80% NH₄HCO₃ 0.25% solution in water, 20% CH₃CN to 60% NH₄HCO₃ 0.25% solution in water, 40% CH₃CN). The desired fractions were collected and concentrated in vacuo yielding product 68 (17 mg, 10%, mixture of diastereoisomers) as a colorless oil.

E47. Preparation of Product 69

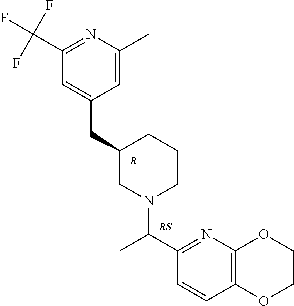

Product 69 was prepared following an analogous procedure to the one described for the synthesis of product 1 using intermediate 19 (100 mg, 0.46 mmol) and 2,3-dihydro[1,4]dioxino[2,3-b]pyridine-6-carbaldehyde (CAS: 615568-24-6) as starting materials. Product 69 was purified by RP HPLC (stationary phase: C18 XBridge 30×100 mm 5 μm, mobile phase: gradient from 60% NH₄HCO₃ 0.25% solution in water, 40% CH₃CN to 43% NH₄HCO₃ 0.25% solution in water, 57% CH₃CN). The desired fractions were collected and concentrated in vacuo yielding product 69 (10 mg, 6%, mixture of diastereoisomers) as a colorless oil.

E62. Preparation of Product 99

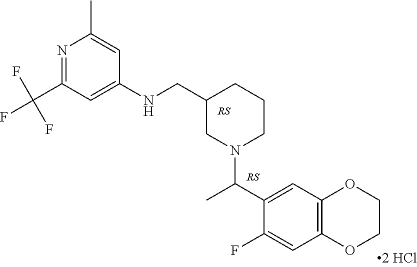

Product 99 was prepared following an analogous procedure to the one described for the synthesis of product 2 using intermediate 61 (110.3 mg, 0.404 mmol) and intermediate 86 (95 mg, 0.484 mmol) as starting materials. Product 99 was purified by phase reverse 49% [25 mM NH₄HCO₃]—51% [MeCN:MeOH (1:1)] to 6% [25 mM NH₄HCO₃]—94% [MeCN:MeOH (1:1)]. The desired fractions were collected and concentrated in vacuo at 60° C. ACN (10 mL×3 times) was added and the solvents were concentrated in vacuo to yield product 99 (60 mg, 32%, mixture of diastereoisomers) as a pale yellow foam. Product 99 (60 mg) was dissolved to DCM (2 mL) and HCl in 1,4-dioxane 4N (2 eq) was added, the solvents were concentrated in vacuo and the crude product was triturated with diethyl ether, the solid was filtered and dried to yield product 99 (59 mg, 27%, 2×HCl salt) as a white solid.

E63. Preparation of Product 110

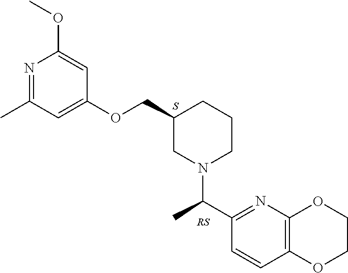

Intermediate 105 (117 mg, 0.5 mmol) and potassium carbonate (187 mg, 1.35 mmol) were added to a stirred solution of intermediate 107 (90 mg, 0.45 mmol) in acetonitrile (3.6 mL) at rt. The mixture was stirred at 80° C. overnight. Water was added and the mixture was extracted with DCM. The organic phase was collected, dried (Na₂SO₄), filtered and evaporated under vacuum. The crude product was purified by flash column chromatography (silica; 7N solution of ammonia in MeOH in DCM 0/100 to 10/90). The desired fractions were collected and concentrated in vacuo to yield product 110 (110 mg, 61%) as a pale yellow oil.

E64. Preparation of Product 111

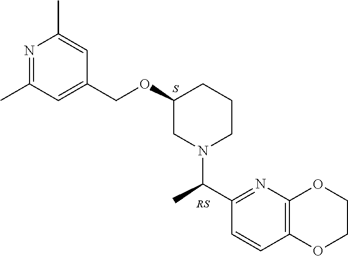

Product 111 was prepared following an analogous procedure to the one described for the synthesis of product 110 using intermediate 109 (97 mg, 0.44 mmol) and intermediate 107 (80 mg, 0.4 mmol) as starting materials.

E65. Preparation of Product 112

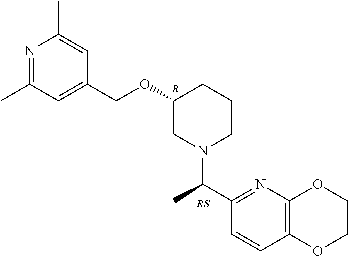

Product 112 was prepared following an analogous procedure to the one described for the synthesis of product 110 using intermediate 111 (97 mg, 0.44 mmol) and intermediate 107 (80 mg, 0.4 mmol) as starting materials.

E66. Preparation of Product 113

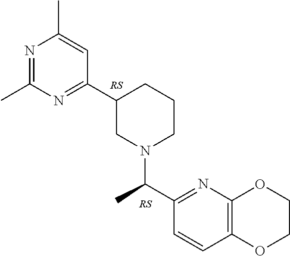

Product 113 was prepared following an analogous procedure to the one described for the synthesis of product 110 using intermediate 113 (100 mg, 0.52 mmol) and intermediate 107 (95 mg, 0.47 mmol) as starting materials. Product 113 was purified by RP HPLC (stationary phase: C18 XBridge 30×100 mm 5 μm, mobile phase: gradient from 80% NH₄HCO₃ 0.25% solution in water, 20% CH₃CN to 60% NH₄HCO₃ 0.25% solution in water, 40% CH₃CN). The desired fractions were collected and the organic solvents were evaporated in vacuo. To the resulting aqueous fraction EtOAc was added and the mixture was washed with a sat sol of NaHCO₃. The organic layer was separated, dried (Na₂SO₄), filtered and concentrated in vacuo to yield product 113 (142 mg, 84%) as a colorless oil.

E67. Preparation of Product 114

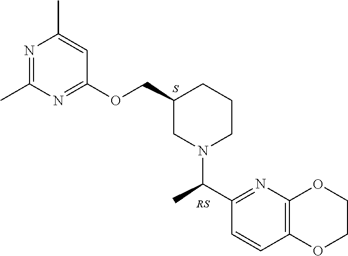

Product 114 was prepared following an analogous procedure to the one described for the synthesis of product 110 using intermediate 116 (150 mg, 0.68 mmol) and intermediate 107 (123 mg, 0.61 mmol) as starting materials.

E68. Preparation of Products 130, 131 and 132

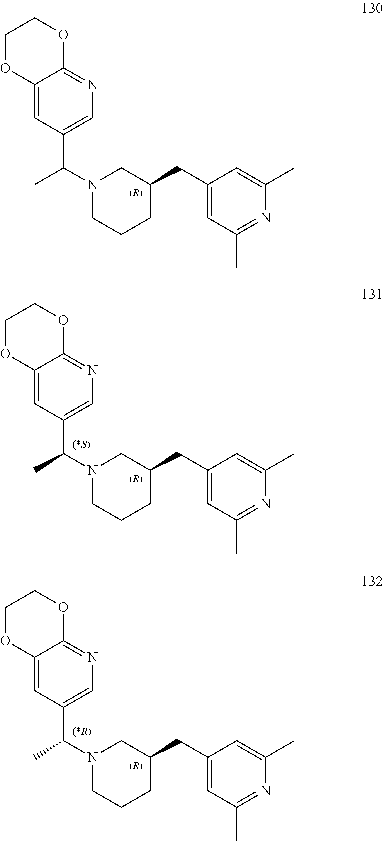

Ti(Oi-Pr)₄ (CAS: 546-68-9; 450 μL, 1.54 mmol) was added dropwise to a stirred solution of intermediate 16 (102 mg, 0.50 mmol) and 2,3-dihydro-[1,4]dioxino[2,3-b]pyridine-7-carbaldehyde (CAS: 95849-26-6; 105 mg, 0.64 mmol) in DCM (2.5 mL) in a sealed tube and under N₂ atmosphere. The reaction mixture was stirred at room temperature for 4 h. The reaction mixture was cooled to 0° C. and methylmagnesium bromide (1.4M in THF, 1.8 mL, 2.52 mmol) was added dropwise over 5 min. The reaction mixture was stirred at room temperature for 19 h. The mixture was treated with NH₄Cl (sat. solution) and DCM. The mixture was filtered through a pad of Celite® and washed with DCM. The filtrate was extracted with DCM. The combined organic layers were dried (MgSO₄), filtered and the solvents were evaporated in vacuo. The crude product was purified by flash column chromatography (SiO₂ amino functionalized, EtOAc in heptane, gradient from 0/100 to 100/0). The desired fractions were collected and concentrated in vacuo to afford product 130 (161 mg, 88%) as a colorless oil.

A purification was performed via chiral SFC (stationary phase: CHIRALPAK AD-H 5 μm 250*30 mm, mobile phase: 90% CO₂, 10% EtOH (0.3% i-PrNH₂)) to give product 131 (58 mg, 32%) and product 132 (51 mg, 28%) as pale yellow oils.

E69. Preparation of Product 133

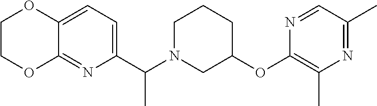 [SMILES: Cc1cnc(OC2CCCN(C(C)c3ccc4c(n3)OCCO4)C2)c(C)n1]

Intermediate 171 (99.2 mg, 0.55 mmol, 93% purity) and Ti(Oi-Pr)₄ (CAS: 546-68-9; 218 μL, 0.74 mmol) were added to a solution of intermediate 1340HCl (150 mg, 0.62 mmol) and DIPEA (212 μL, 1.23 mmol) in DCE (3 mL). The reaction mixture was stirred at 80° C. for 4 h, cooled to room temperature and sodium cyanoborohydride (CAS: 25895-60-7; 58.0 mg, 0.92 mmol) was added. The reaction mixture was stirred for 72 h, quenched with NaHCO₃ (sat. solution) and diluted with DCM. The emulsion was filtered through a pad of Celite®. The filtrate was extracted with DCM. The organic layer was dried (MgSO₄), filtered and the solvents were evaporated in vacuo. The crude product was purified by flash column chromatography (silica, MeOH in DCM, gradient from 0/100 to 05/95). The desired fractions were collected and concentrated in vacuo. A second purification was performed by RP HPLC (stationary phase: C18 XBridge 30×100 mm 5 μm), mobile phase: NH₄HCO₃ (0.25% solution in water)/CH₃CN, gradient from 80/20 to 0/100). The desired fractions were collected and concentrated in vacuo. The residue (45 mg) was purified by flash column chromatography (silica, MeOH in DCM, gradient from 0/100 to 5/95) to give product 133 (35 mg, 15%) as an oil.

E70. Preparation of Product 134

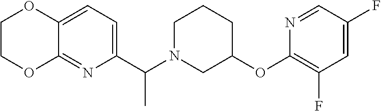 [SMILES: CC(c1ccc2c(n1)OCCO2)N1CCCC(Oc2ncc(F)cc2F)C1]

Intermediate 175 (173 mg, 1.03 mmol) and Ti(Oi-Pr)₄ (CAS: 546-68-9; 415 μL, 1.40 mmol) were added to a solution of intermediate 136 (200 mg, 0.93 mmol) in DCM (4 mL) and the reaction mixture was stirred at room temperature for 18 h. The reaction mixture was cooled to 0° C. and methylmagnesium bromide (1.4M, solution, 3.33 mL, 4.67 mmol) was added and the reaction mixture was stirred for 3 h. The reaction was quenched with MeOH and water, and diluted with DCM. The emulsion was filtered through a pad of Celite®. The filtrate was diluted with NH₄Cl (sat. solution) and extracted with DCM. The organic layer was dried (MgSO₄), filtered and the solvents were evaporated in vacuo. The crude product was purified by flash column chromatography (silica, MeOH in DCM, gradient from 0/100 to 5/95). The desired fractions were collected and concentrated in vacuo. A second purification was performed by RP HPLC (stationary phase: XBridge C18 50×100 mm, 5 μm), mobile phase: NH₄HCO₃ (0.25% solution in water)/CH₃CN, gradient from 80/20 to 0/100) to afford product 134 (259 mg, 74%).

E71. Preparation of Product 135

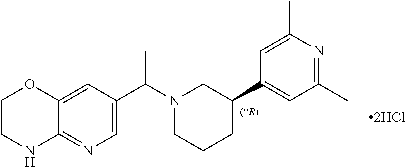 [SMILES: Cc1cc([C@H]2CCCN(C(C)c3cnc4c(c3)OCCN4)C2)cc(C)n1]

Product 135 was prepared following an analogous procedure to the one described for the synthesis of product 134 using intermediate 131 and intermediate 178 as starting materials.

The crude product was purified by flash column chromatography (silica, NH₃ (7M in MeOH)/DCM, gradient from 0/100 to 05/95). The desired fractions were collected and concentrated in vacuo. A second purification was performed by RP HPLC (stationary phase: C18 XBridge 30×100 mm 5 μm), mobile phase: NH₄HCO₃ (0.25% solution in water)/CH₃CN, gradient from 67/33 to 50/50). The desired fractions were collected and concentrated in vacuo. The residue (80 mg) was dissolved in tert-butyl methyl ether, and HCl (2M in Et₂O, 26 mL, 52 mmol) was added under stirring. The resulting precipitate was filtered and the compound was dried in the oven at 50° C. under vacuum to afford product 135 (85 mg, 13%).

E72. Preparation of Product 136

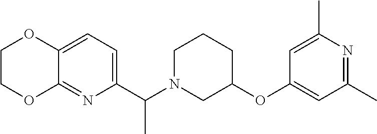 [SMILES: Cc1cc(OC2CCCN(C(C)c3ccc4c(n3)OCCO4)C2)cc(C)n1]

To a solution of intermediate 6 (100 mg, 0.49 mmol) in anhydrous DCM (1.98 mL) were added intermediate 175 (96.1 mg, 0.58 mmol) and Ti(Oi-Pr)₄ (CAS: 546-68-9; 0.21 mL, 0.73 mmol). The reaction mixture was stirred at room temperature for 20 h, cooled to 0° C. and methylmagnesium bromide (1.4M in THF, 1.73 mL, 2.42 mmol) was added dropwise. The reaction mixture was stirred at 0° C. for 5 min and at room temperature for 2 h. NH₄Cl (sat. solution) was added and the product was extracted with DCM. The organic layer was dried (MgSO₄), filtered and the solvents were evaporated in vacuo. The crude product was purified by flash column chromatography (silica, NH₃ (7M in MeOH) in DCM, gradient from 0/100 to 30/70). The residue was purified by RP HPLC (stationary phase: C18 XBridge 30×100 mm 5 μm), mobile phase: NH₄HCO₃ (0.25% solution in water)/CH₃CN, gradient from 90/10 to 65/35) to give product 136 (40.6 mg, 23%) as a yellow oil.

E73. Preparation of Product 137

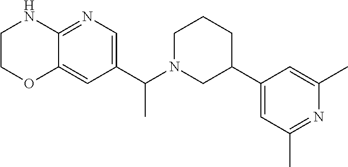 [SMILES: Cc1cc(C2CCCN(C(C)c3cnc4c(c3)OCCN4)C2)cc(C)n1]

Product 137 was prepared following an analogous procedure to the one described for the synthesis of product 136 using intermediate 1 and intermediate 178 as starting materials.

The crude product was purified by flash column chromatography (silica, NH₃ (7M in MeOH) in DCM, gradient from 0/100 to 10/90). The desired fractions were collected and evaporated in vacuo to give compound 137 (60.4 mg, 33%) as a yellow oil.

E74. Preparation of Product 138

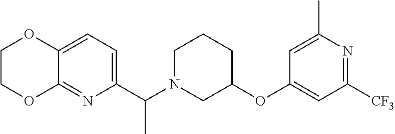 [SMILES: Cc1cc(OC2CCCN(C(C)c3ccc4c(n3)OCCO4)C2)cc(C(F)(F)F)n1]

Intermediate 175 (66.6 mg, 0.40 mmol) and Ti(Oi-Pr)₄ (CAS: 546-68-9; 0.17 mL, 0.58 mmol) were added to a solution of intermediate 7 (100 mg, 0.38 mmol) and the reaction mixture was stirred at room temperature for 16 h. The reaction mixture was cooled to 0° C. and methylmagnesium bromide (1.4M solution, 1.37 mL, 1.92 mmol) was added dropwise. The reaction mixture was stirred at room temperature for 2 h. NH₄Cl (sat. solution) was added and the mixture was extracted with DCM. The organic layer was dried (MgSO₄), filtered and the solvents were evaporated in vacuo. The crude product was purified by flash column chromatography (silica, NH₃ (7M in MeOH) in DCM, gradient from 0/100 to 10/90) The desired fractions were collected and concentrated in vacuo. The residue was purified by RP HPLC (stationary phase: C18 XBridge 30×100 mm 5 μm), mobile phase: NH₄HCO₃ (0.25% solution in water)/CH₃CN, gradient from 54/46 to 36/64). The residue was dissolved in EtOAc and washed with NaHCO₃ (sat. solution). The organic layer was dried (Na₂SO₄), filtered and concentrated in vacuo to give product 138 (90.3 mg, 56%) as a colorless oil.

E75. Preparation of Products 139 and 140

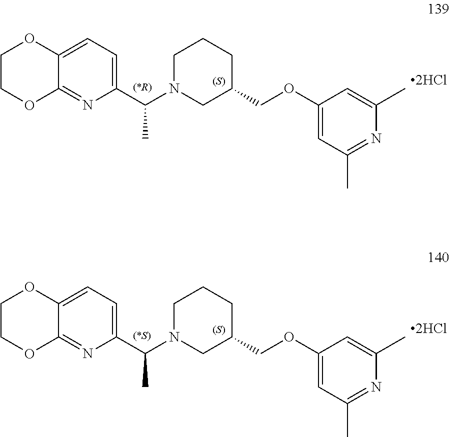

Products 139 and 140 were prepared following an analogous procedure to the one described for the synthesis of product 138 using intermediate 9 and intermediate 175 as starting materials.

The crude product was purified by flash column chromatography (silica, NH₃ (7M in MeOH) in DCM, gradient from 0/100 to 10/90). A purification was performed via chiral SFC (stationary phase: CHIRACEL OJ-H 5 μm 250*30 mm, mobile phase: 70% CO₂, 30% MeOH (0.3% i-PrNH₂)) to afford fraction A (104 mg) and fraction B (97 mg). Fraction A was further purified by reverse phase (stationary phase: YMC-actus Triart C18 10 μm 30*150 mm, mobile phase: NH₄HCO₃ (0.2% in water)/CH₃CN, gradient from 65/35 to 35/65) to give fraction A (61 mg).

Fraction A (61 mg) was dried under vacuum at 50° C. for 16 h and Et₂O (0.2 mL) was added followed by 6N HCl-IPA (0.2 mL). The mixture was stirred at room temperature for 16 h and the solvent was concentrated in vacuo. tert-Butyl methyl ether was added and the mixture was sonicated for 10 min. The solvent was concentrated in vacuo. The process was repeated until the obtention of a solid which was dried under vacuum to afford product 139 (55.3 mg, 13%).

Product 140 (97 mg, 23%) was obtained following an analogous procedure starting from fraction B.

E76. Preparation of Products 141, 142, 143 and 144

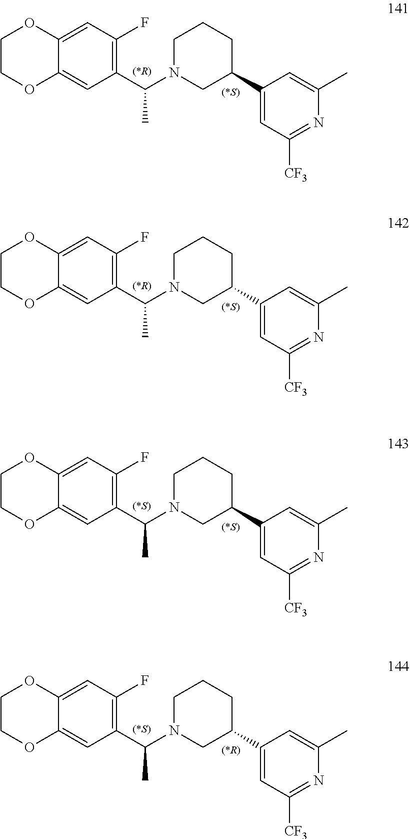

Ti(Oi-Pr)₄ (CAS: 546-68-9; 0.30 mL, 1.02 mmol) was added to a mixture of intermediate 3 (250 mg, 1.02 mmol) and intermediate 86 (201 mg, 1.02 mmol) in DCE (4.15 mL) at room temperature. The reaction mixture was stirred at 80° C. for 16 h in a sealed tube, cooled down and sodium cyanoborohydride (CAS: 25895-60-7; 77.2 mg, 1.23 mmol) was added. The reaction mixture was stirred for 1 h and treated with a NaHCO₃ (sat. solution). the mixture was diluted with DCM and filtered through Celite®. The organic layer dried (Na₂SO₄), filtered and the solvent was evaporated in vacuo. The crude product was purified by flash column chromatography (silica, MeOH in EtOAc, gradient from 0/100 to 10/90). The desired fractions were collected and evaporated in vacuo. a second purification was performed by RP HPLC (stationary phase: C18 XBridge 30×100 mm 5 μm), mobile phase: NH₄HCO₃ (0.25% solution in water)/CH₃CN, gradient from 47/53 to 30/70) to afford a mixture of products (210 mg, 48%) as a colorless oil.

A purification was performed via chiral SFC (stationary phase: CHIRALPAK AD-H 5 μm 250*30 mm, mobile phase: 96% CO₂, 4% i-PrOH (0.3% i-PrNH₂)) to give product 144 (33 mg, 8%) and product 141 (31 mg, 7%) as well as a mixture (74 mg). The mixture was purified via chiral SFC (stationary phase: Chiralcel OD-H 5 μm 250×21.2 mm, mobile phase: 94% CO₂, 6% i-PrOH (0.3% i-PrNH₂)) to deliver product 142 (34 mg, 8%) and product 143 (37 mg, 9%). Product 144 was re-purified by RP HPLC (stationary phase: C18 XBridge 30×100 mm 5 μm), mobile phase: NH₄HCO₃ (0.25% solution in water)/CH₃CN, gradient from 47/53 to 30/70) to deliver product 144 (28 mg, 6%) as a colorless oil.

E77. Preparation of Product 145

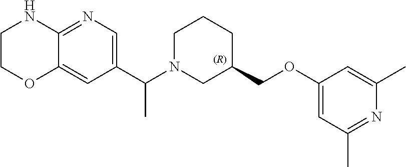

A solution of lithium hydroxide (26.6 mg, 1.11 mmol) in H₂O (1.7 mL) was added to a stirred solution of intermediate 181 (149 mg, 0.32 mmol) in 1,4-dioxane (1.7 mL) in a sealed tube. The reaction mixture was stirred at 80° C. for 16 h. The mixture was diluted with water and extracted with DCM. The organic layer was dried (MgSO₄), filtered and the solvents were evaporated in vacuo. The crude product was purified by flash column chromatography (silica, NH₃ (7M in MeOH) in DCM, gradient from 0/100 to 30/70). A second purification was performed by RP HPLC (stationary phase: C18 XBridge 30×100 mm 5 μm), mobile phase: NH₄HCO₃ (0.25% solution in water)/CH₃CN, gradient from 75/25 to 57/43). The residue was diluted with NaHCO₃ (sat. solution) and extracted with DCM. The organic layer was dried (MgSO₄), filtered and the solvents were evaporated in vacuo to afford product 145 (35.4 mg, 29%) as a colorless oil.

E78. Preparation of Product 146

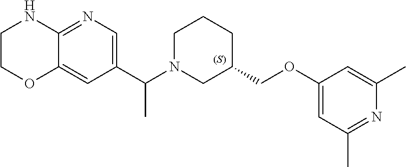

Product 146 was prepared following an analogous procedure to the one described for the synthesis of product 145 using intermediate 182 as starting material.

The residue was purified by RP HPLC (stationary phase: C18 XBridge 30×100 mm 5 μm), mobile phase: NH₄HCO₃ (0.25% solution in water)/CH₃CN, gradient from 75/25 to 57/43). The residue was diluted with NaHCO₃ (sat. solution) and extracted with DCM. The organic layer was dried (Na₂SO₄), filtered and the solvents were evaporated in vacuo to yield product 146 (41.5 mg, 32%) as a colorless oil.

E79. Preparation of Product 147

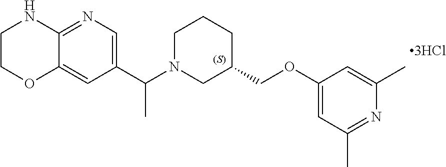

Product 146 (82.3 mg, 0.22 mmol) was dissolved in Et₂O (3 mL) at room temperature and HCl (1M in Et₂O, 2.37 mL, 2.37 mmol) was added dropwise. The reaction mixture was stirred for 30 min. The white precipitate was filtered, washed with Et₂O and dried under the vacuum at 50° C. to yield product 147 (104 mg, 62%) s a white solid.

E80. Preparation of Product 148

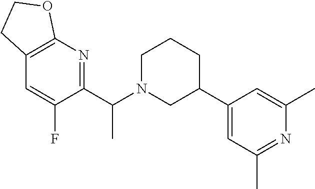 [SMILES: Cc1cc(C2CCCN(C(C)c3nc4c(cc3F)CCO4)C2)cc(C)n1]

Intermediate 97 (30.0 mg, 0.17 mmol) and Ti(Oi-Pr)₄ (CAS: 546-68-9; 60.6 μL, 0.21 mmol) were added to a solution of intermediate 1 (26.3 mg, 0.14 mmol) in THF (0.73 mL) and the reaction mixture was stirred at 80° C. for 12 h. The reaction mixture was cooled to room temperature and sodium cyanoborohydride (CAS: 25895-60-7; 12.1 mg, 0.19 mmol) was added and the mixture was stirred for 2 h. NaHCO₃ (sat. solution) was added and the mixture was extracted with DCM. The organic layer was dried (MgSO₄), filtered and the solvents were evaporated in vacuo. The crude product was purified by flash column chromatography (silica, NH₃ (7M in MeOH) in DCM, gradient from 0/100 to 30/70). A second purification was performed by RP HPLC (stationary phase: C18 XBridge 30×100 mm 5 μm), mobile phase: NH₄HCO₃ (0.25% solution in water)/CH₃CN, gradient from 67/33 to 50/50). The desired fractions were collected and evaporated in vacuo to give product 148 (10 mg, 20%) as a colorless oil.

E81. Preparation of Product 149

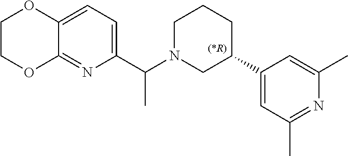 [SMILES: Cc1cc([C@H]2CCCN(C(C)c3ccc4c(n3)OCCO4)C2)cc(C)n1]

Intermediate 131 (41.9 mg, 0.22 mmol) and K₂CO₃ (83.1 mg, 0.60 mmol) were added to a stirred solution of intermediate 107 (40.0 mg, 0.20 mmol) in CH₃CN (1.6 mL). The reaction mixture was stirred overnight at 80° C. Water was added and the mixture was extracted with DCM. The organic was dried (Na₂SO₄), filtered and evaporated in vacuo. The crude product was purified by flash column chromatography (silica, NH₃ (7N in MeOH) in DCM, gradient from 0/100 to 10/90). The desired fractions were collected and concentrated in vacuo to afford product 149 (55 mg, 78%) as a light yellow oil.

E82. Preparation of Product 150

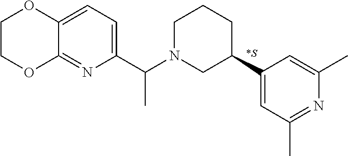 [SMILES: Cc1cc([C@@H]2CCCN(C(C)c3ccc4c(n3)OCCO4)C2)cc(C)n1]

Product 150 was prepared following an analogous procedure to the one described for the synthesis of product 149 using intermediate 107 and intermediate 132 as starting materials.

The crude product was purified by flash column chromatography (silica, NH₃ (7N in MeOH) in DCM, gradient from 0/100 to 10/90). The desired fractions were collected and concentrated in vacuo to yield product 150 (42 mg, 59%) as a light yellow oil.

E83. Preparation of Product 151

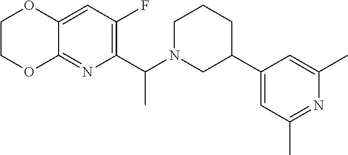 [SMILES: Cc1cc(C2CCCN(C(C)c3nc4c(cc3F)OCCO4)C2)cc(C)n1]

Product 151 was prepared following an analogous procedure to the one described for the synthesis of product 149 using intermediate 129 and intermediate 1 as starting materials.

The crude product was purified by flash column chromatography (silica, NH₃ (7N in MeOH) in DCM, gradient from 0/100 to 10/90). The desired fractions were collected and concentrated in vacuo to yield product 151 (40 mg, 47%) as a light yellow oil.

E84. Preparation of Products 152, 153 and 154

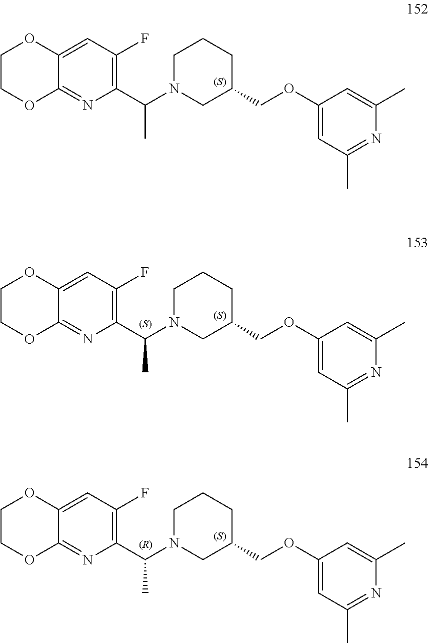

DIPEA (70.5 mL, 409 mmol) was added dropwise to a suspension of intermediate 9.2HCl (20.0 g, 68.2 mmol) in CH₃CN (200 mL) under N₂ atmosphere. The suspension became a clear solution and a solution of intermediate 129 (15.6 g, 71.6 mmol) in CH₃CN (40 mL) was added dropwise. The reaction mixture was stirred at 80° C. for 24 h. The solvent was evaporated in vacuo. The residue was diluted with EtOAc and Na₂CO₃ (sat. solution) was added. The aqueous layer was separated and discarded.

The organic phase was treated with HCl (1N). The 2 phases were separated and the organic layer was discarded. The aqueous layer was basified by the addition of Na₂CO₃ (sat. solution). The organic layer was dried (Na₂SO₄), filtered and evaporated in vacuo.

The crude product was purified by flash column chromatography (silica, MeOH in DCM, gradient from 0/100 to 8/92). The desired fractions were collected and concentrated in vacuo to afford product 152 (18.57 g, 68%).

A purification was performed via chiral SFC (stationary phase: CHIRALPAK AD-H 5 μm 250*30 mm, mobile phase: 85% CO₂, 15% i-PrOH (0.3% i-PrNH₂)) to give product 153 (9.45 g, 35%) and product 154 (8.51 g, 31%). Product 153 was further purified via preparative LC (stationary phase: irregular SiOH 40 μm 120 g, mobile phase: 0.3% NH₄OH, 97% DCM, 3% MeOH) to give product 153 (7.2 g, 26%).

E85. Preparation of Product 155

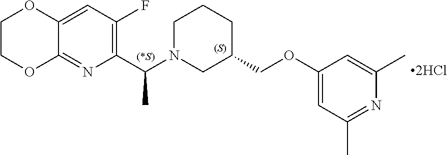

HCl (2N in Et₂O, 35.9 mL, 71.8 mmol) was added to a solution of product 153 (7.20 g, 17.9 mmol) in Et₂O (80 mL). The reaction mixture was stirred at room temperature for 1 h. The solid was filtered off, washed with Et₂O and dried for 5 days at room temperature and at 50° C. for 24 h to give product 155 (7.05 g, 83%) as a white solid.

E86. Preparation of Product 156

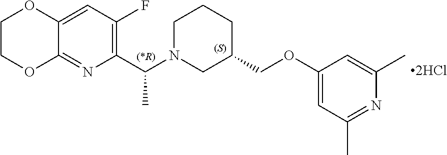

Product 156 was prepared following an analogous procedure to the one described for the synthesis of product 155 using product 154 as starting material.

E87. Preparation of Product 157

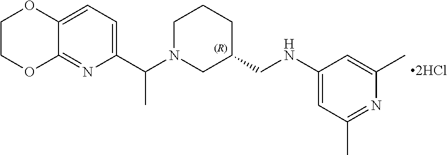

DIPEA (0.11 mL, 0.65 mmol) was added to a mixture of 4-chloro-2,6-dimethylpyrimidine (CAS: 4472-45-1; 67.9 mg, 0.48 mmol) and intermediate 13 (120 mg, 0.43 mmol) in butanol (7.89 mL). The reaction mixture was stirred at 100° C. for 20 h. The solvent was removed. The residue was dissolved in DCM and NaHCO₃ (sat. solution) was added. The layers were separated and the aqueous phase extracted with DCM. The combined organic layers were dried (MgSO₄), filtered and the solvent was evaporated in vacuo. The crude product was purified by flash chromatography (SiO₂, EtOAc in heptane, gradient from 0/100 to 50/50). The desired fractions were collected and concentrated in vacuo to yield as a red oil (93 mg).

The material was taken into DCM and treated with HCl (4N in 1,4-dioxane, 85 μL). The solvents were evaporated in vacuo and the product was triturated with Et₂O to afford product 157 (81 mg, 44%) as a pale red solid.

E88. Preparation of Products 158 and 159

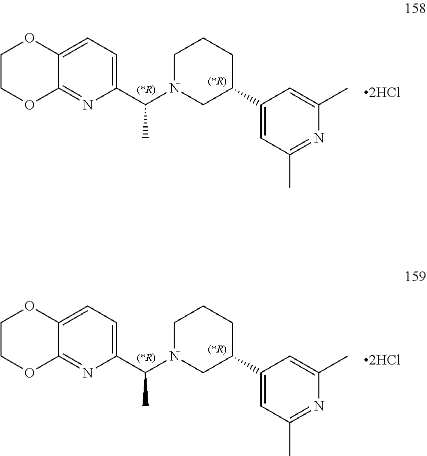 [SMILES: Cc1cc([C@H]2CCCN([C@H](C)c3ccc4c(n3)OCCO4)C2)cc(C)n1 ; "·2HCl" ; Cc1cc([C@H]2CCCN([C@@H](C)c3ccc4c(n3)OCCO4)C2)cc(C)n1 ; "·2HCl"]

Intermediate 107 (329 mg, 1.65 mmol) was dissolved in CH₃CN (13.2 mL) and intermediate 131 (345 mg, 1.81 mmol) followed by K₂CO₃ (683 mg, 4.95 mmol) were added. The reaction mixture was stirred overnight at 80° C. Water was added and the mixture was extracted with DCM. The combined organic layers were dried (Na₂SO₄), filtered and evaporated in vacuo. The crude product was purified by flash column chromatography (silica, NH₃ (7N in MeOH) in DCM, gradient from 0/100 to 10/90). The purification was repeated 3 times. The desired fractions were collected and concentrated in vacuo. A purification was performed via chiral SFC (stationary phase: CHIRACEL OJ-H 5 μm 250*20 mm, mobile phase: 85% CO₂, 15% MeOH (0.3% i-PrNH₂)) to afford fraction A (170 mg) and fraction B (173 mg).

Fraction A (170 mg) and B (173 mg) were separately diluted with Et₂O (0.2 mL) and 6N HCl-IPA (0.2 mL) was added. The mixture was stirred at room temperature for 16 h. The solvent was evaporated in vacuo. tert-Butyl methyl ether was added and the mixtures were sonicated for 10 min. The solvent was evaporated in vacuo. The process was repeated until the obtention of a solid which was dried under vacuum for 6 days to afford product 158 (160 mg, 23%) and product 159 (163 mg, 23%).

E89. Preparation of Products 160 and 161

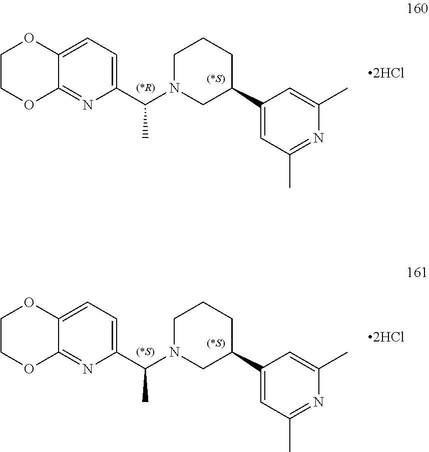 [SMILES: Cc1cc([C@@H]2CCCN([C@H](C)c3ccc4c(n3)OCCO4)C2)cc(C)n1 ; Cc1cc([C@@H]2CCCN([C@@H](C)c3ccc4c(n3)OCCO4)C2)cc(C)n1]

Products 160 and 161 were prepared following an analogous procedure to the one descried for the synthesis of products 158 and 159 using intermediate 107 and intermediate 132 as starting materials.

The crude product was purified by flash column chromatography (silica, NH₃ (7N in MeOH) in DCM, gradient from 0/100 to 10/90). The purification was repeated 3 times. The desired fractions were collected and concentrated in vacuo. A purification was performed via chiral SFC (stationary phase: Chiralpak IC 5 μm 250*21.2 mm, mobile phase: 65% CO₂, 35% i-PrOH (0.3% i-PrNH₂)) to afford fraction A (152 mg) and fraction B (162 mg). Fraction B was re-purified via chiral SFC (stationary phase: Chiralpak IC 5 μm 250*21.2 mm, mobile phase: 65% CO₂, 35% i-PrOH (0.3% i-PrNH₂)) to give 160 mg.

Fractions A (152 mg) and B (160 mg) were diluted with Et₂O (0.2 mL) and 6N HCl-IPA (0.2 mL) was added. The mixtures were stirred at room temperature for 16 h. The solvents were evaporated in vacuo. tert-Butyl methyl ether was added and the mixture were sonicated for 10 min. The solvents were evaporated in vacuo. The process was repeated until the obtention of solids which were dried under vacuum to afford product 160 (197 mg, 33%) and product 161 (177 mg, 29%) as light yellow solids.

E90. Preparation of Products 162 and 163

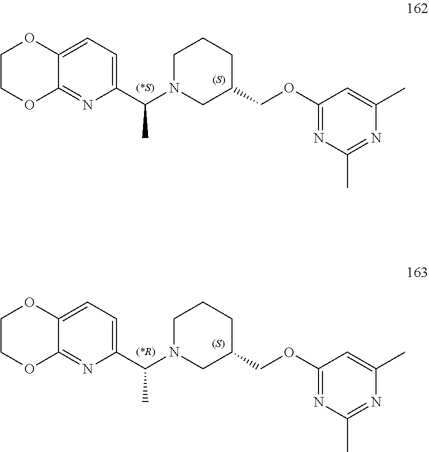

Products 162 and 163 were prepared following an analogous procedure to the one described for the synthesis of products 158 and 159 using intermediate 107 and intermediate 116 as starting materials.

The crude product was purified by flash column chromatography (silica, NH₃ (7N in MeOH) in DCM, gradient from 0/100 to 10/90). The purification was repeated 3 times. The desired fractions were collected and concentrated in vacuo. A purification was performed via chiral SFC (stationary phase: Chiralpak IC 5 μm 250*21.2 mm, mobile phase: 70% CO₂, 30% i-PrOH (0.3% i-PrNH₂)) to afford product 163 (70 mg, 30%) and product 162 (70 mg, 30%). A second purification was performed on product 162 via achiral SFC (stationary phase: CHIRACEL OJ-H 5 μm 250*20 mm, mobile phase: 85% CO₂, 15% MeOH (0.3% i-PrNH₂)) to afford product 162 (60 mg, 25%). Product 163 (70 mg) was diluted with Et₂O (0.1 mL) and 6N HCl-IPA (0.1 mL) was added. The mixture was stirred at room temperature for 16 h. The solvent was evaporated in vacuo. tert-Butyl methyl ether was added and the mixture was sonicated for 10 min. The solvent was evaporated in vacuo. The process was repeated until the obtention of a solid which was dried under vacuum. The residue was purified by RP HPLC (stationary phase: C18 XBridge 30×100 mm 5 μm), mobile phase: NH₄HCO₃ (0.25% solution in water)/CH₃CN, gradient from 80/20 to 0/100). The desired fractions were collected and concentrated in vacuo. The residue was dissolved in EtOAc and washed with NaHCO₃ (sat. solution). The organic phase was dried (Na₂SO₄), filtered and concentrated in vacuo to yield product 163 (14 mg, 6%).

The same treatment was applied to product 162 (60 mg) to afford product 162 (22 mg, 9%).

E91. Preparation of Products 164 and 165

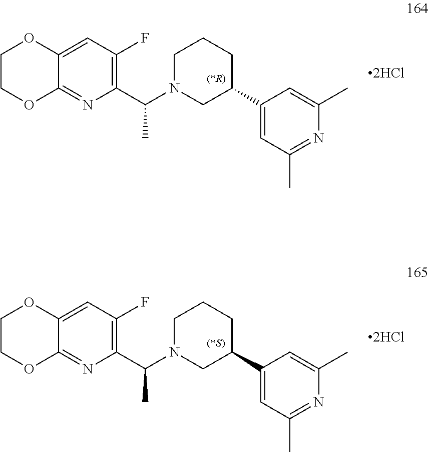 [SMILES: Cc1cc([C@H]2CCCN([C@H](C)c3nc4c(cc3F)OCCO4)C2)cc(C)n1 ; Cc1cc([C@@H]2CCCN([C@@H](C)c3nc4c(cc3F)OCCO4)C2)cc(C)n1]

Products 164 and 165 were prepared following an analogous procedure to the one described for the synthesis of products 158 and 159 using intermediate 129 and intermediate 1 as starting materials.

The crude product was purified by flash column chromatography (silica, NH₃ (7N in MeOH) in DCM, gradient from 0/100 to 10/90). The desired fractions were collected and concentrated in vacuo. A second purification was performed by RP HPLC (stationary phase: C18 XBridge 30×100 mm 5 μm), mobile phase: NH₄HCO₃ (0.25% solution in water)/CH₃CN, gradient from 80/20 to 60/40). The desired fractions were collected and concentrated in vacuo. The residue was dissolved in EtOAc, washed with NaHCO₃ (sat. solution), dried (Na₂SO₄), filtered and concentrated in vacuo to afford fraction A (57.1 mg) and fraction B (43.4 mg) as colorless oils.

Fractions A (57.1 mg) and B (43.4 mg) were dissolved in Et₂O (0.1 mL) then 7N HCl-IPA (0.1 mL) was added. The mixtures were stirred at room temperature for 16 h. The solvent was concentrated in vacuo. tert-Butyl methyl ether was added and the mixtures were sonicated for 10 min. The solvents were evaporated in vacuo. The process was repeated until the obtention of solids which were dried under vacuum to yield product 164 (49 mg, 30%) and product 165 (68 mg, 42%).

E92. Preparation of Products 166 and 167

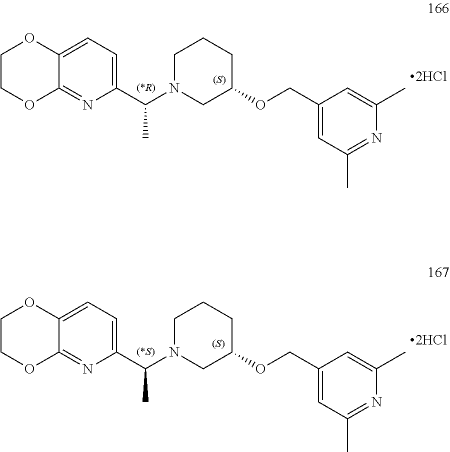

Products 166 and 167 were prepared following an analogous procedure to the one described for the synthesis of products 158 and 159 using intermediate 107 and intermediate 109 as starting materials.

The crude product was purified by flash column chromatography (silica, NH₃ (7N in MeOH) in DCM, gradient from 0/100 to 10/90) (twice). The desired fractions were collected and concentrated in vacuo. A purification was performed via chiral SFC (stationary phase: CHIRACEL OJ-H 5 μm 250*20 mm, mobile phase: 85% CO₂, 15% MeOH (0.3% i-PrNH₂)) to afford fraction A (30 mg) and fraction B (26 mg).

Fractions A (30 mg) and B (26 mg) were dissolved in Et₂O (0.1 mL) and 7N HCl-IPA (0.1 mL) was added. The mixtures were stirred at room temperature for 16 h. The solvents were evaporated in vacuo. tert-Butyl methyl ether was added and the mixtures were sonicated for 10 min. The solvents were evaporated in vacuo. The process was repeated until the obtention of solids, which were dried under vacuum at 50° C. for 72 h to afford product 166 (26 mg, 14%) and product 167 (28 mg, 15%) as light brown solids.

E93. Preparation of Products 168, 169 and 170

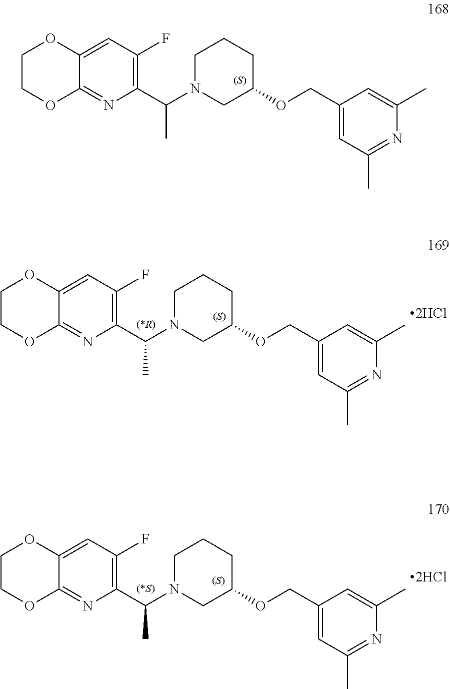

Products 168, 169 and 170 were prepared following an analogous procedure to the one described for the synthesis of products 158 and 159 using intermediate 129 and intermediate 109 as starting materials.

The crude product was purified by flash column chromatography (silica, NH₃ (7N in MeOH) in DCM, gradient from 0/100 to 10/90) (twice). The desired fractions were collected and concentrated in vacuo. The residue was purified by RP HPLC (stationary phase: C18 XBridge 30×100 mm 5 μm), mobile phase: NH₄HCO₃ (0.25% solution in water)/CH₃CN, gradient from 80/20 to 0/100). The desired fractions were collected and concentrated in vacuo. The residue was dissolved in EtOAc, washed with NaHCO₃ (sat. solution), dried (Na₂SO₄), filtered and concentrated in vacuo to give product 168 (88.4 mg, 37%) as a yellow oil.

A purification was performed via chiral SFC (stationary phase: CHIRALPAK AD-H 5 μm 250*30 mm, mobile phase: 84% CO₂, 16% i-PrOH (0.3% i-PrNH₂)) to give fraction A (37 mg) and fraction B (38 mg).

Fractions A (37 mg) and B (38 mg) were dissolved in Et₂O (0.2 mL) and 7N HCl-IPA (0.2 mL) was added. The mixtures were stirred at room temperature for 16 h. The solvents were evaporated in vacuo. tert-Butyl methyl ether was added and the mixtures were sonicated for 10 min. The solvents were evaporated in vacuo. The process was repeated until the obtention of solids which were dried under vacuum at 50° C. for 5 to give product 169 (41.9 mg, 15%) and product 170 (43.6 mg, 16%).

E94. Preparation of Product 171

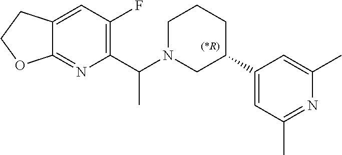 [SMILES: Cc1cc([C@H]2CCCN(C(C)c3nc4c(cc3F)CCO4)C2)cc(C)n1]

K₂CO₃ (283 mg, 2.05 mmol) was added to a mixture of intermediate 180 (138 mg, 0.68 mmol) and intermediate 131 (130 mg, 0.68 mmol) in CH₃CN (4 mL). The reaction mixture was stirred for 48 h at 70° C. The reaction mixture was diluted with EtOAc, filtered through Celite®, washed with EtOAc and the filtrate was evaporated in vacuo. The crude product was purified by flash column chromatography (silica, NH₃ (7N in MeOH) in DCM, gradient from 0/100 to 04/96). The desired fractions were collected and concentrated in vacuo to afford product 171 (170 mg, 70%) as an oil.

E95. Preparation of Product 172

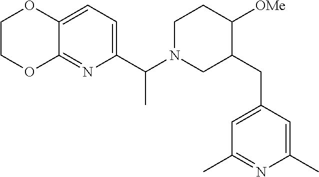 [SMILES: COC1CCN(C(C)c2ccc3c(n2)OCCO3)CC1Cc1cc(C)nc(C)c1]

Product 172 was prepared following an analogous procedure to the one described for the synthesis of product 171 using intermediate 142 and intermediate 107 as starting materials.

The crude product was purified by flash column chromatography (silica, NH₃ (7N in MeOH) in DCM, gradient from 0/100 to 05/95). The desired fractions were collected and concentrated in vacuo to yield product 172 (200 mg, 82%) as an oil.

E96. Preparation of Product 173

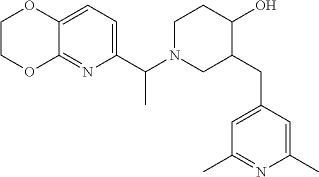 [SMILES: Cc1cc(CC2CN(C(C)c3ccc4c(n3)OCCO4)CCC2O)cc(C)n1]

Product 173 was prepared following an analogous procedure to the one described for the synthesis of product 171 using intermediate 144 and intermediate 107 as starting materials.

The crude product was purified by flash column chromatography (silica, NH₃ (7N in MeOH) in DCM, gradient from 0/100 to 04/96). The desired fractions were collected and concentrated in vacuo to yield product 173 (200 mg, 96%) as an oil.

E97. Preparation of Products 174, 175 and 174.2 HCl, 175.2 HCl

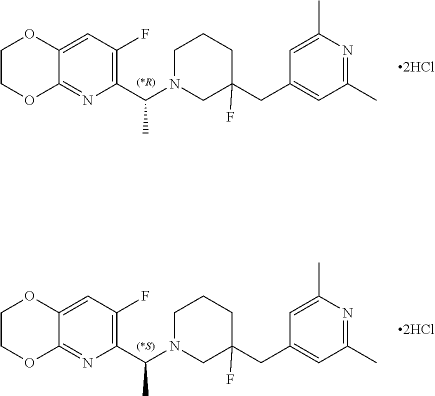 [SMILES: Cc1cc(CC2(F)CCCN([C@H](C)c3nc4c(cc3F)OCCO4)C2)cc(C)n1 ; Cc1cc(CC2(F)CCCN([C@@H](C)c3nc4c(cc3F)OCCO4)C2)cc(C)n1]

Intermediate 162 (204 mg, 0.92 mmol) and K₂CO₃ (381 mg, 2.76 mmol) were added to a stirred solution of intermediate 129 (200 mg, 0.92 mmol) in CH₃CN (8 mL). The reaction mixture was stirred for 36 h at 75° C., treated with water and extracted with EtOAc. The organic layer was dried (Na₂SO₄), filtered and evaporated in vacuo. The crude product was purified by flash column chromatography (silica, MeOH in DCM, gradient from 0/100 to 10/90). The desired fractions were collected and concentrated in vacuo to afford a racemic mixture (165 mg, 44%).

The mixture was purified by RP HPLC (stationary phase: C18 XBridge 30×100 mm 5 μm), mobile phase: [0.1% NH₄CO₃H/NH₄OH pH 9 solution in water]/CH₃CN, gradient from 67/33 to 50/50). The desired fractions were collected and concentrated in vacuo to give product 174 (78 mg, 21%) and product 175 (73 mg, 20%) as oils.

For the Formation of the Hydrochloride Salts:

HCl (6M in i-PrOH, 0.5 mL, 3 mmol) was added to product 174 (78 mg, 0.19 mmol).

The reaction mixture was stirred for 1 h at room temperature. The solvent was evaporated in vacuo. The crude mixture was treated with DIPE and stirred for 2 h. The solid was filtered to give product 174.2HCl (72 mg, 78%) as a white solid.

Product 175.2 HCl was prepared following an analogous procedure.

E.98 Preparation of Products 176, 177 and 176.2 HCl and 177.2 HCl

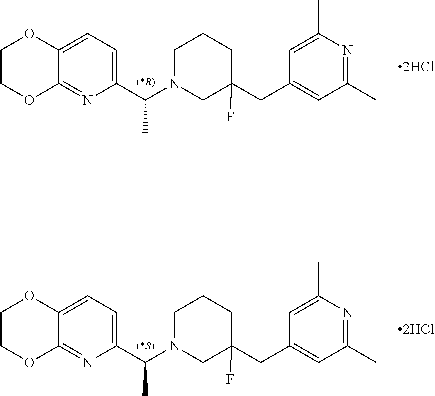 [SMILES: Cc1cc(CC2(F)CCCN([C@H](C)c3ccc4c(n3)OCCO4)C2)cc(C)n1 ; Cc1cc(CC2(F)CCCN([C@@H](C)c3ccc4c(n3)OCCO4)C2)cc(C)n1]

Products 176 and 177 were prepared following an analogous procedure to the one described for the synthesis of products 174 and 175 using intermediate 107 and intermediate 162 as starting materials.

The crude product was purified by flash column chromatography (silica, MeOH in DCM, gradient from 0/100 to 10/90). The desired fractions were collected and concentrated in vacuo to afford a racemic mixture (130 mg, 36%). The mixture was purified by RP HPLC (stationary phase: C18 XBridge 30×100 mm 5 um), mobile phase: [0.1% NH₄CO₃H/NH₄OH pH 9 solution in water]/CH₃CN, gradient from 67/33 to 50/50), The desired fractions were collected and concentrated in vacuo to give product 176 (67 mg, 18%) and product 177 (54 mg, 15%) as oils.

For the formation of the hydrochloride salts:

HCl (6M in Et₂O, 0.5 mL, 3.0 mmol) was added to a solution of product 176 (62 mg, 0.16 mmol) in Et₂O (2 mL). The reaction mixture was stirred for 2 h at room temperature and the solvent was evaporated in vacuo. The residue was washed with Et₂O (several times), filtered and dry under vacuum to afford product 176.2 HCl (44.6 mg, 60%) as a white solid.

Product 177.2 HCl was prepared following an analogous procedure.

E99. Preparation of Product 178, 179 and 180

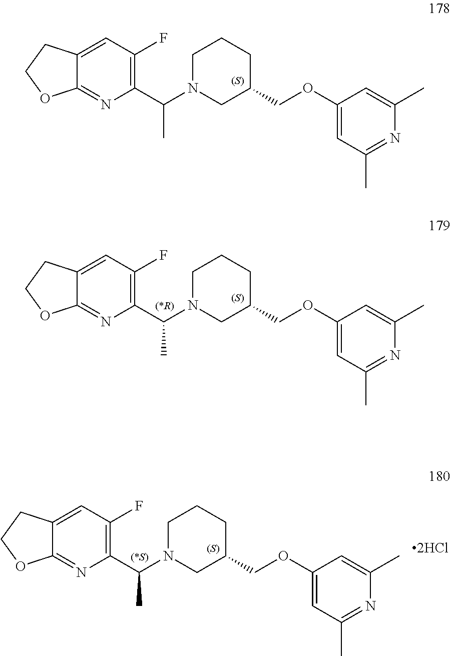

K₂CO₃ (753 mg, 5.45 mmol) was added to a mixture of intermediate 9 (400 mg, 1.82 mmol) and intermediate 180 (366 mg, 1.82 mmol) in CH₃CN (25 mL). The reaction mixture was stirred at 70° C. for 36 h. The reaction mixture was diluted with water and extracted with EtOAc. The organic layer was dried (Na₂SO₄), filtered and the solvents were evaporated in vacuo. The crude product was purified by flash column chromatography (silica, MeOH in DCM, gradient from 0/100 to 5/95). The desired fractions were collected and the solvents were evaporated in vacuo. The residue was triturated with DIPE and the solid was filtered to afford product 178 (370 mg, 50%) as cream solid.

Product 178 was purified via chiral SFC (stationary phase: CHIRALPAK AD-H 5 μm 250*30 mm, mobile phase: 88% CO₂, 12% i-PrOH (0.3% i-PrNH₂)) to give fraction A (136 mg) and fraction B (166 mg).

DIPE (3 mL) was added to fraction A (136 mg, 0.35 mmol). The mixture was stirred for 24 h at room temperature. The crude mixture was filtered to afford product 179 (110 mg. 81%) as a cream solid.

HCl (1M in Et₂O, 0.45 mL, 0.45 mmol) was added to a solution of fraction B (166 mg, 0.43 mmol) in Et₂O (5.2 mL). The mixture was stirred for 30 min at room temperature. The crude mixture was filtered to afford product 180 (80 mg, 41%) as a cream solid.

E100. Preparation of Product 181

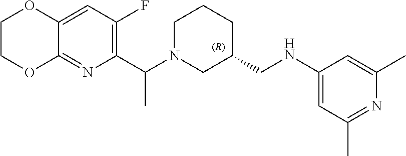

N₂ was bubbled through a solution of 4-bromo-2,6-dimethylpyridine (CAS: 5093-70-9; 107 mg, 0.57 mmol) in 1,4-dioxane (degassed, 6 mL). NaOt-Bu (110 mg, 1.15 mmol), DavePhos (14.7 mg, 37.3 μmol) and Pd₂dba₃ (15.8 mg, 17.2 μmol) were added at room temperature while N₂ was bubbled. Intermediate 186 (178 mg, 0.60 mmol) was added and the reaction mixture was stirred at 100° C. overnight in a sealed tube. The reaction mixture was diluted with NH₄Cl and extracted with EtOAc. The organic layer was dried (MgSO₄), filtered and the solvents were evaporated in vacuo. The crude product was purified by flash column chromatography (silica, NH₃ (7M in MeOH) in DCM, gradient from 0/100 to 2/98). The desired fractions were collected and concentrated in vacuo to yield product 181 (80 mg, 35%) as a yellow solid.

E101. Preparation of Product 182

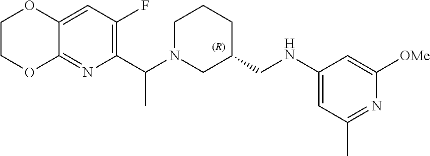

Product 182 was prepared following an analogous procedure to the one described for the synthesis of product 181 using 4-bromo-2-methoxy-6-methylpyridine (CAS: 1083169-00-9) and intermediate 186 as starting materials.

The crude product was purified by flash column chromatography (silica, NH₃ (7M in MeOH) in DCM, gradient from 0/100 to 1/99). The desired fractions were collected and concentrated in vacuo to yield product 182 (141 mg, 59%) as a yellow solid.

E102. Preparation of Products 183 and 184

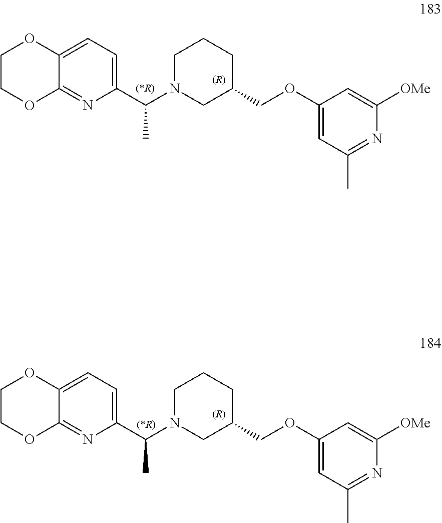

Intermediate 105 (65.1 mg, 0.27 mmol) and K₂CO₃ (104 mg, 0.75 mmol) were added to a stirred solution of intermediate 107 (50.0 mg, 0.25 mmol) in CH₃CN (2 mL). The reaction mixture was stirred overnight at 80° C. Water was added and the mixture was extracted with DCM. The organic phase was dried (Na₂SO₄), filtered and evaporated in vacuo. The crude product was purified by flash column chromatography (silica, NH₃ (7M in MeOH) in DCM, gradient from 0/100 to 10/90). The desired fractions were collected and concentrated in vacuo to afford a racemic mixture. A purification was performed via chiral SFC (stationary phase: CHIRACEL OJ-H 5 μm 250*20 mm, mobile phase: 80% CO₂, 20% MeOH (0.3% i-PrNH₂)) to give product 183 (17 mg, 17%) and product 184 (11 mg, 11%).

E103. Preparation of Products 185 and 186

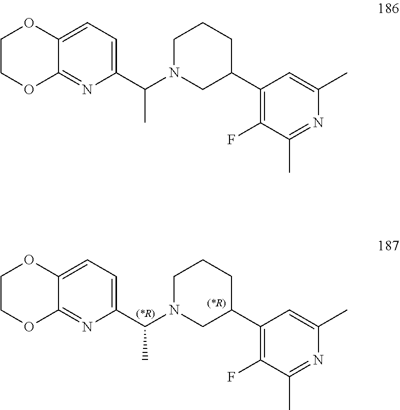 [SMILES: Cc1cc(C2CCCN(C(C)c3ccc4c(n3)OCCO4)C2)c(F)c(C)n1 ; Cc1cc(C2CCCN([C@H](C)c3ccc4c(n3)OCCO4)C2)c(F)c(C)n1]

Intermediate 107 (94.9 mg, 0.48 mmol) and K₂CO₃ (179 mg, 1.30 mmol) were added to a stirred solution of intermediate 150 (90 mg, 0.43 mmol) in CH₃CN (2.25 mL). The reaction mixture was stirred at 80° C. for 18 h. Water was added and the mixture was extracted with EtOAc. The organic phase dried (MgSO₄), filtered and evaporated in vacuo. The crude product was purified by flash column chromatography (silica, MeOH in DCM, gradient from 0/100 to 10/90). The desired fractions were collected and concentrated in vacuo. The residue was purified by RP HPLC (stationary phase: C18 XBridge 30×100 mm 5 um), mobile phase: [0.1% NH₄CO₃H/NH₄OH pH 9 solution in water]/CH₃CN, gradient from 67/33 to 50/50) to give product 185 (8 mg, 5%) and product 186 (20 mg, 12%).

E104. Preparation of Product 187

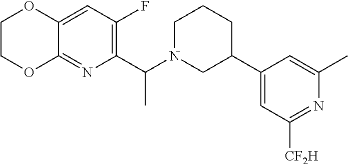 [SMILES: Cc1cc(C2CCCN(C(C)c3nc4c(cc3F)OCCO4)C2)cc(C(F)F)n1]

Product 187 was prepared following an analogous procedure to the one described for the synthesis of products 185 and 186 using intermediate 147 and intermediate 129 as starting materials.

The crude product was purified by flash column chromatography (silica, MeOH in DCM, gradient from 0/100 to 10/90). The desired fractions were collected and concentrated in vacuo. A second purification was performed by RP HPLC (stationary phase: C18 XBridge 30×100 mm 5 um), mobile phase: [0.1% NH₄CO₃H/NH₄OH pH 9 solution in water]/CH₃CN, gradient from 67/33 to 50/50). The desired fractions were collected and concentrated in vacuo to afford product 187 (147 mg, 82%) as a light yellow solid.

E105. Preparation of Product 188

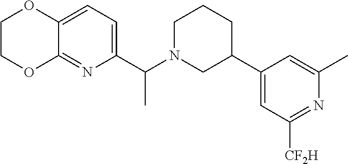 [SMILES: Cc1cc(C2CCCN(C(C)c3ccc4c(n3)OCCO4)C2)cc(C(F)F)n1]

Product 188 was prepared following an analogous procedure to the one described for the synthesis of products 185 and 186 using intermediate 147 and intermediate 107 as starting materials.

The crude product was purified by flash column chromatography (silica, MeOH in DCM, gradient from 0/100 to 10/90). The desired fractions were collected and concentrated in vacuo. A second purification was performed by RP HPLC (stationary phase: C18 XBridge 30×100 mm 5 um), mobile phase: [0.1% NH₄CO₃H/NH₄OH pH 9 solution in water]/CH₃CN, gradient from 67/33 to 50/50). The desired fractions were collected and concentrated in vacuo to afford product 188 (109 mg, 63%) as a light yellow solid.

E106. Preparation of Product 189

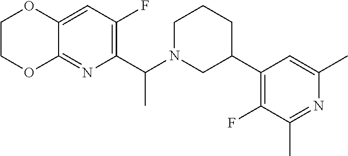 [SMILES: Cc1cc(C2CCCN(C(C)c3nc4c(cc3F)OCCO4)C2)c(F)c(C)n1]

Intermediate 100 (90.9 mg, 0.46 mmol) and Ti(Oi-Pr)₄ (CAS: 546-68-9; 0.17 mL, 0.58 mmol) were added to a stirred solution of intermediate 150 (80.0 mg, 0.38 mmol) in THF (2.81 mL) at room temperature and under N₂ atmosphere. The reaction mixture was stirred at 80° C. overnight, cooled to room temperature and sodium cyanoborohydride (CAS: 25895-60-7; 28.9 mg, 0.46 mmol) was added. The reaction mixture was stirred at 80° C. for 24 h. The mixture was diluted with water and extracted with EtOAc. The organic layer was dried (MgSO₄), filtered and the solvents were evaporated in vacuo. The residue was purified by RP HPLC (stationary phase: C18 XBridge 30×100 mm 5 μm), mobile phase: NH₄HCO₃ (0.25% solution in water)/CH₃CN, gradient from 67/33 to 50/50). The product was further purified by flash chromatography (silica, MeOH in DCM, gradient from 0/100 to 5/95). The desired fractions were collected and concentrated in vacuo to afford product 189 (30 mg, 20%) as a light yellow solid.

E107. Preparation of Product 190

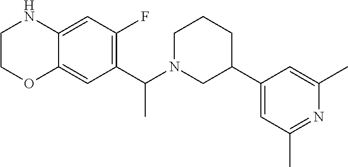 [SMILES: Cc1cc(C2CCCN(C(C)c3cc4c(cc3F)NCCO4)C2)cc(C)n1]

HCl (4M in 1,4-dioxane, 0.42 mL, 1.68 mmol) was added to a solution of intermediate 188 (77.9 mg, 0.17 mmol) in 1,4-dioxane (1.3 mL) in a sealed tube at room temperature. The reaction mixture was stirred at room temperature for 4 h and concentrated in vacuo. The crude mixture was purified by ion exchange chromatography (ISOLUTE SCX2 cartridge). The product was eluted with MeOH, then with NH₃ (7N in MeOH). The desired fractions were collected and concentrated in vacuo to afford product 190 (45.5 mg, 74%) as a white solid.

E108. Preparation of Product 191

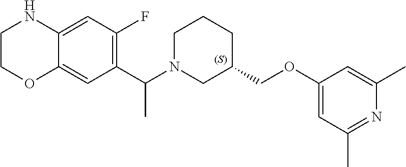

Product 191 was prepared following an analogous procedure to the one described for the synthesis of product 190 using intermediate 189 as starting material.

The crude mixture was purified by ion exchange chromatography (isolute SCX2 cartridge). The product was eluted with MeOH, then with NH₃ (7N in MeOH). The desired fractions were collected and concentrated in vacuo. A second purification was performed by RP HPLC (stationary phase: C18 XBridge 30×100 mm 5 μm), mobile phase: NH₄HCO₃ (0.25% solution in water)/CH₃CN, gradient from 67/33 to 50/50) to give product 191 (20 mg, 33%).

E109. Preparation of Product 192

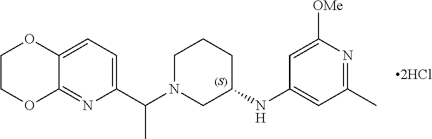

Intermediate 107 (92.0 mg, 0.46 mmol) was added to a solution of intermediate 152 (85 mg, 0.38 mmol) and K₂CO₃ (106 mg, 0.77 mmol) in CH₃CN (5 mL). The reaction mixture was stirred at 60° C. for 20 h. the solvent was removed and the crude product purified reverse phase ([25 mM NH₄HCO₃]/[CH₃CN/MeOH, 1/1], gradient from 70/30 to 27/73). The desired fractions were collected and concentrated in vacuo.

The residue (130 mg) was taken into DCM and treated with HCl (4N in 1,4-dioxane, 2 eq). The solvents were evaporated in vacuo and the product was triturated with Et₂O to give product 192 (128 mg, 73%) as a white solid.

E110. Preparation of Product 193

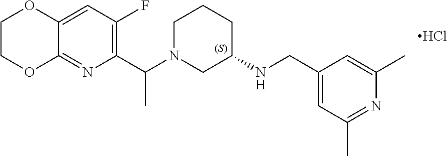

Intermediate 129 (119 mg, 0.55 mmol) was added to a solution of intermediate 168 (100 mg, 0.46 mmol) and K₂CO₃ (126 mg, 0.91 mmol) in CH₃CN (5 mL). The reaction mixture was stirred at 75° C. for 48 h. The solvent was removed and the crude product was purified by flash column chromatography (silica, MeOH in DCM, gradient from 0/100 to 4/96). The desired fractions were collected and concentrated in vacuo. The product was triturated with Et₂₀.

The residue (140 mg) was taken into DCM and treated with HCl (4N in 1,4-dioxane, 1 eq). The solvents were evaporated in vacuo and the product was triturated with DIPE to give product 193 (132 mg, 66%) as a slightly pink solid.

E111. Preparation of Product 194

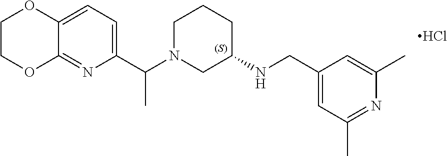

Product 194 was prepared following an analogous procedure to the one described for the synthesis of product 193 using intermediate 168 and intermediate 107 as starting materials.

The crude product purified by flash column chromatography (silica, MeOH in DCM, gradient from 0/100 to 4/96). The desired fractions were collected and concentrated in vacuo and the product was triturated with Et₂₀. The residue (120 mg) was taken into DCM and treated with HCl (4N in 1,4-dioxane, 1 eq). The solvents were evaporated in vacuo and the product was triturated with DIPE to give product 194 (115 mg, 59%) as a slightly pink solid.

E112. Preparation of Product 195

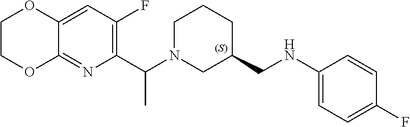

Intermediate 129 (100 mg, 0.46 mmol) was added to a solution of intermediate 154 (79.7 mg, 0.38 mmol) and K₂CO₃ (106 mg, 0.77 mmol) in CH₃CN (3.3 mL). The reaction mixture was stirred at 75° C. for 24 h. The solvent was removed and the crude product was purified by flash column chromatography (silica, EtOAc in heptane, gradient from 0/100 to 80/20). A second purification was performed by reverse phase chromatography ([0.1% HCOOH]/[CH₃CN/MeOH, 1/1]), gradient from 95/5 to 63/37). The desired fractions were collected and concentrated in vacuo to afford product 195 (63 mg, 42%) as a colorless oil.

E113. Preparation of Product 196

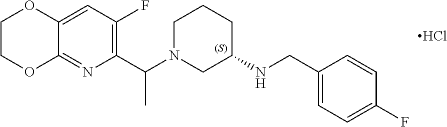

Product 196 was prepared following an analogous procedure to the one described for the synthesis of product 195 using intermediate 156 and intermediate 129 as starting materials.

The crude product purified by flash column chromatography (silica, MeOH in DCM, gradient from 0/100 to 4/96). The desired fractions were collected and concentrated in vacuo. The residue (62 mg) was taken into DCM and treated with HCl (4N in 1,4-dioxane, 1 eq). The solvents were evaporated in vacuo and the product was triturated with Et₂O to yield product 196 (38 mg, 23%) as a white solid.

E114. Preparation of Product 197

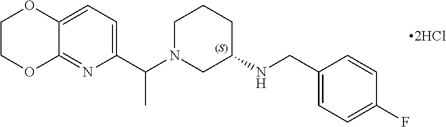

Product 197 was prepared following an analogous procedure to the one described for the synthesis of product 195 using intermediate 107 and intermediate 156 as starting materials.

The crude product purified by flash column chromatography (silica, EtOAc in heptane, gradient from 0/100 to 20/80). A second purification was performed by reverse phase chromatography ([25 mM NH₄HCO₃]/[MeCN/MeOH, 1/1], gradient from 72/28 to 36/64). The desired fractions were collected and concentrated in vacuo. The residue (65 mg) was taken into DCM and treated with HCl (4N in 1,4-dioxane, 2 eq). The solvents were evaporated in vacuo and the product was triturated with Et₂O to yield product 197 (42 mg, 20%) as a white solid.

E115. Preparation of Product 198

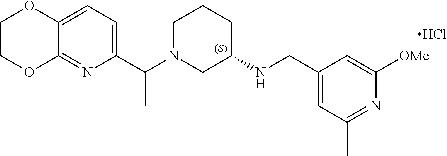

K₂CO₃ (143 mg, 1.04 mmol) was added to a stirred solution of intermediate 190 (171 mg, 0.35 mmol) in MeOH (0.93 mL) and H₂O (0.34 mL). The reaction mixture was stirred at 60° C. for 6 h and the solvent was evaporated in vacuo. The mixture was extracted with EtOAc. The organic layer was dried (MgSO₄), filtered and the solvents were evaporated in vacuo. The crude mixture was purified by reverse phase ([25 mM NH₄HCO₃]/[CH₃CN/MeOH, 1/1], gradient from 72/28 to 36/64). The desired fractions were collected and concentrated in vacuo. The residue (45 mg) was taken into DCM and treated with HCl (4N in 1,4-dioxane, 1 eq). The solvents were evaporated in vacuo and the product was triturated with Et₂O to yield product 198 (32.7 mg, 22%) as a white solid.

The following compounds were prepared following the methods exemplified in the Experimental Part. In case no salt form is indicated, the compound was obtained as a free base. ‘Ex. No.’ refers to the Example number according to which protocol the compound was synthesized. ‘Co. No.’ means compound number.

TABLE 1
(I)

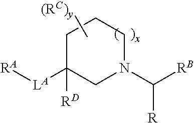

| | | | Salt |
|---|---|---|---|
| Co. No. | Exp. No. | Co. Formula (I) | Form |
| 1 | E1 | 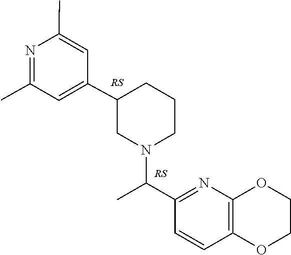 [SMILES: Cc1cc(C2CCCN(C(C)c3ccc4c(n3)OCCO4)C2)cc(C)n1] | |
| 2 | E2 | 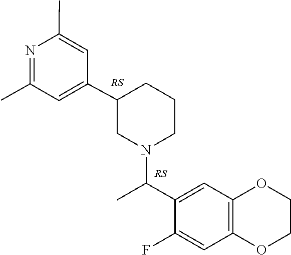 [SMILES: Cc1cc(C2CCCN(C(C)c3cc4c(cc3F)OCCO4)C2)cc(C)n1] | |
| 3 | E3 | 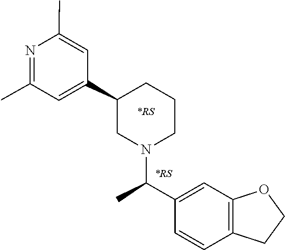 [SMILES: Cc1cc([C@H]2CCCN([C@H](C)c3ccc4c(c3)OCC4)C2)cc(C)n1] | |
| 4 | E3 | 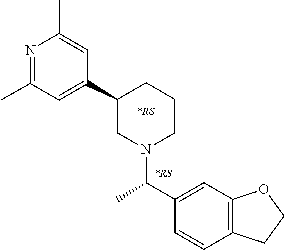 [SMILES: Cc1cc([C@H]2CCCN([C@@H](C)c3ccc4c(c3)OCC4)C2)cc(C)n1] | |
| 5 | E3 | 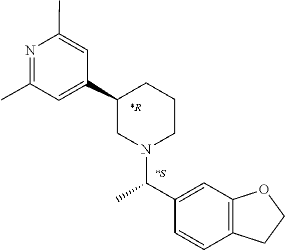 [SMILES: Cc1cc([C@H]2CCCN([C@@H](C)c3ccc4c(c3)OCC4)C2)cc(C)n1] | |
| 6 | E3 | 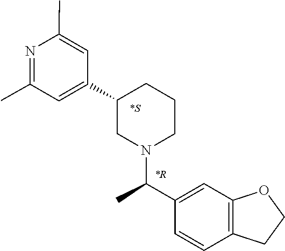 [SMILES: Cc1cc([C@@H]2CCCN([C@H](C)c3ccc4c(c3)OCC4)C2)cc(C)n1] | |
| 7 | E4 | 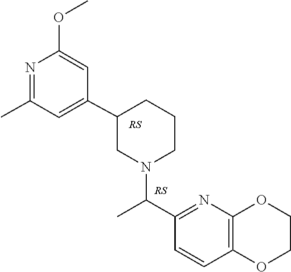 [SMILES: COc1cc(C2CCCN(C(C)c3ccc4c(n3)OCCO4)C2)cc(C)n1] | |
| 8 | E5 | 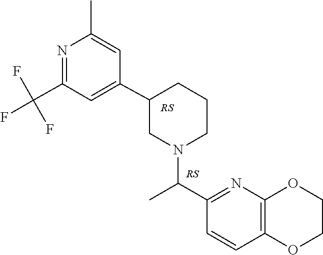 [SMILES: Cc1cc(C2CCCN(C(C)c3ccc4c(n3)OCCO4)C2)cc(C(F)(F)F)n1] | |
| 9 | E6 | 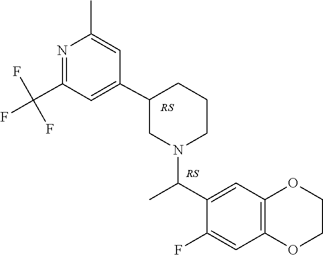 [SMILES: Cc1cc(C2CCCN(C(C)c3cc4c(cc3F)OCCO4)C2)cc(C(F)(F)F)n1] | |
| 10 | E7 | 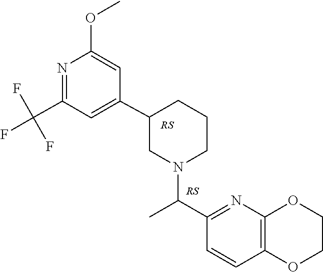 [SMILES: COc1cc(C2CCCN(C(C)c3ccc4c(n3)OCCO4)C2)cc(C(F)(F)F)n1] | |
| 11 | E8 | 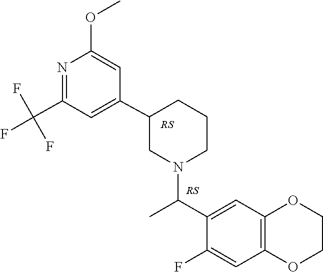 [SMILES: COc1cc(C2CCCN(C(C)c3cc4c(cc3F)OCCO4)C2)cc(C(F)(F)F)n1] | |
| 12 | E9 | 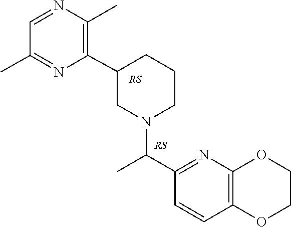 [SMILES: Cc1cnc(C)c(C2CCCN(C(C)c3ccc4c(n3)OCCO4)C2)n1] | |
| 13 | E10 | 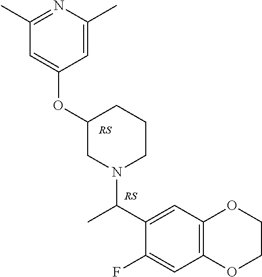 [SMILES: Cc1cc(OC2CCCN(C(C)c3cc4c(cc3F)OCCO4)C2)cc(C)n1] | |
| 14 | E11 | 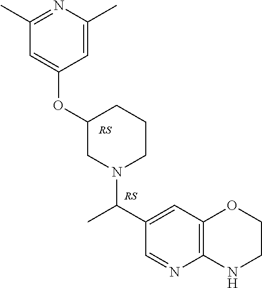 [SMILES: Cc1cc(OC2CCCN(C(C)c3cnc4c(c3)OCCN4)C2)cc(C)n1] | |
| 15 | E12 | 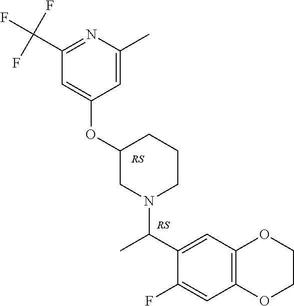 [SMILES: Cc1cc(OC2CCCN(C(C)c3cc4c(cc3F)OCCO4)C2)cc(C(F)(F)F)n1] | |
| 16 | E13 | 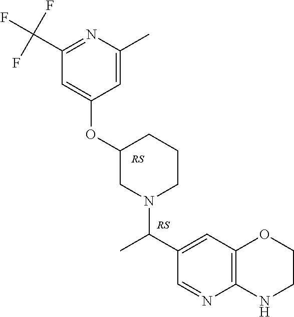 [SMILES: Cc1cc(OC2CCCN(C(C)c3cnc4c(c3)OCCN4)C2)cc(C(F)(F)F)n1] | |
| 17 | E13 | 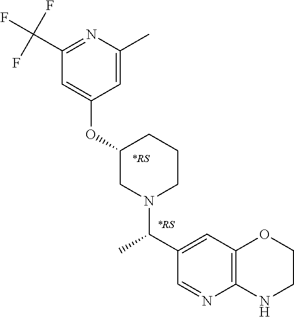 [SMILES: Cc1cc(O[C@@H]2CCCN([C@@H](C)c3cnc4c(c3)OCCN4)C2)cc(C(F)(F)F)n1] | |
| 18 | E14 | 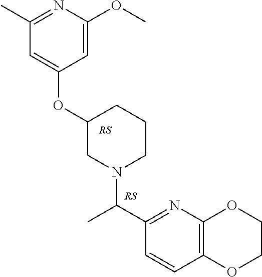 [SMILES: COc1cc(OC2CCCN(C(C)c3ccc4c(n3)OCCO4)C2)cc(C)n1] | |
| 19 | E15 | 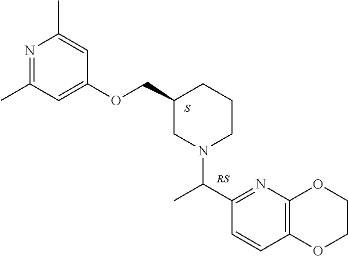 | |
| 20 | E16 | 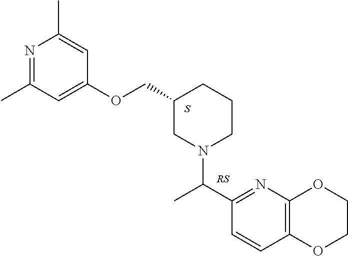 | |
| 21 | E17 | 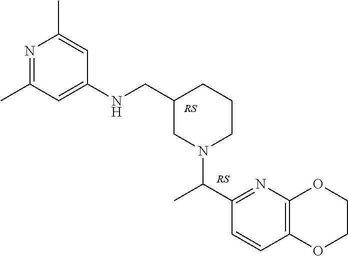 | .2HCl |
| 22 | E18 | 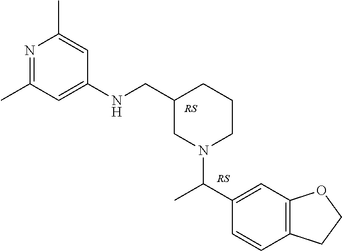 | .2HCl |
| 23 | E19 | 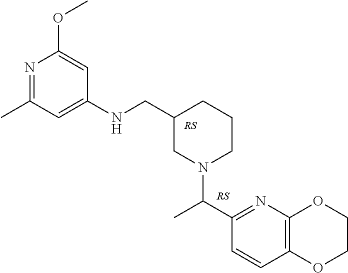 | .2HCl |
| 24 | E20 | 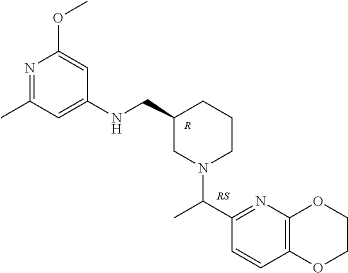 | |
| 25 | E20 | 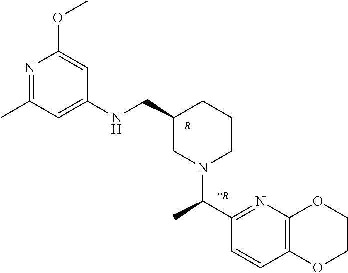 | .2HCl |
| 26 | E20 | 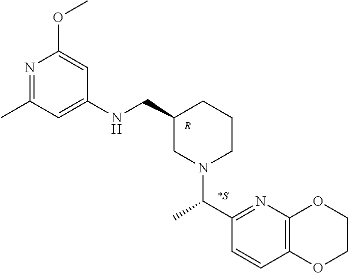 | 0.60HCl |
| 27 | E21 | 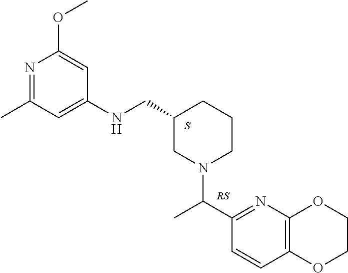 | |
| 28 | E21 | 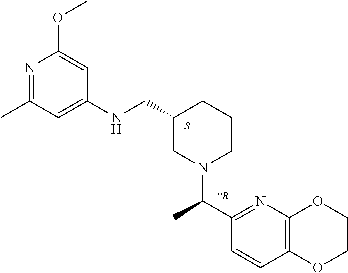 | |
| 29 | E21 | 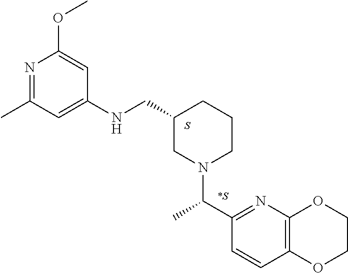 | |
| 30 | E22 | 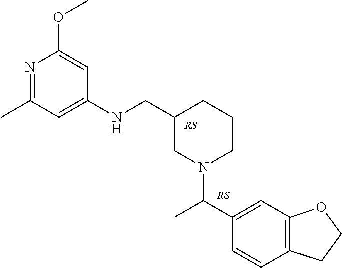 | .2HCl |
| 31 | E23 | 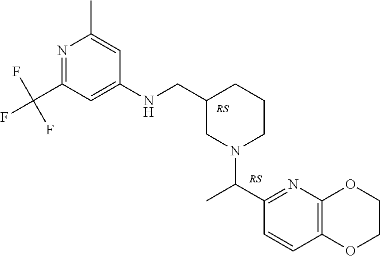 | |
| 32 | E23 | 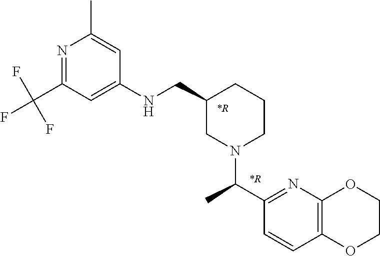 | |
| 33 | E23 | 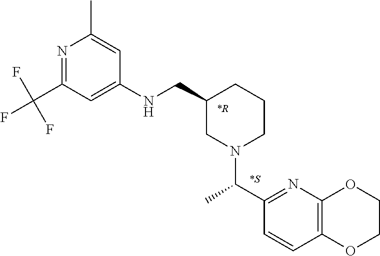 | |
| 34 | E23 | 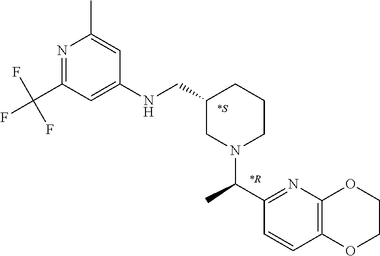 | |
| 35 | E23 | 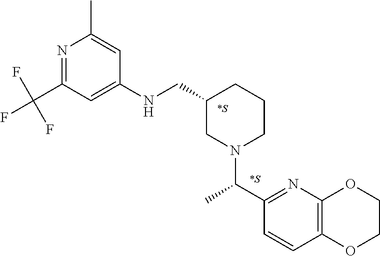 | |
| 36 | E24 | 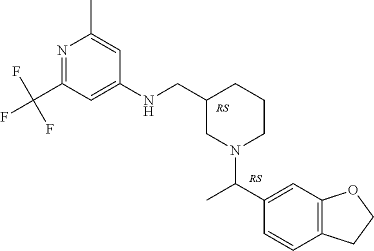 | .2HCl |
| 37 | E25 | 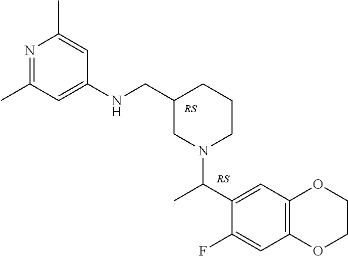 | |
| 38 | E26 | 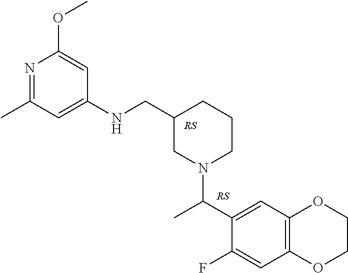 | .HCl |
| 39 | E27 | 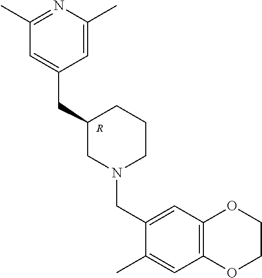 | .2HCl |
| 40 | E28 | 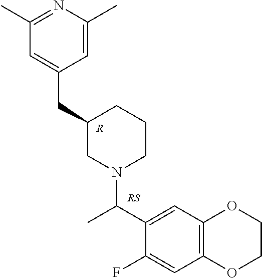 | .2HCl |
| 41 | E28 | 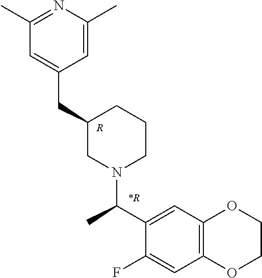 | .2HCl |
| 42 | E28 | 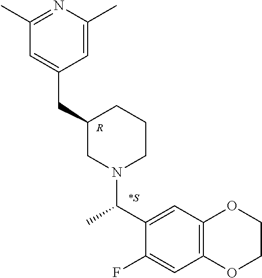 | .2HCl |
| 43 | E29 | 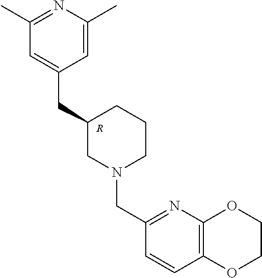 | |
| 44 | E30 | 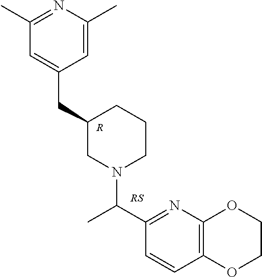 | |
| 45 | E30 | 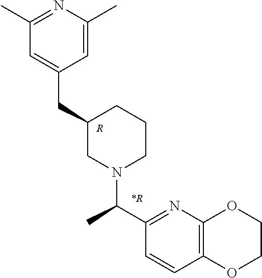 | |
| 46 | E30 | 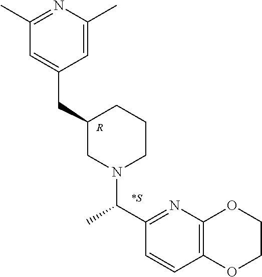 | |
| 47 | E31 | 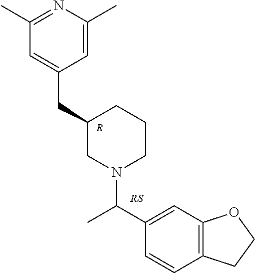 | |
| 48 | E32 | 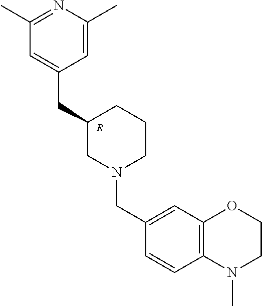 | |
| 49 | E33 | 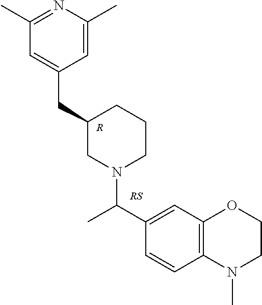 | .2HCl |
| 50 | E34 | 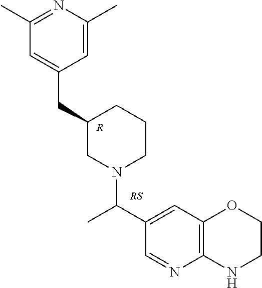 | |
| 51 | E34 | 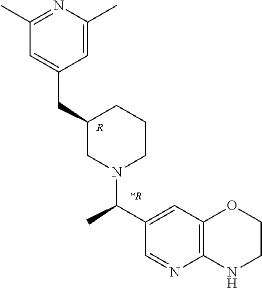 | |
| 52 | E34 | 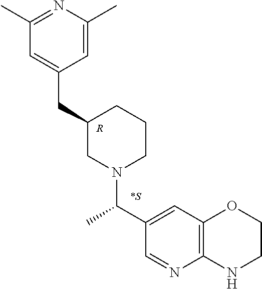 | |
| 53 | E35 | 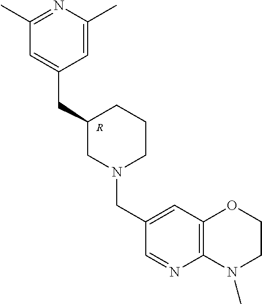 | |
| 54 | E36 | 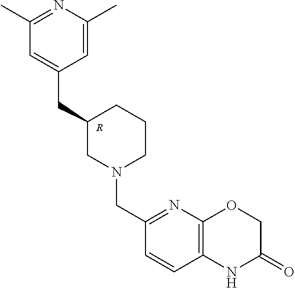 | |
| 55 | E37 | 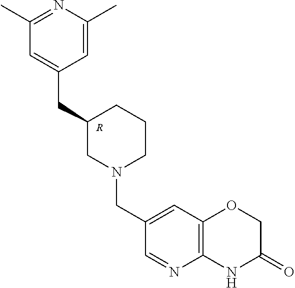 | |
| 56 | E38 | 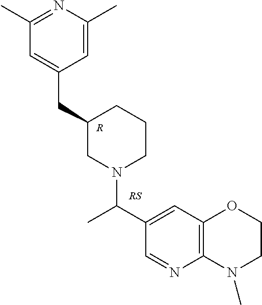 | |
| 57 | E39 | 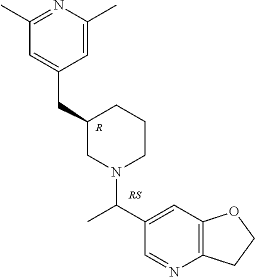 | |
| 58 | E40 | 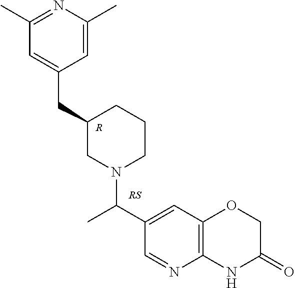 | |
| 59 | E41 | 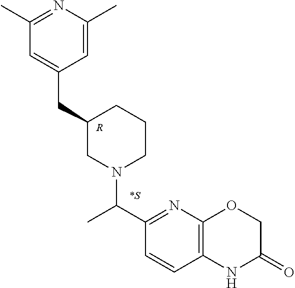 | |
| 60 | E41 | 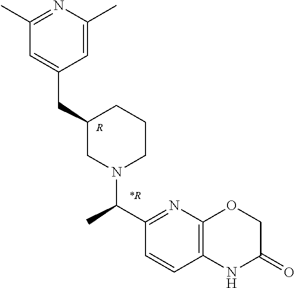 | |
| 61 | E41 | 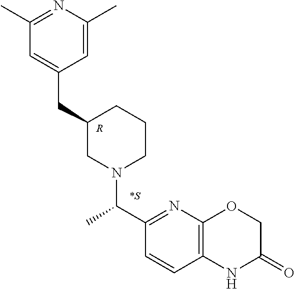 | |
| 62 | E42 | 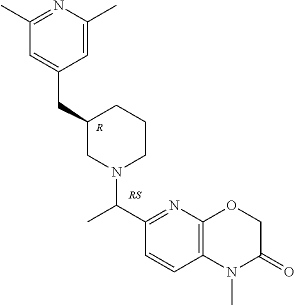 | |
| 63 | E43 | 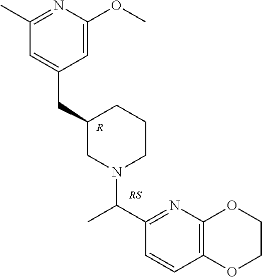 | |
| 64 | E43 | 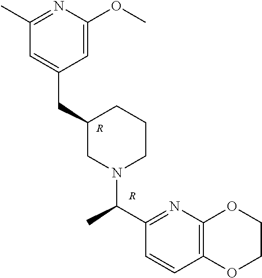 | |
| 65 | E43 | 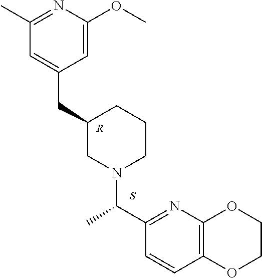 | |
| 66 | E44 | 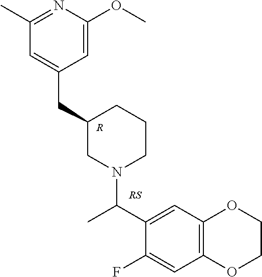 | |
| 67 | E45 | 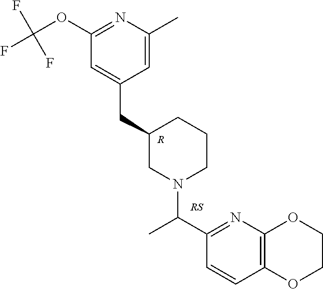 | |
| 68 | E46 | 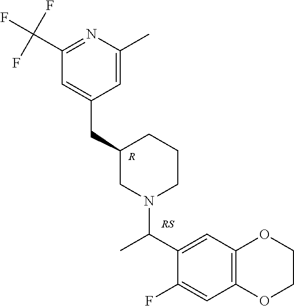 | |
| 69 | E47 | 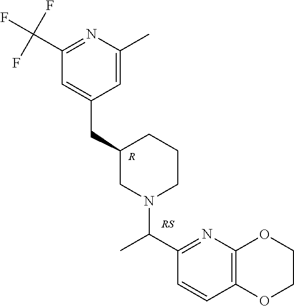 | |
| 99 | E62 | 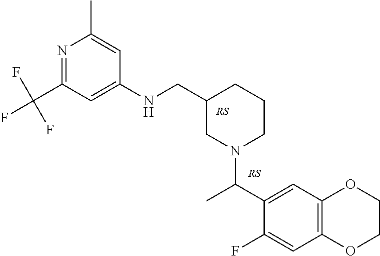 | .2HCl |
| 108 | E16 | 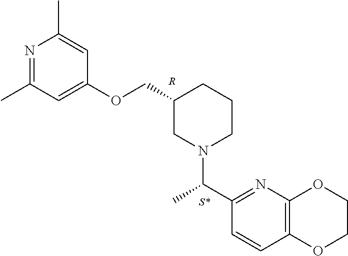 | |
| 109 | E16 | 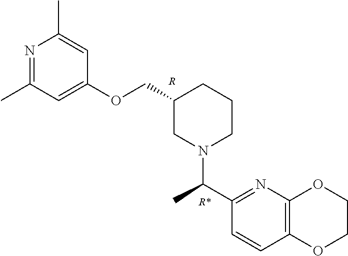 | |
| 110 | E63 | 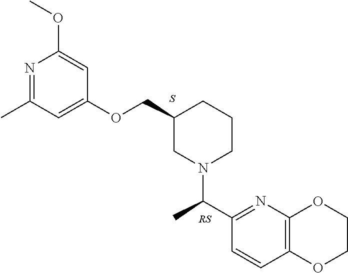 | |
| 111 | E64 | 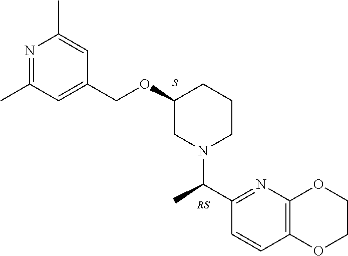 | |
| 112 | E65 | 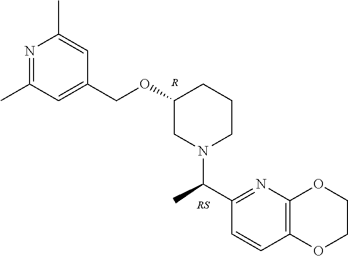 | |
| 113 | E66 | 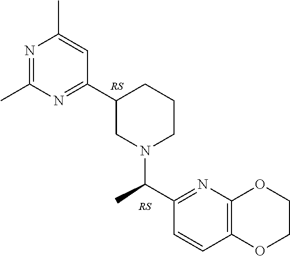 | |
| 114 | E67 | 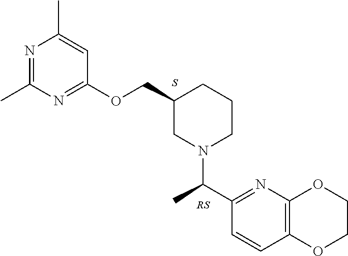 | |
| 125 | I-89 | 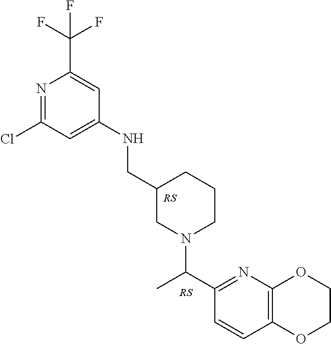 | |
| 126 | I-90 | 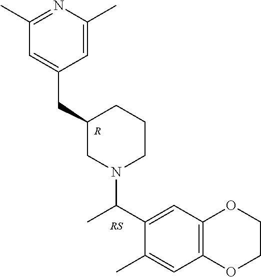 | |
| 127 | I-92 | 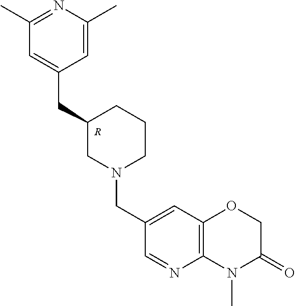 | |
| 130 | E68 | 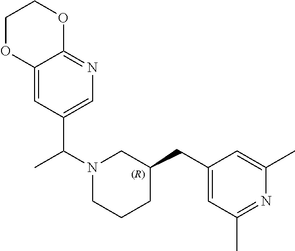 | |
| 131 | E68 | 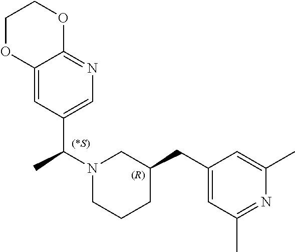 | |
| 132 | E68 | 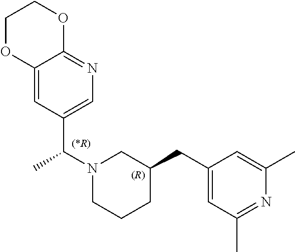 | |
| 133 | E69 | 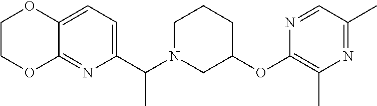 [SMILES: Cc1cnc(OC2CCCN(C(C)c3ccc4c(n3)OCCO4)C2)c(C)n1] | |
| 134 | E70 | 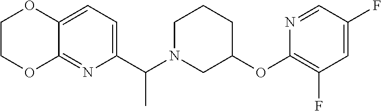 [SMILES: CC(c1ccc2c(n1)OCCO2)N1CCCC(Oc2ncc(F)cc2F)C1] | |
| 135 | E71 | 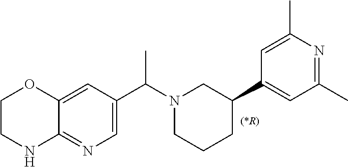 [SMILES: Cc1cc([C@H]2CCCN(C(C)c3cnc4c(c3)OCCN4)C2)cc(C)n1] | 2 HCl |
| 136 | E72 | 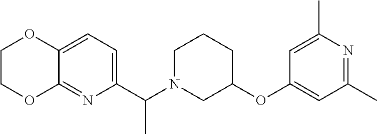 [SMILES: Cc1cc(OC2CCCN(C(C)c3ccc4c(n3)OCCO4)C2)cc(C)n1] | |
| 137 | E73 | 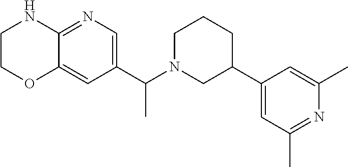 [SMILES: Cc1cc(C2CCCN(C(C)c3cnc4c(c3)OCCN4)C2)cc(C)n1] | |
| 138 | E74 | 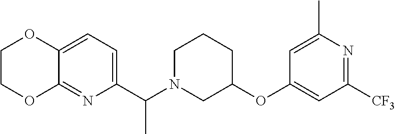 [SMILES: Cc1cc(OC2CCCN(C(C)c3ccc4c(n3)OCCO4)C2)cc(C(F)(F)F)n1] | |
| 139 | E75 | 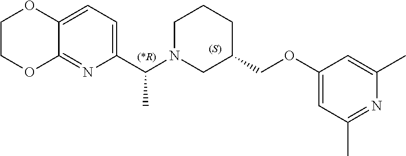 | 2 HCl |
| 140 | E75 | 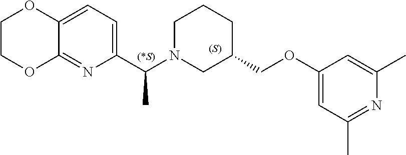 | 2 HCl |
| 141 | E76 | 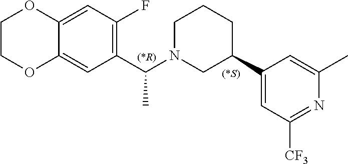 [SMILES: Cc1cc([C@@H]2CCCN([C@H](C)c3cc4c(cc3F)OCCO4)C2)cc(C(F)(F)F)n1] | |
| 142 | E76 | 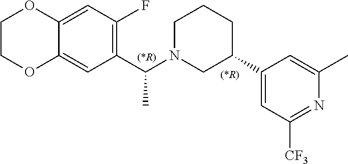 [SMILES: Cc1cc([C@H]2CCCN([C@H](C)c3cc4c(cc3F)OCCO4)C2)cc(C(F)(F)F)n1] | |
| 143 | E76 | 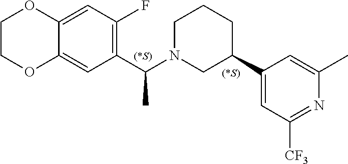 [SMILES: Cc1cc([C@@H]2CCCN([C@@H](C)c3cc4c(cc3F)OCCO4)C2)cc(C(F)(F)F)n1] | |
| 144 | E76 | 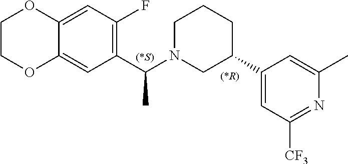 [SMILES: Cc1cc([C@H]2CCCN([C@@H](C)c3cc4c(cc3F)OCCO4)C2)cc(C(F)(F)F)n1] | |
| 145 | E77 | 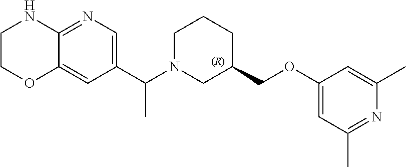 | |
| 146 | E78 | 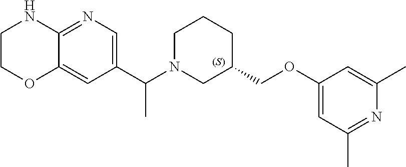 | |
| 147 | E79 | 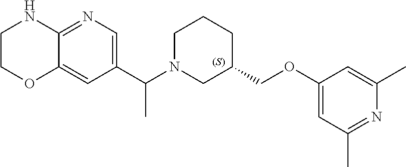 | 3 HCl |
| 148 | E80 | 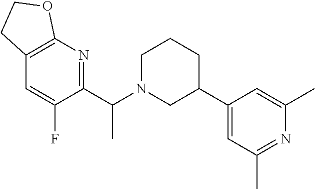 [SMILES: Cc1cc(C2CCCN(C(C)c3nc4c(cc3F)CCO4)C2)cc(C)n1] | |
| 149 | E81 | 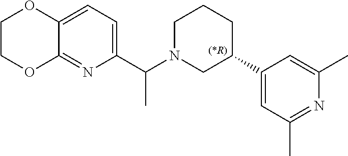 | |
| 150 | E82 | 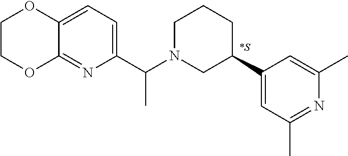 | |
| 151 | E83 | 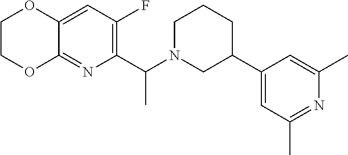 [SMILES: Cc1cc(C2CCCN(C(C)c3nc4c(cc3F)OCCO4)C2)cc(C)n1] | |
| 152 | E84 | 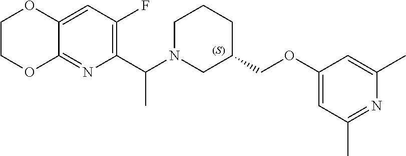 | |
| 153 | E84 | 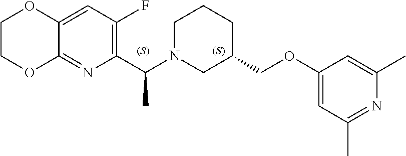 | |
| 154 | E84 | 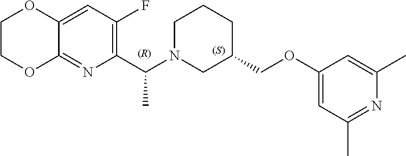 | |
| 155 | E85 | 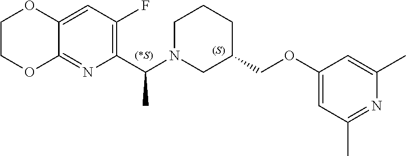 | 2 HCl |
| 156 | E86 | 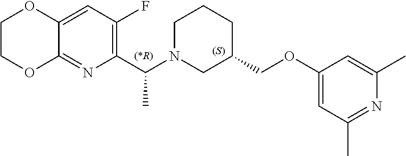 | 2 HCl |
| 157 | E87 | 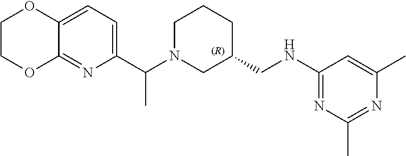 | 2 HCl |
| 158 | E88 | 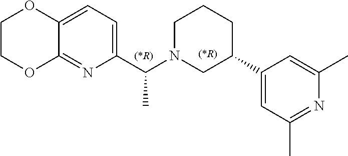 | 2 HCl |
| 159 | E88 | 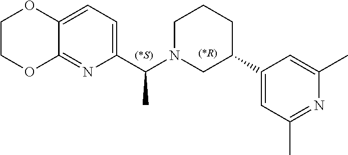 | 2 HCl |
| 160 | E89 | 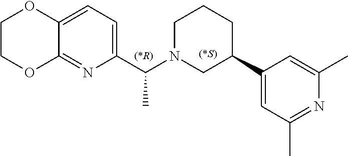 | 2 HCl |
| 161 | E89 | 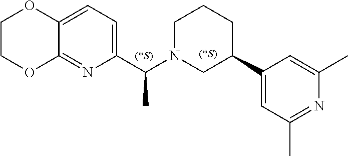 | 2 HCl |
| 162 | E90 | 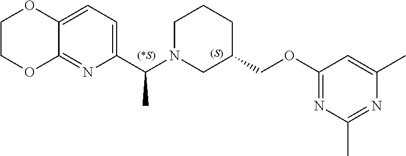 | |
| 163 | E90 | 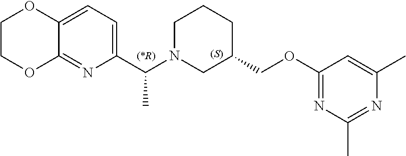 | |
| 164 | E91 | 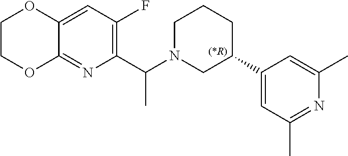 | 2 HCl |
| 165 | E91 | 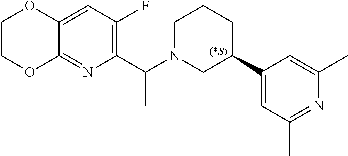 | 2 HCl |
| 166 | E92 | 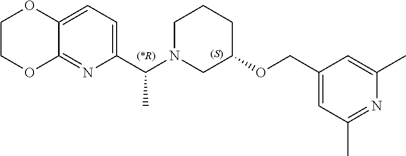 | 2 HCl |
| 167 | E92 | 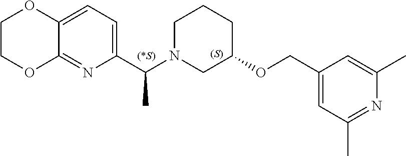 | 2 HCl |
| 168 | E93 | 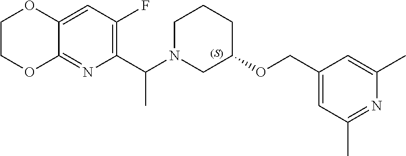 | |
| 169 | E93 | 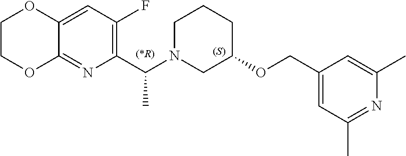 | 2 HCl |
| 170 | E93 | 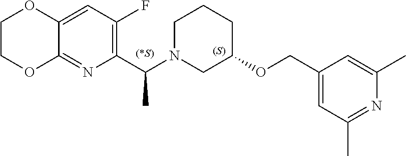 | 2 HCl |
| 171 | E94 | 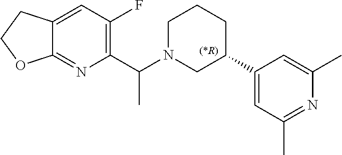 [SMILES: Cc1cc([C@H]2CCCN(C(C)c3nc4c(cc3F)CCO4)C2)cc(C)n1] | |
| 172 | E95 | 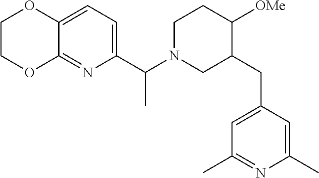 [SMILES: COC1CCN(C(C)c2ccc3c(n2)OCCO3)CC1Cc1cc(C)nc(C)c1] | |
| 173 | E96 | 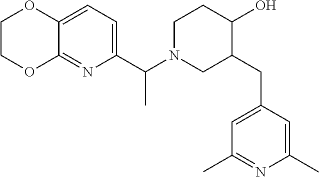 [SMILES: Cc1cc(CC2CN(C(C)c3ccc4c(n3)OCCO4)CCC2O)cc(C)n1] | |
| 174 | E97 | 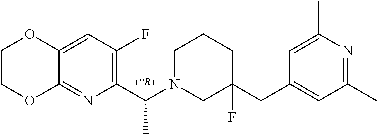 [SMILES: Cc1cc(CC2(F)CCCN([C@H](C)c3nc4c(cc3F)OCCO4)C2)cc(C)n1] | 2 HCl |
| 175 | E97 | 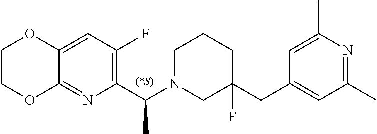 [SMILES: Cc1cc(CC2(F)CCCN([C@@H](C)c3nc4c(cc3F)OCCO4)C2)cc(C)n1] | 2 HCl |
| 176 | E98 | 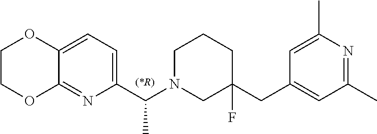 [SMILES: Cc1cc(CC2(F)CCCN([C@H](C)c3ccc4c(n3)OCCO4)C2)cc(C)n1] | 2 HCl |
| 177 | E98 | 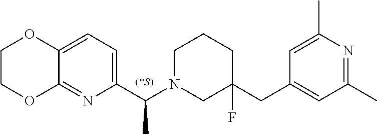 [SMILES: Cc1cc(CC2(F)CCCN([C@@H](C)c3ccc4c(n3)OCCO4)C2)cc(C)n1] | 2 HCl |
| 178 | E99 | 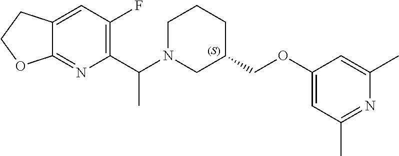 | |
| 179 | E99 | 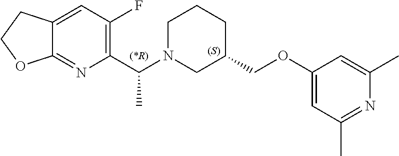 | |
| 180 | E99 | 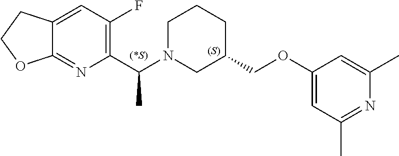 | 2 HCl |
| 181 | E100 | 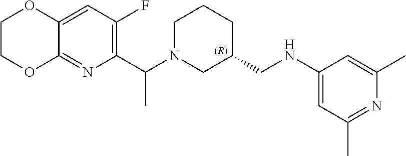 | |
| 182 | E101 | 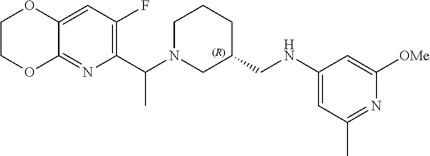 | |
| 183 | E102 | 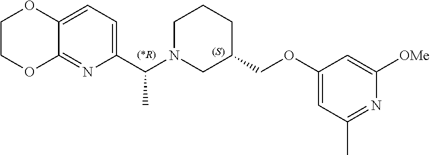 | |
| 184 | E102 | 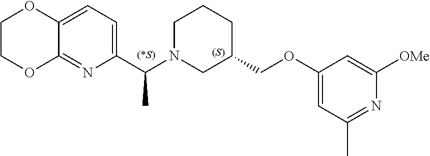 | |
| 185 | E103 | 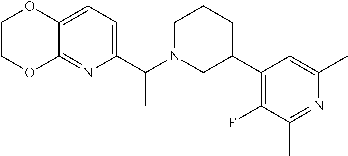 [SMILES: Cc1cc(C2CCCN(C(C)c3ccc4c(n3)OCCO4)C2)c(F)c(C)n1] | |
| 186 | E104 | 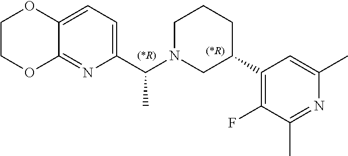 | |
| 187 | E105 | 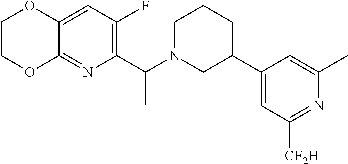 [SMILES: Cc1cc(C2CCCN(C(C)c3nc4c(cc3F)OCCO4)C2)cc(C(F)F)n1] | |
| 188 | E106 | 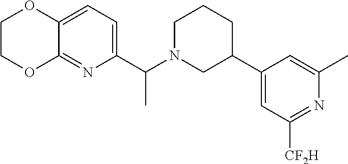 [SMILES: Cc1cc(C2CCCN(C(C)c3ccc4c(n3)OCCO4)C2)cc(C(F)F)n1] | |
| 189 | E107 | 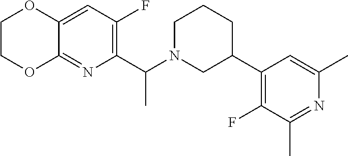 [SMILES: Cc1cc(C2CCCN(C(C)c3nc4c(cc3F)OCCO4)C2)c(F)c(C)n1] | |
| 190 | E108 | 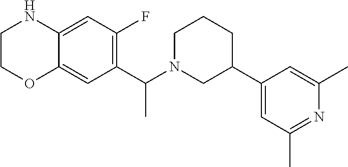 [SMILES: Cc1cc(C2CCCN(C(C)c3cc4c(cc3F)NCCO4)C2)cc(C)n1] | |
| 191 | E109 | 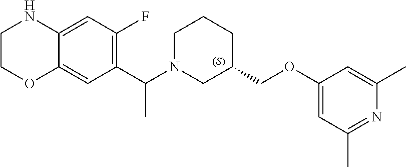 | |
| 192 | E110 | 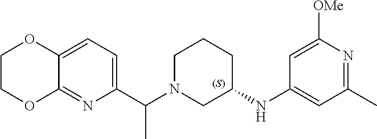 | 2 HCl |
| 193 | E111 | 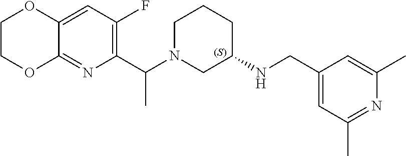 | HCl |
| 194 | E112 | 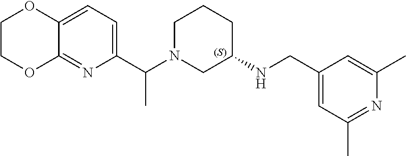 | HCl |
| 195 | E113 | 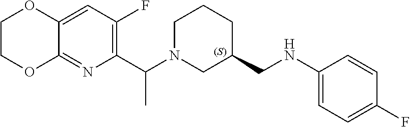 | |
| 196 | E114 | 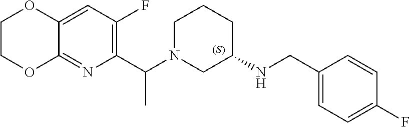 | HCl |
| 197 | E115 | 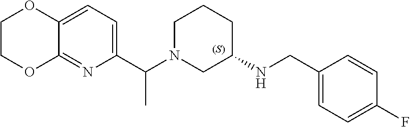 | 2 HCl |
| 198 | E116 | 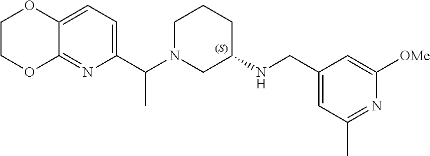 | HCl |

The values of salt stoichiometry or acid content in the compounds as provided herein, are those obtained experimentally. The content of hydrochloric acid reported herein was determined by ¹H NMR integration and/or elemental analysis.

Analytical Part

Melting Points

Values are peak values, and are obtained with experimental uncertainties that are commonly associated with this analytical method.

DSC823e (A): For a number of compounds, melting points were determined with a DSC823e (Mettler-Toledo) apparatus. Melting points were measured with a temperature gradient of 10° C./minute. Maximum temperature was 300° C. Values are peak values (A).

Mettler Toledo MP50 (B): For a number of compounds, melting points were determined in open capillary tubes on a Mettler FP 81HT/FP90 apparatus. Melting points were measured with a temperature gradient of 1, 3, 5 or 10° C./minute. Maximum temperature was 300° C. The melting point was read from a digital display.

LCMS

General Procedure

The High Performance Liquid Chromatography (HPLC) measurement was performed using a LC pump, a diode-array (DAD) or a UV detector and a column as specified in the respective methods. If necessary, additional detectors were included (see table of methods below).

Flow from the column was brought to the Mass Spectrometer (MS) which was configured with an atmospheric pressure ion source. It is within the knowledge of the skilled person to set the tune parameters (e.g. scanning range, dwell time . . . ) in order to obtain ions allowing the identification of the compound's nominal monoisotopic molecular weight (MW) and/or exact mass monoisotopic molecular weight. Data acquisition was performed with appropriate software.

Compounds are described by their experimental retention times (Rt) and ions. If not specified differently in the table of data, the reported molecular ion corresponds to the [M+H]⁺ (protonated molecule) and/or [M−H]⁻ (deprotonated molecule). In case the compound was not directly ionizable the type of adduct is specified (i.e. [M+NH₄]⁺, [M+HCOO]⁻, [M+CH₃COO]⁻ etc. . . . ). For molecules with multiple isotopic patterns (Br, Cl..), the reported value is the one obtained for the lowest isotope mass. All results were obtained with experimental uncertainties that are commonly associated with the method used.

Hereinafter, “SQD” Single Quadrupole Detector, “MSD” Mass Selective Detector, “QTOF” Quadrupole-Time of Flight, “rt” room temperature, “BEH” bridged ethylsiloxane/silica hybrid, HSS” High Strength Silica, “CSH” charged surface hybrid, “UPLC” Ultra Performance Liquid Chromatography, “DAD” Diode Array Detector.

TABLE 2
LC-MS Methods (Flow expressed in mL/min; column temperature (T) in ° C.; Run time in min).

| Method | | | | | Flow | Run |
|---|---|---|---|---|---|---|
| code | Instrument | Column | Mobile phase | Gradient | C̅o̅l̅ T̅ | time |
| 1 | Waters: Acquity ® | Waters: BEH | A: 95% CH₃COONH₄ | From 95% A to | 1 | 5 |
| | IClass UPLC ® - | C18 (1.7 μm, | 6.5 mM + 5% CH₃CN, | 5% A in 4.6 min, | 5̅0̅ | |
| | DAD and Xevo | 2.1 × 50 mm) | B: CH₃CN | held for 0.4 min | | |
| | G2-S QTOF | | | | | |
| 2 | Agilent: | Agilent: | A: 95% CH₃COONH₄ | From 95% A to | 1 | 7 |
| | HP1100-DAD, | Eclipse Plus | 6.5 mM + 5% CH₃CN, | 0% A in 5.0 min, | 6̅0̅ | |
| | MSD G1956B | C18 (3.5 μm, | B: CH₃CN | held in 0.15 min, | | |
| | | 2.1 × 30 mm) | | back to 95% A | | |
| | | | | for 0.15 min, | | |
| | | | | held for 1.7 min | | |
| 3 | Waters: Acquity | Waters: BEH | A: 95% CH₃COONH₄ | 84.2% A for | 0.343 | 6.2 |
| | UPLC ® - DAD and | C18 (1.7 μm, | 7 mM/5% CH₃CN, | 0.49 min, to | 40 | |
| | Quattro Micro ™ | 2.1 × 100 mm) | B: CH₃CN | 10.5% A in | | |
| | | | | 2.18 min, | | |
| | | | | held for 1.94 min, | | |
| | | | | back to 84.2% A | | |
| | | | | in 0.73 min, held | | |
| | | | | for 0.73 min. | | |
| 4 | Waters: Acquity ® | Waters: BEH | A: 95% CH₃COONH₄ | From 95% A to | 1 | 2 |
| | UPLC ® - DAD and | C18 (1.7 μm, | 6.5 mM + 5% CH₃CN, | 40% A in 1.2 min, | 5̅0̅ | |
| | SQD | 2.1 × 50 mm) | B: CH₃CN | to 5% A in | | |
| | | | | 0.6 min, held for | | |
| | | | | 0.2 min | | |
| 5 | Agilent: | YMC: Pack | A: HCOOH 0.1% in | 95% A to 5% A in | 2.6 | 6 |
| | 1100-DAD and | ODS-AQ (3 μm, | water, B: CH₃CN | 4.8 min, held for | 35 | |
| | MSD | 4.6 × 50 mm) | | 1 min, back to | | |
| | | | | 95% A in 0.2 min. | | |
| 6 | Agilent 1260 | YMC-pack | A: 0.1% HCOOH in | From 95% A to | 2.6 | 6.8 |
| | Infinity DAD | ODS-AQ | H₂O B: CH₃CN | 5% A in 4.8 min, | 35 | |
| | TOF-LC/MS | C18 (50 × 4.6 | | held for 1.0 min, | | |
| | G6224A | mm, 3 μm) | | to 95% A in 0.2 | | |
| | | | | min. | | |
| 7 | Waters: Acquity ® | Waters: BEH | A: 95% CH₃COONH₄ | From 95% A to | 0.8 | 2.5 |
| | UPLC ® - DAD and | C18 (1.7 μm, | 6.5 mM + 5% CH₃CN, | 5% A in 2.0 min, | 50 | |
| | SQD | 2.1 × 50 mm) | B: CH₃CN | held for 0.5 min | | |
| 8 | Waters: Acquity ® | Agilent: RRHD | A: 95% CH₃COONH₄ | From 95% A to | 0.8 | 2.5 |
| | IClass UPLC ® - | (1.8 μm, | 6.5 mM + 5% CH₃CN | 5% A in 2.0 min | 50 | |
| | DAD and SQD | 2.1 × 50 mm) | B: CH₃CN | held for 0.5 min | | |
| 9 | Waters: Acquity ® | Agilent: RRHD | A: 95% CH₃COONH₄ | From 95% A to | 0.8 | 5 |
| | IClass UPLC ® - | (1.8 μm, | 6.5 mM + 5% CH₃CN | 5% A in 4.5 min | 50 | |
| | DAD and SQD | 2.1 × 50 mm) | B: CH₃CN | held for 0.5 min | | |
| 10 | Waters: Acquity | Waters: BEH | A: 95% CH₃COONH₄ | From 84.2% A to | 0.343 | 6.1 |
| | UPLC ® H-Class - | C18 (1.7 μm, | | 10.5% A in 2.18 | 40 | |
| | DAD and SQD 2 | 2.1 × 100 mm) | 7 mM/5% CH₃CN, | min, held for | | |
| | | | B: CH₃CN | 1.94 min, back to | | |
| | | | | 84.2% A in 0.73 | | |
| | | | | min, held for | | |
| | | | | 0.73 min. | | |
| 11 | Agilent 1100 HPLC | YMC-pack | A: 0.1% HCOOH in | 100% A held for | 2.6 | 6.2 |
| | DAD LC/MS | ODS-AQ | H2O B: CH₃CN | 0.2. From 100% | 35 | |
| | G1956A | C18 (50 × 0 4.6 | | A to 50% A in | | |
| | | mm, 3 μm) | | 4.5 min, and to | | |
| | | | | 5% A in 0.1 min, | | |
| | | | | held for 1.0 min, | | |
| | | | | to 95% A in 0.2 | | |
| | | | | min. | | |

TABLE 3
Analytical data-melting point (M.p.) and LCMS: [M + H]⁺
means the protonated mass of the free base of the compound,
[M − H]⁻ means the deprotonated mass of the free base of the
compound or the type of adduct specified [M + CH₃COO]⁻).
Rₜ means retention time (in min). For some compounds,
exact mass was determined.

| Co. | M.p. | | | LCMS |
|---|---|---|---|---|
| No. | (° C.) | [M + H]⁺ | Rₜ | Method |
| 1 | n.d. | 354 | 1.45 | 1 |
| 2 | n.d. | 337 | 2.28 | 1 |
| 3 | n.d. | 337 | 2.09 | 1 |
| 4 | n.d. | 337 | 2.13 | 1 |
| 5 | n.d. | 337 | 2.90 | 3 |
| 6 | n.d. | 337 | 2.91 | 3 |
| 7 | n.d. | 370 | 1.82 | 1 |
| 8 | n.d. | 408 | 2.18/2.20 | 1 |
| 9 | n.d. | 425 | 2.99/3.00 | 1 |
| 10 | n.d. | 424 | 2.61 | 1 |
| 11 | n.d. | 441 | 3.33/3.35 | 1 |
| 12 | n.d. | 355 | 1.11 | 1 |
| 13 | n.d. | 387 | 2.34/2.43 | 1 |
| 14 | n.d. | 369 | 1.47/1.52 | 1 |
| 15 | n.d. | 441 | 3.05/3.11 | 1 |
| 16 | n.d. | 423 | 2.14/2.19 | 1 |
| 17 | n.d. | 423 | 2.17 | 1 |
| 18 | n.d. | 386 | 2.01/2.04 | 1 |
| 19 | n.d. | 384 | 1.36 | 1 |
| 20 | n.d. | 384 | 1.38 | 1 |
| 21 | 193.0 (B) | 383 | 1.05 | 5 |
| 22 | 248.4 (B) | 366 | 1.22 | 5 |
| 23 | 266.7 (B) | 399 | 1.22 | 6 |
| 24 | n.d. | 399 | 1.09 | 5 |
| 25 | n.d. | 399 | 1.14 | 1 |
| 26 | n.d. | 399 | 1.18 | 1 |
| 27 | n.d. | 399 | 1.09 | 5 |
| 28 | n.d. | 399 | 2.12 | 3 |
| 29 | n.d. | 399 | 2.08 | 3 |
| 30 | 261.3 (B) | 382 | 1.21 | 5 |
| 31 | n.d. | 437 | 1.39 | 5 |
| 32 | n.d. | 437 | 2.43 | 3 |
| 33 | n.d. | 437 | 2.46 | 3 |
| 34 | n.d. | 437 | 2.46 | 3 |
| 35 | n.d. | 437 | 2.48 | 3 |
| 36 | n.d. | 420 | 1.62 | 5 |
| 37 | n.d. | 400 | 1.36 | 5 |
| 38 | 164.7 (B) | 416 | 1.34/1.37 | 5 |
| 39 | n.d. | 367 | 2.08 | 1 |
| 40 | n.d. | 385 | 1.12 | 4 |
| 41 | n.d. | 385 | 3.10 | 3 |
| 42 | n.d. | 385 | 3.07 | 3 |
| 43 | n.d. | 354 | 1.39 | 1 |
| 44 | n.d. | 368 | 1.26/1.27 | 1 |
| 45 | n.d. | 368 | 2.13 | 3 |
| 46 | n.d. | 368 | 2.18 | 3 |
| 47 | n.d. | 351 | 1.74/1.78 | 1 |
| 48 | n.d. | 366 | 2.73 | 2 |
| 49 | n.d. | 380 | 2.84 | 2 |
| 50 | n.d. | 367 | 1.29 | 1 |
| 51 | n.d. | 367 | 1.98 | 3 |
| 52 | n.d. | 367 | 2.00 | 3 |
| 53 | n.d. | 367 | 1.51 | 1 |
| 54 | n.d. | 367 | 1.09 | 1 |
| 55 | n.d. | 367 | 1.39 | 1 |
| 56 | n.d. | 381 | 1.47/1.50 | 1 |
| 57 | n.d. | 352 | 1.63 | 1 |
| 58 | n.d. | 380 | 1.45/1.48 | 1 |
| 59 | n.d. | 381 | 1.08 | 1 |
| 60 | n.d. | 381 | 1.88 | 3 |
| 61 | 62.45 (A) | 381 | 1.86 | 3 |
| 62 | n.d. | 395 | 1.91 | 1 |
| 63 | n.d. | 384 | 1.70/1.73 | 1 |
| 64 | n.d. | 384 | 1.71 | 1 |
| 65 | n.d. | 384 | 1.70 | 1 |
| 66 | n.d. | 401 | 2.67/2.69 | 1 |
| 67 | n.d. | 438 | 2.15/2.18 | 1 |
| 68 | n.d. | 439 | 2.84 | 1 |
| 69 | n.d. | 422 | 1.98/1.99 | 1 |
| 99 | 235.3 (B) | 454 | 1.66/1.68 | 5 |
| 108 | n.d. | 384 | 2.24 | 3 |
| 109 | n.d. | 384 | 2.24 | 3 |
| 110 | n.d. | 400 | 1.81 | 1 |
| 111 | n.d. | 384 | 1.45 | 1 |
| 112 | n.d. | 384 | 1.46 | 1 |
| 113 | n.d. | 355 | 1.16 | 1 |
| 114 | n.d. | 385 | 1.31/1.33 | 1 |
| 126 | n.d. | 381.2 | 1.93 | 1 |
| 127 | n.d. | 381.2 | 1.82 | 1 |
| 130 | n.d. | 368.2 | 1.57 | 1 |
| 131 | n.d. | 368.2 | 2.36 | 3 |
| 132 | n.d. | 368.2 | 2.36 | 3 |
| 133 | n.d. | 371.2 | 1.74/1.76 | 1 |
| 134 | n.d. | 377.9 | 3.04 | 3 |
| 134 | n.d. | 378.2 | 2.13/2.15 | 1 |
| 135 | n.d. | 353.2 | 1.28 | 1 |
| 136 | n.d. | 370.2 | 1.57-1.62 | 1 |
| 137 | n.d. | 353 | 0.86-0.88 | 7 |
| 137 | n.d. | 353.2 | 1.3 | 1 |
| 138 | n.d. | 424.2 | 2.33-2.36 | 1 |
| 139 | n.d. | 384.2 | 1.5 | 1 |
| 139 | n.d. | 384.2 | 2.36 | 3 |
| free | | 442.5 | | |
| base | | [M + CH3COO]− | | |
| 139 | n.d. | 384.2 | 1.37 | 1 |
| 139 | n.d. | 384.2 | 1.37 | 1 |
| 140 | n.d. | 384.2 | 1.49 | 1 |
| 140 | n.d. | 384.2 | 2.33 | 3 |
| free | | 442.5 | | |
| base | | [M + CH3COO]− | | |
| 140 | n.d. | 384.2 | 1.36 | 1 |
| 141 | n.d. | 425.1855 | 2.95 | 1 |
| 141 | n.d. | 425.1 | 3.57 | 3 |
| | | 483.1 | | |
| | | [M + CH3COO]− | | |
| 142 | n.d. | 425.1846 | 2.96 | 1 |
| 142 | n.d. | 425 | 3.57 | 3 |
| | | 480.0 | | |
| | | [M + CH3COO]− | | |
| 143 | n.d. | 425.1848 | 2.98 | 1 |
| 143 | n.d. | 425.1 | 3.57 | 3 |
| | | 483.2 | | |
| | | [M + CH3COO]− | | |
| 144 | n.d. | 425.1851 | 2.98 | 1 |
| 145 | n.d. | 383.2 | 1.23 | 1 |
| 146 | n.d. | 383.2 | 1.3 | 1 |
| 146 | n.d. | 383.2 | 1.23 | 1 |
| 147 | n.d. | 383.2 | 1.29 | 1 |
| 148 | n.d. | 356.2 | 1.73-1.80 | 1 |
| 149 | n.d. | 354.2 | 1.40-1.42 | 1 |
| 149 | n.d. | 354.1 | 2.28 | 3 |
| 149 | n.d. | 354.2 | 1.4 | 1 |
| 150 | n.d. | 354.2 | 1.41-1.42 | 1 |
| 150 | n.d. | 354.1 | 2.3 | 3 |
| 150 | n.d. | 354.2 | 1.42-1.43 | 1 |
| 151 | n.d. | 372.2 | 1.77/1.78 | 1 |
| 152 | n.d. | 402.2 | 1.18 and 1.20 | 8 |
| 152 | n.d. | 402.3 | 2.61, 2.66 | 3 |
| | | 462.2 | | |
| | | [M + CH3COO]− | | |
| 152 | n.d. | 402.3 | 1.98 and 2.02 | 9 |
| 152 | n.d. | 402.3 | 2.68 | 3 |
| | | 460.3 | | |
| | | [M + CH3COO]− | | |
| 152 | n.d. | 402.2 | 1.78/1.81 | 1 |
| 152 | n.d. | 402.2 | 1.74/1.76 | 1 |
| 153 | n.d. | 402.2 | 2.63 | 3 |
| | | 462.3 | | |
| | | [M + CH3COO]− | | |
| 153 | n.d. | 402.2 | 2.62 | 3 |
| 153 | n.d. | 402.7 | 2.45 | 10 |
| 153 | n.d. | 402.2 | 1.73 | 1 |
| 154 | n.d. | 402.2 | 2.65 | 3 |
| 154 | n.d. | 402.9 | 2.53 | 10 |
| 154 | n.d. | 402.2 | 1.78 | 1 |
| 155 | n.d. | 402.2 | 1.78 | 1 |
| 155 | n.d. | 402.2 | 1.86 | 1 |
| 155 | 148.18 (A) | 402.2 | 1.82 | 1 |
| | 164.66 (A) | | | |
| 155 | 255.69 (A) | 402.2 | 1.73 | 1 |
| 156 | n.d. | 402.2 | 1.86 | 1 |
| 156 | n.d. | 402.2 | 1.84 | 1 |
| 156 | n.d. | 402.2 | 1.76 | 1 |
| 157 | n.d. | 384.1 | 1.77/1.80 | 11 |
| 158 | n.d. | 354.2 | 1.42 | 1 |
| 159 | n.d. | 354.2 | 1.41 | 1 |
| 160 | n.d. | 354.2 | 1.44 | 1 |
| 161 | n.d. | 354.2 | 1.47 | 1 |
| 162 | n.d. | 385.2 | 1.34 | 1 |
| 163 | n.d. | 385.2 | 1.36 | 1 |
| 164 | n.d. | 372.2 | 1.77 | 1 |
| 165 | n.d. | 372.2 | 1.78 | 1 |
| 166 | n.d. | 384.2 | 1.43 | 1 |
| 167 | n.d. | 384.2 | 1.47 | 1 |
| 168 | n.d. | 402.3 | 2.62, 2.71 | 3 |
| | | 460.2 | | |
| | | [M + CH3COO]− | | |
| 168 | n.d. | 402.2 | 1.78/1.87 | 1 |
| 169 | n.d. | 402.2 | 1.85 | 1 |
| 170 | n.d. | 402.2 | 1.78 | 1 |
| 171 | n.d. | 356.6 | 2.51 | 10 |
| 171 | n.d. | 356.2 | 1.72 | 1 |
| 172 | n.d. | 398.2 | 1.41/1.42/1.47 | 1 |
| 173 | n.d. | 384.2 | 0.86/0.88/0.94 | 1 |
| 174 | n.d. | 404.4 | 1.31 | 7 |
| 174 | n.d. | 404.4 | 1.31 | 7 |
| free | | | | |
| base | | | | |
| 175 | n.d. | 404.4 | 1.28 | 7 |
| 175 | n.d. | 404.4 | 1.28 | 7 |
| free | | | | |
| base | | | | |
| 176 | n.d. | 386.3 | 1.23 | 8 |
| 176 | n.d. | 386.3 | 1.15 | 7 |
| free | | | | |
| base | | | | |
| 177 | n.d. | 386.4 | 1.19 | |
| 177 | n.d. | 386.3 | 1.11 | 7 |
| free | | | | |
| base | | | | |
| 178 | 140.57/−51.10 | 386.3 | 2.55, 2.58 | 3 |
| | J/g (A) | 446.2 | | |
| | | [M + CH3COO]− | | |
| 178 | n.d. | 386.3 | 1.12 | 7 |
| 178 | n.d. | 386.3 | 1.11 | 7 |
| 178 | n.d. | 386.3 | 1.11 | 7 |
| 179 | 145.86/−78.37 | 386.4 | 1.12 | 7 |
| | J/g (A) | | | |
| | 145.86/−72.80 | | | |
| | J/g (A) | | | |
| 179 | n.d. | 386.2 | 2.59 | 3 |
| | | 446.0 | | |
| | | [M + CH3COO]− | | |
| 180 | 226.27/−254.17 | 386.3 | 1.12 | 7 |
| | J/g (A) | | | |
| | 226.27/−227.02 | | | |
| | J/g (A) | | | |
| 180 | n.d. | 386.2 | 2.59 | 3 |
| free | | 446.1 | | |
| base | | [M + CH3COO]− | | |
| 181 | n.d. | 401.23 | 1.16/1.18 | 1 |
| 182 | n.d. | 417.23 | 1.55 | 1 |
| 183 | n.d. | 400.2 | 1.83 | 1 |
| 183 | n.d. | 400.6 | 2.83 | 3 |
| | | 458.3 | | |
| | | [M + CH3COO]− | | |
| 184 | n.d. | 400.2 | 1.79 | 1 |
| 184 | n.d. | 400.6 | 2.81 | 3 |
| | | 458.3 | | |
| | | [M + CH3COO]− | | |
| 185 | n.d. | 372.2 | 1.74, 1.77 | 1 |
| 186 | n.d. | 372.2 | 1.78 | 1 |
| 187 | n.d. | 408.2 | 2.31 | 1 |
| 188 | n.d. | 390.2 | 1.92/1.95 | 1 |
| 189 | n.d. | 390.2 | 2.23 | 1 |
| 190 | n.d. | 370.2 | 1.75 | 1 |
| 191 | n.d. | 400.2 | 1.72 | 1 |
| 192 | n.d. | 385.2 | 1.07 | 5 |
| 193 | n.d. | 401.2 | 0.93 | 5 |
| 194 | n.d. | 383.2 | 0.67 | 5 |
| 195 | n.d. | 390.2 | 2.08 | 5 |
| 196 | n.d. | 390.2 | 1.4 | 5 |
| 197 | n.d. | 372.2 | 1.28 | 5 |
| 198 | n.d. | 399.2 | 1.31 | 5 |

Optical Rotations

Optical rotations were measured on a Perkin-Elmer 341 polarimeter with a sodium lamp and reported as follows: [α]° (k, c g/100 ml, solvent, T ° C.).

[α]_{λ}^{T}=(100α)/(l×c):

where l is the path length in dm and c is the concentration in g/100 ml for a sample at a temperature T (° C.) and a wavelength λ (in nm). If the wavelength of light used is 589 nm (the sodium D line), then the symbol D might be used instead. The sign of the rotation (+ or −) should always be given. When using this equation, the concentration and solvent are always provided in parentheses after the rotation. The rotation is reported using degrees and no units of concentration are given (it is assumed to be g/100 mL).

TABLE 4
Optical Rotation data.

| Co. | α_{D} | Wavelength | Concentration | | Temp. |
|---|---|---|---|---|---|
| No. | (°) | (nm) | w/v % | Solvent | (° C.) |
| 39 | −5.3 | 589 | 0.5 | DMF | 20 |
| 43 | −15.3 | 589 | 0.95 | DMF | 20 |
| 45 | +3.8 | 589 | 1.1 | DMF | 20 |
| 46 | −45.1 | 589 | 1 | DMF | 20 |
| 48 | −18.7 | 589 | 0.53 | DMF | 20 |
| 51 | +12.7 | 589 | 0.71 | DMF | 20 |
| 52 | −16.6 | 589 | 1.02 | DMF | 20 |
| 53 | −14.0 | 589 | 0.85 | DMF | 20 |
| 60 | +12.0 | 589 | 2.01 | DMF | 20 |
| 61 | −28.1 | 589 | 1.2 | DMF | 20 |
| 127 | −8.5 | 589 | 1.37 | DMF | 20 |
| 131 | +2.2 | 589 | 1.2 | DMF | 20 |
| 132 | −35.6 | 589 | 0.51 | DMF | 20 |

SFCMS-Methods

General Procedure for SFC-MS Methods

The SFC measurement was performed using an Analytical Supercritical fluid chromatography (SFC) system composed by a binary pump for delivering carbon dioxide (CO₂) and modifier, an autosampler, a column oven, a diode array detector equipped with a high-pressure flow cell standing up to 400 bars. If configured with a Mass Spectrometer (MS) the flow from the column was brought to the (MS). It is within the knowledge of the skilled person to set the tune parameters (e.g. scanning range, dwell time . . . ) in order to obtain ions allowing the identification of the compound's nominal monoisotopic molecular weight (MW). Data acquisition was performed with appropriate software.

TABLE 5
Analytical SFC-MS Methods (Flow expressed in mL/min; column
temperature (T) in ° C.; run time in minutes; backpressure (BPR) in bars.

| | | | | | Run |
|---|---|---|---|---|---|
| Method | | | | Flow | time |
| code | Column | Mobile phase | Gradient | Col T | BPR |
| 1 | Daicel Chiralpak ® | A:CO₂ | 20% | 3.5 | 3 |
| | AD-3 column | B:MeOH | B hold | 35 | 103 |
| | (3 μm, 100 × 4.6 mm) | (+0.3% iPrNH₂) | 3 min, | | |
| 2 | Daicel Chiralpak ® | A:CO₂ | 20% | 3.5 | 4 |
| | AD-3 column | B:MeOH | B hold | 35 | 103 |
| | (3 μm, 100 × 4.6 mm) | (+0.3% iPrNH₂) | 4 min, | | |
| 3 | Daicel Chiralpak ® | A:CO₂ | 10% | 3.5 | 6 |
| | AD-3 column | B:EtOH | B hold | 35 | 103 |
| | (3 μm, 100 × 4.6 mm) | (+0.3% iPrNH₂) | 6 min, | | |
| 4 | Daicel Chiralpak ® | A:CO₂ | 15% | 3.5 | 3 |
| | AD-3 column | B:EtOH | B hold | 35 | 103 |
| | (3 μm, 100 × 4.6 mm) | (+0.3% iPrNH₂) | 3 min, | | |
| 5 | Daicel Chiralpak ® | A:CO₂ | 20% | 3.5 | 3 |
| | AD-3 column | B:EtOH | B hold | 35 | 103 |
| | (3 μm, 100 × 4.6 mm) | (+0.3% iPrNH₂) | 3 min, | | |
| 6 | Daicel Chiralpak ® | A:CO₂ | 10% | 3.5 | 6 |
| | AD-3 column | B:iPrOH | B hold | 35 | 103 |
| | (3 μm, 100 × 4.6 mm) | (+0.3% iPrNH₂) | 6 min, | | |
| 7 | Daicel Chiralpak ® | A:CO₂ | 15% | 3.5 | 3 |
| | AD-3 column | B:iPrOH | B hold | 35 | 103 |
| | (3 μm, 100 × 4.6 mm) | (+0.3% iPrNH₂) | 3 min, | | |
| 8 | Daicel Chiralce1 ® | A:CO₂ | 10% | 3.5 | 3 |
| | OJ-3 column | B:MeOH | B hold | 35 | 103 |
| | (3 μm, 100 × 4.6 mm) | (+0.3% iPrNH₂) | 3 min, | | |
| 9 | Daicel Chiralce1 ® | A:CO₂ | 20% | 3.5 | 3 |
| | OJ-3 column | B:MeOH | B hold | 35 | 103 |
| | (3 μm, 100 × 4.6 mm) | (+0.3% iPrNH₂) | 3 min, | | |
| 10 | Daicel Chiralce1 ® | A:CO₂ | 25% | 3.5 | 3 |
| | OJ-3 column | B:MeOH | B hold | 35 | 103 |
| | (3 μm, 100 × 4.6 mm) | (+0.3% iPrNH₂) | 3min, | | |
| 11 | Daicel Chiralce1 ® | A:CO₂ | 10% | 3.5 | 6 |
| | OJ-3 column | B:EtOH | B hold | 35 | 103 |
| | (3 μm, 100 × 4.6 mm) | (+0.3% iPrNH₂) | 6 min, | | |
| 12 | Daicel Chiralce1 ® | A:CO₂ | 20% | 3.5 | 3 |
| | OJ-3 column | B:iPrOH | B hold | 35 | 103 |
| | (3 μm, 100 × 4.6 mm) | (+0.3% iPrNH₂) | 3 min, | | |
| 13 | Daicel Chiralce1 ® | A:CO₂ | 25% | 3.5 | 3 |
| | OJ-3 column | B:iPrOH | B hold | 35 | 103 |
| | (3 μm, 100 × 4.6 mm) | (+0.3% iPrNH₂) | 3 min, | | |
| 14 | Daicel Chiralpak ® | A:CO₂ | 40% | 3.5 | 3 |
| | IC-3 column | B:iPrOH | B hold | 35 | 103 |
| | (3 μm, 100 × 4.6 mm) | (+0.3% iPrNH₂) | 3 min, | | |
| 15 | Daicel Chiralpak ® | A:CO₂ | 50% | 3.5 | 6 |
| | IC-3 column | B:iPrOH | B hold | 35 | 103 |
| | (3 μm, 100 × 4.6 mm) | (+0.3% iPrNH₂) | 6 min, | | |
| 16 | Daicel Chiralce1 ® | A:CO₂ | 20% | 3.5 | 3 |
| | OD-3 column | B:iPrOH | B hold | 35 | 103 |
| | (3 μm, 100 × 4.6 mm) | (+0.3% iPrNH₂) | 3 min, | | |
| 17 | Phenomenex Lux | A:CO₂ | 30% | 3.5 | 3 |
| | cellulose 4 column | B:EtOH | B hold | 35 | 103 |
| | (3 μm, 100 × 4.6 mm) | (+0.3% iPrNH₂) | 3 min, | | |
| 18 | Phenomenex Lux | A:CO₂ | 30% | 3.5 | 6 |
| | cellulose 4 column | B:iPrOH | B hold | 35 | 103 |
| | (3 μm, 100 × 4.6 mm) | (+0.3% iPrNH₂) | 6 min, | | |
| 19 | Daicel Chiralpak ® | A:CO₂ | 30% | 3.5 | 3 |
| | IC-3 column | B:iPrOH | B hold | 35 | 103 |
| | (3 μm, 100 × 4.6 mm) | (+0.3% iPrNH₂) | 3 min, | | |
| 20 | Daicel Chiralpak ® | A:CO₂ | 5% | 3.5 | 6 |
| | AD-3 column | B:iPrOH | B hold | 35 | 103 |
| | (3 μm, 100 × 4.6 mm) | (+0.3% iPrNH₂) | 6 min, | | |
| 21 | Daicel Chiralce1 ® | A:CO₂ | 10% | 3.5 | 3 |
| | OD-3 column | B:iPrOH | B hold | 35 | 103 |
| | (3 μm, 100 × 4.6 mm) | (+0.3% iPrNH₂) | 3 min, | | |
| 22 | Daicel Chiralcel ® | A:CO₂ | 15% | 3.5 | 3 |
| | OJ-3 column | B:MeOH | B hold | 35 | 103 |
| | (3 μm, 100 × 4.6 mm) | (+0.3% iPrNH₂) | 3 min, | | |
| 23 | Daicel Chiralpak ® | A:CO₂ | 35% | 3.5 | 3 |
| | IC-3 column | B:iPrOH | B hold | 35 | 103 |
| | (3 μm, 100 × 4.6 mm) | (+0.3% iPrNH₂) | 3 min, | | |
| 24 | Daicel Chiralpak ® | A:CO₂ | 20% | 3.5 | 3 |
| | AD-3 column | B:iPrOH | B hold | 35 | 103 |
| | (3 μm, 100 × 4.6 mm) | (+0.3% iPrNH₂) | 3 min, | | |
| 25 | Phenomenex Lux | A:CO₂ | 30% | 3.5 | 6 |
| | cellu1ose2 column | B:MeOH | B hold | 35 | 103 |
| | (3 μm, 100 × 4.6 mm) | (+0.3% iPrNH₂) | 6 min, | | |

TABLE 6
Analytical SFC data Rt means retention time (in minutes), [M + H]⁺
means the protonated mass of the compound, method refers to the
method used for (SFC) MS analysis of enantiomerically pure compounds.

| Co. | | | UV | | Isomer Elution |
|---|---|---|---|---|---|
| Nr. | Rt | [M + H]⁺ | Area % | Method | Order |
| 5 | 1.32 | 337 | 100 | 7 | A |
| 6 | 1.53 | 337 | 100 | 7 | B |
| 25 | 1.67 | 399 | 100 | 15 | A |
| 26 | 2.62 | 399 | 100 | 15 | B |
| 28 | 2.05 | 399 | 100 | 15 | A |
| 29 | 2.82 | 399 | 100 | 15 | B |
| 32 | 1.44 | 437 | 100 | 18 | A |
| 33 | 1.79 | 437 | 100 | 18 | B |
| 34 | 0.70 | 437 | 100 | 9 | A |
| 35 | 0.87 | 437 | 99.5 | 9 | B |
| 41 | 0.92 | 385 | 100 | 16 | A |
| 42 | 1.16 | 385 | 98.4 | 16 | B |
| 45 | 1.52 | 368 | 100 | 14 | A |
| 46 | 2.01 | 368 | 99.7 | 14 | B |
| 51 | 1.67 | 367 | 100 | 2 | A |
| 52 | 2.31 | 367 | 100 | 2 | B |
| 60 | 1.86 | 381 | 99.3 | 11 | A |
| 61 | 2.40 | 381 | 99.4 | 11 | B |
| 108 | 0.54 | 384 | 100 | 10 | A |
| 109 | 1.17 | 384 | 100 | 10 | B |
| 130 | 2.82, | 368 | 53.64, | 3 | |
| | 3.21 | | 46.36 | | |
| 131 | 2.81 | 368 | 100.00 | 3 | A |
| 132 | 3.21 | 368 | 2.13 | 3 | B |
| 134 | 1.29, | 378 | 18.35, | 8 | |
| | 1.38, | | 30.88, | | |
| | 1.52, | | 21.29, | | |
| | 2.24 | | 29.48 | | |
| 139 | 0.60 | 384 | 100.00 | 10 | A |
| free | | | | | |
| base | | | | | |
| 140 | 1.13 | 384 | 99.94 | 10 | B |
| free | | | | | |
| base | | | | | |
| 141 | 1.24 | 425 | 100.00 | 20 | C |
| 142 | 1.14 | 425 | 100.00, | 21 | A |
| 143 | 1.32 | 425 | 96.21 | 21 | B |
| 149 | 0.89, | 354 | 51.15, | 22 | |
| | 1.42 | | 48.85 | | |
| 150 | 1.78, | 354 | 50.63, | 23 | |
| | 2.07 | | 49.37 | | |
| 152 | 1.34, | 402 | 52.54 | 7 | |
| | 1.78 | | 47.46 | | |
| 152 | 1.26, | 402 | 48.96 | 7 | |
| | 1.64 | | 51.04 | | |
| 153 | 1.71 | 402 | 98.91 | 7 | B |
| 153 | 1.56 | 402 | 98.76 | 7 | B |
| 153 | 1.63 | 402 | 100.00 | 7 | B |
| 154 | 1.18 | 402 | 100.00 | 7 | A |
| 154 | 1.24 | 402 | 100.00 | 7 | A |
| 158 | 0.89 | 354 | 100.00, | 22 | A |
| 159 | 1.42 | 354 | 99.67 | 22 | B |
| 160 | 1.78 | 354 | 100.00 | 23 | A |
| 161 | 2.07 | 354 | 100.00 | 23 | B |
| 168 | 0.81, | 402 | 48.64, | 24 | |
| | 1.00 | | 51.36 | | |
| 169 | 0.81 | 402 | 100.00 | 24 | A |
| 170 | 1.00 | 402 | 99.71 | 24 | B |
| 171 | 1.48, | 356, | 51.71, | 25 | |
| | 1.81 | 387 | 48.29 | | |
| 178 | 1.57, | 386 | 46.90, | 7 | |
| | 1.84 | | 53.10 | | |
| 179 | 1.56 | 386 | 100.00 | 7 | A |
| 180 | 1.82 | 386 | 98.33 | 7 | B |
| free | | | | | |
| base | | | | | |
| 183 | 0.97 | 400 | 100.00 | 9 | A |
| 184 | 1.33 | 400 | 100.00 | 9 | B |

NMR

For a number of compounds, ¹H NMR spectra were recorded on a Bruker Avanice III with a 300 MHz Ultrashield magnet, on a Bruker DPX-400 spectrometer operating at 400 MHz, on a Bruker Avanice I operating at 500 MHz, on a Bruker DPX-360 operating at 360 MHz, or on a Bruker Avanice 600 spectrometer operating at 600 MHz, using CHLOROFORM-d (deuterated chloroform, CDCl₃) or DMSO-d₆ (deuterated DMSO, dimethyl-d₆ sulfoxide) as solvent. Chemical shifts (6) are reported in parts per million (ppm) relative to tetramethylsilane (TMS), which was used as internal standard.

TABLE 6A
¹H NMR results

| Co. | |
|---|---|
| No. | ¹H NMR result |
| 155 | ¹H NMR (500 MHz, DMSO-d6) δ ppm 1.14-1.27 (m, 1 H) 1.62 (d, J = 6.71 |
| | Hz, 3 H) 1.74-1.94 (m, 3 H) 2.61 (s, 6 H) 2.64-2.82 (m, 3 H) 3.42 (br d, |
| | J = 12.05 Hz, 1 H) 3.54 (br s, 1 H) 4.06-4.22 (m, 2 H) 4.26-4.35 (m, 2 H) |
| | 4.49-4.75 (m, 1 H) 7.28 (s, 2 H) 7.53 (d, J = 9.61 Hz, 1 H) 11.25 (br s, 1 H) |
| | 15.31 (br s, 1 H) |
| 170 | ¹H NMR (400 MHz, DMSO-d6) δ ppm 1.49-1.80 (m, 5 H) 1.81-2.32 |
| | (m, 3 H) 2.61-2.88 (m, 7 H) 3.08 (br t, J = 11.91 Hz, 1 H) 3.55-3.80 (m, 1 H) |
| | 4.29-4.54 (m, 4 H) 4.65-4.91 (m, 3 H) 7.43-7.65 (br s, 1 H) 7.92 (br s, 2 H) |
| | 9.89-10.47 (m 1 H) 10.91-11.39 (m, 1 H) |
| 182 | ¹H NMR (400 MHz, CHLOROFORM-d) δ ppm 1.42 (dd, J = 6.94, 2.31 Hz, 3 H) |
| | 1.45-1.59 (m, 1 H) 1.64-1.74 (m, 2 H) 1.76-1.94 (m, 1 H) 1.95-2.07 |
| | (m, 1 H), 2.13-2.24 (m, 1 H) 2.30 (s, 3 H) 2.74-3.11 (m, 4 H) 3.86 (d, |
| | J = 0.92 Hz, 3 H) 4.04 (ddd, J = 10.23, 6.99, 1.50 Hz, 1 H) 4.17-4.49 (m, 5 H) |
| | 5.63 (d, J = 1.62, 1 H) 5.97 (dd, J = 5.32, 1.39 Hz, 1 H) 6.95 |
| | (dd, J = 9.13, 5.43 Hz, 1 H) |
| 132 | ¹H NMR (400 MHz, CHLOROFORM-d) δ ppm 0.83-0.95 (m, 1 H) 1.31 |
| | (d, J = 6.94 Hz, 3 H) 1.38-1.98 (m, 7 H) 2.29-2.54 (m, 7 H) 2.65-2.82 |
| | (m, 2 H) 3.46 (d, J = 6.78 Hz, 1 H) 4.21-4.31 (m, 2 H) 4.36-4.48 (m, 2 H) |
| | 6.74 (s, H) 7.13 (d, J = 1.85 Hz, 1 H) 7.69 (d, J = 2.08 Hz, 1 H) |
| 146 | ¹H NMR (500 MHz, CHLOROFORM-d) δ ppm 1.01-0.21 (m, 1 H) |
| | 1.31-1.41 (m, 3 H) 1.45-1.71 (m, 3 H) 1.82-2.23 (m, 3 H) 2.41-2.52 |
| | (m, 6 H) 2.63-3.04 (m, 2 H) 3.28-3.47 (m, 1 H) 3.52-3.63 (m, 2 H) 3.70-3.91 |
| | (m, 2 H) 4.16-4.29 (m, 2 H) 4.72 (br s, 1 H) 6.40-6.55 (m, 2 H) 6.88-7.06 |
| | (m, 1 H) 7.50-7.61 (m, 1 H) |
| 163 | ¹H NMR (400 MHz, CHLOROFORM-d) δ ppm 0.99-1.16 (m, 1 H) 1.36 |
| | (d, J = 6.94 Hz, 3 H) 1.45-1.83 (m, 3 H) 1.87-2.23 (m, 3 H) 2.39 (s, 3 H) |
| | 2.56 (s, 3 H) 2.68-3.12 (m, 2 H) 3.54 (q, J = 6.94 Hz, 1 H) 4.10-4.22 |
| | (m, 2 H) 4.22-4.31 (m, 2 H) 4.37-4.51 (m, 2 H) 6.35 (s, 1 H) 6.92 |
| | (d, J = 8.09 Hz, 1 H) 7.11 (d, J = 8.09 Hz, 1 H) |
| 158 | ¹H NMR (500 MHz, DMSO-d6) δ ppm 1.66 (d, J = 6.65 Hz, 4 H) 1.85-2.17 |
| | (m, 3 H) 2.71 (s, 6 H) 2.80-3.19 (m, 2 H) 3.52-3.83 (m, 2 H) 4.30 |
| | (dd, J = 4.48, 3.61 Hz, 2 H) 4.49-4.52 (m, 3 H) 7.22 (d, J = 8.09 Hz, 1 H) |
| | 7.67 (s, 2 H) 11.14 (br s, 2 H) 14.29-16.64 (m, 1 H) |

### Pharmacological Examples

1) OGA—Biochemical Assay

The assay is based on the inhibition of the hydrolysis of fluorescein mono-β-D-N-Acetyl-Glucosamine (FM-GlcNAc) (Mariappa et al. 2015, Biochem J 470:255) by the recombinant human Meningioma Expressed Antigen 5 (MGEA5), also referred to as O-GlcNAcase (OGA). The hydrolysis FM-GlcNAc (Marker Gene technologies, cat #M1485) results in the formation of β-D-N-glucosamineacetate and fluorescein. The fluorescence of the latter can be measured at excitation wavelength 485 nm and emission wavelength 538 nm. An increase in enzyme activity results in an increase in fluorescence signal. Full length OGA enzyme was purchased at OnGene (cat #TP322411). The enzyme was stored in 25 mM Tris.HCl, pH 7.3, 100 mM glycine, 10% glycerol at −20° C. Thiamet G and GlcNAcStatin were tested as reference compounds (Yuzwa et al. 2008 Nature Chemical Biology 4:483; Yuzwa et al. 2012 Nature Chemical Biology 8:393). The assay was performed in 200 mM Citrate/phosphate buffer supplemented with 0.005% Tween-20. 35.6 g Na₂HPO₄2H₂O (Sigma, #C0759) were dissolved in 1 L water to obtain a 200 mM solution. 19.2 g citric acid (Merck, #1.06580) was dissolved in 1 L water to obtain a 100 mM solution. pH of the sodiumphosphate solution was adjusted with the citric acid solution to 7.2. The buffer to stop the reaction consists of a 500 mM Carbonate buffer, pH 11.0. 734 mg FM-GlcNAc were dissolved in 5.48 mL DMSO to obtain a 250 mM solution and was stored at −20° C. OGA was used at a 2 nM concentration and FM-GlcNAc at a 100 uM final concentration. Dilutions were prepared in assay buffer.

50 nl of a compound dissolved in DMSO was dispensed on Black Proxiplate TM 384 Plus Assay plates (Perkin Elmer, #6008269) and 3 μl fl-OGA enzyme mix added subsequently. Plates were pre-incubated for 60 min at room temperature and then 2 μl FM-GlcNAc substrate mix added. Final DMSO concentrations did not exceed 1%. Plates were briefly centrifuged for 1 min at 1000 rpm and incubate at room temperature for 6 h. To stop the reaction 5 μl STOP buffer were added and plates centrifuge again 1 min at 1000 rpm. Fluorescence was quantified in the Thermo Scientific Fluoroskan Ascent or the PerkinElmer EnVision with excitation wavelength 485 nm and emission wavelength 538 nm.

For analysis a best-fit curve is fitted by a minimum sum of squares method. From this an IC₅₀ value and Hill coefficient was obtained. High control (no inhibitor) and low control (saturating concentrations of standard inhibitor) were used to define the minimum and maximum values.

2) OGA—Cellular Assay

HEK293 cells inducible for P301L mutant human Tau (isoform 2N4R) were established at Janssen. Thiamet-G was used for both plate validation (high control) and as reference compound (reference EC₅₀ assay validation). OGA inhibition is evaluated through the immunocytochemical (ICC) detection of O-GlcNAcylated proteins by the use of a monoclonal antibody (CTD110.6; Cell Signaling, #9875) detecting 0-GlcNAcylated residues as previously described (Dorfmueller et al. 2010 Chemistry & biology, 17:1250). Inhibition of OGA will result in an increase of O-GlcNAcylated protein levels resulting in an increased signal in the experiment. Cell nuclei are stained with Hoechst to give a cell culture quality control and a rough estimate of immediate compounds toxicity, if any. ICC pictures are imaged with a Perkin Elmer Opera Phenix plate microscope and quantified with the provided software Perkin Elmer Harmony 4.1.

Cells were propagated in DMEM high Glucose (Sigma, #D5796) following standard procedures. 2 days before the cell assay cells are split, counted and seeded in Poly-D-Lysine (PDL) coated 96-wells (Greiner, #655946) plate at a cell density of 12,000 cells per cm² (4,000 cells per well) in 100 μl of Assay Medium (Low Glucose medium is used to reduce basal levels of GlcNAcylation) (Park et al. 2014 The Journal of biological chemistry 289:13519). At the day of compound test medium from assay plates was removed and replenished with 90 μl of fresh Assay Medium. 10 μl of compounds at a 10 fold final concentration were added to the wells. Plates were centrifuged shortly before incubation in the cell incubator for 6 hours. DMSO concentration was set to 0.2%. Medium is discarded by applying vacuum. For staining of cells medium was removed and cells washed once with 100 μl D-PBS (Sigma, #D8537). From next step onwards unless other stated assay volume was always 50 μl and incubation was performed without agitation and at room temperature. Cells were fixed in 50 μl of a 4% paraformaldehyde (PFA, Alpha aesar, #043368) PBS solution for 15 minutes at room temperature. The PFA PBS solution was then discarded and cells washed once in 10 mM Tris Buffer (LifeTechnologies, #15567-027), 150 mM NaCl (LifeTechnologies, #24740-0110, 0.1% Triton X (Alpha aesar, #A16046), pH 7.5 (ICC buffer) before being permeabilized in same buffer for 10 minutes. Samples are subsequently blocked in ICC containing 5% goat serum (Sigma, #G9023) for 45-60 minutes at room temperature. Samples were then incubated with primary antibody (1/1000 from commercial provider, see above) at 4° C. overnight and subsequently washed 3 times for 5 minutes in ICC buffer. Samples were incubated with secondary fluorescent antibody (1/500 dilution, Lifetechnologies, #A-21042) and nuclei stained with Hoechst 33342 at a final concentration of 1 μg/ml in ICC (Lifetechnologies, #H3570) for 1 hour. Before analysis samples were washed 2 times manually for 5 minutes in ICC base buffer.

Imaging is performed using Perkin Elmer Phenix Opera using a water 20× objective and recording 9 fields per well. Intensity readout at 488 nm is used as a measure of O-GlcNAcylation level of total proteins in wells. To assess potential toxicity of compounds nuclei were counted using the Hoechst staining. IC₅₀-values are calculated using parametric non-linear regression model fitting. As a maximum inhibition Thiamet G at a 200 uM concentration is present on each plate. In addition, a concentration response of Thiamet G is calculated on each plate.

TABLE 7
Results in the biochemical and cellular assays.

| | Enzymatic | Enzymatic | Cellular | Cellular |
|---|---|---|---|---|
| Co. | hOGA; | Eₘₐₓ | hOGA; | Eₘₐₓ |
| No. | pIC₅₀ | (%) | pEC₅₀ | (%) |
| 1 | 7.7 | 102 | 7.0 | 77 |
| 2 | 8.3 | 100 | 7.6 | 89 |
| 3 | 6.1 | 97 | | |
| 4 | 8.3 | 103 | 7.7 | 70 |
| 5 | 8.5 | 102 | | |
| 6 | 6.9 | 101 | | |
| 7 | 7.9 | 103 | 7.0 | 77 |
| 8 | 7.8 | 100 | 6.7 | 86 |
| 9 | 8.0 | 102 | 7.3 | 92 |
| 10 | 7.6 | 100 | 6.01 | 52 |
| 11 | 7.7 | 103 | 6.5 | 70 |
| 12 | 6.3 | 96 | | |
| 14 | 8.3 | 101 | 6.2 | 60 |
| 15 | 7.7 | 103 | <6 | 16 |
| 17 | 7.1 | 99 | | |
| 18 | 7.3 | 100 | 6.1 | 57 |
| 19 | 8.1 | 102 | 7.5 | 86 |
| 20 | 6.5 | 98 | | |
| 21 | 8.3 | 103 | 7.8 | 88 |
| 22 | 8.1 | 104 | 8.1 | 82 |
| 23 | 8.0 | 100 | 8.1 | 81 |
| 24 | 8.1 | 102 | 8.1 | 69 |
| 25 | 7.1 | 103 | | |
| 26 | 8.6 | 101 | 8.3 | 85 |
| 27 | 6.7 | 102 | | |
| 28 | 6.0 | 99 | | |
| 29 | 7.1 | 97 | | |
| 30 | 8.1 | 104 | 8.2 | 81 |
| 32 | 6.4 | 99 | | |
| 33 | 8.6 | 102 | 7.5 | 83 |
| 34 | 6.7 | 100 | | |
| 35 | 5.6 | 88 | | |
| 36 | 7.1 | 101 | | |
| 36 | 8.1 | 101 | 6.9 | 88 |
| 37 | 8.3 | 102 | | |
| 38 | 8.2 | 102 | | |
| 39 | 6.1 | 92 | | |
| 40 | 8.7 | 101 | 7.8 | 96 |
| 41 | 6.0 | 94 | | |
| 42 | 8.7 | 99 | 8.2 | 86 |
| 43 | 7.2 | 101 | 6.3 | 60 |
| 44 | 8.5 | 101 | 7.7 | 91 |
| 45 | 6.3 | 98 | | |
| 46 | 8.5 | 101 | 7.8 | 90 |
| 47 | 8.4 | 103 | 7.8 | 81 |
| 48 | 6.3 | 93 | <6 | 5 |
| 49 | 6.9 | 103 | <6 | |
| 50 | 8.0 | 103 | 7.4 | 85 |
| 51 | 6.7 | 101 | | |
| 52 | 8.9 | 104 | 8.2 | 92 |
| 53 | 6.1 | 94 | | |
| 54 | 5.5 | 78 | | |
| 55 | 5.9 | 90 | | |
| 56 | 7.8 | 102 | 6.5 | 78 |
| 57 | 7.9 | 101 | 6.8 | 78 |
| 58 | 6.9 | 102 | | |
| 59 | 6.9 | 102 | <6 | 23 |
| 60 | 5.5 | 78 | | |
| 61 | 7.4 | 102 | <6 | 45 |
| 62 | 5.5 | 77 | | |
| 63 | 8.1 | 101 | 7.5 | 69 |
| 64 | 8.4 | 101 | 7.8 | 97 |
| 65 | 6.7 | 102 | | |
| 66 | 8.2 | 102 | 7.5 | 85 |
| 67 | 8.1 | 102 | 7.1 | 89 |
| 68 | 8.2 | 102 | 7.4 | 79 |
| 69 | 7.9 | 102 | 7.3 | 90 |
| 99 | 8.28 | 105 | 7.6 | 65 |
| 108 | <5 | 37 | <6 | −8 |
| 109 | 6.6 | 95 | <6 | 37 |
| 110 | 7.9 | 102 | 6.5 | 68 |
| 111 | 7.9 | 98 | 6.5 | 29 |
| 112 | 6.7 | 98 | <6 | 8 |
| 113 | 7.0 | 100 | 6.1 | 55 |
| 114 | 7.8 | 100 | 6.8 | 66 |
| 126 | <5 | 1 | | |
| 127 | <5 | 10 | | |
| 130 | 8.1 | 101 | 6.8 | 83 |
| 131 | 6.3 | 97 | | |
| 132 | 8.3 | 103 | 7.1 | 77 |
| 133 | 7.8 | 103 | 6.3 | 62 |
| 134 | 6.8 | 96 | | |
| 135 | 8.1 | 101 | 7.6 | 68 |
| 136 | 8.4 | 101 | 6.2 | 60 |
| 137 | 7.7 | 101 | 7.3 | 83 |
| 138 | 6.9 | 101 | <6 | 16 |
| 139 | 8.1 | 98 | 7.7 | 88 |
| 140 | 5.4 | 72 | <6 | 2 |
| 141 | 6.3 | 98 | | |
| 142 | 5.0 | 52 | | |
| 143 | 5.6 | 87 | | |
| 144 | 8.5 | 99 | | |
| 145 | 6.6 | 99 | <6 | 35 |
| 146 | 8.2 | 99 | 8.0 | 79 |
| 147 | 8.2 | 100 | 8.0 | 80 |
| 148 | 7.8 | 98 | 8.0 | 78 |
| 149 | 8.0 | 100 | 7.5 | 93 |
| 150 | 5.7 | 92 | <6 | 3 |
| 151 | 8.2 | 102 | 8.16 | 85 |
| 152 | 8.4 | 102 | 8.4 | 94 |
| 153 | 8.8 | 102 | 8.6 | 94 |
| 154 | 7.3 | 101 | 6.9 | 86 |
| 155 | 8.9 | 99 | 8.59 | 95 |
| 156 | 6.6 | 97 | 6.5 | 55 |
| 157 | 8.3 | 101 | 7.8 | 83 |
| 158 | 8.1 | 102 | 7.4 | 89 |
| 159 | 5.9 | 91 | <6 | 0 |
| 160 | 5.7 | 89 | <6 | 5 |
| 161 | 5.5 | 79 | <6 | −4 |
| 162 | 6.1 | 93 | <6 | 3 |
| 163 | 8.1 | 100 | 7.34 | 82 |
| 164 | 8.5 | 100 | 8.2 | 86 |
| 165 | 7.2 | 100 | 7.0 | 80 |
| 166 | 8.0 | 98 | 7.0 | 67 |
| 167 | 6.8 | 97 | <6 | 26 |
| 168 | 8.5 | 99 | 7.5 | 69 |
| 169 | 5.7 | 85 | <6 | −5 |
| 170 | 8.5 | 98 | 7.9 | 79 |
| 171 | 8.6 | 100 | 8.6 | 78 |
| 172 | 7.4 | 99 | 6.9 | 78 |
| 173 | 7.2 | 99 | 6.2 | 64 |
| 174 | 8.2 | 99 | 7.2 | 75 |
| 175 | 6.0 | 92 | <6 | 1 |
| 176 | 7.8 | 99 | 6.9 | 69 |
| 177 | 5.6 | 81 | <6 | −8 |
| 178 | 8.6 | 95 | 8.6 | 104 |
| 179 | 6.7 | 100 | 6.6 | 72 |
| 180 | 8.8 | 99 | 9.0 | 79 |
| 181 | 8.4 | 101 | 8.8 | 92 |
| 182 | 8.4 | 99 | 8.8 | 106 |
| 183 | 8.1 | 99 | 7.3 | 65 |
| 184 | 5.7 | 87 | <6 | −17 |
| 185 | 7.8 | 100 | 7.3 | 65 |
| 186 | 5.7 | 83 | <6 | −6 |
| 187 | 8.1 | 96 | 7.9 | 92 |
| 188 | 8.0 | 102 | 7.0 | 79 |
| 189 | 8.1 | 100 | 7.8 | 88 |
| 190 | 8.0 | 101 | 7.3 | 82 |
| 191 | 8.3 | 95 | 7.8 | 81 |
| 192 | 8.2 | 99 | 7.7 | 91 |
| 193 | 8.5 | 99 | 7.5 | 95 |
| 194 | 7.7 | 97 | 6.6 | 71 |
| 195 | 7.8 | 100 | 7.1 | 112 |
| 196 | 7.4 | 95 | 6.6 | 70 |
| 197 | 6.4 | 98 | 6.2 | 66 |
| 198 | 7.4 | 94 | 6.4 | 82 |

Ex Vivo Oga Occupancy Assay Using [³H]-Ligand

Drug Treatment and Tissue Preparation

Male NMRI or C57Bl6j mice were treated by oral (p.o.) administration of vehicle or compound. Animals were sacrificed 24 hours after administration. Brains were immediately removed from the skull, hemispheres were separated and the right hemisphere, for ex vivo OGA occupancy assay, was rapidly frozen in dry-ice cooled 2-methylbutane (−40° C.). Twenty m-thick sagittal sections were cut using a Leica CM 3050 cryostat-microtome (Leica, Belgium), thaw-mounted on microscope slides (SuperFrost Plus Slides, Thermo Fisher Scientific) and stored at −20° C. until use. After thawing, sections were dried under a cold stream of air. The sections were not washed prior to incubation. The 10 minutes incubation with 3 nM [³H]-ligand was rigorously controlled. All brain sections (from compound-treated and vehicle-treated animals) were incubated in parallel. After incubation, the excess of [³H]-ligand was washed off in ice-cold buffer (PBS 1× and 1% BSA) 2 times 10 minutes, followed by a quick dip in distilled water. The sections were then dried under a stream of cold air.

Quantitative autoradiography and data analysis Radioactivity in the forebrain area of brain slices was measured using a β-imager with M3 vision analysis software (Biospace Lab, Paris). Specific binding was calculated as the difference between total binding and non-specific binding measured in Thiamet-G (10 μM) treated sections. Specific binding in sections from drug treated animals was normalised to binding in sections from vehicle treated mice to calculate percentage of OGA occupancy by the drug.

TABLE 8
OCCUPANCY OF SELECTED COMPOUNDS

| Co. | Time | Dose | Occupancy |
|---|---|---|---|
| No. | (h) | (mg/kg) | (% +/− sd) |
| 155 | 24 | 25 | 92.67 +/− 2.08 |
| 135 | 24 | 25 | 20.67 +/− 3.21 |

1. A compound of Formula (I) 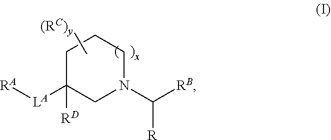
or a tautomer or a stereoisomeric form thereof, wherein
R^{A} is a heteroaryl radical selected from the group consisting of pyridin-2-yl, pyridin-3-yl, pyridin-4-yl, pyridazin-3-yl, pyrimidin-4-yl, pyrimidin-5-yl, and pyrazin-2-yl; or is an aryl radical selected from phenyl; each of which may be optionally substituted with 1, 2 or 3 substituents each independently selected from the group consisting of halo; cyano; C₁₋₄alkyl optionally substituted with 1, 2, or 3 independently selected halo substituents;
—C(O)NR^{a}R^{aa}; NR^{a}R^{aa}; and C₁₋₄alkyloxy optionally substituted with 1, 2, or 3 independently selected halo substituents; wherein R^{a} and R^{aa} are each independently selected from the group consisting of hydrogen and C₁₋₄alkyl optionally substituted with 1, 2, or 3 independently selected halo substituents;
L^{A} is selected from the group consisting of a covalent bond, —CH₂—, —O—, —OCH₂—, —CH₂O—, —NH—, —N(CH₃)—, —NHCH₂— and —CH₂NH—;
x represents 1;
R is H or CH₃; and
R^{B} is a bicyclic radical of formula (b-1), (b-2) or (b-3) 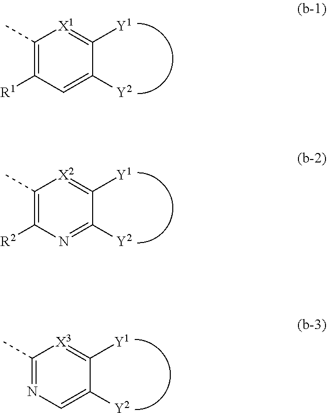
wherein
R¹ and R² are each selected from the group consisting of hydrogen, fluoro and methyl;
X¹, X² and X³ each represent CH, CF or N;
—Y¹—Y²— forms a bivalent radical selected from the group consisting of
—O(CH₂)ₘO— (c-1);
—O(CH₂)ₙ— (c-2);
—(CH₂)ₙO— (c-3);
—O(CH₂)ₚNR³— (c-4);
—NR³(CH₂)_{P}O— (c-5);
—O(CH₂)(CO)NR³— (c-6);
—NR³(CO)(CH₂)O— (c-7);
—(CH₂)ₙNR³(CO)— (c-8);
—(CO)NR³(CH₂)ₙ— (c-9); and
—N═CH(CO)NR³— (c-10);
wherein
m is 1 or 2;
n and p each independently represent 2 or 3;
each R³ is independently H or C₁₋₄alkyl;
R^{C} is selected from the group consisting of fluoro, methyl, hydroxy, methoxy, trifluoromethyl, and difluoromethyl;
R^{D} is selected from the group consisting of hydrogen, fluoro, methyl, hydroxy, methoxy, trifluoromethyl, and difluoromethyl; and
y represents 0, 1 or 2;
with the provisos that
a) R^{C} is not hydroxy or methoxy when present at the carbon atom adjacent to the nitrogen atom of the piperidinediyl or pyrrolidinediyl ring;
b) R^{C} or R^{D} cannot be selected simultaneously from hydroxy or methoxy when R^{C} is present at the carbon atom adjacent to C—R^{D};
c) R^{D} is not hydroxy or methoxy when L^{A} is —O—, —OCH₂—, —CH₂O—, —NH—, —N(CH₃)—, —NH(CH₂)— or —(CH₂)NH—;
or a pharmaceutically acceptable addition salt or a solvate thereof.

2. The compound according to claim 1, wherein
R^{A} is a heteroaryl radical selected from the group consisting of pyridin-2-yl, pyridin-3-yl, pyridin-4-yl, pyridazin-3-yl, pyrimidin-4-yl, pyrimidin-5-yl, and pyrazin-2-yl; or is an aryl radical selected from phenyl; each of which may be optionally substituted with 1, 2 or 3 substituents each independently selected from the group consisting of halo; C₁₋₄alkyl optionally substituted with 1, 2, or 3 independently selected halo substituents; and C₁₋₄alkyloxy optionally substituted with 1, 2, or 3 independently selected halo substituents.

3. The compound according to claim 1, wherein L^{A} is selected from the group consisting of a covalent bond, —CH₂—, —O—, —OCH₂—, —CH₂O—, and —NHCH₂—.

4. The compound according to claim 1, wherein R^{B} is a bicyclic radical of formula (b-1) or (b-2).

5. The compound according to claim 1, wherein R^{B} is a bicyclic radical of formula (b-1) or (b-2), wherein R¹ is selected from the group consisting of hydrogen, fluoro and methyl; R² is hydrogen or fluoro; X¹ is N or CH; and X² is CH.

6. The compound according to claim 1, wherein R^{B} is a bicyclic radical of formula (b-1) or (b-2), wherein R¹ is selected from the group consisting of hydrogen, fluoro and methyl; R² is hydrogen or fluoro; X¹ is N or CH; X² is CH; and —Y¹—Y²— forms a bivalent radical selected from the group consisting of (c-1), (c-2), (c-4) and (c-6), wherein m is 2; n is 2 or 3; and p is 2.

7. The compound according to claim 1, wherein R^{B} is selected from the group consisting of

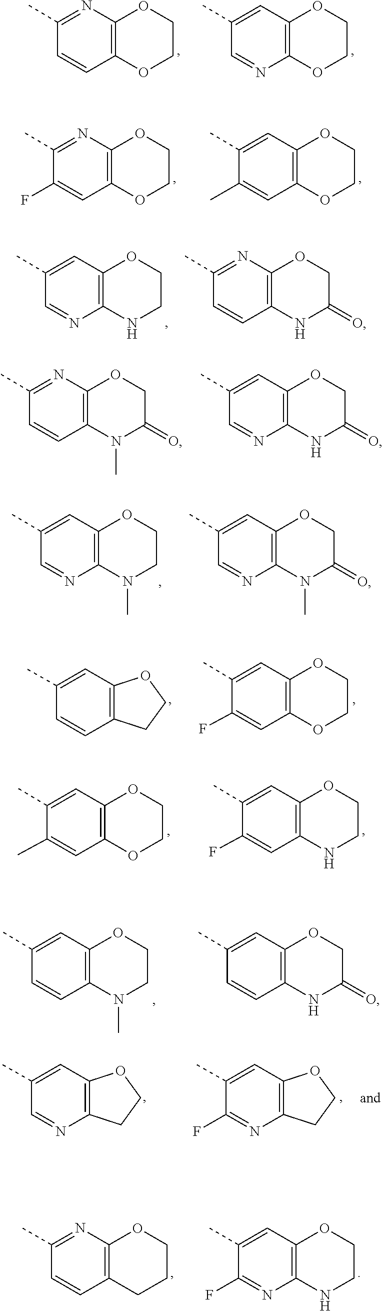

8. The compound according to claim 1, wherein R^{D} is selected from the group consisting of hydrogen, fluoro, and methyl; and y represents 0 or 1.

9. The compound according to claim 1, wherein R^{A} is a heteroaryl radical selected from the group consisting of pyridin-2-yl, pyridin-3-yl, pyridin-4-yl, pyridazin-3-yl, pyrimidin-4-yl, pyrimidin-5-yl, and pyrazin-2-yl; each of which may be optionally substituted with 1, 2 or 3 substituents each independently selected from the group consisting of halo; cyano; C₁₋₄alkyl optionally substituted with 1, 2, or 3 independently selected halo substituents; —C(O)NR^{a}R^{aa}; NR^{a}R^{aa}; and C₁₋₄alkyloxy optionally substituted with 1, 2, or 3 independently selected halo substituents; wherein R^{a} and R^{aa} are each independently selected from the group consisting of hydrogen and C₁₋₄alkyl optionally substituted with 1, 2, or 3 independently selected halo substituents.

10. A pharmaceutical composition comprising a prophylactically or a therapeutically effective amount of a compound according to claim 1 and a pharmaceutically acceptable carrier.

11. (canceled)

12. (canceled)

13. (canceled)

14. A method of preventing or treating a disorder selected from the group consisting of tauopathy, in particular a tauopathy selected from the group consisting of Alzheimer's disease, progressive supranuclear palsy, Down's syndrome, frontotemporal lobe dementia, frontotemporal dementia with Parkinsonism-17, Pick's disease, corticobasal degeneration, and agryophilic grain disease; or a neurodegenerative disease accompanied by a tau pathology, in particular a neurodegenerative disease selected from amyotrophic lateral sclerosis or frontotemporal lobe dementia caused by C9ORF72 mutations, comprising administering to a subject in need thereof, a prophylactically or a therapeutically effective amount of a compound according to claim 1.

15. (canceled)
